(19) United States
(12) Patent Application Publication
(10) Pub. No.: US 2017/0349638 A1
(43) Pub. Date: Dec. 7, 2017

(54) COMPANION DIAGNOSTIC TOOL FOR PEPTIDOMIMETIC MACROCYCLES

(71) Applicant: AILERON Therapeutics, Inc., Cambridge, MA (US)

(72) Inventor: Manuel Aivado, Chester Springs, PA (US)

(21) Appl. No.: 15/463,826

(22) Filed: Mar. 20, 2017

Related U.S. Application Data
(60) Provisional application No. 62/311,071, filed on Mar. 21, 2016.

(51) Int. Cl. C07K 14/47; A61K 38/17; A61K 9/00; C12Q 1/68; A61K 38/12

(52) U.S. Cl. CPC C07K 14/47; C12Q 1/6886; A61K 38/12; A61K 9/0019; A61K 38/17; C12Q 2600/156; C12Q 2600/158

(57) ABSTRACT

The present invention provides diagnostic tools, systems, and methods for detecting wild type p53 and p53-associated mutations for the treatment of disease with peptidomimetic macrocycles.

### CROSS REFERENCE

This application claims the benefit of U.S. Provisional Application No. 62/222,481 filed Sep. 23, 2015, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 27, 2017, is named 35224_812_201_SL.TXT and is 2,106,303 bytes in size.

### BACKGROUND OF THE INVENTION

Tumor suppressor p53 mediates cell cycle arrest, senescence, and apoptosis in response to DNA damage and cellular stress to prevent the development of cancer. The E3 ubiquitin ligase MDM2 (HDM2) negatively regulates p53 function via the ubiquitylation-proteasomal pathway. The loss of p53 activity, either by deletion, mutation, or MDM2 overexpression, is the most common defect in human cancers.

### SUMMARY OF THE INVENTION

In some embodiments, the invention provides a method for treating a condition in a subject in need thereof, the method comprising: a) performing an assay to determine a mutational status of a gene in the subject that modulates the p53 pathway and b) administering to the subject a therapeutically-effective amount of a peptidomimetic macrocycle or a pharmaceutically-acceptable salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 describes the synthesis of Fmoc-Me-6-Chloro-Tryptophan & Fmoc-6-Chloro-Tryptophan.

FIG. 2 shows human wild type P53 coding and protein sequence (SEQ ID NO: 2582).

FIG. 3 shows a structure of peptidomimetic macrocycle 46 (Table 14), a p53 peptidomimetic macrocycle, complexed with MDMX (Primary SwissProt accession number Q7ZUW7; Entry MDM4_DANRE).

FIG. 4 shows overlaid structures of p53 peptidomimetic macrocycles 142 (Table 14) and SP43 bound to MDMX (Primary SwissProt accession number Q7ZUW7; Entry MDM4_DANRE).

FIGS. 5A-F describe the results of a cell viability assay, a competition ELISA assay, GRIP assay, Kd data, p21 activation assay, fluorescence polarization competition binding and circular helicity data for exemplary peptidomimetic macrocycles of the invention. FIG. 5A-F discloses SEQ ID NOS 42-182, respectively, in order of appearance.

FIGS. 6A-D provide data from a variety of peptidomimetic macrocycles. FIG. 6A-D discloses SEQ ID NOS 46, 167, 181, 218, 221, 348, 293, 294, 387, 537, 533, 547, 605, 548, 598, 283, 378, 664, 46, 167, 181, 218, 221, 348, 293, 294, 387, 537, 533, 547, 605, 548, 598, 283, 378, 664, 706, 703, 708, 710, 693, 511, 628, 707, 720, 610, 609, 646, 695, 735, 379, 731, 666, 591, 718, 706, 703, 708, 710, 693, 511, 628, 707, 720, 610, 609, 646, 695, 735, 379, 731, 666, 591 and 718, respectively, in order of appearance.

FIGS. 7A-B provide data from a variety of peptidomimetic macrocycles.

FIG. 8 shows the effect of SP154, a peptidomimetic macrocycle, on tumor growth in a mouse MCF-7 xenograft model.

FIG. 9 shows the effect of SP249, a peptidomimetic macrocycle, on tumor growth in a mouse MCF-7 xenograft model.

FIG. 10 shows the effect of SP315, a peptidomimetic macrocycle, on tumor growth in a mouse MCF-7 xenograft model.

FIG. 11 shows the effect of SP252, a point mutation of SP154, on tumor growth in a mouse MCF-7 xenograft model.

FIG. 12 shows a plot of solubility for peptidomimetic macrocycles with varying C-terminal extensions.

FIG. 13 shows the binding affinity of compound 1 to human mutant and wild type p53.

### DETAILED DESCRIPTION OF THE INVENTION

The human transcription factor protein p53 induces cell cycle arrest and apoptosis in response to DNA damage and cellular stress, and thereby plays a critical role in protecting cells from malignant transformation. The E3 ubiquitin ligase MDM2 (also known as HDM2) negatively regulates p53 function through a direct binding interaction that neutralizes the p53 transactivation activity, leads to export from the nucleus of p53 protein, and targets p53 for degradation via the ubiquitylation-proteasomal pathway. Loss of p53 activity, either by deletion, mutation, or MDM2 overexpression, is the most common defect in human cancers. Tumors that express wild type p53 are vulnerable to pharmacologic agents that stabilize or increase the concentration of active p53. In this context, inhibition of the activities of MDM2 can restore p53 activity and resensitize cancer cells to apoptosis in vitro and in vivo.

MDMX (MDM4) has more recently been identified as a similar negative regulator of p53, and studies have revealed significant structural homology between the p53 binding interfaces of MDM2 and MDMX. The p53-MDM2 and p53-MDMX protein-protein interactions are mediated by the same 15-residue alpha-helical transactivation domain of p53, which inserts into hydrophobic clefts on the surface of MDM2 and MDMX. Three residues within this domain of p53 (F19, W23, and L26) are essential for binding to MDM2 and MDMX.

Provided herein are p53-based peptidomimetic macrocycles that modulate an activity of p53. Also provided herein are p53-based peptidomimetic macrocycles that inhibit the interactions between p53, MDM2 and/or MDMX proteins. Further, provided herein are p53-based peptidomimetic macrocycles that can be used for treating diseases including, but not limited to, cancer and other hyperproliferative diseases.

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analog) and a second naturally-occurring or non-naturally-occurring amino acid residue (or analog) within the same molecule. Peptidomimetic macrocycle include embodiments where the macrocycle-forming linker connects the α carbon of the first amino acid residue (or analog) to the α carbon of the second amino acid residue (or analog). The peptidomimetic macrocycles optionally include one or more non-peptide bonds between one or more amino acid residues and/or amino acid analog residues, and optionally include one or more non-naturally-occurring amino acid residues or amino acid analog residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild type sequence corresponding to the macrocycle.

As used herein, the term “stability” refers to the maintenance of a defined secondary structure in solution by a peptidomimetic macrocycle of the invention as measured by circular dichroism, NMR or another biophysical measure, or resistance to proteolytic degradation in vitro or in vivo. Non-limiting examples of secondary structures contemplated in this invention are α-helices, β-turns, and β-pleated sheets.

As used herein, the term “helical stability” refers to the maintenance of a helical structure by a peptidomimetic macrocycle of the invention as measured by circular dichroism or NMR. For example, in some embodiments, the peptidomimetic macrocycles of the invention exhibit at least a 1.25, 1.5, 1.75 or 2-fold increase in α-helicity as determined by circular dichroism compared to a corresponding uncrosslinked macrocycle.

The term “α-amino acid” or simply “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | | | | Side-chain | Hydro- |
|---|---|---|---|---|---|
| | 3-Letter | 1-Letter | Side-chain | charge | pathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive(10%) | −3.2 |
| | | | | neutral(90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include, without limitation, small hydrophobic amino acids and large hydrophobic amino acids. “Small hydrophobic amino acids” are glycine, alanine, proline, and analogs thereof. “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof. “Charged amino acids” are lysine, arginine, histidine, aspartate, glutamate, and analogs thereof.

The term “amino acid analog” refers to a molecule which is structurally similar to an amino acid and which can be substituted for an amino acid in the formation of a peptidomimetic macrocycle. Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “amino acid analog” or “non-natural amino acid” refers to a molecule which is structurally similar to an amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine, or substitution of the carboxy group with an ester). Non-natural amino acids include structures according to the following:

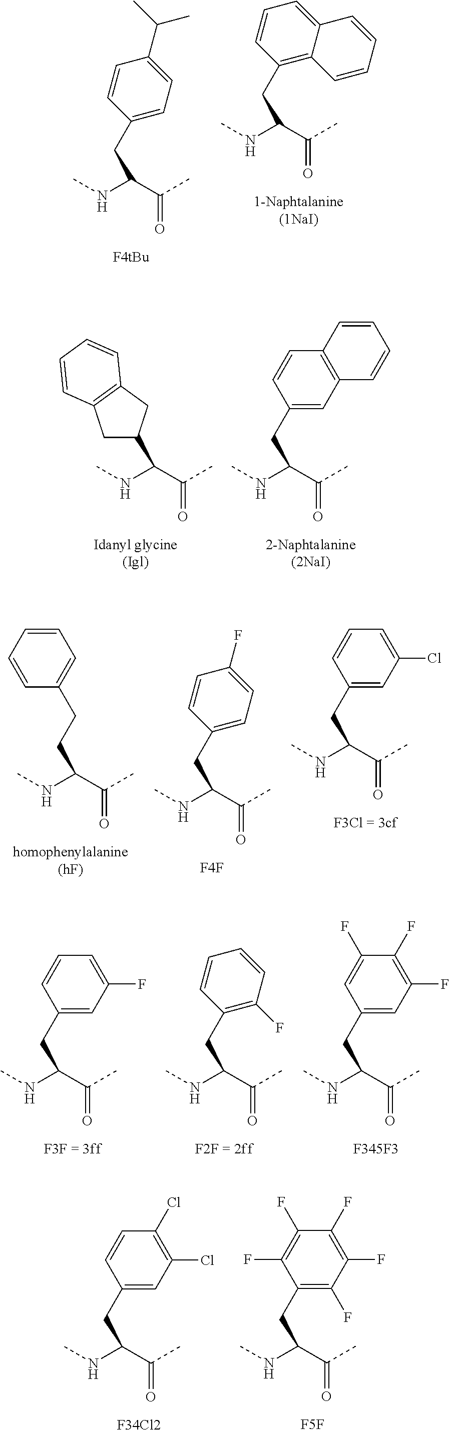

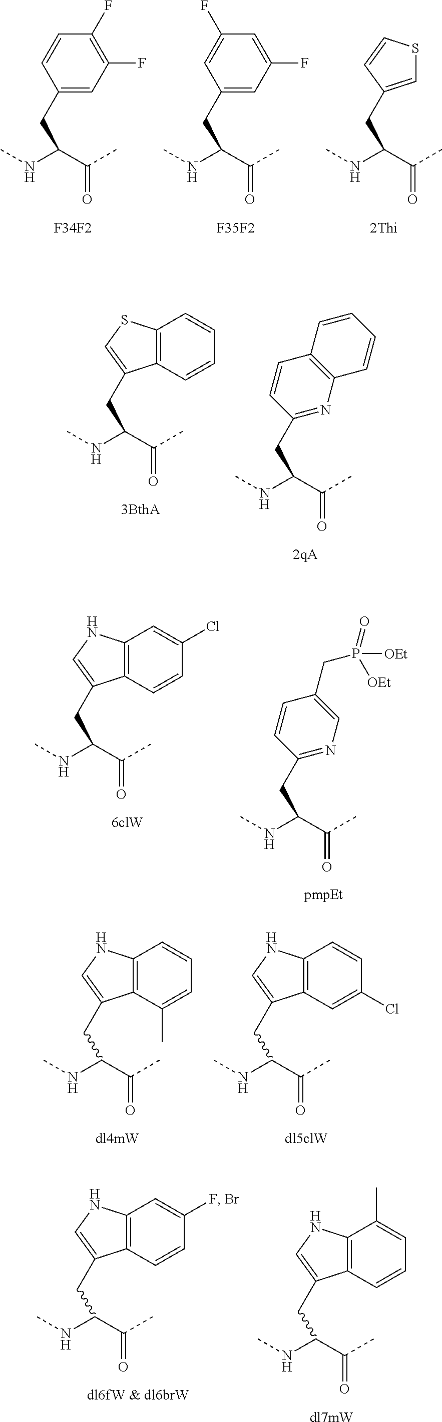

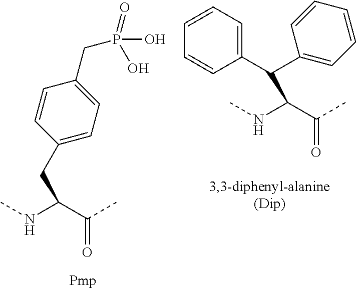

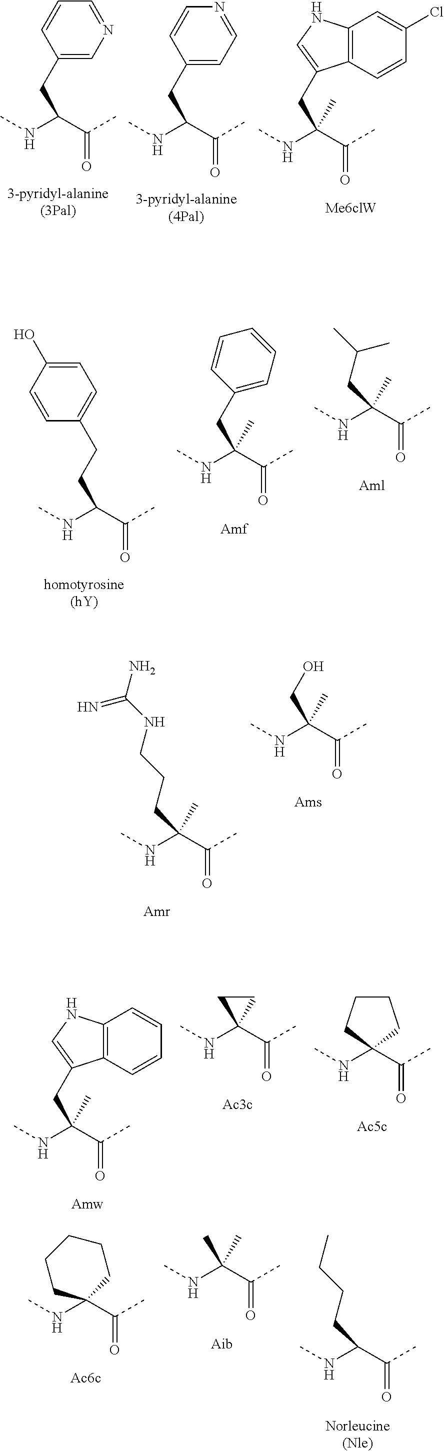

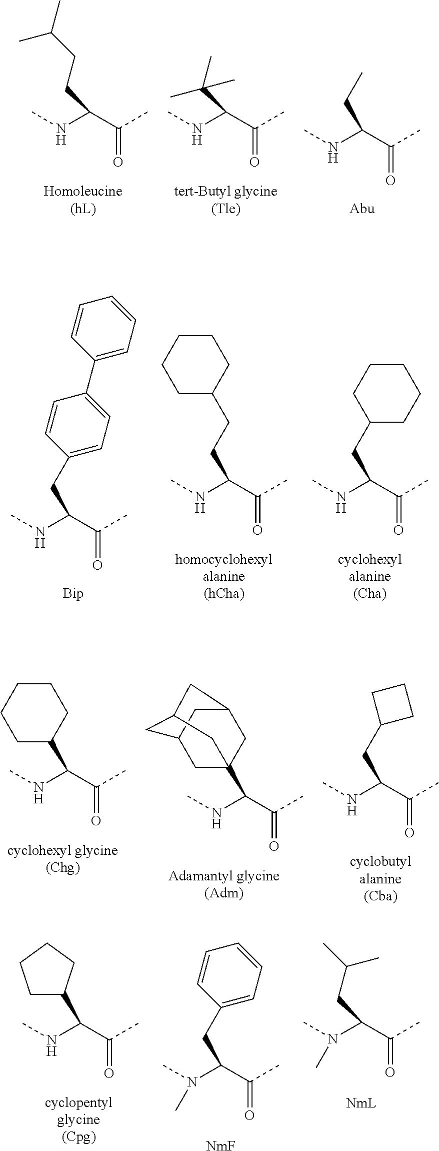

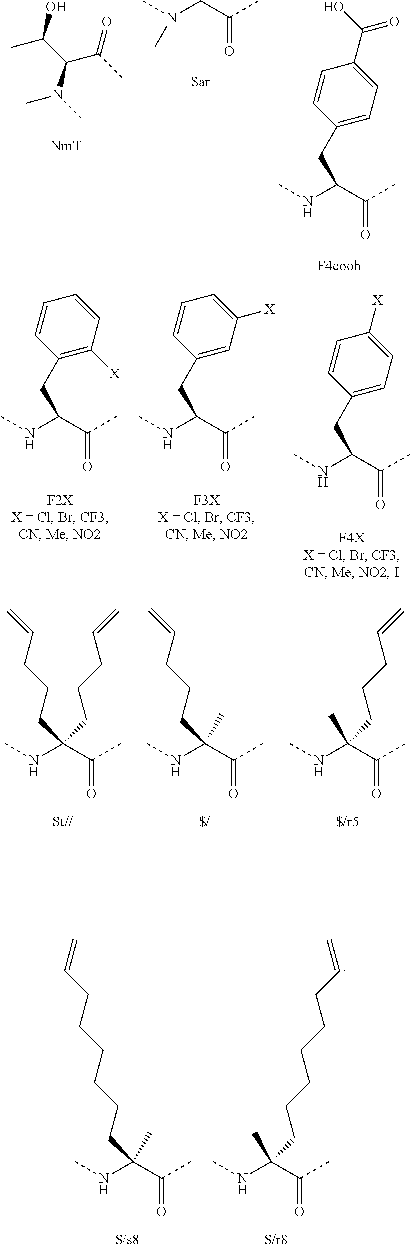

A “non-essential” amino acid residue is a residue that can be altered from the wild type sequence of a polypeptide without abolishing or substantially altering its essential biological or biochemical activity (e.g., receptor binding or activation). An “essential” amino acid residue is a residue that, when altered from the wild type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is preferably replaced with another amino acid residue from the same side chain family. Other examples of acceptable substitutions are substitutions based on isosteric considerations (e.g. norleucine for methionine) or other properties (e.g. 2-thienylalanine for phenylalanine).

Amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydroisoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluorobutyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH-dicyclohexylammonium salt; cyclopentyl-Gly-OH-dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; O-aminobutyric acid; O-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-β-thienyl)glycine; L-2-amino-3-(dimethylamino)-propionic acid; L-2-aminocaproic acid dicyclohexyl-ammonium salt; L-2-indanylglycine; L-allylglycine-dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs further include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-amino adipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

The term “capping group” refers to the chemical moiety occurring at either the carboxy or amino terminus of the polypeptide chain of the subject peptidomimetic macrocycle. The capping group of a carboxy terminus includes an unmodified carboxylic acid (ie —COOH) or a carboxylic acid with a substituent. For example, the carboxy terminus may be substituted with an amino group to yield a carboxamide at the C-terminus. Various substituents include but are not limited to primary and secondary amines, including pegylated secondary amines. Representative secondary amine capping groups for the C-terminus include:

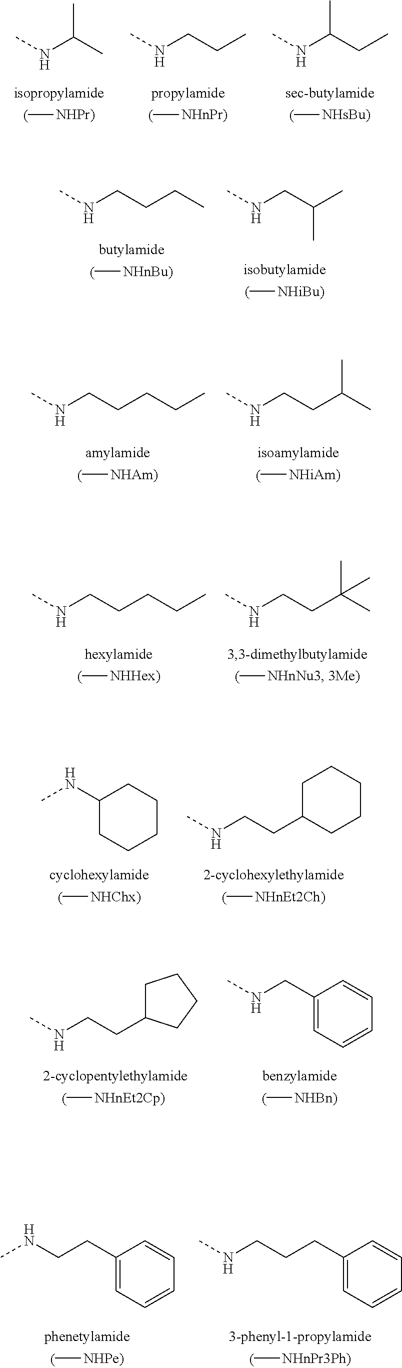

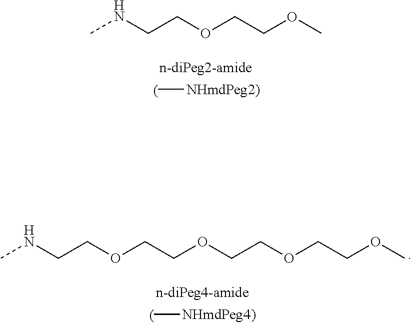

The capping group of an amino terminus includes an unmodified amine (i.e. —NH₂) or an amine with a substituent. For example, the amino terminus may be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Representative capping groups for the N-terminus include:

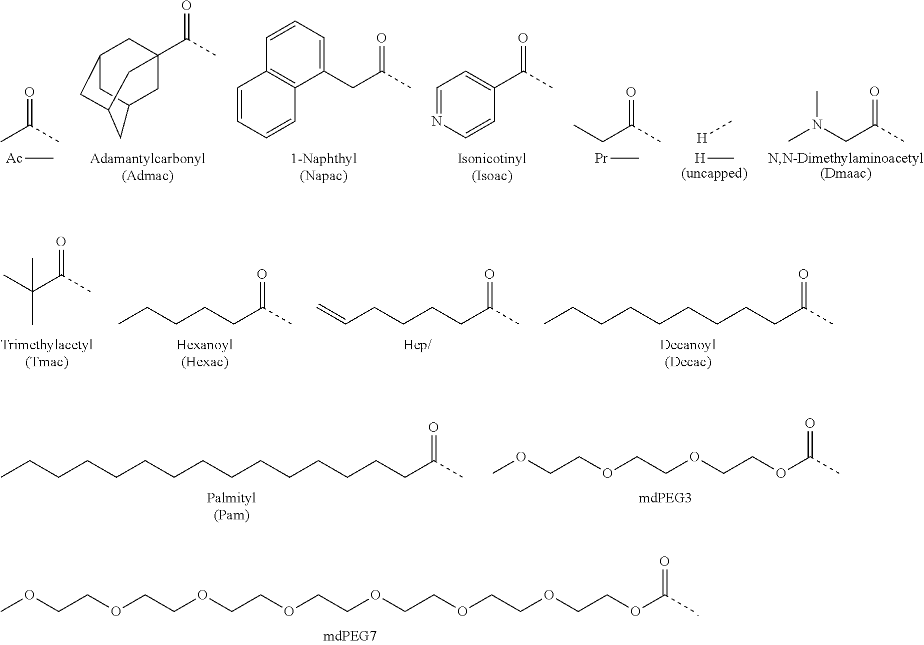

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

The symbol “ [symbol] ” when used as part of a molecular structure refers to a single bond or a trans or cis double bond.

The term “amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,α di-substituted amino acid).

The term “α,α di-substituted amino” acid refers to a molecule or moiety containing both an amino group and a carboxyl group bound to a carbon (the α-carbon) that is attached to two natural or non-natural amino acid side chains.

The term “polypeptide” encompasses two or more naturally or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “first C-terminal amino acid” refers to the amino acid which is closest to the C-terminus. The term “second C-terminal amino acid” refers to the amino acid attached at the N-terminus of the first C-terminal amino acid.

The term “macrocyclization reagent” or “macrocycle-forming reagent” as used herein refers to any reagent which may be used to prepare a peptidomimetic macrocycle of the invention by mediating the reaction between two reactive groups. Reactive groups may be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents may additionally include, for example, Ru reagents known in the art such as Cp*RuCl(PPh₃)₂, [Cp*RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Acc. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms in it.

The term “alkylene” refers to a divalent alkyl (i.e., —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms in it. The term “lower alkenyl” refers to a C₂-C₆ alkenyl chain. In the absence of any numerical designation, “alkenyl” is a chain (straight or branched) having 2 to 20 (inclusive) carbon atoms in it.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms in it. The term “lower alkynyl” refers to a C₂-C₆ alkynyl chain. In the absence of any numerical designation, “alkynyl” is a chain (straight or branched) having 2 to 20 (inclusive) carbon atoms in it.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl,

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH3, and —CH₂—CH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, one or more compounds of this invention contain one or more asymmetric centers and thus occur as racemates and racemic mixtures, single enantiomers, individual diastereomers and diastereomeric mixtures. In one embodiment, isomeric forms of these compounds are included in the present invention unless expressly provided otherwise. In some embodiments, one or more compounds of this invention are also represented in multiple tautomeric forms, in such instances, one or more compounds of the invention includes all tautomeric forms of the compounds described herein (e.g., if alkylation of a ring system results in alkylation at multiple sites, one or more compounds of the invention includes all such reaction products). All such isomeric forms of such compounds are included in the present invention unless expressly provided otherwise. All crystal forms of the compounds described herein are included in the present invention unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the invention can be practiced with the variable equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

The term “on average” represents the mean value derived from performing at least three independent replicates for each data point.

The term “biological activity” encompasses structural and functional properties of a macrocycle of the invention. Biological activity is, for example, structural stability, alpha-helicity, affinity for a target, resistance to proteolytic degradation, cell penetrability, intracellular stability, in vivo stability, or any combination thereof.

The term “binding affinity” refers to the strength of a binding interaction, for example between a peptidomimetic macrocycle and a target. Binding affinity can be expressed, for example, as an equilibrium dissociation constant (“K_{D}”), which is expressed in units which are a measure of concentration (e.g. M, mM, μM, nM etc). Numerically, binding affinity and K_{D} values vary inversely, such that a lower binding affinity corresponds to a higher K_{D} value, while a higher binding affinity corresponds to a lower K_{D} value. Where high binding affinity is desirable, “improved” binding affinity refers to higher binding affinity and therefore lower K_{D} values.

The term “ratio of binding affinities” refers to the ratio of dissociation constants (K_{D} values) of a first binding interaction (the numerator), versus a second binding interaction (denominator). Consequently, a “reduced ratio of binding affinities” to Target 1 versus Target 2 refers to a lower value for the ratio expressed as K_{D}(Target 1)/K_{D}(Target 2). This concept can also be characterized as “improved selectivity” for Target 1 versus Target 2, which can be due either to a decrease in the K_{D} value for Target 1 or an increase in the value for the K_{D} value for Target 2.

The term “in vitro efficacy” refers to the extent to which a test compound, such as a peptidomimetic macrocycle, produces a beneficial result in an in vitro test system or assay. In vitro efficacy can be measured, for example, as an “IC₅₀” or “EC₅₀” value, which represents the concentration of the test compound which produces 50% of the maximal effect in the test system.

The term “ratio of in vitro efficacies” or “in vitro efficacy ratio” refers to the ratio of IC₅₀ or EC₅₀ values from a first assay (the numerator) versus a second assay (the denominator). Consequently, an improved in vitro efficacy ratio for Assay 1 versus Assay 2 refers to a lower value for the ratio expressed as IC₅₀(Assay 1)/IC₅₀(Assay 2) or alternatively as EC₅₀(Assay 1)/EC₅₀(Assay 2). This concept can also be characterized as “improved selectivity” in Assay 1 versus Assay 2, which can be due either to a decrease in the IC₅₀ or EC₅₀ value for Target 1 or an increase in the value for the IC₅₀ or EC₅₀ value for Target 2.

The details of one or more particular embodiments of the invention are set forth in the accompanying drawings and the description below. Other features, objects, and advantages of the invention will be apparent from the description and drawings, and from the claims.

In some embodiments, the peptide sequences are derived from the p53 protein.

A non-limiting exemplary list of suitable p53 peptides for use in the present invention is given below.

TABLE 1
(SEQ ID NOS 1-18, respectively, in order of appearance)

| Sequence (bold = critical residue; X = cross-linked amino acid) | | | | | | | | | | | | | | | | | | | Design Notes |
|---|---|---|---|---|---|---|---|---|---|---|---|---|---|---|---|---|---|---|---|
| Ac- | | Gln | Ser | Gln | Gln | Thr | Phe | Ser | Asn | Leu | Trp | Arg | Leu | Leu | Pro | Gln | Asn | -NH2 | linear |
| Ac- | X | Gln | Ser | Gln | X | Thr | Phe | Ser | Asn | Leu | Trp | Arg | Leu | Leu | Pro | Gln | Asn | -NH2 | i-> i + 4 |
| | | | | | | | | | | | | | | | | | | | x-link #1 |
| Ac- | | X | Ser | Gln | Gln | X | Phe | Ser | Asn | Leu | Trp | Arg | Leu | Leu | Pro | Gln | Asn | -NH2 | i-> i + 4 |
| | | | | | | | | | | | | | | | | | | | x-link #2 |
| Ac- | | Gln | Ser | X | Gln | Thr | Phe | X | Asn | Leu | Trp | Arg | Leu | Leu | Pro | Gln | Asn | -NH2 | i-> i + 4 |
| | | | | | | | | | | | | | | | | | | | x-link #3 |
| Ac- | | Gln | Ser | Gln | X | Thr | Phe | Ser | X | Leu | Trp | Arg | Leu | Leu | Pro | Gln | Asn | -NH2 | i-> i + 4 |
| | | | | | | | | | | | | | | | | | | | x-link #4 |
| Ac- | | Gln | Ser | Gln | Gln | X | Phe | Ser | Asn | X | Trp | Arg | Leu | Leu | Pro | Gln | Asn | -NH2 | i-> i + 4 |
| | | | | | | | | | | | | | | | | | | | x-link #5 |
| Ac- | | Gln | Ser | Gln | Gln | Thr | Phe | X | Asn | Leu | Trp | X | Leu | Leu | Pro | Gln | Asn | -NH2 | i-> i + 4 |
| | | | | | | | | | | | | | | | | | | | x-link #6 |
| Ac- | | Gln | Ser | Gln | Gln | Thr | Phe | Ser | X | Leu | Trp | Arg | X | Leu | Pro | Gln | Asn | -NH2 | i-> i + 4 |
| | | | | | | | | | | | | | | | | | | | x-link #7 |
| Ac- | | Gln | Ser | Gln | Gln | Thr | Phe | Ser | Asn | Leu | Trp | X | Leu | Leu | Pro | X | Asn | -NH2 | i-> i + 4 |
| | | | | | | | | | | | | | | | | | | | x-link #8 |
| Ac- | | Gln | Ser | Gln | Gln | Thr | Phe | Ser | Asn | Leu | Trp | Arg | X | Leu | Pro | Gln | X | -NH2 | i-> i + 4 |
| | | | | | | | | | | | | | | | | | | | x-link #9 |
| Ac- | X | Gln | Ser | Gln | Gln | Thr | Phe | X | Asn | Leu | Trp | Arg | Leu | Leu | Pro | Gln | Asn | -NH2 | i-> i + 7 |
| | | | | | | | | | | | | | | | | | | | x-link #1 |
| Ac- | | X | Ser | Gln | Gln | Thr | Phe | Ser | X | Leu | Trp | Arg | Leu | Leu | Pro | Gln | Asn | -NH2 | i-> i + 7 |
| | | | | | | | | | | | | | | | | | | | x-link #2 |
| Ac- | | Gln | X | Gln | Gln | Thr | Phe | Ser | Asn | X | Trp | Arg | Leu | Leu | Pro | Gln | Asn | -NH2 | i-> i + 7 |
| | | | | | | | | | | | | | | | | | | | x-link #3 |
| Ac- | | Gln | Ser | Gln | X | Thr | Phe | Ser | Asn | Leu | Trp | X | Leu | Leu | Pro | Gln | Asn | -NH2 | i-> i + 7 |
| | | | | | | | | | | | | | | | | | | | x-link #4 |
| Ac- | | Gln | Ser | Gln | Gln | X | Phe | Ser | Asn | Leu | Trp | Arg | X | Leu | Pro | Gln | Asn | -NH2 | i-> i + 7 |
| | | | | | | | | | | | | | | | | | | | x-link #5 |
| Ac- | | Gln | Ser | Gln | Gln | Thr | Phe | X | Asn | Leu | Trp | Arg | Leu | Leu | X | Gln | Asn | -NH2 | i-> i + 7 |
| | | | | | | | | | | | | | | | | | | | x-link #6 |
| Ac- | | Gln | Ser | Gln | Gln | Thr | Phe | Ser | X | Leu | Trp | Arg | Leu | Leu | Pro | X | Asn | -NH2 | i-> i + 7 |
| | | | | | | | | | | | | | | | | | | | x-link #7 |
| Ac- | | Gln | Ser | Gln | Gln | Thr | Phe | Ser | Asn | X | Trp | Arg | Leu | Leu | Pro | Gln | X | -NH2 | i-> i + 7 |
| | | | | | | | | | | | | | | | | | | | x-link #8 |

TABLE 2
(SEQ ID NOS 19-31, respectively, in order of appearance)

| | |
|---|---|
| Sequence (bold = critical residue; X = cross-linked amino acid) | Design Notes |

| | | | | | | | | | | | | | | | | |
|---|---|---|---|---|---|---|---|---|---|---|---|---|---|---|---|---|
| Ac- | | Leu | Trp | Phe | Glu | His | Tyr | Trp | Ala | Gln | Leu | Thr | Ser | | -NH2 | linear |
| Ac- | X | Leu | Trp | Phe | X | His | Tyr | Trp | Ala | Gln | Leu | Thr | Ser | | -NH2 | i-> i + 4 |
| | | | | | | | | | | | | | | | | x-link #1 |
| Ac- | | X | Trp | Phe | Glu | X | Tyr | Trp | Ala | X | Leu | X | Ser | | -NH2 | i-> i + 4 |
| | | | | | | | | | | | | | | | | x-link #2 |
| Ac- | | Leu | X | Phe | Glu | His | X | Trp | Ala | Gln | Leu | Thr | Ser | | -NH2 | i-> i + 4 |
| | | | | | | | | | | | | | | | | x-link #3 |
| Ac- | | Leu | Trp | Phe | Glu | His | Tyr | Trp | X | Gln | Leu | Thr | Ser | | -NH2 | i-> i + 4 |
| | | | | | | | | | | | | | | | | x-link #4 |
| Ac- | | Leu | Trp | Phe | Glu | His | Tyr | Trp | Ala | X | Leu | X | Ser | | -NH2 | i-> i + 4 |
| | | | | | | | | | | | | | | | | x-link #5 |
| Ac- | | Leu | Trp | Phe | Glu | His | Tyr | Trp | X | Gln | Leu | Thr | X | | -NH2 | i-> i + 4 |
| | | | | | | | | | | | | | | | | x-link #6 |
| Ac- | | Leu | Trp | Phe | Glu | His | Tyr | Trp | Ala | X | Leu | Thr | Ser | X | -NH2 | i-> i + 4 |
| | | | | | | | | | | | | | | | | x-link #7 |
| Ac- | | X | Trp | Phe | Glu | His | Tyr | Trp | X | Gln | Leu | Thr | Ser | | -NH2 | i-> i + 7 |
| | | | | | | | | | | | | | | | | x-link #1 |
| Ac- | | Gln | X | Phe | Glu | His | Tyr | Trp | Ala | X | Leu | Thr | Ser | | -NH2 | i-> i + 7 |
| | | | | | | | | | | | | | | | | x-link #2 |
| Ac- | | Gln | Trp | Phe | X | His | Tyr | Trp | Ala | Gln | Leu | X | Ser | | -NH2 | i-> i + 7 |
| | | | | | | | | | | | | | | | | x-link #3 |
| Ac- | | Gln | Trp | Phe | Glu | X | Tyr | Trp | Ala | Gln | Leu | Thr | X | | -NH2 | i-> i + 7 |
| | | | | | | | | | | | | | | | | x-link #4 |
| Ac- | | Gln | Trp | Phe | Glu | His | X | Trp | Ala | Gln | Leu | Thr | Ser | X | -NH2 | i-> i + 7 |
| | | | | | | | | | | | | | | | | x-link #5 |

TABLE 3
(SEQ ID NOS 32-37, respectively, in order of appearance)

| | | | | | | | | | | | | Design |
|---|---|---|---|---|---|---|---|---|---|---|---|---|
| Sequence (bold = critical residue; X = cross-linked amino acid) | | | | | | | | | | | | Notes |
| Ac- | | Phe | Met | Aib/His/ | Tyr | 6-C1trp | Glu | Ac3/Gln/ | Leu | | -NH2 | linear |
| | | | | Asn | | | | Leu | | | | |
| Ac- | X | Phe | Met | Aib/His/ | X | 6-C1trp | Glu | Ac3/Gln/ | Leu | | -NH2 | i-> i + 4 |
| | | | | Asn | | | | Leu | | | | x-link #1 |
| Ac- | | Phe | X | Aib/His/ | Tyr | 6-C1trp | X | Ac3/Gln/ | Leu | | -NH2 | i-> i + 4 |
| | | | | Asn | | | | Leu | | | | x-link #2 |
| Ac- | | Phe | Met | X | Tyr | 6-C1trp | Glu | X | Leu | | -NH2 | i-> i + 4 |
| | | | | | | | | | | | | x-link #3 |
| Ac- | X | Phe | Met | Aib/His/ | Tyr | 6-C1trp | Glu | X | Leu | | -NH2 | i-> i + 7 |
| | | | | Asn | | | | | | | | x-link #1 |
| Ac- | | Phe | X | Aib/His/ | Tyr | 6-C1trp | Glu | Ac3/Gln/ | Leu | X | -NH2 | i-> i + 7 |
| | | | | Asn | | | | Leu | | | | x-link #2 |

In Table 3 and elsewhere, “Aib” represents a 2-aminoisobutyric acid residue, while “Ac3c” represents an aminocyclopropane carboxylic acid residue.

### Peptidomimetic Macrocycles

In some embodiments, a peptidomimetic macrocycle of the invention has the formula:

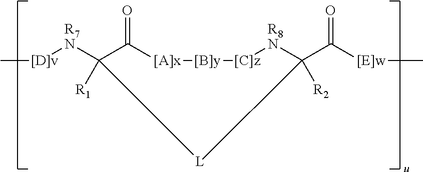

wherein:
each A, C, D, and E is independently a natural or non-natural amino acid;
B is a natural or non-natural amino acid, amino acid analog,

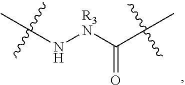

[—NH-L₃-CO—], [—NH-L₃-SO₂—], or [—NH-L₃-];
terminal D and E independently optionally include a capping group;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-;
L is a macrocycle-forming linker of the formula-L₁-L₂-;
each L and L′ is independently a macrocycle-forming linker of the formula

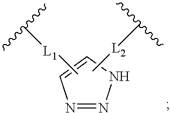

L₁, L₂ and L₃ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
each R₄ is alkylene, alkenylene, alkynylene, heteroalkylene, cycloalkylene, heterocycloalkylene, arylene, or heteroarylene;
each K is O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v and w are independently integers from 1-1000, for example 1-500, 1-200, 1-100, 1-50, 1-30, 1-20, or 1-10;
u is an integer from 1-10, for example 1-5, 1-3 or 1-2;
x, y and z are independently integers from 0-10; for example the sum of x+y+z is 2, 3, or 6;
and n is an integer from 1-5.

In some embodiments, a peptidomimetic macrocycle has the formula:

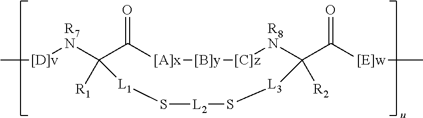

wherein:
each A, C, D, and E is independently an amino acid;
B is an amino acid,

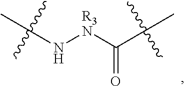

[—NH-L₄-CO—], [—NH-L₄-SO₂—], or [—NH-L₄-];
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
L₁, L₂, L₃ and L₄ are independently alkylene, alkenylene, alkynylene, heteroalkylene, cycloalkylene, heterocycloalkylene, cycloarylene, heterocycloarylene or [—R₄—K—R₄—]ₙ, each being unsubstituted or substituted with R₅;
each K is O, S, SO, SO₂, CO, CO₂, or CONR₃;
each R₄ is alkylene, alkenylene, alkynylene, heteroalkylene, cycloalkylene, heterocycloalkylene, arylene, or heteroarylene;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v and w are independently integers from 1-1000, for example 1-500, 1-200, 1-100, 1-50, 1-30, 1-20 or 1-10;
u is an integer from 1-10, for example 1-5, 1-3 or 1-2;
x, y and z are independently integers from 0-10, for example the sum of x+y+z is 2, 3, or 6;
and n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments, the peptidomimetic macrocycles are claimed with the proviso that when u=1 and w=2, the first C-terminal amino acid represented by E is not an Arginine (R) and/or the second C-terminal amino acid represented by E is not a Threonine (T). For instance, when u=1 and w=2, the first C-terminal amino acid and/or the second C-terminal amino acid represented by E do not comprise a positively charged side chain or a polar uncharged side chain. In some embodiments, when u=1 and w=2, the first C-terminal amino acid and/or the second C-terminal amino acid represented by E comprise a hydrophobic side chain. For example, when w=2, the first C-terminal amino acid and/or the second N-terminal amino acid represented by E comprise a hydrophobic side chain, for example a large hydrophobic side chain.

In some embodiments, w is between 3 and 1000. For example, the third amino acid represented by E comprises a large hydrophobic side chain.

In some embodiments any peptidomimetic macrocycle disclosed, L₁ and L₂, either alone or in combination, do not form an all hydrocarbon chain or a thioether. In other embodiments any peptidomimetic macrocycle disclosed, L₁ and L₂, either alone or in combination, do not form an all hydrocarbon chain or a triazole.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9 or 10. In some embodiments, the sum of x+y+z is 3 or 6. In some embodiments, the sum of x+y+z is 3. In other embodiments, the sum of x+y+z is 6. Each occurrence of A, B, C, D or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

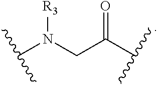

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

Peptidomimetic macrocycles are also provided of the formula:

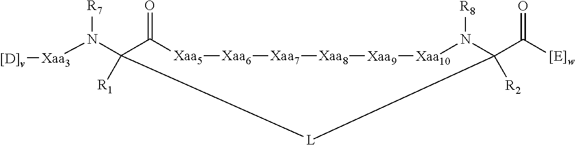

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39), where each X is an amino acid;
each D and E is independently an amino acid;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L or L′ is independently a macrocycle-forming linker of the formula -L₁-L₂-;
L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
each R₄ is alkylene, alkenylene, alkynylene, heteroalkylene, cycloalkylene, heterocycloalkylene, arylene, or heteroarylene;
each K is O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v is an integer from 1-1000, for example 1-500, 1-200, 1-100, 1-50, 1-30, 1-20 or 1-10;
w is an integer from 3-1000, for example 3-500, 3-200, 3-100, 3-50, 3-30, 3-20, or 3-10; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments of any of the Formulas described herein, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39). In other embodiments, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39). In other embodiments, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39). In other embodiments, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39). In other embodiments, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39).

In some embodiments, a peptidomimetic macrocycle has the Formula:

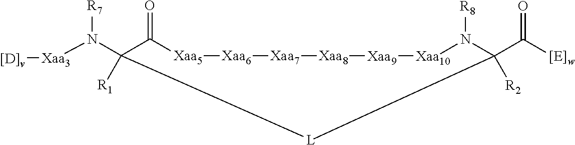

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂(SEQ ID NO: 41), where each X is an amino acid;
each D is independently an amino acid;
each E is independently an amino acid, for example an amino acid selected from Ala (alanine), D-Ala (D-alanine), Aib (α-aminoisobutyric acid), Sar (N-methyl glycine), and Ser (serine);
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L or L′ is independently a macrocycle-forming linker of the formula -L₁-L₂-;
each L and L′ is independently a macrocycle-forming linker of the formula

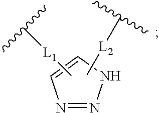

L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
each R₄ is alkylene, alkenylene, alkynylene, heteroalkylene, cycloalkylene, heterocycloalkylene, arylene, or heteroarylene;
each K is O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v is an integer from 1-1000, for example 1-500, 1-200, 1-100, 1-50, 1-30, 1-20, or 1-10;
w is an integer from 3-1000, for example 3-500, 3-200, 3-100, 3-50, 3-30, 3-20, or 3-10; and
n is an integer from 1-5.

In some embodiments of the above Formula, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 41). In other embodiments of the above Formula, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 41) In other embodiments of the above Formula, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 41). In other embodiments of the above Formula, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 41) In other embodiments of the above Formula, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 41).

In some embodiments, w is an integer from 3-10, for example 3-6, 3-8, 6-8, or 6-10. In some embodiments, w is 3. In other embodiments, w is 6. In some embodiments, v is an integer from 1-10, for example 2-5. In some embodiments, v is 2.

In one embodiment of any of the Formulas described herein, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 3. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges. Similarly, when u is greater than 1, each compound of the invention may encompass peptidomimetic macrocycles which are the same or different. For example, a compound of the invention may comprise peptidomimetic macrocycles comprising different linker lengths or chemical compositions.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

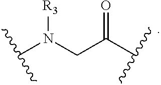

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In one embodiment, the peptidomimetic macrocycle is:

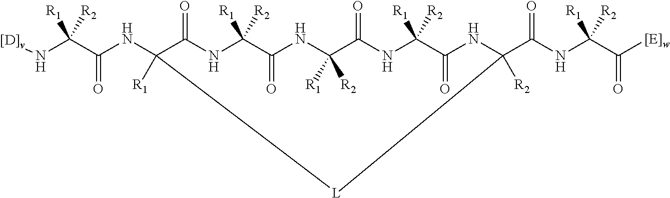

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle is:

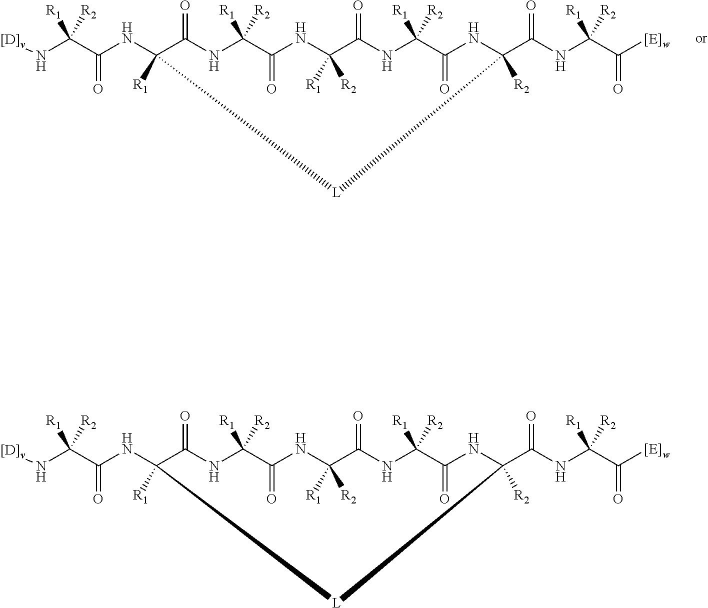

Peptidomimetic macrocycles are also provided of the formula:

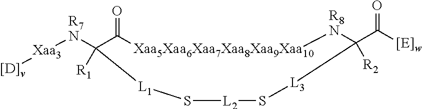

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 39), where each X is an amino acid;
each D and E is independently an amino acid;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
L₁, L₂, L₃ and L₄ are independently alkylene, alkenylene, alkynylene, heteroalkylene, cycloalkylene, heterocycloalkylene, cycloarylene, heterocycloarylene or [—R₄—K—R₄—]ₙ, each being unsubstituted or substituted with R₅;
each K is O, S, SO, SO₂, CO, CO₂, or CONR₃;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
each R₄ is alkylene, alkenylene, alkynylene, heteroalkylene, cycloalkylene, heterocycloalkylene, arylene, or heteroarylene;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v is an integer from 1-1000, for example 1-500, 1-200, 1-100, 1-50, 1-30, 1-20 or 1-10;
w is an integer from 3-1000, for example 3-500, 3-200, 3-100, 3-50, 3-30, 3-20, or 3-10; and
n is an integer from 1-5.

Peptidomimetic macrocycles are also provided of the formula:

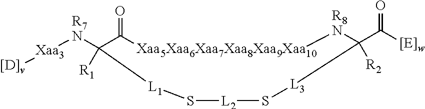

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 41), where each X is an amino acid;
each D and E is independently an amino acid;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
L₁, L₂, L₃ and L₄ are independently alkylene, alkenylene, alkynylene, heteroalkylene, cycloalkylene, heterocycloalkylene, cycloarylene, heterocycloarylene or [—R₄—K—R₄—]ₙ, each being unsubstituted or substituted with R₅;
each K is O, S, SO, SO₂, CO, CO₂, or CONR₃;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
each R₄ is alkylene, alkenylene, alkynylene, heteroalkylene, cycloalkylene, heterocycloalkylene, arylene, or heteroarylene;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
v is an integer from 1-1000, for example 1-500, 1-200, 1-100, 1-50, 1-30, 1-20, or 1-10;
w is an integer from 3-1000, for example 3-500, 3-200, 3-100, 3-50, 3-30, 3-20, or 3-10; and
n is an integer from 1-5.

In one embodiment, the peptidomimetic macrocycle is:

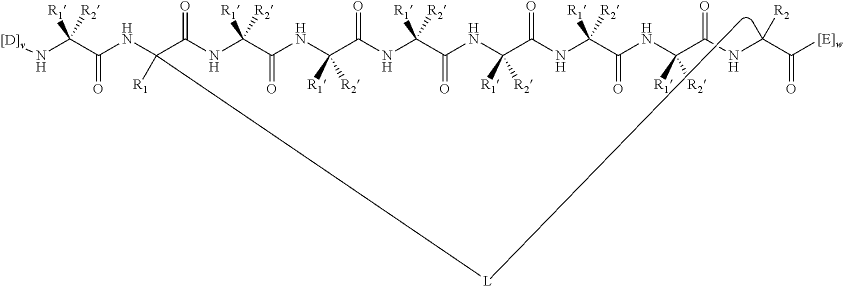

wherein each R₁ and R₂ is independently independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle is:

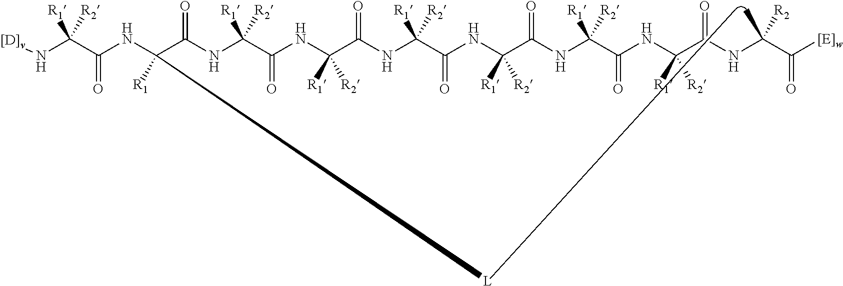

wherein each R₁′ and R₂′ is independently an amino acid.

In other embodiments, the peptidomimetic macrocycle is a compound of any of the formulas shown below:

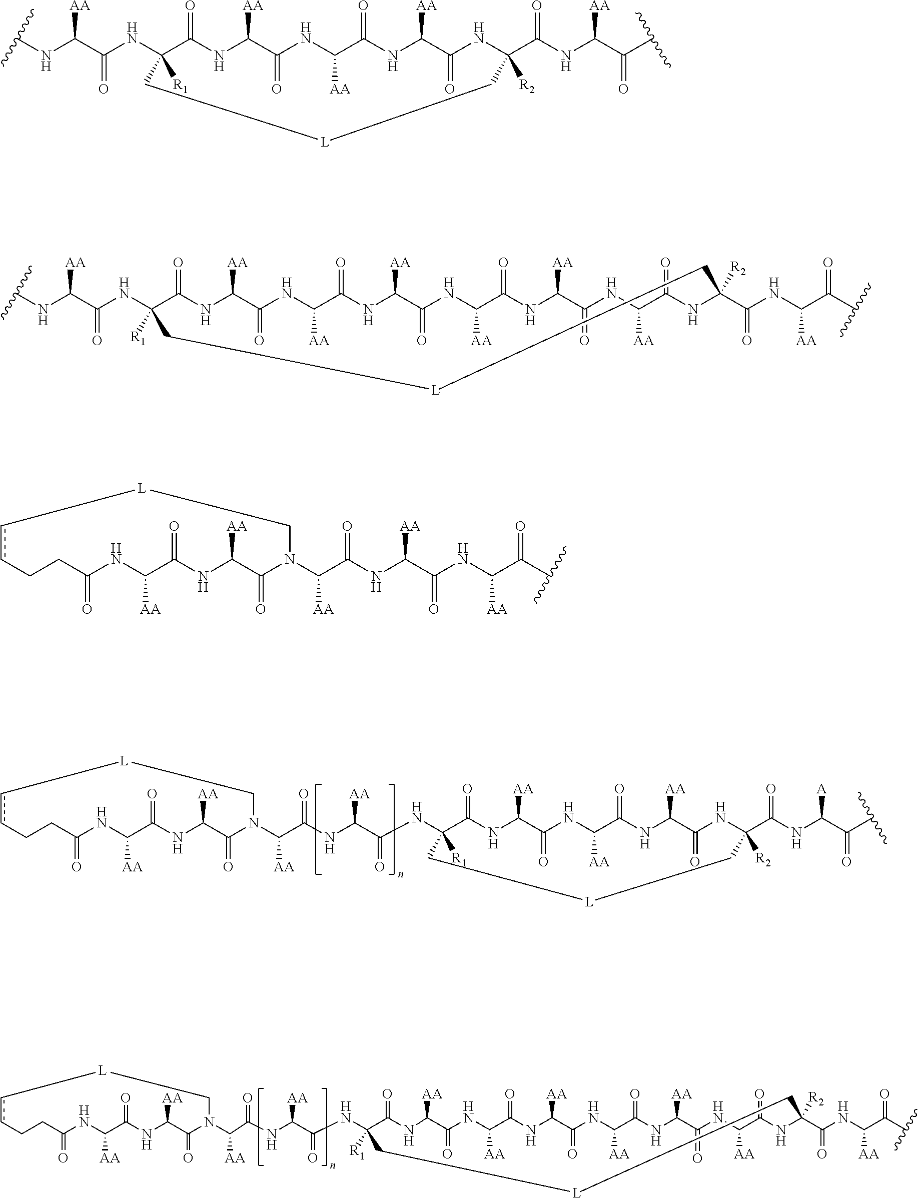

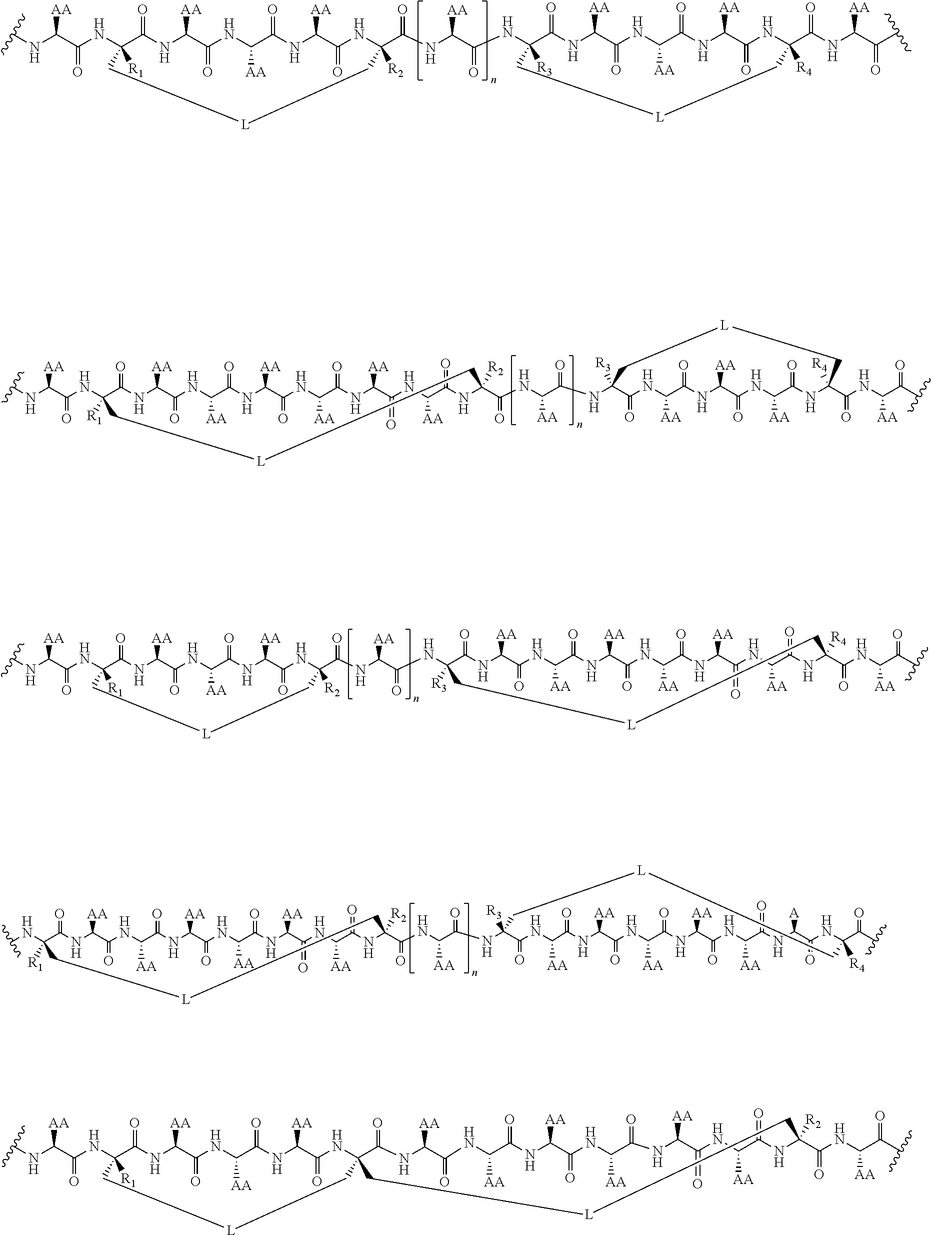

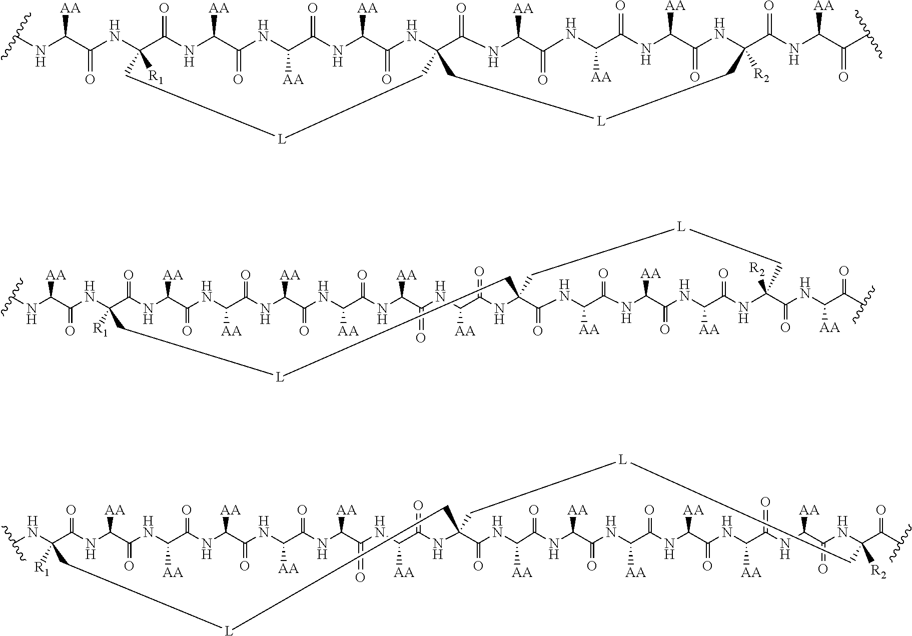

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, n is 0. In other embodiments, n is less than 50.

Exemplary embodiments of the macrocycle-forming linker L are shown below.

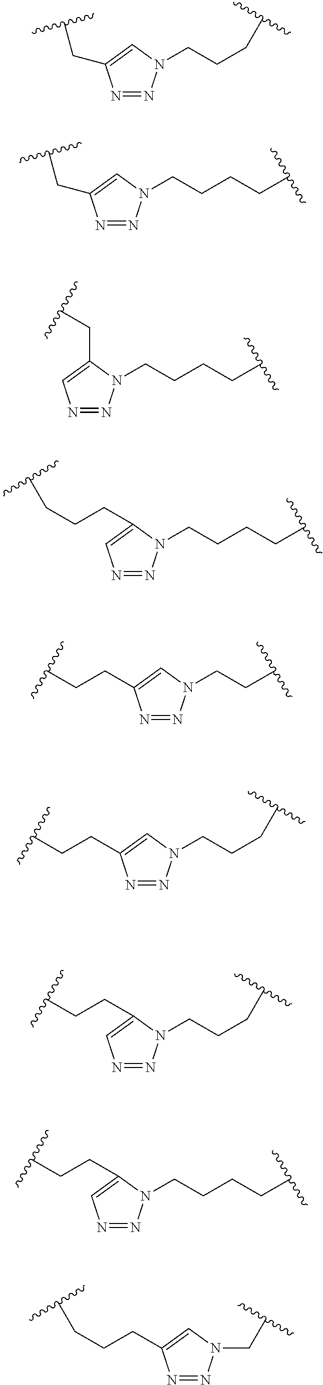

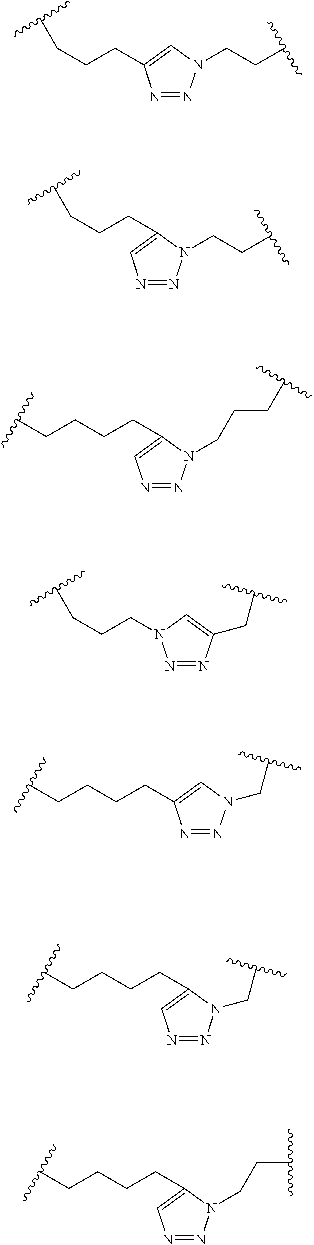

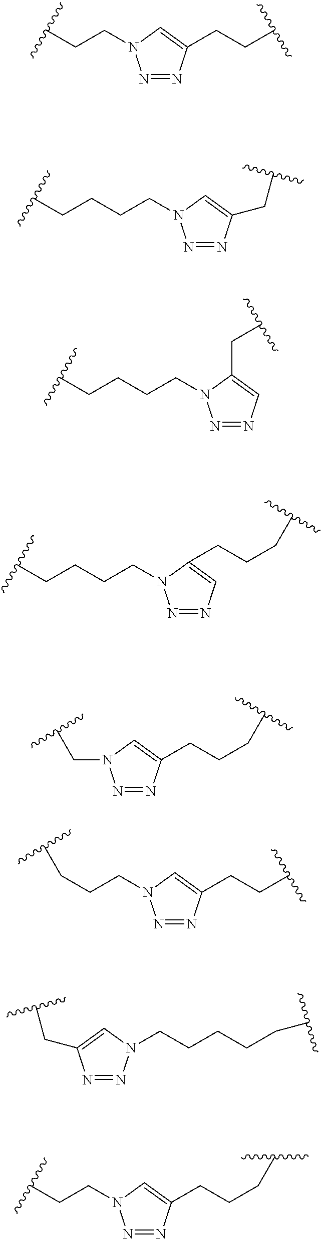

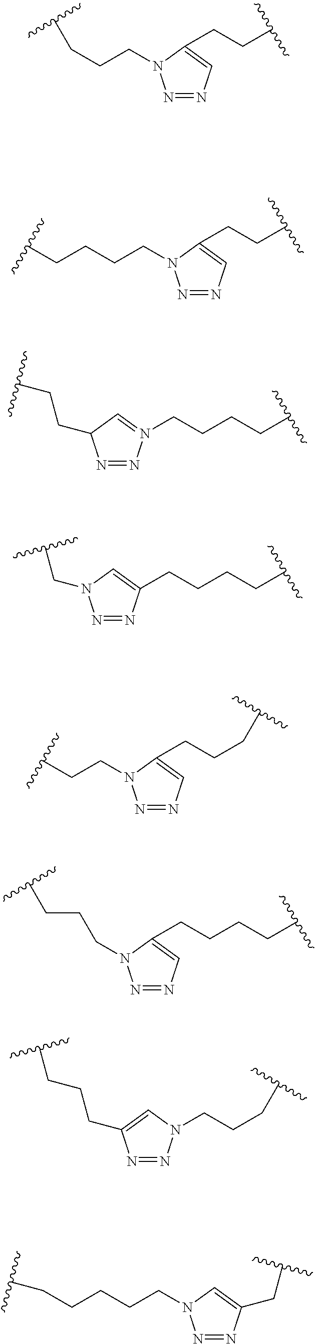

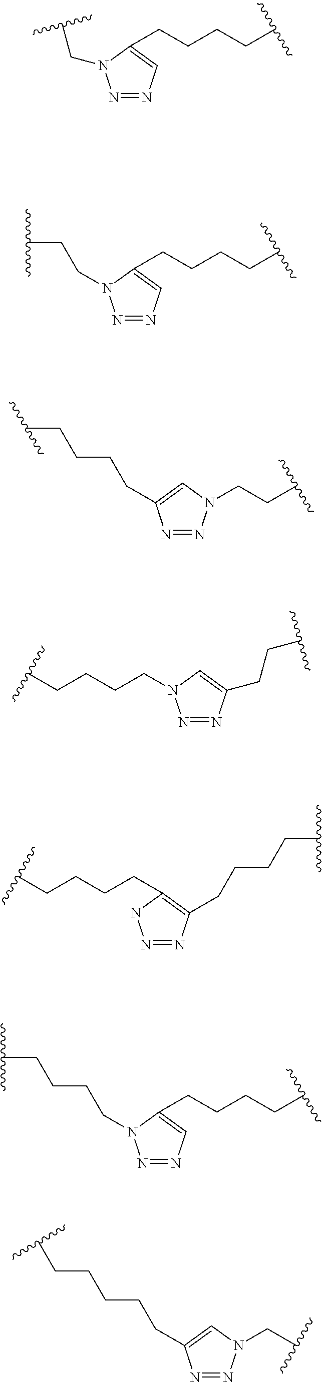

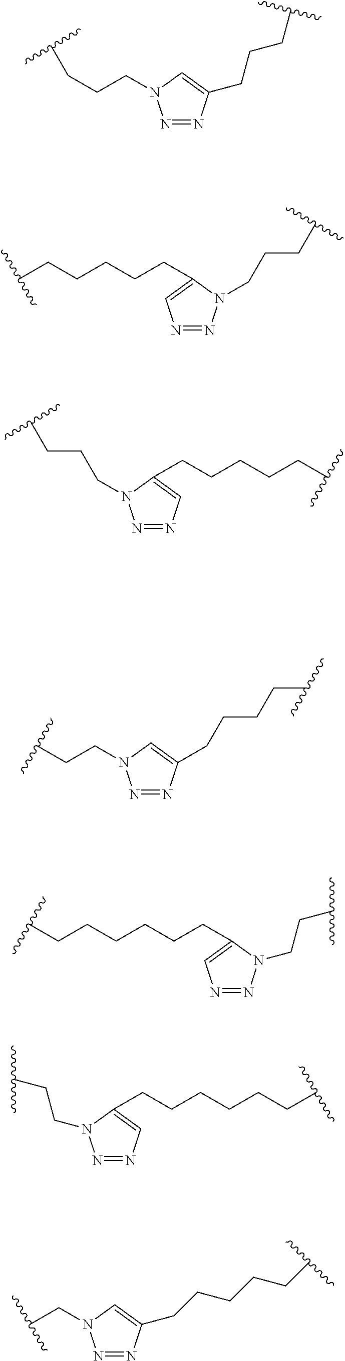

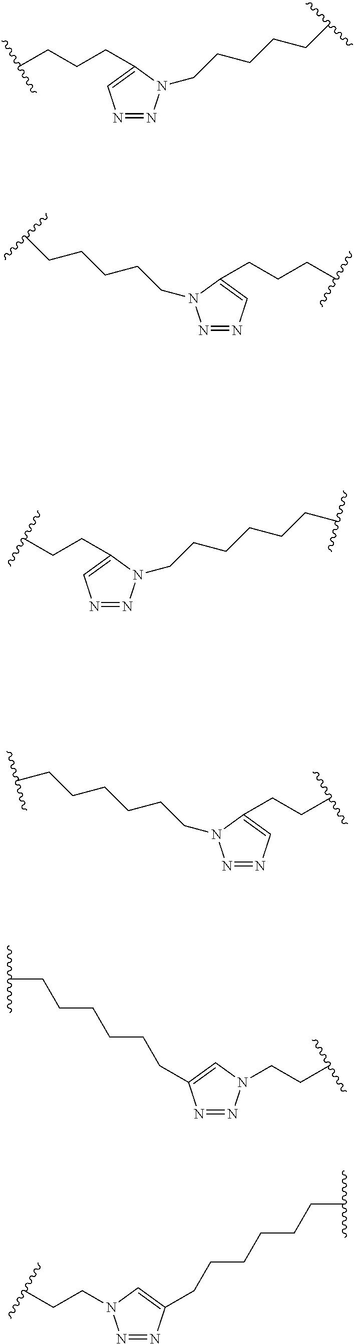 [SMILES: *CCCCCn1nncc1CCC* ; *CCCCCn1nncc1CCC* ; *CCCCCCn1nncc1CC* ; *CCCCCCn1nncc1CC* ; *CCCCCCc1cn(CC*)nn1 ; *CCCCCCc1cn(CC*)nn1]

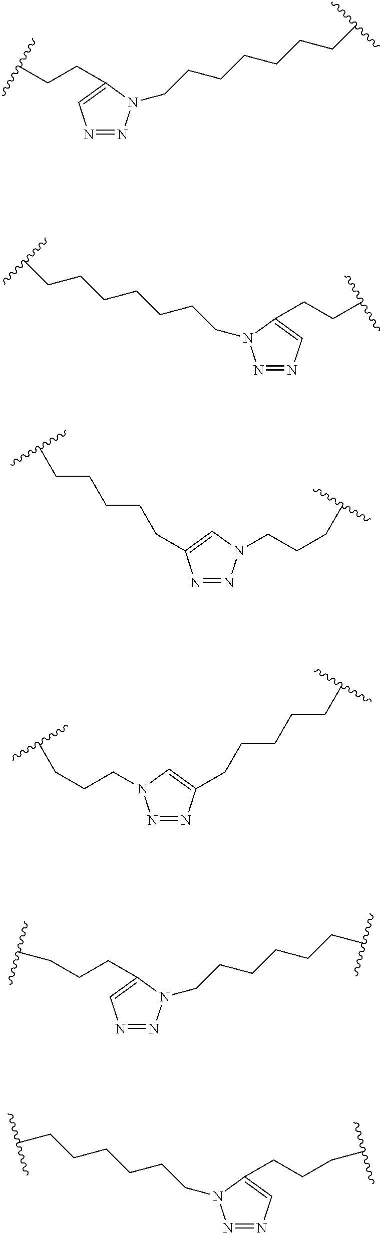 [SMILES: *CCCCCCCn1nncc1CC* ; *CCCCCCCn1nncc1CC* ; *CCCCCc1cn(CCC*)nn1 ; *CCCCCc1cn(CCC*)nn1 ; *CCCCCCn1nncc1CCC* ; *CCCCCCn1nncc1CCC*]

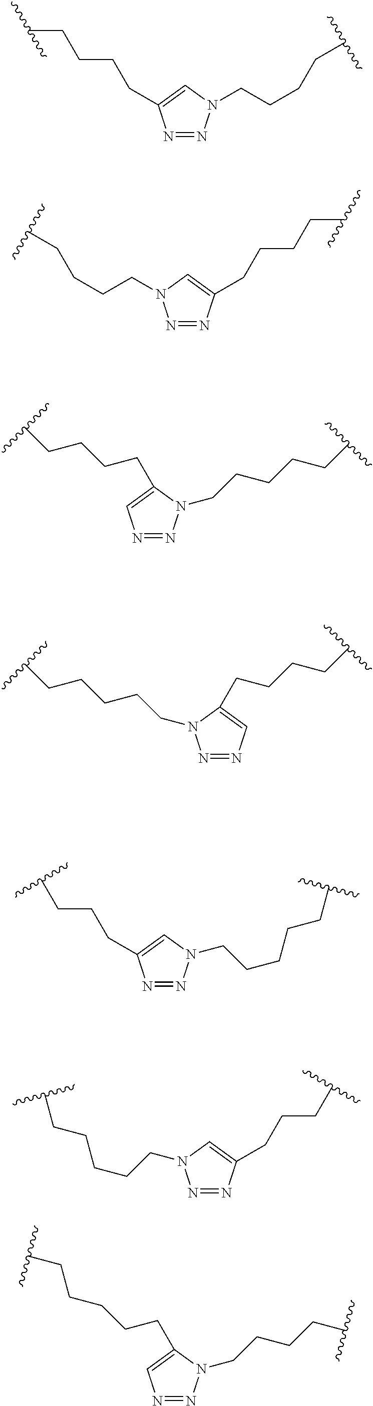

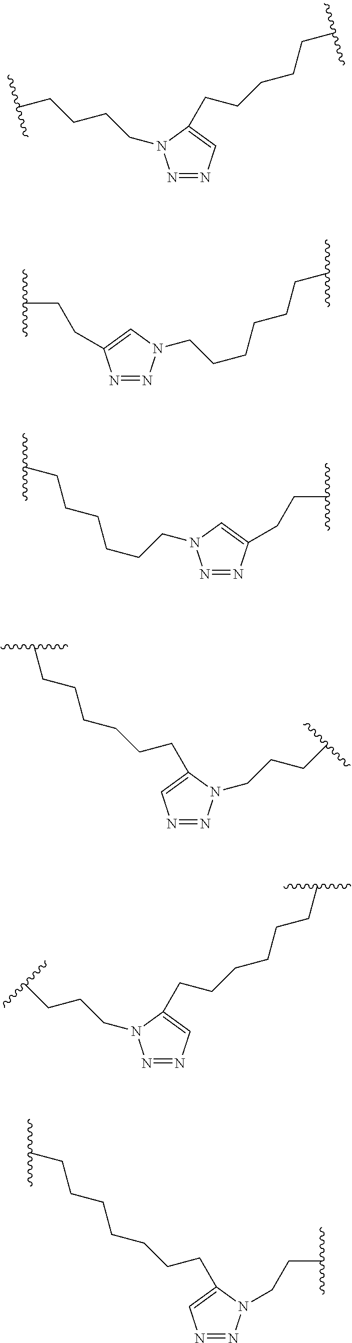 [SMILES: *CCCCCc1cnnn1CCCC* ; *CCCCCCn1cc(CC*)nn1 ; *CCCCCCn1cc(CC*)nn1 ; *CCCCCCc1cnnn1CCC* ; *CCCCCCc1cnnn1CCC* ; *CCCCCCCc1cnnn1CC*]

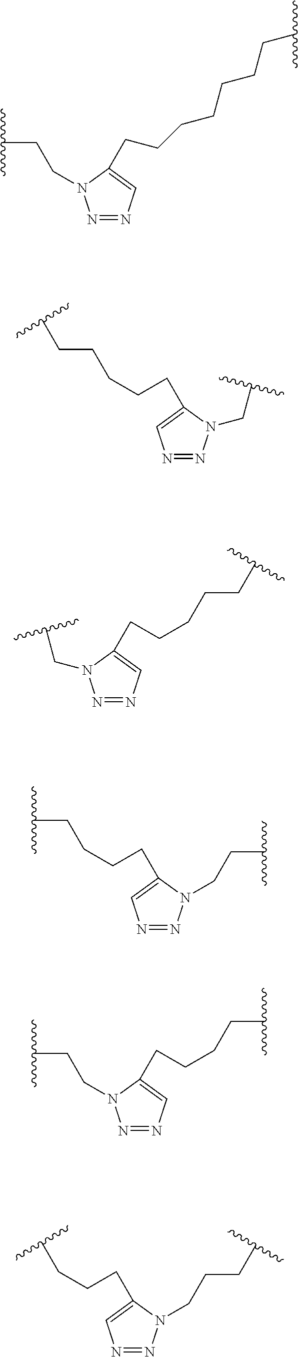 [SMILES: *CCCCCCCc1cnnn1CC* ; *CCCCCc1cnnn1C* ; *CCCCCc1cnnn1C* ; *CCCCc1cnnn1CC* ; *CCCCc1cnnn1CC* ; *CCCc1cnnn1CCC*]

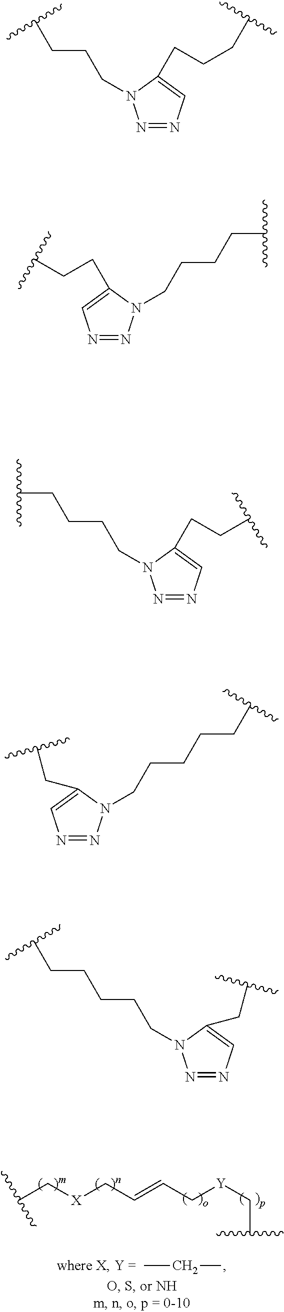

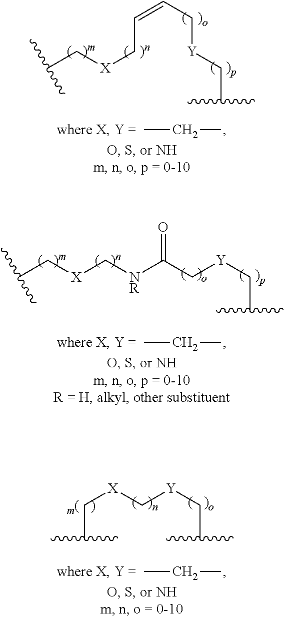

In other embodiments, D and/or E in the compound are further modified in order to facilitate cellular uptake. In some embodiments, lipidating or PEGylating a peptidomimetic macrocycle facilitates cellular uptake, increases bioavailability, increases blood circulation, alters pharmacokinetics, decreases immunogenicity and/or decreases the needed frequency of administration.

In other embodiments, at least one of [D] and [E] in the compound of formulas disclosed represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers. In an embodiment, u is 2.

In the peptidomimetic macrocycles of the invention, any of the macrocycle-forming linkers described herein may be used in any combination with any of the sequences shown in Tables 1-3, 9-13, 23-25, 27-28 and also with any of the R— substituents indicated herein.

In some embodiments, the peptidomimetic macrocycle comprises at least one α-helix motif. For example, A, B and/or C in the compounds disclosed include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix. In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix. Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds. Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms. Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In other embodiments, the invention provides peptidomimetic macrocycles:

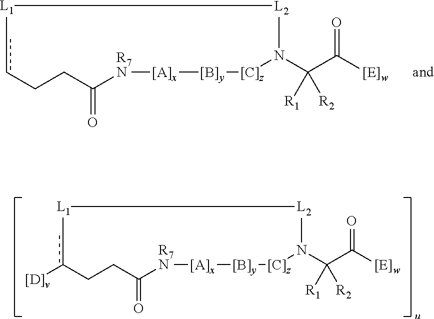

wherein:
each A, C, D, and E is independently a natural or non-natural amino acid, and the terminal D and E independently optionally include a capping group;
B is a natural or non-natural amino acid, amino acid analog,

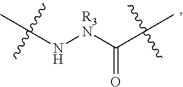

[—NH-L₃-CO—], [—NH-L₃-SO₂—], or [—NH-L₃-];
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;
R₃ is hydrogen, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
L is a macrocycle-forming linker of the formula -L₁-L₂-;
L₁ and L₂ are independently alkylene, alkenylene, alkynylene, heteroalkylene, cycloalkylene, heterocycloalkylene, cycloarylene, heterocycloarylene, or [—R₄—K—R₄—]ₙ, each being optionally substituted with R₅;
each R₄ is alkylene, alkenylene, alkynylene, heteroalkylene, cycloalkylene, heterocycloalkylene, arylene, or heteroarylene;
each K is O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅;
v and w are independently integers from 1-1000;
u is an integer from 1-10;
x, y and z are independently integers from 0-10; and
n is an integer from 1-5.

In one example, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments of the invention, x+y+z is at least 1. In other embodiments of the invention, x+y+z is at least 2. In other embodiments of the invention, x+y+z is 1, 2, 3, 4, 5, 6, 7, 8, 9 or 10. Each occurrence of A, B, C, D or E in a macrocycle or macrocycle precursor of the invention is independently selected. For example, a sequence represented by the formula [A]ₓ, when x is 3, encompasses embodiments where the amino acids are not identical, e.g. Gln-Asp-Ala as well as embodiments where the amino acids are identical, e.g. Gln-Gln-Gln. This applies for any value of x, y, or z in the indicated ranges.

In some embodiments, the peptidomimetic macrocycle of the invention comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

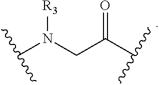

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, the peptidomimetic macrocycle has improved binding affinity to MDM2 or MDMX relative to a corresponding peptidomimetic macrocycle where w is 0, 1 or 2. In other instances, the peptidomimetic macrocycle has a reduced ratio of binding affinities to MDMX versus MDM2 relative to a corresponding peptidomimetic macrocycle where w is 0, 1 or 2. In still other instances, the peptidomimetic macrocycle has improved in vitro anti-tumor efficacy against p53 positive tumor cell lines relative to a corresponding peptidomimetic macrocycle where w is 0, 1 or 2. In some embodiments, the peptidomimetic macrocycle shows improved in vitro induction of apoptosis in p53 positive tumor cell lines relative to a corresponding peptidomimetic macrocycle where w is 0, 1 or 2. In other instances, the peptidomimetic macrocycle of claim 1, wherein the peptidomimetic macrocycle has an improved in vitro anti-tumor efficacy ratio for p53 positive versus p53 negative or mutant tumor cell lines relative to a corresponding peptidomimetic macrocycle where w is 0, 1 or 2. In still other instances, the peptidomimetic macrocycle has improved in vivo anti-tumor efficacy against p53 positive tumors relative to a corresponding peptidomimetic macrocycle where w is 0, 1 or 2. In yet other instances, the peptidomimetic macrocycle has improved in vivo induction of apoptosis in p53 positive tumors relative to a corresponding peptidomimetic macrocycle where w is 0, 1 or 2. In some embodiments, the peptidomimetic macrocycle has improved cell permeability relative to a corresponding peptidomimetic macrocycle where w is 0, 1 or 2. In other cases, the peptidomimetic macrocycle has improved solubility relative to a corresponding peptidomimetic macrocycle where w is 0, 1 or 2.

In some embodiments, Xaa₅ is Glu or an amino acid analog thereof. In some embodiments, Xaa₅ is Glu or an amino acid analog thereof and wherein the peptidomimetic macrocycle has an improved property, such as improved binding affinity, improved solubility, improved cellular efficacy, improved cell permeability, improved in vivo or in vitro anti-tumor efficacy, or improved induction of apoptosis relative to a corresponding peptidomimetic macrocycle where Xaa₅ is Ala.

In some embodiments, the peptidomimetic macrocycle has improved binding affinity to MDM2 or MDMX relative to a corresponding peptidomimetic macrocycle where Xaa₅ is Ala. In other embodiments, the peptidomimetic macrocycle has a reduced ratio of binding affinities to MDMX vs MDM2 relative to a corresponding peptidomimetic macrocycle where Xaa₅ is Ala. In some embodiments, the peptidomimetic macrocycle has improved solubility relative to a corresponding peptidomimetic macrocycle where Xaa₅ is Ala, or the peptidomimetic macrocycle has improved cellular efficacy relative to a corresponding peptidomimetic macrocycle where Xaa₅ is Ala.

In some embodiments, Xaa₅ is Glu or an amino acid analog thereof and wherein the peptidomimetic macrocycle has improved biological activity, such as improved binding affinity, improved solubility, improved cellular efficacy, improved helicity, improved cell permeability, improved in vivo or in vitro anti-tumor efficacy, or improved induction of apoptosis relative to a corresponding peptidomimetic macrocycle where Xaa₅ is Ala.

In one embodiment, a compound disclosed herein can contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). In another embodiment, a compound disclosed herein can have one or more carbon atoms replaced with a silicon atom. All isotopic variations of the compounds disclosed herein, whether radioactive or not, are contemplated herein.

The term “liquid cancer” as used herein refers to cancer cells that are present in body fluids, such as blood, lymph, and bone marrow. Liquid cancers include leukemia, myeloma and liquid lymphomas. Liquid lymphomas include lymphomas that contain cysts or liquid areas. Liquid cancers as used herein do not include solid tumors, such as sarcomas and carcinomas or solid lymphomas that do not contain cysts or liquid areas.

The term “adverse event” (AE) as used herein includes any noxious, pathological, or unintended change in anatomical, physiological, or metabolic functions as indicated by physical signs, symptoms, and/or laboratory changes occurring in any phase of the clinical study whether or not temporally associated with the administration of study medication and whether or not considered related to the study medication. This definition includes an exacerbation of pre-existing medical conditions or events, intercurrent illnesses, hypersensitivity reactions, drug interactions, or clinically significant laboratory findings. An AE does not include the following: (i) medical or surgical procedures, e.g., tooth extraction, transfusion, surgery (The medical condition that leads to the procedure is to be recorded as an AE); (ii) pre-existing conditions or procedures present or detected at the start of the study that do not worsen; (iii) hospitalization for elective surgeries or for other situations in which an untoward medical event has not occurred; (iv) abnormal laboratory value, unless it is clinically significant according to the Investigator, requires intervention, or results in a delay, discontinuation or change in the dose of study drug; (v) overdose of study drug or concomitant medication unaccompanied by signs/symptoms; if sign/symptoms occur, the final diagnosis should be recorded as an AE; (vi) pregnancy by itself, unless a complication occurs during pregnancy leading to hospitalization; in this case, the medical condition that leads to the hospitalization is to be recorded as the AE; and (vii) significant worsening of the disease under investigation which is captured as an efficacy parameter in this study and, thus, is not recorded as an AE.

The term serious adverse event (SAE) as used herein refers to an adverse event that results in any of the following outcomes: (i) death; (ii) life-threatening adverse experience (i.e., immediate risk of death from the event as it occurred; this does not include an adverse event that, had it occurred in a more serious form, might have caused death); (iii) persistent or significant disability/incapacitation; (iv) hospitalization or prolongation of existing hospitalization; and (v) congenital anomaly/birth defect. Important medical events that can not result in death, be life-threatening, or require hospitalization can be considered serious when, based on medical judgment, they can jeopardize the patient or can require medical or surgical intervention to prevent one of the outcomes listed in this definition. Hospitalizations due to the underlying disease will not be reported as an SAE unless there is reason to suspect a causal relationship with the study drug.

An AE or suspected adverse reaction is considered “unexpected” (referred to as Unexpected Adverse Event (UAE) if it is not listed in the peptidomimetic macrocycle Investigator's Brochure or is not listed at the specificity or severity that has been observed; or, is not consistent with the risk information described in the protocol or elsewhere. For example, under this definition, hepatic necrosis would be unexpected (by virtue of greater severity) if the Investigator's Brochure referred only to elevated hepatic enzymes or hepatitis. Similarly, cerebral thromboembolism and cerebral vasculitis would be unexpected (by virtue of greater specificity) if the Investigator's Brochure listed only cerebral vascular accidents. “Unexpected,” as used in this definition, also refers to AEs or suspected adverse reactions that are mentioned in the Investigator's Brochure as occurring with a class of drugs or as anticipated from the pharmacological properties of the peptidomimetic macrocycle but are not specifically mentioned as occurring with the peptidomimetic macrocycle.

A “Dose-Limiting Toxicity” (DLT) as used herein is defined as any Grade ≧3 AE that is considered to be possibly, probably, or definitely related to the study drug, with the following exceptions: (1) for nausea, emesis, diarrhea, rash, or mucositis, only Grade ≧3 AE that do not respond within 48 hours to standard supportive/pharmacological treatment will be considered DLT; (2) for electrolyte imbalances, only Grade ≧3 AE that do not respond to correction within 24 hours will be considered DLT; (3) for infusion reactions, only a Grade 3 reaction which caused hospitalization or Grade 4 will be considered DLT. In addition, specific hematologic DLTs are defined as:
Thrombocytopenia—Grade 4 of any duration, Grade 3 for ≧7 days, or Grade 3 associated with clinically significant bleeding;
Neutropenia—Grade 4 for ≧3 days, or any Grade ≧3 febrile neutropenia

The above criteria can be used to make individual patient determinations regarding dose reductions, interruptions or discontinuation throughout the course of the trial, but DLTs occurring during Cycle 1 will be used to inform safety and tolerability assessments for dose escalation decisions. The DLT-evaluable population will include all patients in Phase 1 Dose Escalation who experience a DLT during the first cycle of treatment.

The “Maximum Tolerated Dose” (MTD) as used herein is defined as the dose at which ≦1 of 6 patients experiences a treatment-related toxicity that qualifies as a DLT, with the next higher dose having ≧2 of up to 6 patients experiencing a DLT. The MTD can not be established until all patients enrolled in the cohort have completed Cycle 1, discontinued treatment or had a dose reduction. Previously established tolerability of a dose level will be reevaluated if DLTs are observed in later cycles.

The “Optimal Biological Dose” (OBD) as used herein is defined as the dose at which for each treatment arm, the safety review committee identifies before the MTD is reached. Such OBD would be derived from the evaluation of available safety, PK, PD, and.or preliminary efficacy information from the dose escalation portion of the study

“Measurable disease” (MD) as used herein is defined by the presence of at least one measurable CTC or MNBC.

Measurable CTCs and MNBCs are defined as those from a biological sample that can be accurately counted.

“Non-measurable Disease” as used herein include all other lesions.

“Complete response” (CR) as used herein is defined as the disappearance of all target CTCs and/or MNBCs.

“Partial response (PR)” as used herein is defined as at least a 30% decrease in the number of CTCs and/or MNBCs.

“Progressive disease (PD)” as used herein is defined as at least a 20% increase in the number of CTCs and/or MNBCs.

“Stable disease” (SD) as used herein is defined as neither sufficient shrinkage to qualify for PR nor sufficient increase to qualify for PD, taking as reference the smallest sum diameters while on study.

The term “subject” or “patient” encompasses mammals and non-mammals. Examples of mammals include, but are not limited to, humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

Unless otherwise stated, any compounds (including peptidomimetic macrocycles, macrocycle precursors, and other compositions) are also meant to encompass compounds which differ only in the presence of one or more isotopically enriched atoms. For example, compounds having the described structures except for the replacement of a hydrogen by a deuterium or tritium, or the replacement of a carbon by ¹³C- or ¹⁴C-enriched carbon are within the scope of this disclosure.

In some embodiments, the compounds disclosed herein can contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). In other embodiments, one or more carbon atoms are replaced with a silicon atom. All isotopic variations of the compounds disclosed herein, whether radioactive or not, are contemplated herein.

The circulating half-life of the peptidomimetic macrocycles in human blood can be about 1-24 h. For example the circulating half-life of the peptidomimetic macrocycles in human blood can me about 2-24 h, 4-24 h, 6-24 h, 8-24 h, 10-24 h, 12-24 h, 14-24 h, 16-24 h, 18-24 h, 20-24 h, 22-24 h, 1-20 h, 4-20 h, 6-20 h, 8-20 h, 10-20 h, 12-20 h, 14-20 h, 16-20 h, 18-20 h, 1-16 h, 4-16 h, 6-16 h, 8-16 h, 10-16 h, 12-16 h, 14-16 h, 1-12 h, 4-12 h, 6-12 h, 8-12 h, 10-12 h, 1-8 h, 4-8 h, 6-8 h, or 1-4 h. In some examples, the circulating half-life of the peptidomimetic macrocycles in human blood can be bout 1-12 h, for example about 1 h, 2 h, 3 h, 4 h, 5 h, 6 h, 7 h, 8 h, 9 h, 10 h, 11 h, or 12 h. In some examples, the circulating half-life of the peptidomimetic macrocycles in human blood is about 2 h. In some examples, the circulating half-life of the peptidomimetic macrocycles in human blood is about 4 h. In some examples, the circulating half-life of the peptidomimetic macrocycles in human blood is about 6 h. In some examples, the circulating half-life of the peptidomimetic macrocycles in human blood is about 8 h. In some examples, the circulating half-life of the peptidomimetic macrocycles in human blood is about 10 h.

The half-life of the peptidomimetic macrocycles in biological tissue can be about 1-24 h. For example the circulating half-life of the peptidomimetic macrocycles in human blood can me about 1-24 h, 5-24 h, 10-24 h, 15-24 h, 20-24 h, 1-22 h, 5-22 h, 10-22 h, 15-22 h, 20-22 h, 1-20 h, 5-20 h, 15-20 h, 1-18 h, 5-18 h, 10-18 h, 15-18 h, 1-16 h, 5-16 h, 10-16 h, 15- 16 h, 1-14 h, 5-14 h, 10-14 h, 1-12 h, 5-12 h, 10-12 h, 1-10 h, 5-10 h, 1-8 h, 5-8 h, 1-6 h, 5- 6 h, or 1-4 h. In some examples, the circulating half-life of the peptidomimetic macrocycles in human blood can be bout 5-20 h, for example about 5 h, 6 h, 7 h, 8 h, 9 h, 10 h, 11 h, 12 h, 13 h, 14 h, 15 h, 16 h, 17 h, 18 h, 19 h or 20 h. In some examples, the circulating half-life of the peptidomimetic macrocycles in human blood is about 2 h. In some examples, the circulating half-life of the peptidomimetic macrocycles in human blood is about 4 h. In some examples, the circulating half-life of the peptidomimetic macrocycles in human blood is about 6 h. In some examples, the circulating half-life of the peptidomimetic macrocycles in human blood is about 8 h. In some examples, the circulating half-life of the peptidomimetic macrocycles in human blood is about 10 h.

The circulating half-life of the peptidomimetic macrocycles in human blood can be greater than, equal to, or less than the half-life of the peptidomimetic macrocycles in biological tissue. In some examples, the circulating half-life of the peptidomimetic macrocycles in human blood can be greater than the half-life of the peptidomimetic macrocycles in biological tissue. In some examples, the circulating half-life of the peptidomimetic macrocycles in human blood can be equal to the half-life of the peptidomimetic macrocycles in biological tissue. In some examples, the half-life of the peptidomimetic macrocycles in biological tissue is greater than the circulating half-life of the peptidomimetic macrocycles in human blood. This can facilitate administration of the peptidomimetic macrocycles at a lower dose and/or at lower frequency. In some embodiments, the half-life of the peptidomimetic macrocycles in biological tissue is at least 1 h, at least 2 h, at least 3 h, at least 4 h, at least 5 h, at least 6 h, at least 7 h, at least 8 h, at least 9 h, at least 10 h, at least 11 h, or at least 12 h greater than the than the circulating half-life of the peptidomimetic macrocycles in human blood. In some examples, the circulating half-life of the peptidomimetic macrocycles in human blood is about 4 h and the half-life of the in biological tissue is about 10 h. In some examples, the circulating half-life of the peptidomimetic macrocycles in human blood is about 6 h and the half-life of the in biological tissue is about 10 h.

The details of one or more particular embodiments of the invention are set forth in the accompanying drawings and the description below. Other features, objects, and advantages of the invention will be apparent from the description and drawings, and from the claims.

### Preparation of Peptidomimetic Macrocycles

Peptidomimetic macrocycles of the invention may be prepared by any of a variety of methods known in the art. For example, any of the residues indicated by “X” in Tables 1-3 and 9 may be substituted with a residue capable of forming a crosslinker with a second residue in the same molecule or a precursor of such a residue.

Peptidomimetic macrocycles can be prepared by any of a variety of methods known in the art. For example, any of the residues indicated by “$” or “$r8” in Tables 10-13 can be substituted with a residue capable of forming a crosslinker with a second residue in the same molecule or a precursor of such a residue.

Various methods to effect formation of peptidomimetic macrocycles are known in the art. For example, a preparation of a peptidomimetic macrocycles disclosed is described in Schafmeister et al., J. Am. Chem. Soc. 122:5891-5892 (2000); Schafmeister & Verdin, J. Am. Chem. Soc. 122:5891 (2005); Walensky et al., Science 305:1466-1470 (2004); U.S. Pat. No. 7,192,713 and PCT application WO 2008/121767. The α,α-disubstituted amino acids and amino acid precursors disclosed in the cited references may be employed in synthesis of the peptidomimetic macrocycle precursor polypeptides. For example, the “S5-olefin amino acid” is (S)-α-(2′-pentenyl) alanine and the “R8 olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle. In various embodiments, the following amino acids may be employed in the synthesis of the peptidomimetic macrocycle:

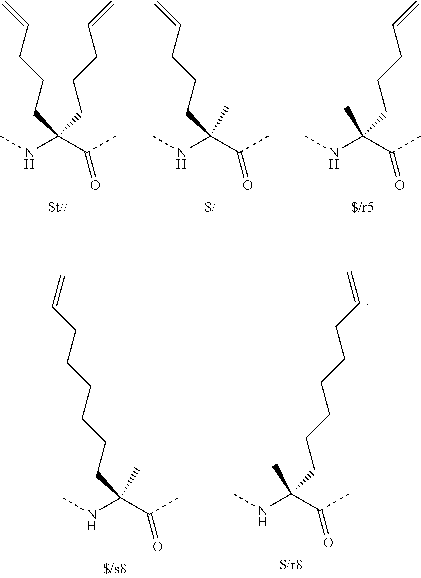

Methods for the preparation of disclosed macrocycles are described, for example, in U.S. Pat. No. 7,202,332. Additional methods of forming peptidomimetic macrocycles which are envisioned as suitable to perform the present invention include those disclosed by Mustapa, M. Firouz Mohd et al., J. Org. Chem (2003), 68, pp. 8193-8198; Yang, Bin et al. Bioorg Med. Chem. Lett. (2004), 14, pp. 1403-1406; U.S. Pat. No. 5,364,851; U.S. Pat. No. 5,446,128; U.S. Pat. No. 5,824,483; U.S. Pat. No. 6,713,280; and U.S. Pat. No. 7,202,332. In such embodiments, aminoacid precursors are used containing an additional substituent R— at the alpha position. Such aminoacids are incorporated into the macrocycle precursor at the desired positions, which may be at the positions where the crosslinker is substituted or, alternatively, elsewhere in the sequence of the macrocycle precursor. Cyclization of the precursor is then affected according to the indicated method.

Peptidomimetic macrocycles disclosed can be prepared by any of a variety of methods known in the art. For example, macrocycles having residues indicated by “$4rn6” or “$4a5” in Tables 23-25 can be substituted with a residue capable of forming a crosslinker with a second residue in the same molecule or a precursor of such a residue.

In some embodiments, the synthesis of these peptidomimetic macrocycles involves a multi-step process that features the synthesis of a peptidomimetic precursor containing an azide moiety and an alkyne moiety; followed by contacting the peptidomimetic precursor with a macrocyclization reagent to generate a triazole-linked peptidomimetic macrocycle. Such a process is described, for example, in U.S. application Ser. No. 12/037,041, filed on Feb. 25, 2008. Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985.

In some embodiments of the macrocycles disclosed, an azide is linked to the α-carbon of a residue and an alkyne is attached to the α-carbon of another residue. In some embodiments, the azide moieties are azido-analogs of amino acids L-lysine, D-lysine, alpha-methyl-L-lysine, alpha-methyl-D-lysine, L-ornithine, D-ornithine, alpha-methyl-L-ornithine or alpha-methyl-D-ornithine. In another embodiment, the alkyne moiety is L-propargylglycine. In yet other embodiments, the alkyne moiety is an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid and (R)-2-amino-2-methyl-8-nonynoic acid.

In some embodiments, provided herein is a method for synthesizing a peptidomimetic macrocycle disclosed, the method comprising the steps of contacting a peptidomimetic precursor of formulas:

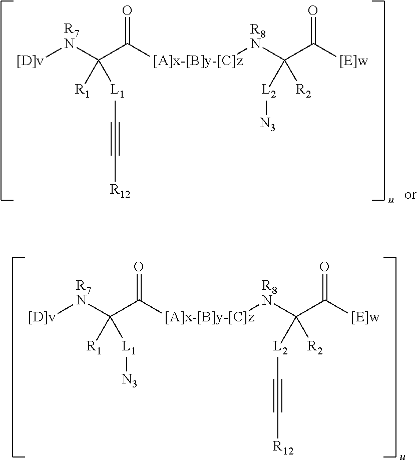

with a macrocyclization reagent;
wherein v, w, x, y, z, A, B, C, D, E, R₁, R₂, R₇, R₈, L₁ and L₂ are as defined above; R₁₂ is —H when the macrocyclization reagent is a Cu reagent and R₁₂ is —H or alkyl when the macrocyclization reagent is a Ru reagent; and further wherein said contacting step results in a covalent linkage being formed between the alkyne and azide moiety in the precursor. For example, R₁₂ may be methyl when the macrocyclization reagent is a Ru reagent.

In some embodiments, provided herein is a method for synthesizing a peptidomimetic macrocycle disclosed, the method comprising the steps of contacting a peptidomimetic precursor of formula:

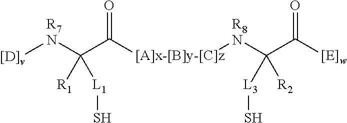

with a compound formula X-L₂-Y,
wherein v, w, x, y, z, A, B, C, D, E, R₁, R₂, R₇, R₈, L₁ and L₂ are as defined for the compound previously disclosed; and X and Y are each independently a reactive group capable of reacting with a thiol group;
and further wherein said contacting step results in a covalent linkage being formed between the two thiol groups.

In the peptidomimetic macrocycles disclosed herein, at least one of R₁ and R₂ is alkyl, alkenyl, alkynyl, arylalkyl, cycloalkyl, cycloalkylalkyl, heteroalkyl, or heterocycloalkyl, unsubstituted or substituted with halo-. In some embodiments, both R₁ and R₂ are independently alkyl, alkenyl, alkynyl, arylalkyl, cycloalkyl, cycloalkylalkyl, heteroalkyl, or heterocycloalkyl, unsubstituted or substituted with halo-. In some embodiments, at least one of A, B, C, D or E is an α,α-disubstituted amino acid. In one example, B is an α,α-disubstituted amino acid. For instance, at least one of A, B, C, D or E is 2-aminoisobutyric acid.

For example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl. The macrocyclization reagent may be a Cu reagent or a Ru reagent.

In some embodiments, the peptidomimetic precursor is purified prior to the contacting step. In other embodiments, the peptidomimetic macrocycle is purified after the contacting step. In still other embodiments, the peptidomimetic macrocycle is refolded after the contacting step. The method may be performed in solution, or, alternatively, the method may be performed on a solid support.

Also envisioned herein is performing the method disclosed herein in the presence of a target macromolecule that binds to the peptidomimetic precursor or peptidomimetic macrocycle under conditions that favor said binding. In some embodiments, the method is performed in the presence of a target macromolecule that binds preferentially to the peptidomimetic precursor or peptidomimetic macrocycle under conditions that favor said binding. The method may also be applied to synthesize a library of peptidomimetic macrocycles.

In some embodiments, an alkyne moiety of the peptidomimetic precursor for making a compound disclosed is a sidechain of an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid, and (R)-2-amino-2-methyl-8-nonynoic acid. In other embodiments, an azide moiety of the peptidomimetic precursor for making a compound disclosed is a sidechain of an amino acid selected from the group consisting of ε-azido-L-lysine, ε-azido-D-lysine, ε-azido-α-methyl-L-lysine, ε-azido-α-methyl-D-lysine, δ-azido-α-methyl-L-ornithine, and δ-azido-α-methyl-D-ornithine.

In some embodiments, a thiol group of the peptidomimetic precursor for making a compound disclosed is a sidechain of an amino acid selected from the group consisting of L-cysteine, D-cysteine, L-N-methylcysteine, D-N-methylcysteine, L-homocysteine, D-homocysteine, L-N-methylhomocysteine, D-N-methylhomocysteine, α-methyl-L-cysteine, α-methyl-D-cysteine, α-methyl-L-homocysteine, α-methyl-D-homocysteine, L-penicillamine, D-penicillamine, L-N-methylpenicillamine, D-N-methylpenicillamine and all forms suitably protected for liquid or solid phase peptide synthesis.

In some embodiments, x+y+z is 3, and A, B and C are independently natural or non-natural amino acids. In other embodiments, x+y+z is 6, and A, B and C are independently natural or non-natural amino acids.

In some embodiments, the contacting step is performed in a solvent selected from the group consisting of protic solvent, aqueous solvent, organic solvent, and mixtures thereof. For example, the solvent may be chosen from the group consisting of H₂O, THF, THF/H₂O, tBuOH/H₂O, DMF, DIPEA, CH₃CN or CH₂Cl₂, ClCH₂CH₂Cl or a mixture thereof. The solvent may be a solvent which favors helix formation.

Alternative but equivalent protecting groups, leaving groups or reagents are substituted, and certain of the synthetic steps are performed in alternative sequences or orders to produce the desired compounds. Synthetic chemistry transformations and protecting group methodologies (protection and deprotection) useful in synthesizing the compounds described herein include, for example, those such as described in Larock, Comprehensive Organic Transformations, VCH Publishers (1989); Greene and Wuts, Protective Groups in Organic Synthesis, 2d. Ed., John Wiley and Sons (1991); Fieser and Fieser, Fieser and Fieser's Reagents for Organic Synthesis, John Wiley and Sons (1994); and Paquette, ed., Encyclopedia of Reagents for Organic Synthesis, John Wiley and Sons (1995), and subsequent editions thereof.

The peptidomimetic macrocycles disclosed herein are made, for example, by chemical synthesis methods, such as described in Fields et al., Chapter 3 in Synthetic Peptides: A User's Guide, ed. Grant, W. H. Freeman & Co., New York, N.Y., 1992, p. 77. Hence, for example, peptides are synthesized using the automated Merrifield techniques of solid phase synthesis with the amine protected by either tBoc or Fmoc chemistry using side chain protected amino acids on, for example, an automated peptide synthesizer (e.g., Applied Biosystems (Foster City, Calif.), Model 430A, 431, or 433).

One manner of producing the peptidomimetic precursors and peptidomimetic macrocycles described herein uses solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but is removable by base. Side chain functional groups are protected as necessary with base stable, acid labile groups.

Longer peptidomimetic precursors are produced, for example, by conjoining individual synthetic peptides using native chemical ligation. Alternatively, the longer synthetic peptides are biosynthesized by well known recombinant DNA and protein expression techniques. Such techniques are provided in well-known standard manuals with detailed protocols. To construct a gene encoding a peptidomimetic precursor disclosed herein, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptidomimetic precursors are made, for example, in a high-throughput, combinatorial fashion using, for example, a high-throughput polychannel combinatorial synthesizer (e.g., Thuramed TETRAS multichannel peptide synthesizer from CreoSalus, Louisville, Ky. or Model Apex 396 multichannel peptide synthesizer from AAPPTEC, Inc., Louisville, Ky.).

The following synthetic schemes are provided solely to illustrate the present invention and are not intended to limit the scope of the invention, as described herein.

Synthetic schemes 1-5 describe the preparation of the peptidomimetic macrocycles disclosed. To simplify the drawings, the illustrative schemes depict azido amino acid analogs ε-azido-α-methyl-L-lysine and ε-azido-α-methyl-D-lysine, and alkyne amino acid analogs L-propargylglycine, (S)-2-amino-2-methyl-4-pentynoic acid, and (S)-2-amino-2-methyl-6-heptynoic acid. Thus, in the following synthetic schemes, each R₁, R₂, R₇ and R₈ is —H; each L₁ is —(CH₂)₄—; and each L₂ is —(CH₂)—. However, as noted throughout the detailed description above, many other amino acid analogs can be employed in which R₁, R₂, R₇, R₈, L₁ and L₂ can be independently selected from the various structures disclosed herein.

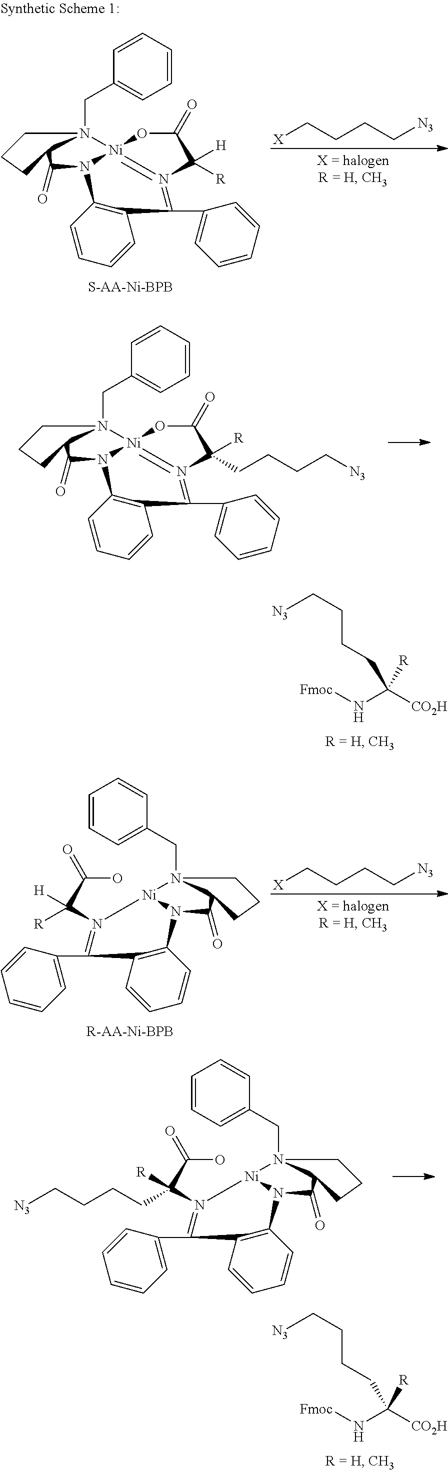

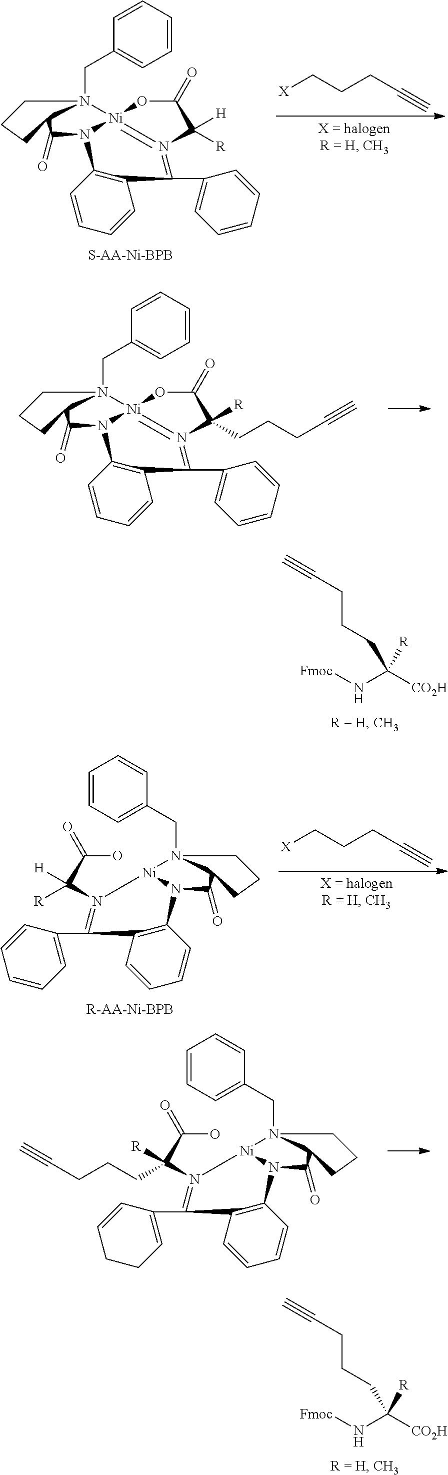

Synthetic Scheme 1 describes the preparation of several compounds useful for preparing compounds disclosed herein. Ni(II) complexes of Schiff bases derived from the chiral auxiliary (S)-2-[N—(N′-benzylpropyl)amino]benzophenone (BPB) and amino acids such as glycine or alanine are prepared as described in Belokon et al. (1998), Tetrahedron Asymm. 9:4249-4252. The resulting complexes are subsequently reacted with alkylating reagents comprising an azido or alkynyl moiety to yield enantiomerically enriched compounds disclosed herein. If desired, the resulting compounds can be protected for use in peptide synthesis.

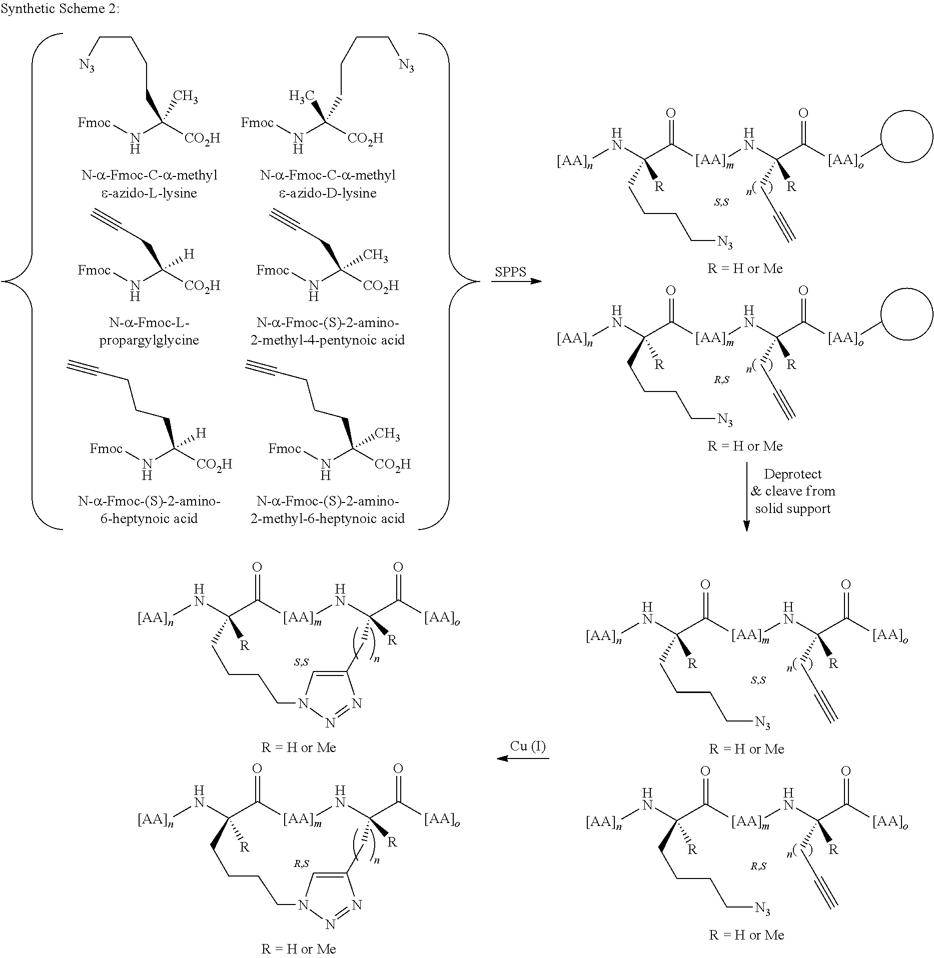

In the general method for the synthesis of peptidomimetic macrocycles disclosed shown in Synthetic Scheme 2, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Cu(I) in organic or aqueous solutions (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). In one embodiment, the triazole forming reaction is performed under conditions that favor α-helix formation. In one embodiment, the macrocyclization step is performed in a solvent chosen from the group consisting of H₂O, THF, CH₃CN, DMF, DIPEA, tBuOH or a mixture thereof. In another embodiment, the macrocyclization step is performed in DMF. In some embodiments, the macrocyclization step is performed in a buffered aqueous or partially aqueous solvent.

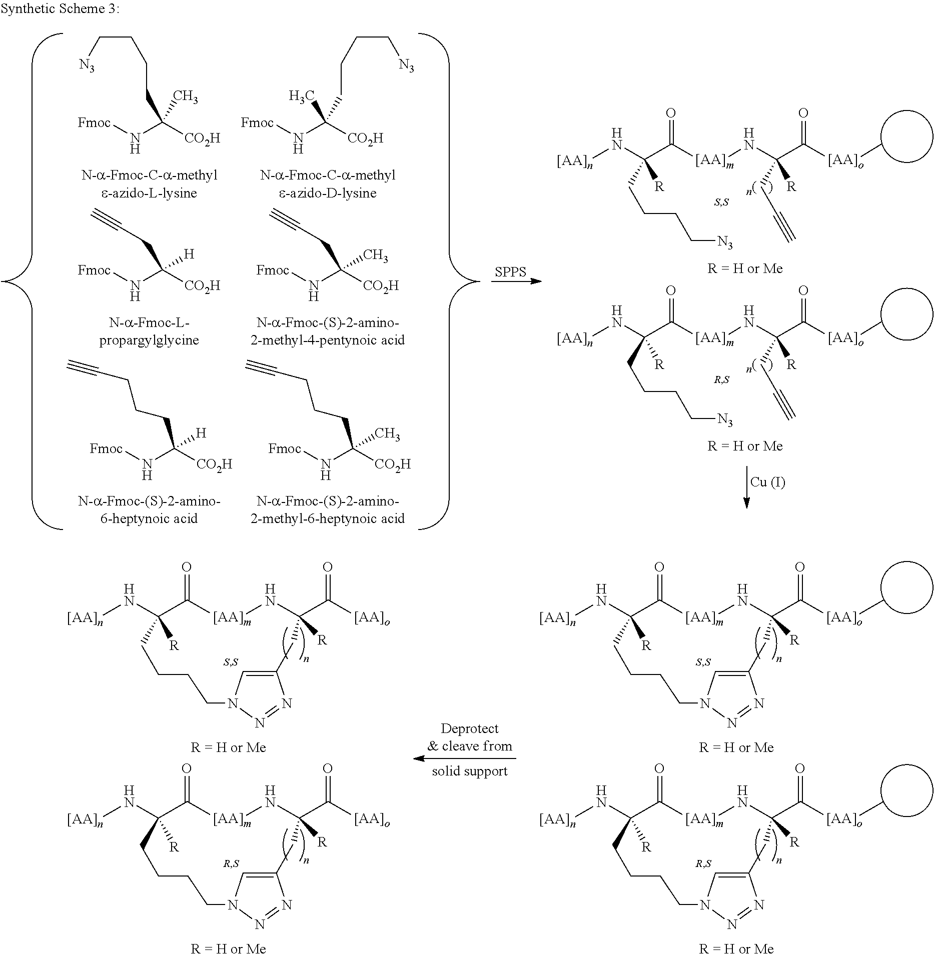

In the general method for the synthesis of peptidomimetic macrocycles disclosed shown in Synthetic Scheme 3, the peptidomimetic precursor contains an azide moiety and an alkyne moiety and is synthesized by solid-phase peptide synthesis (SPPS) using the commercially available amino acid N-σ-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is reacted with a macrocyclization reagent such as a Cu(I) reagent on the resin as a crude mixture (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). The resultant triazole-containing peptidomimetic macrocycle is then deprotected and cleaved from the solid-phase resin by standard conditions (e.g., strong acid such as 95% TFA). In some embodiments, the macrocyclization step is performed in a solvent chosen from the group consisting of CH₂Cl₂, ClCH₂CH₂Cl, DMF, THF, NMP, DIPEA, 2,6-lutidine, pyridine, DMSO, H₂O or a mixture thereof. In some embodiments, the macrocyclization step is performed in a buffered aqueous or partially aqueous solvent.

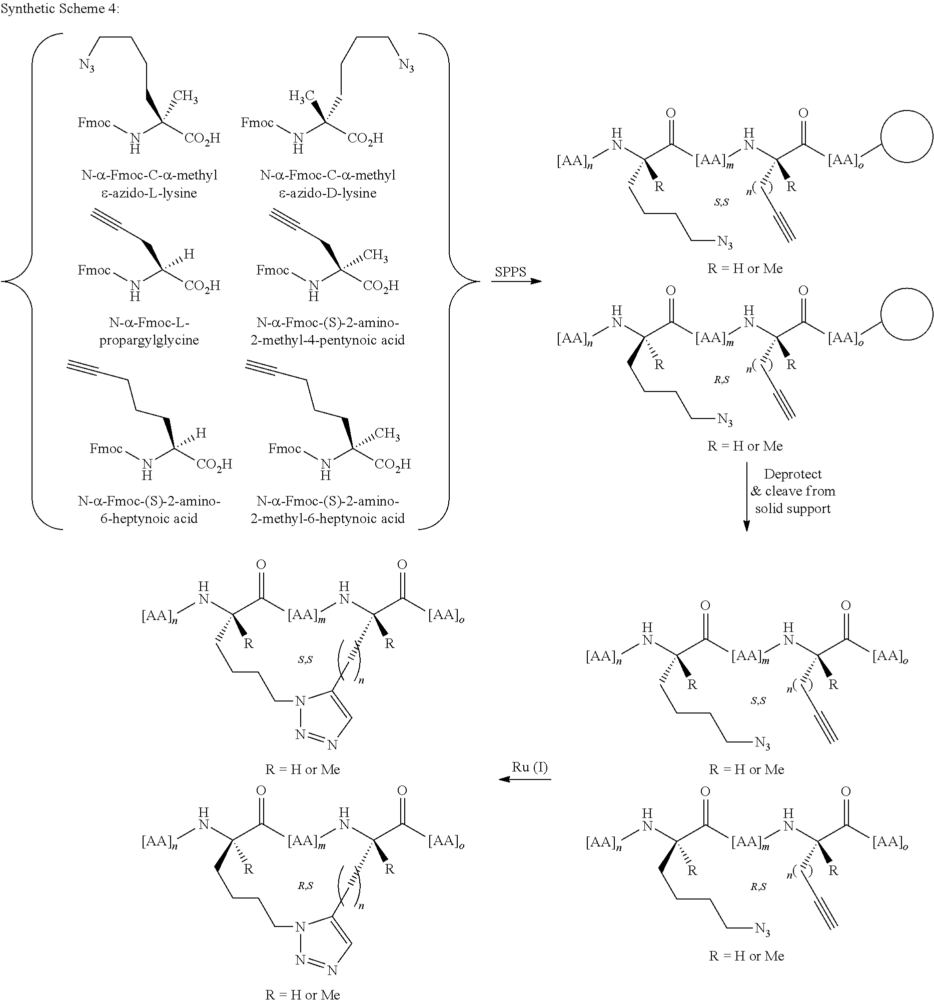

In the general method for the synthesis of peptidomimetic macrocycles disclosed shown in Synthetic Scheme 4, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Ru(II) reagents, for example Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of DMF, CH₃CN and THF.

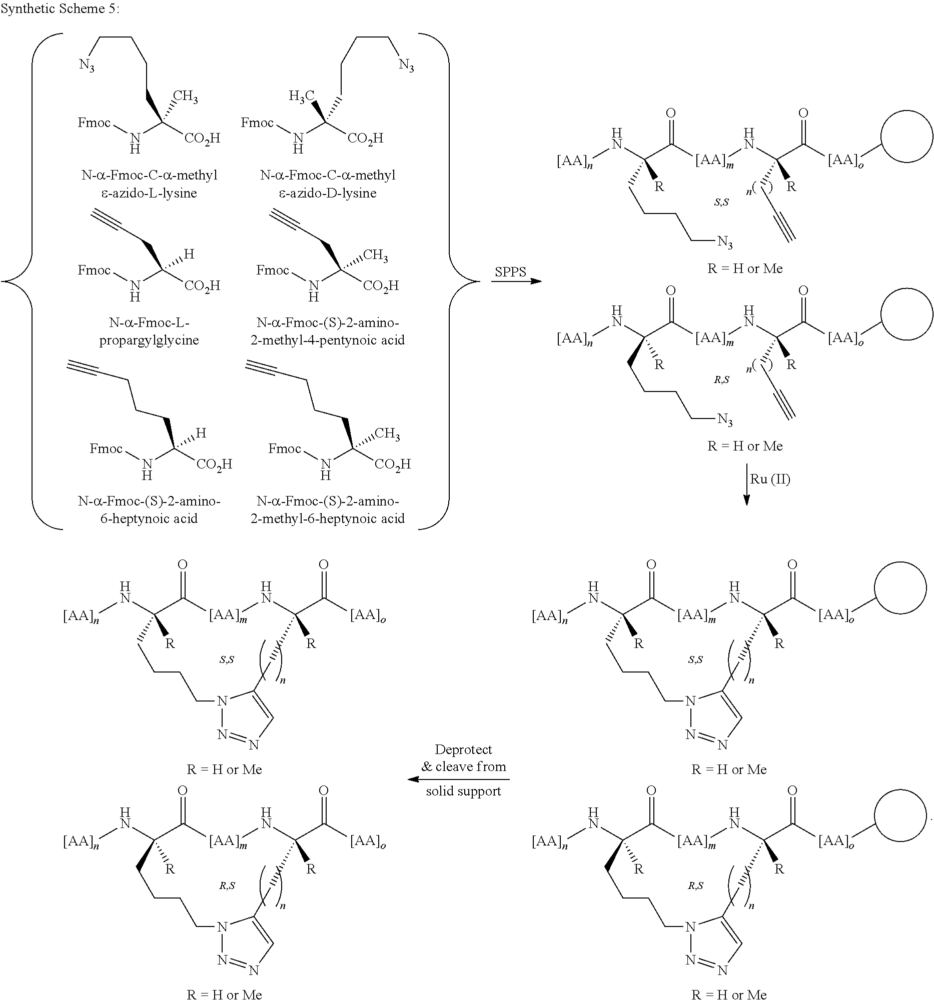

In the general method for the synthesis of peptidomimetic macrocycles of disclosed shown in Synthetic Scheme 5, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is reacted with a macrocyclization reagent such as a Ru(II) reagent on the resin as a crude mixture. For example, the reagent can be Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of CH₂Cl₂, ClCH₂CH₂Cl, CH₃CN, DMF, and THF.

In some embodiments, a peptidomimetic macrocycle disclosed comprises a halogen group substitution on a triazole moiety, for example an iodo substitution. Such peptidomimetic macrocycles may be prepared from a precursor having the partial structure and using the cross-linking methods taught herein. Crosslinkers of any length, as described herein, may be prepared comprising such substitutions. In one embodiment, the peptidomimetic macrocycle is prepared according to the scheme shown below. The reaction is performed, for example, in the presence of CuI and an amine ligand such as TEA or TTTA. See, e.g., Hein et al. Angew. Chem., Int. Ed. 2009, 48, 8018-8021.

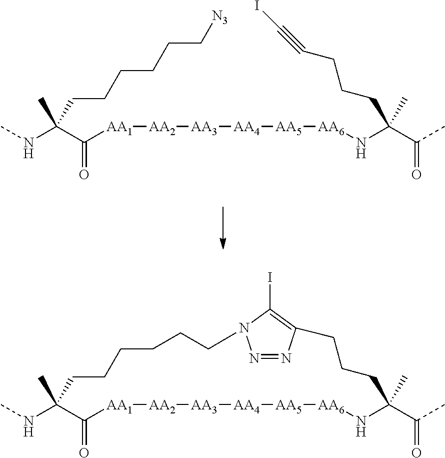

In other embodiments, an iodo-substituted triazole is generated according to the scheme shown below. For example, the second step in the reaction scheme below is performed using, for example, CuI and N-bromosuccinimide (NBS) in the presence of THF (see, e.g. Zhang et al., J. Org. Chem. 2008, 73, 3630-3633). In other embodiments, the second step in the reaction scheme shown below is performed, for example, using CuI and an iodinating agent such as ICl (see, e.g. Wu et al., Synthesis 2005, 1314-1318.)

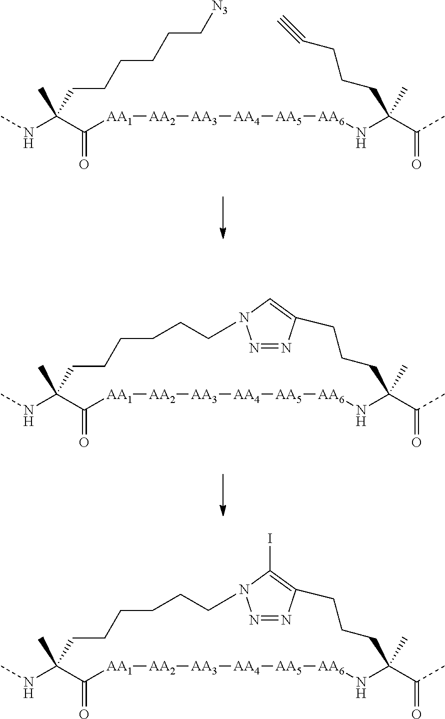

In some embodiments, an iodo-substituted triazole moiety is used in a cross-coupling reaction, such as a Suzuki or Sonogashira coupling, to afford a peptidomimetic macrocycle comprising a substituted crosslinker. Sonogashira couplings using an alkyne as shown below may be performed, for example, in the presence of a palladium catalyst such as Pd(PPh₃)₂Cl₂, CuI, and in the presence of a base such as triethylamine. Suzuki couplings using an arylboronic or substituted alkenyl boronic acid as shown below may be performed, for example, in the presence of a catalyst such as Pd(PPh₃)₄, and in the presence of a base such as K₂CO₃.

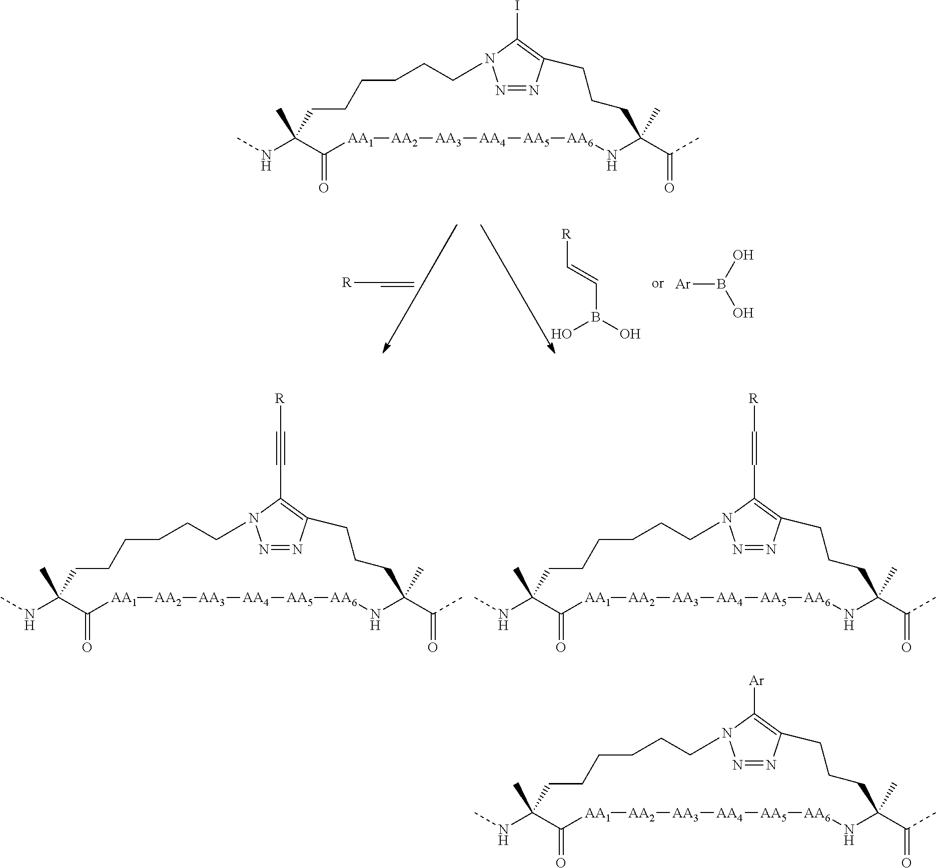

Any suitable triazole substituent group which reacts with the iodo-substituted triazole can be used in Suzuki couplings described herein. Example triazole substituents for use in Suzuki couplings are shown below:

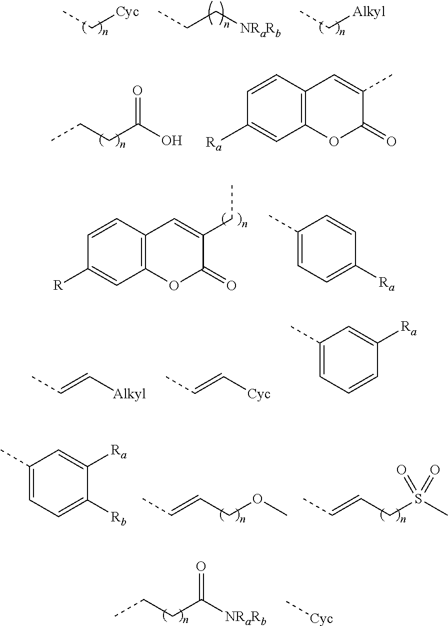

wherein “Cyc” is a suitable aryl, cycloalkyl, cycloalkenyl, heteroaryl, or heterocyclyl group, unsubstituted or optionally substituted with an Rₐ or R_{b} group as described below.

In some embodiments, the substituent is:

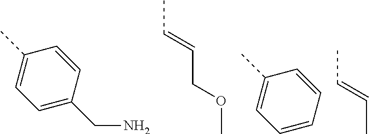

Any suitable substituent group which reacts with the iodo-substituted triazole can be used in Sonogashira couplings described herein. Example triazole substituents for use in Sonogashira couplings are shown below:

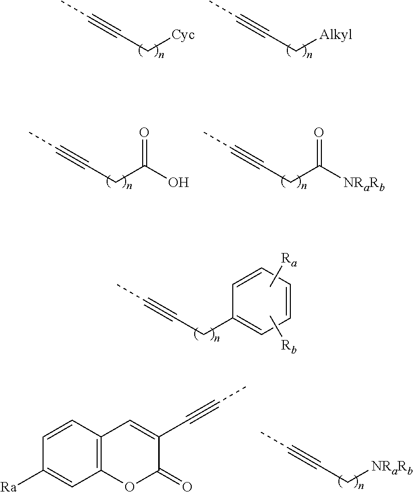

wherein “Cyc” is a suitable aryl, cycloalkyl, cycloalkenyl, heteroaryl, or heterocyclyl group, unsubstituted or optionally substituted with an Rₐ or R_{b} group as described below.

In some embodiments, the triazole substituent is:

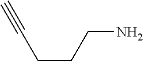

In some embodiments, the Cyc group shown above is further substituted by at least one Rₐ or R_{b} substituent. In some embodiments, at least one of Rₐ and R_{b} is independently:

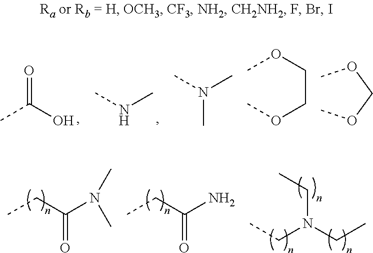

In other embodiments, the triazole substituent is

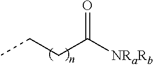

and at least one of Rₐ and R_{b} is alkyl (including hydrogen, methyl, or ethyl), or:

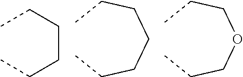

The present invention contemplates the use of non-naturally-occurring amino acids and amino acid analogs in the synthesis of the peptidomimetic macrocycles disclosed described herein. Any amino acid or amino acid analog amenable to the synthetic methods employed for the synthesis of stable triazole containing peptidomimetic macrocycles can be used in the present invention. For example, L-propargylglycine is contemplated as a useful amino acid in the present invention. However, other alkyne-containing amino acids that contain a different amino acid side chain are also useful in the invention. For example, L-propargylglycine contains one methylene unit between the α-carbon of the amino acid and the alkyne of the amino acid side chain. The invention also contemplates the use of amino acids with multiple methylene units between the α-carbon and the alkyne. Also, the azido-analogs of amino acids L-lysine, D-lysine, alpha-methyl-L-lysine, and alpha-methyl-D-lysine are contemplated as useful amino acids in the present invention. However, other terminal azide amino acids that contain a different amino acid side chain are also useful in the invention. For example, the azido-analog of L-lysine contains four methylene units between the α-carbon of the amino acid and the terminal azide of the amino acid side chain. The invention also contemplates the use of amino acids with fewer than or greater than four methylene units between the α-carbon and the terminal azide. The following Table 4 shows some amino acids useful in the preparation of peptidomimetic macrocycles disclosed herein.

TABLE 4

| | |
|---|---|
| 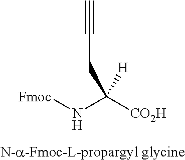 | 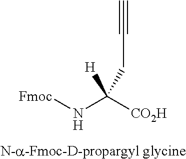 |
| 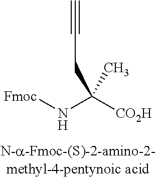 | 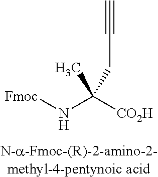 |
| 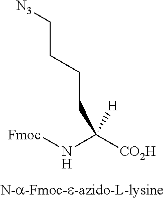 | 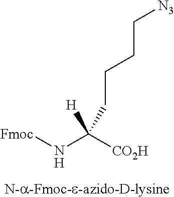 |
| 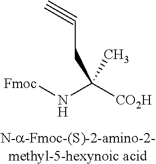 | 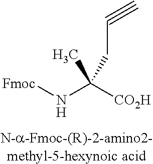 |
| 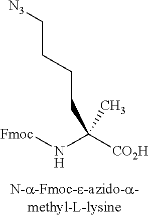 | 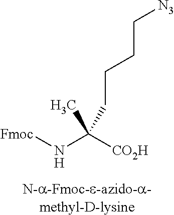 |
| 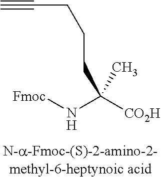 | 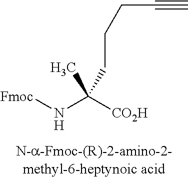 |
| 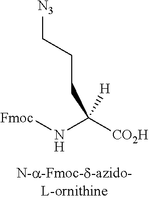 | 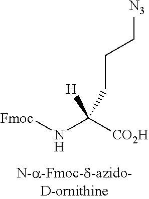 |
| 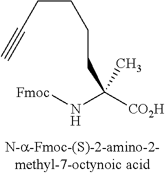 | 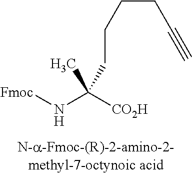 |
| 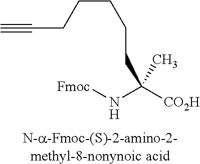 | 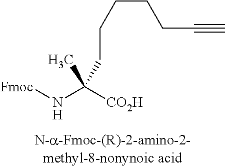 |
| 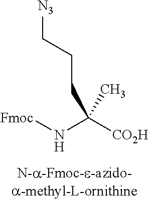 | 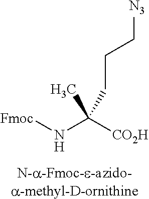 |

In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-propargylglycine, α-methyl-D-propargylglycine, ε-azido-alpha-methyl-L-lysine, and ε-azido-alpha-methyl-D-lysine. In some embodiments the amino acid analogs are N-alkylated, e.g., N-methyl-L-propargylglycine, N-methyl-D-propargylglycine, N-methyl-ε-azido-L-lysine, and N-methyl-ε-azido-D-lysine.

The preparation of macrocycles disclosed is described, for example, in U.S. application Ser. No. 11/957,325, filed on Dec. 17, 2007 and herein incorporated by reference. Synthetic Schemes 6-9 describe the preparation of such compounds disclosed. To simplify the drawings, the illustrative schemes depict amino acid analogs derived from L- or D-cysteine, in which L₁ and L₃ are both —(CH₂)—. However, as noted throughout the detailed description above, many other amino acid analogs can be employed in which L₁ and L₃ can be independently selected from the various structures disclosed herein. The symbols “[AA]ₘ”, “[AA]ₙ”, “[AA]ₒ” represent a sequence of amide bond-linked moieties such as natural or unnatural amino acids. As described previously, each occurrence of “AA” is independent of any other occurrence of “AA”, and a formula such as “[AA]ₘ” encompasses, for example, sequences of non-identical amino acids as well as sequences of identical amino acids.

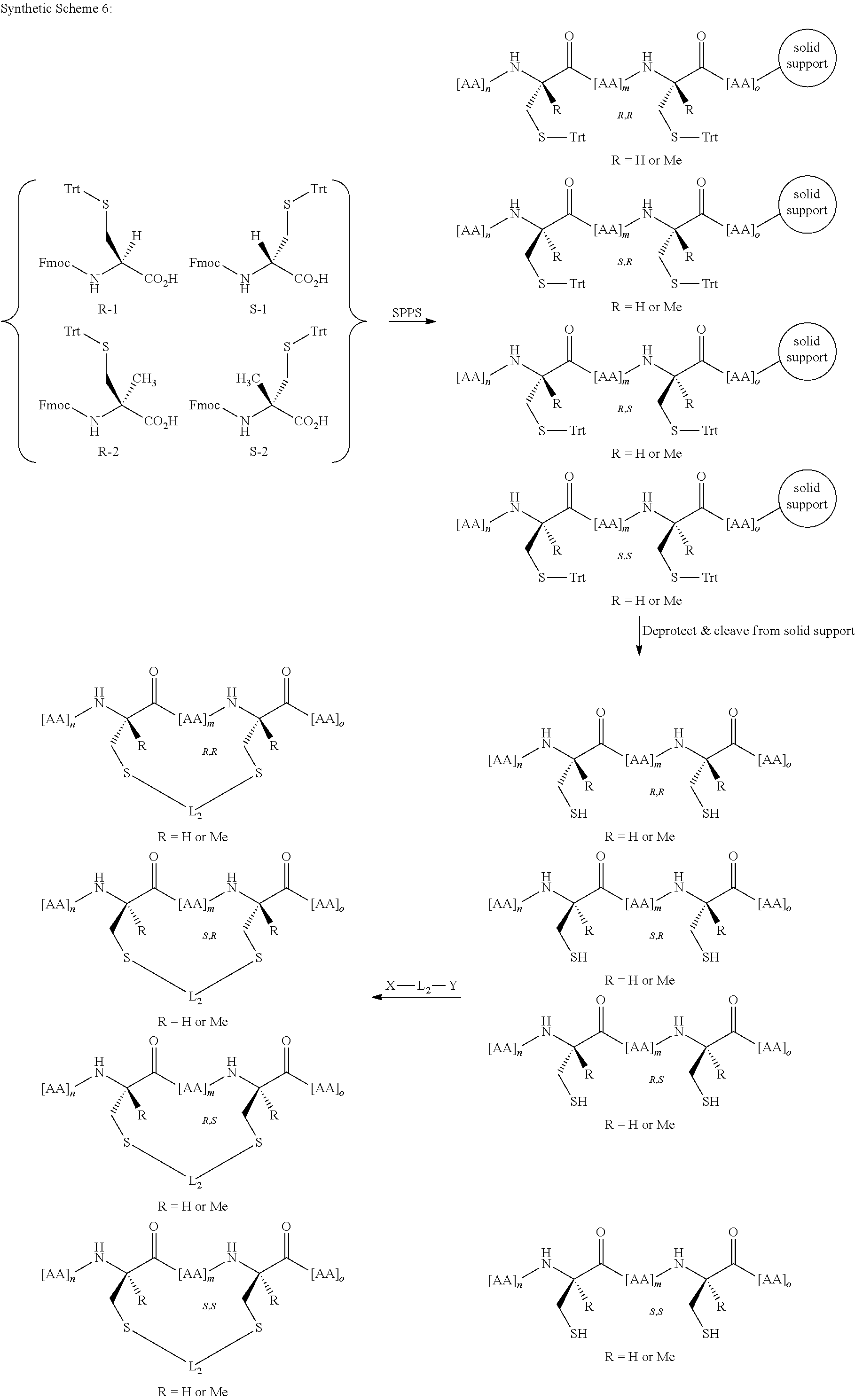

In Scheme 6, the peptidomimetic precursor contains two —SH moieties and is synthesized by solid-phase peptide synthesis (SPPS) using commercially available N-α-Fmoc amino acids such as N-α-Fmoc-S-trityl-L-cysteine or N-α-Fmoc-S-trityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-trityl monomers by known methods (“Bioorganic Chemistry: Peptides and Proteins”, Oxford University Press, New York: 1998, the entire contents of which are incorporated herein by reference). The precursor peptidomimetic is then deprotected and cleaved from the solid-phase resin by standard conditions (e.g., strong acid such as 95% TFA). The precursor peptidomimetic is reacted as a crude mixture or is purified prior to reaction with X-L₂-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6 M guanidinium HCl, pH 8 (Brunel et al. (2005), Chem. Commun. (20):2552-2554). In other embodiments, the solvent used for the alkylation reaction is DMF or dichloroethane.

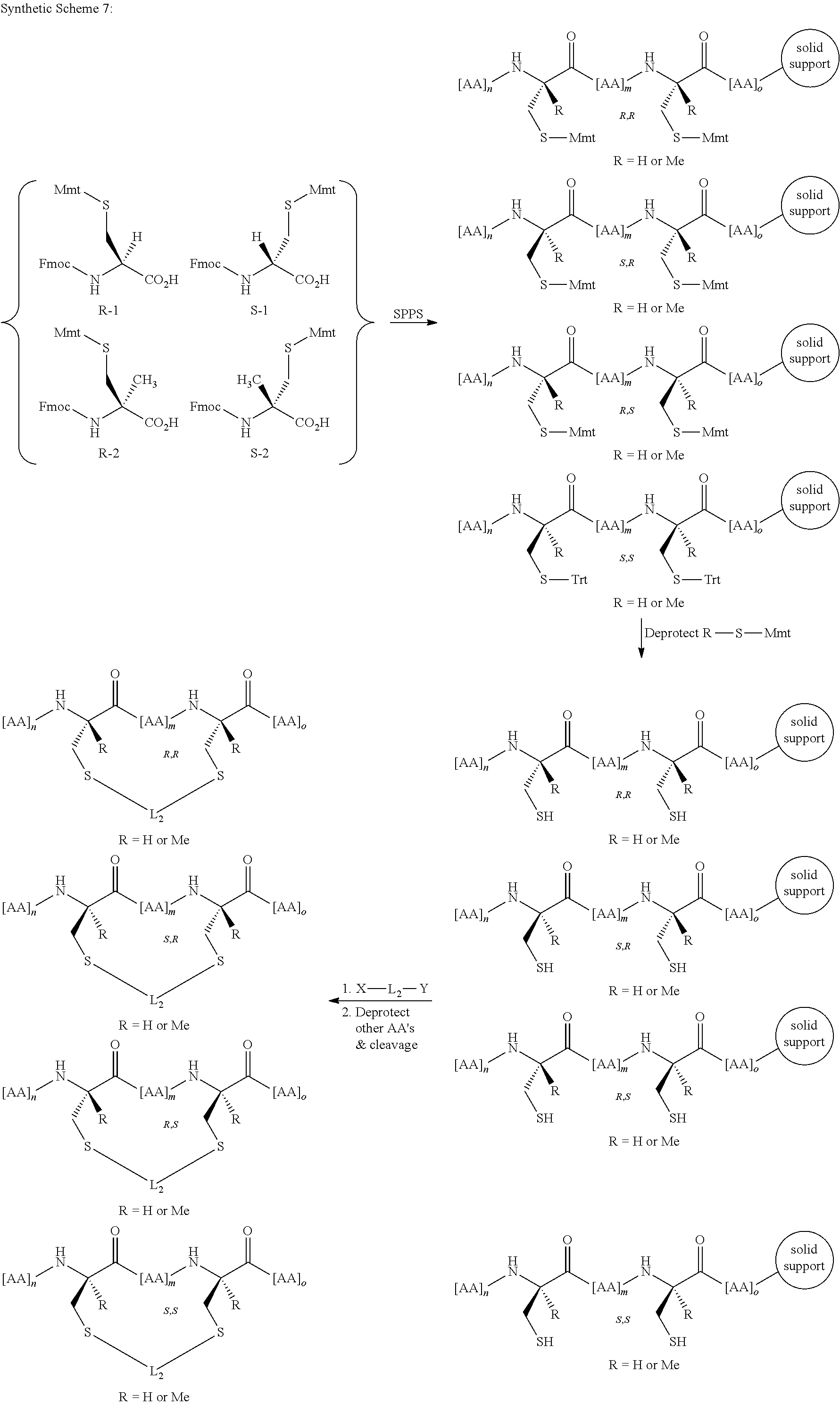

In Scheme 7, the precursor peptidomimetic contains two or more —SH moieties, of which two are specially protected to allow their selective deprotection and subsequent alkylation for macrocycle formation. The precursor peptidomimetic is synthesized by solid-phase peptide synthesis (SPPS) using commercially available N-α-Fmoc amino acids such as N-α-Fmoc-S-p-methoxytrityl-L-cysteine or N-α-Fmoc-S-p-methoxytrityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl monomers by known methods (Bioorganic Chemistry: Peptides and Proteins, Oxford University Press, New York: 1998, the entire contents of which are incorporated herein by reference). The Mmt protecting groups of the peptidomimetic precursor are then selectively cleaved by standard conditions (e.g., mild acid such as 1% TFA in DCM). The precursor peptidomimetic is then reacted on the resin with X-L₂-Y in an organic solution. For example, the reaction takes place in the presence of a hindered base such as diisopropylethylamine. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH or NH₃/DMF (Or et al. (1991), J. Org. Chem. 56:3146-3149). In other embodiments, the alkylation reaction is performed in DMF or dichloroethane. The peptidomimetic macrocycle is then deprotected and cleaved from the solid-phase resin by standard conditions (e.g., strong acid such as 95% TFA).

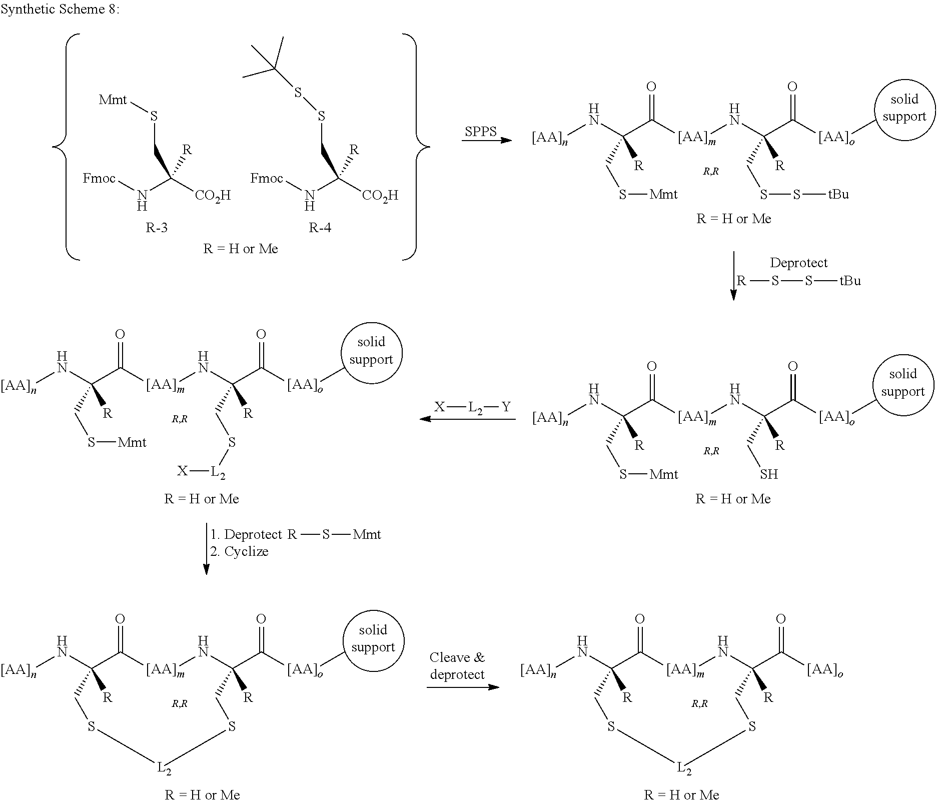

In Scheme 8, the peptidomimetic precursor contains two or more —SH moieties, of which two are specially protected to allow their selective deprotection and subsequent alkylation for macrocycle formation. The peptidomimetic precursor is synthesized by solid-phase peptide synthesis (SPPS) using commercially available N-α-Fmoc amino acids such as N-α-Fmoc-S-p-methoxytrityl-L-cysteine, N-α-Fmoc-S-p-methoxytrityl-D-cysteine, N-α-Fmoc-S—S-t-butyl-L-cysteine, and N-α-Fmoc-S—S-t-butyl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl or N-α-Fmoc-S-S-t-butyl monomers by known methods (Bioorganic Chemistry: Peptides and Proteins, Oxford University Press, New York: 1998, the entire contents of which are incorporated herein by reference). The S-S-tButyl protecting group of the peptidomimetic precursor is selectively cleaved by known conditions (e.g., 20% 2-mercaptoethanol in DMF, reference: Galande et al. (2005), J. Comb. Chem. 7:174-177). The precursor peptidomimetic is then reacted on the resin with a molar excess of X-L₂-Y in an organic solution. For example, the reaction takes place in the presence of a hindered base such as diisopropylethylamine. The Mmt protecting group of the peptidomimetic precursor is then selectively cleaved by standard conditions (e.g., mild acid such as 1% TFA in DCM). The peptidomimetic precursor is then cyclized on the resin by treatment with a hindered base in organic solutions. In some embodiments, the alkylation reaction is performed in organic solutions such as NH₃/MeOH or NH₃/DMF (Or et al. (1991), J. Org. Chem. 56:3146-3149). The peptidomimetic macrocycle is then deprotected and cleaved from the solid-phase resin by standard conditions (e.g., strong acid such as 95% TFA).

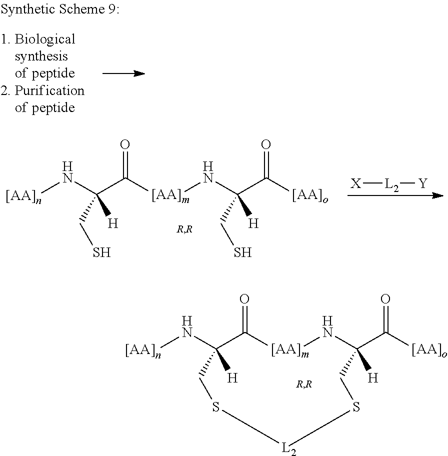

In Scheme 9, the peptidomimetic precursor contains two L-cysteine moieties. The peptidomimetic precursor is synthesized by known biological expression systems in living cells or by known in vitro, cell-free, expression methods. The precursor peptidomimetic is reacted as a crude mixture or is purified prior to reaction with X-L2-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6 M guanidinium HCl, pH 8 (Brunel et al. (2005), Chem. Commun. (20):2552-2554). In other embodiments, the alkylation is performed in DMF or dichloroethane. In another embodiment, the alkylation is performed in non-denaturing aqueous solutions, and in yet another embodiment the alkylation is performed under conditions that favor α-helical structure formation. In yet another embodiment, the alkylation is performed under conditions that favor the binding of the precursor peptidomimetic to another protein, so as to induce the formation of the bound α-helical conformation during the alkylation.

Various embodiments for X and Y are envisioned which are suitable for reacting with thiol groups. In general, each X or Y is independently selected from the general category shown in Table 5. For example, X and Y are halides such as —Cl, —Br or —I. Any of the macrocycle-forming linkers described herein may be used in any combination with any of the sequences shown and also with any of the R— substituents indicated herein.

TABLE 5
Examples of Reactive Groups Capable of Reacting
with Thiol Groups and Resulting Linkages

| X or Y | Resulting Covalent Linkage |
|---|---|
| acrylamide | Thioether |
| halide (e.g. alkyl or aryl halide) | Thioether |
| sulfonate | Thioether |
| aziridine | Thioether |
| epoxide | Thioether |
| haloacetamide | Thioether |
| maleimide | Thioether |
| sulfonate ester | Thioether |

The present invention contemplates the use of both naturally-occurring and non-naturally-occurring amino acids and amino acid analogs in the synthesis of the peptidomimetic macrocycles disclosed. Any amino acid or amino acid analog amenable to the synthetic methods employed for the synthesis of stable bis-sulfhydryl containing peptidomimetic macrocycles can be used in the present invention. For example, cysteine is contemplated as a useful amino acid in the present invention. However, sulfur-containing amino acids other than cysteine that contain a different amino acid side chain are also useful. For example, cysteine contains one methylene unit between the α-carbon of the amino acid and the terminal —SH of the amino acid side chain. The invention also contemplates the use of amino acids with multiple methylene units between the α-carbon and the terminal —SH. Non-limiting examples include α-methyl-L-homocysteine and α-methyl-D-homocysteine. In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-cysteine and α-methyl-D-cysteine.

The invention includes macrocycles in which macrocycle-forming linkers are used to link two or more —SH moieties in the peptidomimetic precursors to form the peptidomimetic macrocycles disclosed herein. As described above, the macrocycle-forming linkers impart conformational rigidity, increased metabolic stability and/or increased cell penetrability. Furthermore, in some embodiments, the macrocycle-forming linkages stabilize the α-helical secondary structure of the peptidomimetic macrocyles. The macrocycle-forming linkers are of the formula X-L₂-Y, wherein both X and Y are the same or different moieties, as defined above. Both X and Y have the chemical characteristics that allow one macrocycle-forming linker-L₂- to bis alkylate the bis-sulfhydryl containing peptidomimetic precursor. As defined above, the linker-L₂- includes alkylene, alkenylene, alkynylene, heteroalkylene, cycloalkylene, heterocycloalkylene, cycloarylene, or heterocycloarylene, or —R₄—K—R₄—, all of which can be optionally substituted with an R₅ group, as defined above. Furthermore, one to three carbon atoms within the macrocycle-forming linkers-L₂-, other than the carbons attached to the —SH of the sulfhydryl containing amino acid, are optionally substituted with a heteroatom such as N, S or O.

The L₂ component of the macrocycle-forming linker X-L₂-Y may be varied in length depending on, among other things, the distance between the positions of the two amino acid analogs used to form the peptidomimetic macrocycle. Furthermore, as the lengths of L₁ and/or L₃ components of the macrocycle-forming linker are varied, the length of L₂ can also be varied in order to create a linker of appropriate overall length for forming a stable peptidomimetic macrocycle. For example, if the amino acid analogs used are varied by adding an additional methylene unit to each of L₁ and L₃, the length of L₂ are decreased in length by the equivalent of approximately two methylene units to compensate for the increased lengths of L₁ and L₃.

In some embodiments, L₂ is an alkylene group of the formula —(CH₂)ₙ—, where n is an integer between about 1 and about 15. For example, n is 1, 2, 3, 4, 5, 6, 7, 8, 9 or 10. In other embodiments, L₂ is an alkenylene group. In still other embodiments, L₂ is an aryl group.

Table 6 shows additional embodiments of X-L₂-Y groups. Each X and Y in this table, is, for example, independently Cl—, Br— or I—.

TABLE 6

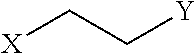

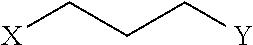

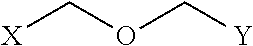

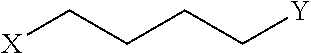

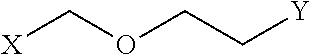

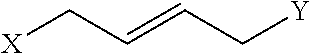

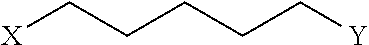

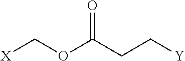

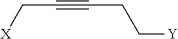

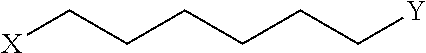

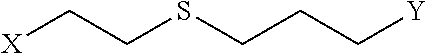

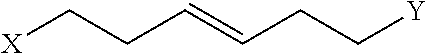

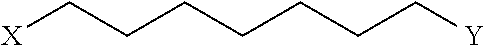

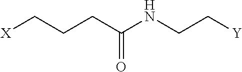

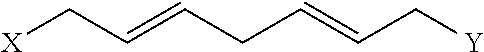

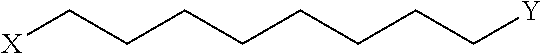

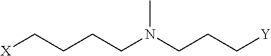

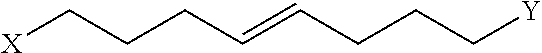

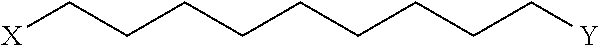

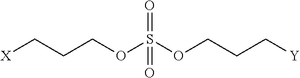

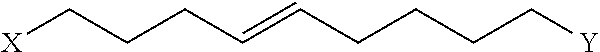

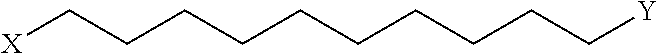

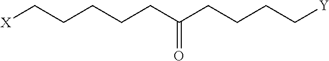

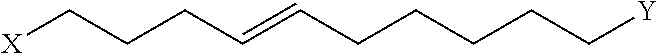

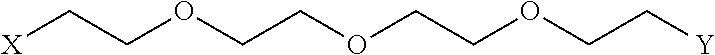

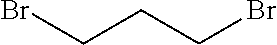

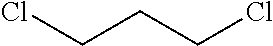

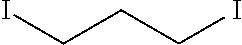

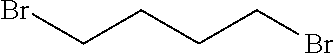

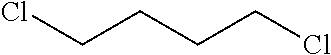

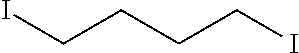

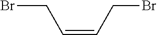

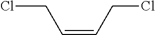

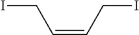

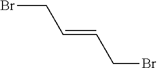

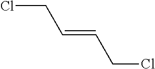

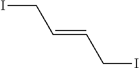

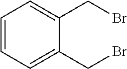

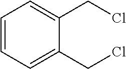

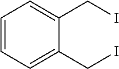

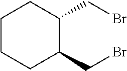

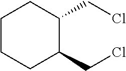

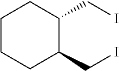

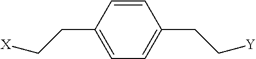

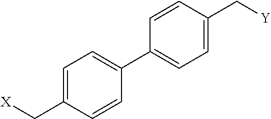

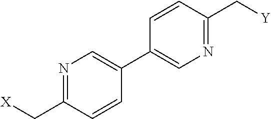

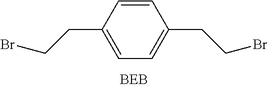 [SMILES: BrCCc1ccc(CCBr)cc1]

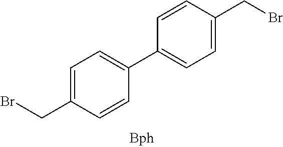 [SMILES: BrCc1ccc(-c2ccc(CBr)cc2)cc1]

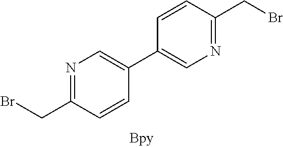 [SMILES: BrCc1ccc(-c2ccc(CBr)nc2)cn1]

Additional methods of forming peptidomimetic macrocycles which are envisioned as suitable include those disclosed by Mustapa, M. Firouz Mohd et al., J. Org. Chem (2003), 68, pp. 8193-8198; Yang, Bin et al. Bioorg Med. Chem. Lett. (2004), 14, pp. 1403-1406; U.S. Pat. No. 5,364,851; U.S. Pat. No. 5,446,128; U.S. Pat. No. 5,824,483; U.S. Pat. No. 6,713,280; and U.S. Pat. No. 7,202,332. In such embodiments, aminoacid precursors are used containing an additional substituent R— at the alpha position. Such aminoacids are incorporated into the macrocycle precursor at the desired positions, which can be at the positions where the crosslinker is substituted or, alternatively, elsewhere in the sequence of the macrocycle precursor. Cyclization of the precursor is then affected according to the indicated method.

In some embodiments, the —NH moiety of the amino acid is protected using a protecting group, including without limitation -Fmoc and -Boc. In other embodiments, the amino acid is not protected prior to synthesis of the peptidomimetic macrocycle.

### Assays

The properties of the peptidomimetic macrocycles of the invention are assayed, for example, by using the methods described below. In some embodiments, a peptidomimetic macrocycle of the invention has improved biological properties relative to a corresponding polypeptide lacking the substituents described herein.

### Assay to Determine α-Helicity

In solution, the secondary structure of polypeptides with α-helical domains will reach a dynamic equilibrium between random coil structures and α-helical structures, often expressed as a “percent helicity”. Thus, for example, alpha-helical domains are predominantly random coils in solution, with α-helical content usually under 25%. Peptidomimetic macrocycles with optimized linkers, on the other hand, possess, for example, an alpha-helicity that is at least two-fold greater than that of a corresponding uncrosslinked polypeptide. In some embodiments, macrocycles will possess an alpha-helicity of greater than 50%. To assay the helicity of peptidomimetic macrocyles, the compounds are dissolved in an aqueous solution (e.g. 50 mM potassium phosphate solution at pH 7, or distilled H₂O, to concentrations of 25-50 μM). Circular dichroism (CD) spectra are obtained on a spectropolarimeter (e.g., Jasco J-710) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g. [Φ]222obs) by the reported value for a model helical decapeptide (Yang et al. (1986), Methods Enzymol. 130:208)).

### Assay to Determine Melting Temperature (Tₘ)

A peptidomimetic macrocycle of the invention comprising a secondary structure such as an α-helix exhibits, for example, a higher melting temperature than a corresponding uncrosslinked polypeptide. Typically peptidomimetic macrocycles of the invention exhibit Tₘ of >60° C. representing a highly stable structure in aqueous solutions. To assay the effect of macrocycle formation on melting temperature, peptidomimetic macrocycles or unmodified peptides are dissolved in distilled H₂O (e.g. at a final concentration of 50 μM) and the Tₘ is determined by measuring the change in ellipticity over a temperature range (e.g. 4 to 95° C.) on a spectropolarimeter (e.g., Jasco J-710) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

### Protease Resistance Assay

The amide bond of the peptide backbone is susceptible to hydrolysis by proteases, thereby rendering peptidic compounds vulnerable to rapid degradation in vivo. Peptide helix formation, however, typically buries the amide backbone and therefore may shield it from proteolytic cleavage. The peptidomimetic macrocycles of the present invention may be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln [S] versus time (k=−1×slope).

### Ex Vivo Stability Assay

Peptidomimetic macrocycles with optimized linkers possess, for example, an ex vivo half-life that is at least two-fold greater than that of a corresponding uncrosslinked polypeptide, and possess an ex vivo half-life of 12 hours or more. For ex vivo serum stability studies, a variety of assays may be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure may be used: The samples are extracted by transferring 100 μl of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis.

### In Vitro Binding Assays

To assess the binding and affinity of peptidomimetic macrocycles and peptidomimetic precursors to acceptor proteins, a fluorescence polarization assay (FPA) issued, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

For example, fluoresceinated peptidomimetic macrocycles (25 nM) are incubated with the acceptor protein (25-1000 nM) in binding buffer (140 mM NaCl, 50 mM Tris-HCl, pH 7.4) for 30 minutes at room temperature. Binding activity is measured, for example, by fluorescence polarization on a luminescence spectrophotometer (e.g. Perkin-Elmer LS50B). Kd values may be determined by nonlinear regression analysis using, for example, Graphpad Prism software (GraphPad Software, Inc., San Diego, Calif.). A peptidomimetic macrocycle of the invention shows, in some instances, similar or lower Kd than a corresponding uncrosslinked polypeptide.

### In Vitro Displacement Assays to Characterize Antagonists of Peptide-Protein Interactions

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCl, pH 7.4) for 30 minutes at room temperature. Antagonist binding activity is measured, for example, by fluorescence polarization on a luminescence spectrophotometer (e.g. Perkin-Elmer LS50B). Kd values may be determined by nonlinear regression analysis using, for example, Graphpad Prism software (GraphPad Software, Inc., San Diego, Calif.).

Any class of molecule, such as small organic molecules, peptides, oligonucleotides or proteins can be examined as putative antagonists in this assay.

### Assay for Protein-Ligand Binding by Affinity Selection-Mass Spectrometry

To assess the binding and affinity of test compounds for proteins, an affinity-selection mass spectrometry assay is used, for example. Protein-ligand binding experiments are conducted according to the following representative procedure outlined for a system-wide control experiment using 1 μM peptidomimetic macrocycle plus 5 μM hMDM2. A 1 μL DMSO aliquot of a 40 μM stock solution of peptidomimetic macrocycle is dissolved in 19 μL of PBS (Phosphate-buffered saline: 50 mM, pH 7.5 Phosphate buffer containing 150 mM NaCl). The resulting solution is mixed by repeated pipetting and clarified by centrifugation at 10 000 g for 10 min. To a 4 μL aliquot of the resulting supernatant is added 4 μL of 10 μM hMDM2 in PBS. Each 8.0 μL experimental sample thus contains 40 pmol (1.5 μg) of protein at 5.0 μM concentration in PBS plus 1 μM peptidomimetic macrocycle and 2.5% DMSO. Duplicate samples thus prepared for each concentration point are incubated for 60 min at room temperature, and then chilled to 4° C. prior to size-exclusion chromatography-LC-MS analysis of 5.0 μL injections. Samples containing a target protein, protein-ligand complexes, and unbound compounds are injected onto an SEC column, where the complexes are separated from non-binding component by a rapid SEC step. The SEC column eluate is monitored using UV detectors to confirm that the early-eluting protein fraction, which elutes in the void volume of the SEC column, is well resolved from unbound components that are retained on the column. After the peak containing the protein and protein-ligand complexes elutes from the primary UV detector, it enters a sample loop where it is excised from the flow stream of the SEC stage and transferred directly to the LC-MS via a valving mechanism. The (M+3H)³⁺ ion of the peptidomimetic macrocycle is observed by ESI-MS at the expected m/z, confirming the detection of the protein-ligand complex.

### Assay for Protein-Ligand Kd Titration Experiments

To assess the binding and affinity of test compounds for proteins, a protein-ligand K_{d} titration experiment is performed, for example. Protein-ligand K_{d} titrations experiments are conducted as follows: 2 μL DMSO aliquots of a serially diluted stock solution of titrant peptidomimetic macrocycle (5, 2.5, . . . , 0.098 mM) are prepared then dissolved in 38 μL of PBS. The resulting solutions are mixed by repeated pipetting and clarified by centrifugation at 10 000 g for 10 min. To 4.0 μL aliquots of the resulting supernatants is added 4.0 μL of 10 μM hMDM2 in PBS. Each 8.0 μL experimental sample thus contains 40 pmol (1.5 μg) of protein at 5.0 μM concentration in PBS, varying concentrations (125, 62.5, . . . , 0.24 μM) of the titrant peptide, and 2.5% DMSO. Duplicate samples thus prepared for each concentration point are incubated at room temperature for 30 min, then chilled to 4° C. prior to SEC-LC-MS analysis of 2.0 μL injections. The (M+H)¹⁺, (M+2H)²⁺, (M+3H)³⁺, and/or (M+Na)¹⁺ ion is observed by ESI-MS; extracted ion chromatograms are quantified, then fit to equations to derive the binding affinity K_{d} as described in “A General Technique to Rank Protein-Ligand Binding Affinities and Determine Allosteric vs. Direct Binding Site Competition in Compound Mixtures.” Annis, D. A.; Nazef, N.; Chuang, C. C.; Scott, M. P.; Nash, H. M. J. Am. Chem. Soc. 2004, 126, 15495-15503; also in “ALIS: An Affinity Selection-Mass Spectrometry System for the Discovery and Characterization of Protein-Ligand Interactions” D. A. Annis, C.-C. Chuang, and N. Nazef. In Mass Spectrometry in Medicinal Chemistry. Edited by Wanner K, Höfner G: Wiley-VCH; 2007:121-184. Mannhold R, Kubinyi H, Folkers G (Series Editors): Methods and Principles in Medicinal Chemistry.

### Assay for Competitive Binding Experiments by Affinity Selection-Mass Spectrometry

To determine the ability of test compounds to bind competitively to proteins, an affinity selection mass spectrometry assay is performed, for example. A mixture of ligands at 40 μM per component is prepared by combining 2 μL aliquots of 400 μM stocks of each of the three compounds with 14 μL of DMSO. Then, 1 μL aliquots of this 40 μM per component mixture are combined with 1 μL DMSO aliquots of a serially diluted stock solution of titrant peptidomimetic macrocycle (10, 5, 2.5, . . . , 0.078 mM). These 2 μL samples are dissolved in 38 μL of PBS. The resulting solutions were mixed by repeated pipetting and clarified by centrifugation at 10 000 g for 10 min. To 4.0 μL aliquots of the resulting supernatants is added 4.0 μL of 10 μM hMDM2 in PBS. Each 8.0 μL experimental sample thus contains 40 pmol (1.5 μg) of protein at 5.0 μM concentration in PBS plus 0.5 μM ligand, 2.5% DMSO, and varying concentrations (125, 62.5, . . . , 0.98 μM) of the titrant peptidomimetic macrocycle. Duplicate samples thus prepared for each concentration point are incubated at room temperature for 60 min, then chilled to 4° C. prior to SEC-LC-MS analysis of 2.0 μL injections. Additional details on these and other methods are provided in “A General Technique to Rank Protein-Ligand Binding Affinities and Determine Allosteric vs. Direct Binding Site Competition in Compound Mixtures.” Annis, D. A.; Nazef, N.; Chuang, C. C.; Scott, M. P.; Nash, H. M. J. Am. Chem. Soc. 2004, 126, 15495-15503; also in “ALIS: An Affinity Selection-Mass Spectrometry System for the Discovery and Characterization of Protein-Ligand Interactions” D. A. Annis, C.-C. Chuang, and N. Nazef. In Mass Spectrometry in Medicinal Chemistry. Edited by Wanner K, Höfner G: Wiley-VCH; 2007:121-184. Mannhold R, Kubinyi H, Folkers G (Series Editors): Methods and Principles in Medicinal Chemistry.

### Binding Assays in Intact Cells

It is possible to measure binding of peptides or peptidomimetic macrocycles to their natural acceptors in intact cells by immunoprecipitation experiments. For example, intact cells are incubated with fluoresceinated (FITC-labeled) compounds for 4 hrs in the absence of serum, followed by serum replacement and further incubation that ranges from 4-18 hrs. Cells are then pelleted and incubated in lysis buffer (50 mM Tris [pH 7.6], 150 mM NaCl, 1% CHAPS and protease inhibitor cocktail) for 10 minutes at 4° C. Extracts are centrifuged at 14,000 rpm for 15 minutes and supernatants collected and incubated with 10 μl goat anti-FITC antibody for 2 hrs, rotating at 4° C. followed by further 2 hrs incubation at 4° C. with protein A/G Sepharose (50 μl of 50% bead slurry). After quick centrifugation, the pellets are washed in lysis buffer containing increasing salt concentration (e.g., 150, 300, 500 mM). The beads are then re-equilibrated at 150 mM NaCl before addition of SDS-containing sample buffer and boiling. After centrifugation, the supernatants are optionally electrophoresed using 4%-12% gradient Bis-Tris gels followed by transfer into Immobilon-P membranes. After blocking, blots are optionally incubated with an antibody that detects FITC and also with one or more antibodies that detect proteins that bind to the peptidomimetic macrocycle.

### Cellular Penetrability Assays

A peptidomimetic macrocycle is, for example, more cell penetrable compared to a corresponding uncrosslinked macrocycle. Peptidomimetic macrocycles with optimized linkers possess, for example, cell penetrability that is at least two-fold greater than a corresponding uncrosslinked macrocycle, and often 20% or more of the applied peptidomimetic macrocycle will be observed to have penetrated the cell after 4 hours. To measure the cell penetrability of peptidomimetic macrocycles and corresponding uncrosslinked macrocycle, intact cells are incubated with fluoresceinated peptidomimetic macrocycles or corresponding uncrosslinked macrocycle (10 μM) for 4 hrs in serum free media at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan® HCS Reader.

### Cellular Efficacy Assays

The efficacy of certain peptidomimetic macrocycles is determined, for example, in cell-based killing assays using a variety of tumorigenic and non-tumorigenic cell lines and primary cells derived from human or mouse cell populations. Cell viability is monitored, for example, over 24-96 hrs of incubation with peptidomimetic macrocycles (0.5 to 50 μM) to identify those that kill at EC₅₀<10 μM. Several standard assays that measure cell viability are commercially available and are optionally used to assess the efficacy of the peptidomimetic macrocycles. In addition, assays that measure Annexin V and caspase activation are optionally used to assess whether the peptidomimetic macrocycles kill cells by activating the apoptotic machinery. For example, the Cell Titer-glo assay is used which determines cell viability as a function of intracellular ATP concentration.

### In Vivo Stability Assay

To investigate the in vivo stability of the peptidomimetic macrocycles, the compounds are, for example, administered to mice and/or rats by IV, IP, PO or inhalation routes at concentrations ranging from 0.1 to 50 mg/kg and blood specimens withdrawn at 0′, 5′, 15′, 30′, 1 hr, 4 hrs, 8 hrs and 24 hours post-injection. Levels of intact compound in 25 μL of fresh serum are then measured by LC-MS/MS as above.

### In Vivo Efficacy in Animal Models

To determine the anti-oncogenic activity of peptidomimetic macrocycles of the invention in vivo, the compounds are, for example, given alone (IP, IV, PO, by inhalation or nasal routes) or in combination with sub-optimal doses of relevant chemotherapy (e.g., cyclophosphamide, doxorubicin, etoposide). In one example, 5×10⁶ RS4;11 cells (established from the bone marrow of a patient with acute lymphoblastic leukemia) that stably express luciferase are injected by tail vein in NOD-SCID mice 3 hrs after they have been subjected to total body irradiation. If left untreated, this form of leukemia is fatal in 3 weeks in this model. The leukemia is readily monitored, for example, by injecting the mice with D-luciferin (60 mg/kg) and imaging the anesthetized animals (e.g., Xenogen In Vivo Imaging System, Caliper Life Sciences, Hopkinton, Mass.). Total body bioluminescence is quantified by integration of photonic flux (photons/sec) by Living Image Software (Caliper Life Sciences, Hopkinton, Mass.). Peptidomimetic macrocycles alone or in combination with sub-optimal doses of relevant chemotherapeutics agents are, for example, administered to leukemic mice (10 days after injection/day 1 of experiment, in bioluminescence range of 14-16) by tail vein or IP routes at doses ranging from 0.1 mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma that stably expresses luciferase. These in vivo tests optionally generate preliminary pharmacokinetic, pharmacodynamic and toxicology data.

### Clinical Trials

To determine the suitability of the peptidomimetic macrocycles of the invention for treatment of humans, clinical trials are performed. For example, patients diagnosed with cancer and in need of treatment are selected and separated in treatment and one or more control groups, wherein the treatment group is administered a peptidomimetic macrocycle of the invention, while the control groups receive a placebo or a known anti-cancer drug. The treatment safety and efficacy of the peptidomimetic macrocycles of the invention can thus be evaluated by performing comparisons of the patient groups with respect to factors such as survival and quality-of-life. In this example, the patient group treated with a peptidomimetic macrocyle show improved long-term survival compared to a patient control group treated with a placebo.

### Pharmaceutical Compositions and Routes of Administration

The peptidomimetic macrocycles of the invention also include pharmaceutically acceptable derivatives or prodrugs thereof. A “pharmaceutically acceptable derivative” means any pharmaceutically acceptable salt, ester, salt of an ester, pro-drug or other derivative of a compound of this invention which, upon administration to a recipient, is capable of providing (directly or indirectly) a compound of this invention. Particularly favored pharmaceutically acceptable derivatives are those that increase the bioavailability of the compounds of the invention when administered to a mammal (e.g., by increasing absorption into the blood of an orally administered compound) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

In some embodiments, the peptidomimetic macrocycles of the invention are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

When the compositions of this invention comprise a combination of a peptidomimetic macrocycle and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from the compounds of this invention. Alternatively, those agents are part of a single dosage form, mixed together with the compounds of this invention in a single composition.

### Methods of Use

In one aspect, the present invention provides novel peptidomimetic macrocycles that are useful in competitive binding assays to identify agents which bind to the natural ligand(s) of the proteins or peptides upon which the peptidomimetic macrocycles are modeled. For example, in the p53/HDMX system, labeled peptidomimetic macrocycles based on p53 can be used in a HDMX binding assay along with small molecules that competitively bind to HDMX. Competitive binding studies allow for rapid in vitro evaluation and determination of drug candidates specific for the p53/HDMX system. Such binding studies may be performed with any of the peptidomimetic macrocycles disclosed herein and their binding partners.

Further provided are methods for the generation of antibodies against the peptidomimetic macrocycles. In some embodiments, these antibodies specifically bind both the peptidomimetic macrocycle and the precursor peptides, such as p53, to which the peptidomimetic macrocycles are related. Such antibodies, for example, disrupt the native protein-protein interaction, for example, binding between p53 and HDMX.

In other aspects, the present invention provides for both prophylactic and therapeutic methods of treating a subject at risk of (or susceptible to) a disorder or having a disorder associated with aberrant (e.g., insufficient or excessive) expression or activity of the molecules including p53, HDM2 or HDMX.

In another embodiment, a disorder is caused, at least in part, by an abnormal level of p53 or HDM2 or HDMX, (e.g., over or under expression), or by the presence of p53 or HDM2 or HDMX exhibiting abnormal activity. As such, the reduction in the level and/or activity of p53 or HDM2 or HDMX, or the enhancement of the level and/or activity of p53 or HDM2 or HDMX, by peptidomimetic macrocycles derived from p53, is used, for example, to ameliorate or reduce the adverse symptoms of the disorder.

In another aspect, the present invention provides methods for treating or preventing a disease including hyperproliferative disease and inflammatory disorder by interfering with the interaction or binding between binding partners, for example, between p53 and HDM2 or p53 and HDMX. These methods comprise administering an effective amount of a compound of the invention to a warm blooded animal, including a human. In some embodiments, the administration of the compounds of the present invention induces cell growth arrest or apoptosis.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

In some embodiments, the peptidomimetics macrocycles of the invention is used to treat, prevent, and/or diagnose cancers and neoplastic conditions. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, the peptidomimetics macrocycles are novel therapeutic agents for controlling breast cancer, ovarian cancer, colon cancer, lung cancer, metastasis of such cancers and the like.

Examples of cancers or neoplastic conditions include, but are not limited to, a fibrosarcoma, myosarcoma, liposarcoma, chondrosarcoma, osteogenic sarcoma, chordoma, angio sarcoma, endotheliosarcoma, lymphangiosarcoma, lymphangioendothelio sarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, lung cancer, breast cancer, skin cancer, melanoma, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, leukemia, lymphoma, or Kaposi sarcoma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991), Crit Rev. Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the skin include, but are not limited to proliferative skin disease such as melanomas, including mucosal melanoma, superficial spreading melanoma, nodular melanoma, lentigo (e.g. lentigo maligna, lentigo maligna melanoma, or acral lentiginous melanoma), amelanotic melanoma, desmoplastic melanoma, melanoma with features of a Spitz nevus, melanoma with small nevus-like cells, polypoid melanoma, and soft-tissue melanoma; basal cell carcinomas including micronodular basal cell carcinoma, superficial basal cell carcinoma, nodular basal cell carcinoma (rodent ulcer), cystic basal cell carcinoma, cicatricial basal cell carcinoma, pigmented basal cell carcinoma, aberrant basal cell carcinoma, infiltrative basal cell carcinoma, nevoid basal cell carcinoma syndrome, polypoid basal cell carcinoma, pore-like basal cell carcinoma, and fibroepithelioma of Pinkus; squamus cell carcinomas including acanthoma (large cell acanthoma), adenoid squamous cell carcinoma, basaloid squamous cell carcinoma, clear cell squamous cell carcinoma, signet-ring cell squamous cell carcinoma, spindle cell squamous cell carcinoma, Marjolin's ulcer, erythroplasia of Queyrat, and Bowen's disease; or other skin or subcutaneous tumors.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

In other or further embodiments, the peptidomimetics macrocycles described herein are used to treat, prevent or diagnose conditions characterized by overactive cell death or cellular death due to physiologic insult, etc. Some examples of conditions characterized by premature or unwanted cell death are or alternatively unwanted or excessive cellular proliferation include, but are not limited to hypocellular/hypoplastic, acellular/aplastic, or hypercellular/hyperplastic conditions. Some examples include hematologic disorders including but not limited to fanconi anemia, aplastic anemia, thalaessemia, congenital neutropenia, and myelodysplasia.

In other or further embodiments, the peptidomimetics macrocycles of the invention that act to decrease apoptosis are used to treat disorders associated with an undesirable level of cell death. Thus, in some embodiments, the anti-apoptotic peptidomimetics macrocycles of the invention are used to treat disorders such as those that lead to cell death associated with viral infection, e.g., infection associated with infection with human immunodeficiency virus (HIV). A wide variety of neurological diseases are characterized by the gradual loss of specific sets of neurons. One example is Alzheimer's disease (AD). Alzheimer's disease is characterized by loss of neurons and synapses in the cerebral cortex and certain subcortical regions. This loss results in gross atrophy of the affected regions. Both amyloid plaques and neurofibrillary tangles are visible in brains of those afflicted by AD. Alzheimer's disease has been identified as a protein misfolding disease, due to the accumulation of abnormally folded A-beta and tau proteins in the brain. Plaques are made up of β-amyloid. β-amyloid is a fragment from a larger protein called amyloid precursor protein (APP). APP is critical to neuron growth, survival and post-injury repair. In AD, an unknown process causes APP to be cleaved into smaller fragments by enzymes through proteolysis. One of these fragments is fibrils of β-amyloid, which form clumps that deposit outside neurons in dense formations known as senile plaques. Plaques continue to grow into insoluble twisted fibers within the nerve cell, often called tangles. Disruption of the interaction between β-amyloid and its native receptor is therefore important in the treatment of AD. The anti-apoptotic peptidomimetics macrocycles of the invention are used, in some embodiments, in the treatment of AD and other neurological disorders associated with cell apoptosis. Such neurological disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis. In other or further embodiments, the anti-apoptotic peptidomimetics macrocycles of the invention are used to treat all such disorders associated with undesirable cell death.

Some examples of neurologic disorders that are treated with the peptidomimetics macrocycles described herein include but are not limited to Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann Straussler-Scheinker Syndrome, Bovine Spongiform Encephalitis, a prion-mediated disease, and Huntington's Disease.

In another embodiment, the peptidomimetics macrocycles described herein are used to treat, prevent or diagnose inflammatory disorders. Numerous types of inflammatory disorders exist. Certain inflammatory diseases are associated with the immune system, for example, autoimmune diseases. Autoimmune diseases arise from an overactive immune response of the body against substances and tissues normally present in the body, i.e. self antigens. In other words, the immune system attacks its own cells. Autoimmune diseases are a major cause of immune-mediated diseases. Rheumatoid arthritis is an example of an autoimmune disease, in which the immune system attacks the joints, where it causes inflammation (i.e. arthritis) and destruction. It can also damage some organs, such as the lungs and skin. Rheumatoid arthritis can lead to substantial loss of functioning and mobility. Rheumatoid arthritis is diagnosed with blood tests especially the rheumatoid factor test. Some examples of autoimmune diseases that are treated with the peptidomimetics macrocycles described herein include, but are not limited to, acute disseminated encephalomyelitis (ADEM), Addison's disease, ankylosing spondylitis, antiphospholipid antibody syndrome (APS), autoimmune hemolytic anemia, autoimmune hepatitis, autoimmune inner ear disease, Bechet's disease, bullous pemphigoid, coeliac disease, Chagas disease, Churg-Strauss syndrome, chronic obstructive pulmonary disease (COPD), Crohn's disease, dermatomyositis, diabetes mellitus type 1, endometriosis, Goodpasture's syndrome, Graves' disease, Guillain-Barré syndrome (GBS), Hashimoto's disease, Hidradenitis suppurativa, idiopathic thrombocytopenic purpura, inflammatory bowl disease (IBD), interstitial cystitis, lupus erythematosus, morphea, multiple sclerosis, myasthenia gravis, narcolepsy, neuromyotonia, pemphigus vulgaris, pernicious anaemia, Polymyositis, polymyalgia rheumatica, primary biliary cirrhosis, psoriasis, rheumatoid arthritis, schizophrenia, scleroderma, Sjögren's syndrome, temporal arteritis (also known as “giant cell arteritis”), Takayasu's arteritis, Vasculitis, Vitiligo, and Wegener's granulomatosis.

Some examples of other types of inflammatory disorders that are treated with the peptidomimetics macrocycles described herein include, but are not limited to, allergy including allergic rhinitis/sinusitis, skin allergies (urticaria/hives, angioedema, atopic dermatitis), food allergies, drug allergies, insect allergies, and rare allergic disorders such as mastocytosis, asthma, arthritis including osteoarthritis, rheumatoid arthritis, and spondyloarthropathies, primary angitis of the CNS, sarcoidosis, organ transplant rejection, fibromyalgia, fibrosis, pancreatitis, and pelvic inflammatory disease.

Examples of cardiovascular disorders (e.g., inflammatory disorders) that are treated or prevented with the peptidomimetics macrocycles of the invention include, but are not limited to, aortic valve stenosis, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve or other implantable devices. Preferred cardiovascular disorders include atherosclerosis, myocardial infarction, aneurism, and stroke.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Formulation and Administration

### Mode of Administration

An effective amount of a peptidomimetic macrocycles of the disclosure can be administered in either single or multiple doses by any of the accepted modes of administration. In some embodiments, the peptidomimetic macrocycles of the disclosure are administered parenterally, for example, by subcutaneous, intramuscular, intrathecal, intravenous or epidural injection. For example, the peptidomimetic macrocycle is administered intravenously, intraarterially, subcutaneously or by infusion. In some examples, the peptidomimetic macrocycle is administered intravenously. In some examples, the peptidomimetic macrocycle is administered intraarterially.

Regardless of the route of administration selected, the peptidomimetic macrocycles of the present disclosure, and/or the pharmaceutical compositions of the present disclosure, are formulated into pharmaceutically-acceptable dosage forms. The peptidomimetic macrocycles according to the disclosure can be formulated for administration in any convenient way for use in human or veterinary medicine, by analogy with other pharmaceuticals.

In one aspect, the disclosure provides pharmaceutical formulation comprising a therapeutically-effective amount of one or more of the peptidomimetic macrocycles described above, formulated together with one or more pharmaceutically acceptable carriers (additives) and/or diluents. In one embodiment, one or more of the peptidomimetic macrocycles described herein are formulated for parenteral administration for parenteral administration, one or more peptidomimetic macrocycles disclosed herein can be formulated as aqueous or nonaqueous solutions, dispersions, suspensions or emulsions or sterile powders which can be reconstituted into sterile injectable solutions or dispersions just prior to use. Such formulations can comprise sugars, alcohols, antioxidants, buffers, bacteriostats, solutes which render the formulation isotonic with the blood of the intended recipient or suspending or thickening agents. These compositions can also contain adjuvants such as preservatives, wetting agents, emulsifying agents and dispersing agents. Prevention of the action of microorganisms upon the subject compounds can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin. If desired the formulation can be diluted prior to use with, for example, an isotonic saline solution or a dextrose solution. In some examples, the peptidomimetic macrocycle is formulated as an aqueous solution and is administered intravenously.

### Amount and Frequency of Administration

Dosing can be determined using various techniques. The selected dosage level can depend upon a variety of factors including the activity of the particular peptidomimetic macrocycle employed, the route of administration, the time of administration, the rate of excretion or metabolism of the particular peptidomimetic macrocycle being employed, the duration of the treatment, other drugs, compounds and/or materials used in combination with the particular peptidomimetic macrocycle employed, the age, sex, weight, condition, general health and prior medical history of the patient being treated, and like factors well known in the medical arts. The dosage values can also vary with the severity of the condition to be alleviated. For any particular subject, specific dosage regimens can be adjusted over time according to the individual need and the professional judgment of the person administering or supervising the administration of the compositions.

A physician or veterinarian can prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In some embodiments, a suitable daily dose of a peptidomimetic macrocycle of the disclosure can be that amount of the peptidomimetic macrocycle which is the lowest dose effective to produce a therapeutic effect. Such an effective dose will generally depend upon the factors described above. The precise time of administration and amount of any particular peptidomimetic macrocycle that will yield the most effective treatment in a given patient will depend upon the activity, pharmacokinetics, and bioavailability of a particular peptidomimetic macrocycle, physiological condition of the patient (including age, sex, disease type and stage, general physical condition, responsiveness to a given dosage and type of medication), route of administration, and the like.

Dosage can be based on the amount of the peptidomimetic macrocycle per kg body weight of the patient. Alternatively, the dosage of the subject disclosure can be determined by reference to the plasma concentrations of the peptidomimetic macrocycle. For example, the maximum plasma concentration (Cmax) and the area under the plasma concentration-time curve from time 0 to infinity (AUC) can be used.

In some embodiments, the subject is a human subject and the amount of the peptidomimetic macrocycle administered is 0.01-100 mg per kilogram body weight of the human subject. For example, in various examples, the amount of the peptidomimetic macrocycle administered is about 0.01-50 mg/kg, about 0.01-20 mg/kg, about 0.01-10 mg/kg, about 0.1-100 mg/kg, about 0.1-50 mg/kg, about 0.1-20 mg/kg, about 0.1-10 mg/kg, about 0.5-100 mg/kg, about 0.5-50 mg/kg, about 0.5-20 mg/kg, about 0.5-10 mg/kg, about 1-100 mg/kg, about 1-50 mg/kg, about 1-20 mg/kg, about 1-10 mg/kg body weight of the human subject. In one embodiment, about 0.5 mg-10 mg of the peptidomimetic macrocycle per kilogram body weight of the human subject is administered. In some examples the amount of the peptidomimetic macrocycle administered is about 0.16 mg, about 0.32 mg, about 0.64 mg, about 1.28 mg, about 3.56 mg, about 7.12 mg, about 14.24 mg, or about 20 mg per kilogram body weight of the human subject. In some examples the amount of the peptidomimetic macrocycle administered is about 0.16 mg, about 0.32 mg, about 0.64 mg, about 1.28 mg, about 3.56 mg, about 7.12 mg, or about 14.24 mg per kilogram body weight of the human subject. In some examples the amount of the peptidomimetic macrocycle administered is about 0.16 mg per kilogram body weight of the human subject. In some examples the amount of the peptidomimetic macrocycle administered is about 0.32 mg per kilogram body weight of the human subject. In some examples the amount of the peptidomimetic macrocycle administered is about 0.64 mg per kilogram body weight of the human subject. In some examples the amount of the peptidomimetic macrocycle administered is about 1.28 mg per kilogram body weight of the human subject. In some examples the amount of the peptidomimetic macrocycle administered is about 3.56 mg per kilogram body weight of the human subject. In some examples the amount of the peptidomimetic macrocycle administered is about 7.12 mg per kilogram body weight of the human subject. In some examples the amount of the peptidomimetic macrocycle administered is about 14.24 mg per kilogram body weight of the human subject.

In some embodiments about 0.5-about 20 mg or about 0.5-about 10 mg of the peptidomimetic macrocycle per kilogram body weight of the human subject is administered two times a week. For example about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered about twice a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered two times a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered two times a week. In some examples, the amount of the peptidomimetic macrocycle administered is about 1.25 mg, about 2.5 mg, about 5 mg or about 10 mg per kilogram body weight of the human subject and the peptidomimetic macrocycle is administered two times a week.

In some embodiments about 0.5-about 20 mg or about 0.5-about 10 mg of the peptidomimetic macrocycle per kilogram body weight of the human subject is administered once a week. For example about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered once a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered once a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once a week. In some examples, the amount of the peptidomimetic macrocycle administered is about 1.25 mg, about 2.5 mg, about 5 mg or about 10 mg per kilogram body weight of the human subject and the peptidomimetic macrocycle is administered once a week

In some embodiments about 0.5-about 20 mg or about 0.5-about 10 mg of the peptidomimetic macrocycle per kilogram body weight of the human subject is administered 3, 4, 5, 6, or 7 times a week. For example, about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administered 3, 4, 5, 6, or 7 times a week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered 3, 4, 5, 6, or 7 times a week. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered 3, 4, 5, 6, or 7 times a week. In some examples, the amount of the peptidomimetic macrocycle administered is about 1.25 mg, about 2.5 mg, about 5 mg, or about 10 mg per kilogram body weight of the human subject and the peptidomimetic macrocycle is administered 3, 4, 5, 6, or 7 times a week.

In some embodiments, about 0.5-about 20 mg or about 0.5-about 10 mg of the peptidomimetic macrocycle per kilogram body weight of the human subject is administered once every 2, 3, or 4 weeks. For example, about 0.5-about 1 mg, about 0.5-about 5 mg, about 0.5-about 10 mg, about 0.5-about 15 mg, about 1-about 5 mg, about 1-about 10 mg, about 1-about 15 mg, about 1-about 20 mg, about 5-about 10 mg, about 1-about 15 mg, about 5-about 20 mg, about 10-about 15 mg, about 10-about 20 mg, or about 15-about 20 mg of the peptidomimetic macrocycle per kilogram body weight of the human subject is administrated 3, 4, 5, 6, or 7 once every 2 or 3 week. In some examples about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.25 mg, about 12.5 mg, about 12.75 mg, about 13 mg, about 13.25 mg, about 13.5 mg, about 13.75 mg, about 14 mg, about 14.25 mg, about 14.5 mg, about 14.75 mg, about 15 mg, about 15.25 mg, about 15.5 mg, about 15.75 mg, about 16 mg, about 16.5 mg, about 17 mg, about 17.5 mg, about 18 mg, about 18.5 mg, about 19 mg, about 19.5 mg, or about 20 mg of the peptidomimetic macrocycle per kilogram body weight of the human subject is administered once every 2 or 3 weeks. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once every 2 weeks. In some examples, the amount of the peptidomimetic macrocycle administered is about 1.25 mg, about 2.5 mg, about 5 mg or about 10 mg per kilogram body weight of the human subject and the peptidomimetic macrocycle is administered once every 2 weeks. In some examples, the amount of the peptidomimetic macrocycle administered is about 1.25 mg, about 2.5 mg, about 5 mg, about 10 mg, or about 20 mg per kilogram body weight of the human subject and the peptidomimetic macrocycle is administered once every 3 weeks. In some examples, the amount of the peptidomimetic macrocycle administered is about 1.25 mg, about 2.5 mg, about 5 mg, or about 10 mg per kilogram body weight of the human subject and the peptidomimetic macrocycle is administered once every 3 weeks.

In some embodiments, the peptidomimetic macrocycle is administered gradually over a period of time. A desired amount of peptidomimetic macrocycle can, for example can be administered gradually over a period of from about 0.1 h-24 h. In some cases a desired amount of peptidomimetic macrocycle is administered gradually over a period of 0.1 h, 0.5 h, 1 h, 1.5 h, 2 h, 2.5 h, 3 h, 3.5 h, 4 h, 4.5 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h. In some examples, a desired amount of peptidomimetic macrocycle is administered gradually over a period of 0.25-12 h, for example over a period of 0.25-1 h, 0.25-2 h, 0.25-3 h, 0.25-4 h, 0.25-6 h, 0.25-8 h, 0.25-10 h. In some examples, a desired amount of peptidomimetic macrocycle is administered gradually over a period of 0.25-2 h. In some examples, a desired amount of peptidomimetic macrocycle is administered gradually over a period of 0.25-1 h. In some examples, a desired amount of peptidomimetic macrocycle is administered gradually over a period of 0.25 h, 0.3 h, 0.4 h, 0.5 h, 0.6 h, 0.7 h, 0.8 h, 0.9 h, 1.0 h, 1.1 h, 1.2 h, 1.3 h, 1.4 h, 1.5 h, 1.6 h, 1.7 h, 1.8 h, 1.9 h, or 2.0 h.

In some examples, a desired amount of peptidomimetic macrocycle is administered gradually over a period of 1 h. In some examples, a desired amount of peptidomimetic macrocycle is administered gradually over a period of 2 h.

Administration of the peptidomimetic macrocycles can continue as long as necessary. In some embodiments, one or more peptidomimetic macrocycle of the disclosure is administered for more than 1 day, more than 1 week, more than 1 month, more than 2 months, more than 3 months, more than 4 months, more than 5 months, more than 6 months, more than 7 months, more than 8 months, more than 9 months, more than 10 months, more than 11 months, more than 12 months, more than 13 months, more than 14 months, more than 15 months, more than 16 months, more than 17 months, more than 18 months, more than 19 months, more than 20 months, more than 21 months, more than 22 months, more than 23 months, or more than 24 months. In some embodiments, one or more peptidomimetic macrocycle of the disclosure is administered for less than 1 week, less than 1 month, less than 2 months, less than 3 months, less than 4 months, less than 5 months, less than 6 months, less than 7 months, less than 8 months, less than 9 months, less than 10 months, less than 11 months, less than 12 months, less than 13 months, less than 14 months, less than 15 months, less than 16 months, less than 17 months, less than 18 months, less than 19 months, less than 20 months, less than 21 months, less than 22 months, less than 23 months, or less than 24 months.

In some embodiments, the peptidomimetic macrocycle is administered on day 1, 8, 15 and 28 of a 28 day cycle. In some embodiments, the peptidomimetic macrocycle is administered on day 1, 8, 15 and 28 of a 28 day cycle and administration is continued for two cycles. In some embodiments, the peptidomimetic macrocycle is administered on day 1, 8, 15 and 28 of a 28 day cycle and administration is continued for three cycles. In some embodiments, the peptidomimetic macrocycle is administered on day 1, 8, 15 and 28 of a 28 day cycle and administration is continued for 4, 5, 6, 7, 8, 9, 10, or more cycles.

In some embodiments, the peptidomimetic macrocycle is administered on day 1, 8, 11 and 21 of a 21-day cycle. In some embodiments, the peptidomimetic macrocycle is administered on day 1, 8, 11 and 21 of a 21-day cycle and administration is continued for two cycles. In some embodiments, the peptidomimetic macrocycle is administered on day 1, 8, 11 and 21 of a 21-day cycle and administration is continued for three cycles. In some embodiments, the peptidomimetic macrocycle is administered on day 1, 8, 11 and 21 of a 21-day cycle and administration is continued for 4, 5, 6, 7, 8, 9, 10, or more cycles.

In some embodiments, one or more peptidomimetic macrocycle of the disclosure is administered chronically on an ongoing basis. In some embodiments administration of one or more peptidomimetic macrocycle of the disclosure is continued until documentation of disease progression, unacceptable toxicity, or patient or physician decision to discontinue administration.

### Method and Uses

In one aspect, the disclosure provides a method of treating a cancer in a subject, the method comprising administering to the subject a therapeutically-effective amount of a peptidomimetic macrocycle or a pharmaceutically-acceptable salt thereof, wherein the peptidomimetic macrocycle binds to MDM2 and/or MDMX proteins. In some embodiments, the peptidomimetic macrocycle can disrupt the interaction between p53 and MDM2 and MDMX. In some embodiments, treatment according to the method disclosed herein can result in re-activation of the p53 pathway, decreased cancer cell proliferation, increased p53 protein, increased p21, and/or increased apoptosis in the human subject.

In one aspect, the disclosure provides a method of treating a cancer, determined to lack a p53 deactivating mutation, in a subject the method comprising administering to the subject a therapeutically-effective amount of a peptidomimetic macrocycle or a pharmaceutically-acceptable salt thereof, wherein the peptidomimetic macrocycle binds to MDM2 and/or MDMX proteins. In some embodiments, the peptidomimetic macrocycle can disrupt the interaction between p53 and MDM2 and MDMX. The method further can comprise confirming the lack of the p53 deactivating mutation in the subject prior to the administration of the peptidomimetic macrocycle. In some embodiments, treatment according to the method disclosed herein can result in re-activation of the p53 pathway, decreased cancer cell proliferation, increased p53 protein, increased p21, and/or increased apoptosis in the human subject.

In one aspect, the disclosure provides a method of treating cancer in a subject expressing wild type p53, the method comprising administering to the subject a therapeutically-effective amount of a peptidomimetic macrocycle or a pharmaceutically-acceptable salt thereof, wherein the peptidomimetic macrocycle binds to MDM2 and/or MDMX proteins. In some embodiments, the peptidomimetic macrocycle can disrupt the interaction between p53 and MDM2 and MDMX. The method further can comprise confirming the wild type p53 status of the subject prior to the administration of the peptidomimetic macrocycle. In some embodiments, treatment according to the method disclosed herein can result in re-activation of the p53 pathway, decreased cancer cell proliferation, increased p53 protein, increased p21, and/or increased apoptosis in the human subject.

In some embodiments, the methods for treating a cancer provided herein inhibit, reduce, diminish, arrest, or stabilize a cancer cell associated with cancer. In other embodiments, the methods for treating cancer provided herein inhibit, reduce, diminish, arrest, or stabilize the symptoms associated with the cancer or two or more symptoms thereof. In some examples, the methods for treating cancer provided herein cause the reduction in the number of cancer cells and/or one or more symptoms associated with the cancer. In other examples, the methods for treating cancer provided herein maintain the number of cancer cells so that they do not increase, or so that the number of cancer cells increases by less than the increase of a number of cancer cells after administration of a standard therapy as measured by, for example, conventional methods available to one of skill in the art, such as ultrasound, CT Scan, MRI, dynamic contrast-enhanced MRI, or PET Scan. In some examples, the methods for treating cancer provided herein decrease the number of cancer cells. In some examples, the methods for treating cancer provided herein reduce the formation of cancer cells. In some examples, the methods for treating cancer provided herein eradicate, remove, or control primary, regional and/or metastatic cancer cells associated with the cancer. In some examples, the methods for treating cancer provided herein decrease the number or size of metastases associated with the cancer. In some examples, the methods for treating cancer provided herein reduce the number of cancer cells in a subject by an amount in the range of about 5-about 10%, about 5-about 20%, about 10-about 20%, about 15-about 20%, about 10-about 30%, about 20-about 30%, about 20-about 40%, about 30-about 40%, about 10-about 50%, about 20-about 50%, about 30-about 50%, about 40-about 50%, about 10-about 60%, about 20-about 60%, about 30-about 60%, about 40-about 60%, about 50-about 60%, about 10-about 70%, about 20-about 70%, about 30-about 70%, about 40-about 70%, about 50-about 70%, about 60-about 70%, about 10-about 80%, about 20-about 80%, about 30-about 80%, about 40-about 80%, about 50-about 80%, about 60-about 80%, about 70-about 80%, about 10-about 90%, about 20-about 90%, about 30-about 90%, about 40-about 90%, about 50-about 90%, about 60-about 90%, about 70-about 90%, about 80-about 90%, about 10-about 100%, about 20%-about 100%, about 30-about 100%, about 40-about 100%, about 50-about 100%, about 60-about 100%, about 70-about 100%, about 80-about 100%, about 90-about 100%, about 95-about 100%, or any range in between, relative to the number of cancer cells in a subject prior to administration of the peptidomimetic macrocycles as assessed by, for example, CT Scan, MRI, DCE-MRI, or PET Scan. In certain embodiments, the methods herein reduce the number of cancer cells in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100%, relative to the number of cancer cells prior to administration of the peptidomimetic macrocycle as assessed by, for example, CT Scan, MRI, DCE-MRI, or PET Scan.

In some embodiments, the methods provided herein reduce the cancer cell perfusion in a subject by an amount in the range of about 5-about 10%, about 5-about 20%, about 10-about 20%, about 15-about 20%, about 10-about 30%, about 20-about 30%, about 20-about 40%, about 30-about 40%, about 10-about 50%, about 20-about 50%, about 30-about 50%, about 40-about 50%, about 10-about 60%, about 20-about 60%, about 30-about 60%, about 40-about 60%, about 50-about 60%, about 10-about 70%, about 20-about 70%, about 30-about 70%, about 40-about 70%, about 50-about 70%, about 60-about 70%, about 10-about 80%, about 20-about 80%, about 30-about 80%, about 40-about 80%, about 50-about 80%, about 60-about 80%, about 70-about 80%, about 10-about 90%, about 20-about 90%, about 30-about 90%, about 40-about 90%, about 50-about 90%, about 60-about 90%, about 70-about 90%, about 80-about 90%, about 10-about 100%, about 20%-about 100%, about 30-about 100%, about 40-about 100%, about 50-about 100%, about 60-about 100%, about 70-about 100%, about 80-about 100%, about 90-about 100%, about 95-about 100%, or any range in between, relative to cancer cell perfusion prior to administration of the peptidomimetic macrocycle, as assessed by, for example, MRI, DCE-MRI, or PET Scan. In certain embodiments, the methods provided herein reduce the cancer cell perfusion in a subject by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100%, relative to cancer cell perfusion prior to administration of the peptidomimetic macrocycle as assessed by, for example, MRI, DCE-MRI, or PET Scan.

In some embodiments, the methods provided herein inhibit or decrease cancer cell metabolism in a subject in the range of about 5-about 10%, about 5-about 20%, about 10-about 20%, about 15-about 20%, about 10-about 30%, about 20-about 30%, about 20-about 40%, about 30-about 40%, about 10-about 50%, about 20-about 50%, about 30-about 50%, about 40-about 50%, about 10-about 60%, about 20-about 60%, about 30-about 60%, about 40-about 60%, about 50-about 60%, about 10-about 70%, about 20-about 70%, about 30-about 70%, about 40-about 70%, about 50-about 70%, about 60-about 70%, about 10-about 80%, about 20-about 80%, about 30-about 80%, about 40-about 80%, about 50-about 80%, about 60-about 80%, about 70-about 80%, about 10-about 90%, about 20-about 90%, about 30-about 90%, about 40-about 90%, about 50-about 90%, about 60-about 90%, about 70-about 90%, about 80-about 90%, about 10-about 100%, about 20%-about 100%, about 30-about 100%, about 40-about 100%, about 50-about 100%, about 60-about 100%, about 70-about 100%, about 80-about 100%, about 90-about 100%, about 95-about 100%, or any range in between, relative to cancer cell metabolism prior to administration of the peptidomimetic macrocycle, as assessed by, for example, MRI, DCE-MRI, or PET Scan. In certain embodiments, the methods provided herein inhibit or decrease cancer cell metabolism in a subject as assessed by, for example, PET scanning. In specific embodiments, the methods provided herein inhibit or decrease cancer cell metabolism in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, relative to cancer cell metabolism prior to administration of the peptidomimetic macrocycle.

In other aspect, the disclosure provides a method for increasing the survival time of a subject with cancer determined to lack a p53 deactivating mutation and/or with cancer expressing wild type p53, the method comprising administering to the subject a therapeutically effective amount of a peptidomimetic macrocycle or a pharmaceutically acceptable salt thereof, wherein the peptidomimetic macrocycle binds to MDM2 and/or MDMX proteins. In some examples, the survival time of the subject is at least 30 days longer than the expected survival time of the subject if the subject was not treated according to the methods provided herein. In some examples, the survival time of the subject is at 1 month-about 5 years longer than the expected survival time of the subject if the subject was not treated according to the methods provided herein. For example, the survival time of the subject is at least 3 months, at least 6 months, at least 9 months, at least 12 months, at least 15 months, at least 18 months, at least 21 months, or at least 24 months longer than the expected survival time of the subject if the subject was not treated according to the methods disclosed herein disclosure.

In one aspect, the disclosure provides a method to assessed presence, absence or amount of the biomarker biomarkers in a subject suffering with cancer. In some examples, the biomarkers include patient biomarkers, for example, the p53 status of the subject and the MDM2 and MDMX expression levels in the subject.

The method of the disclosure can also optionally include studying and/or evaluating the safety and/or tolerability of the peptidomimetic macrocycles disclosed herein in the subject.

Also provided herein is a method to re-activate the p53 pathway in a subject with a cancer lacking a p53 deactivating mutation and/or expressing wild type p53, the method comprising administering to the subject a therapeutically effective amount of a peptidomimetic macrocycle or a pharmaceutically acceptable salt thereof, wherein the peptidomimetic macrocycle binds to MDM2 and/or MDMX proteins.

Also provided herein is a method to decrease cancer cell proliferation in a human subject with a cancer lacking a p53 deactivating mutation and/or expressing wild type p53, the method comprising administering to the subject a therapeutically effective amount of a peptidomimetic macrocycle or a pharmaceutically acceptable salt thereof, wherein the peptidomimetic macrocycle binds to MDM2 and/or MDMX proteins.

Also provided herein is a method to increased p53 protein in a subject with a cancer lacking a p53 deactivating mutation and/or expressing wild type p53, the method comprising administering to the subject a therapeutically effective amount of a peptidomimetic macrocycle or a pharmaceutically acceptable salt thereof, wherein the peptidomimetic macrocycle binds to MDM2 and/or MDMX proteins.

Also provided herein is a method to increased p21 in a subject with a cancer lacking a p53 deactivating mutation and/or expressing wild type p53, the method comprising administering to the subject a therapeutically effective amount of a peptidomimetic macrocycle or a pharmaceutically acceptable salt thereof, wherein the peptidomimetic macrocycle binds to MDM2 and/or MDMX proteins.

Also provided herein is a method to increased apoptosis in a subject with a cancer lacking a p53 deactivating mutation and/or expressing wild type p53, the method comprising administering to the subject a therapeutically effective amount of a peptidomimetic macrocycle or a pharmaceutically acceptable salt thereof, wherein the peptidomimetic macrocycle binds to MDM2 and/or MDMX proteins.

In some embodiments, the disclosure also provides a method to determine the dose limiting toxicities (DLTs) and/or maximum tolerated dose (MTD or OBD) or the optimal biological dose (OBD) of the peptidomimetic macrocycles disclosed herein in subject with a cancer (e.g., a lymphoma) lacking a p53 deactivating mutation and/or expressing wild type p53.

The methods of the disclosure can optionally include pharmacokinetic analysis of the peptidomimetic macrocycles disclosed herein. Accordingly, the methods can further comprise collecting one or more biological sample from the subject at one or more specific time point and analyzing the one or more biological sample for levels of the peptidomimetic macrocycles and/or it metabolites. The biological sample can be a blood sample from the subject, for example, a blood sample from a human subject. The one or more specific time point can include time points before, after and/or during the administration of the peptidomimetic macrocycle to the subject. In some embodiments one or more biological sample includes biological samples collected before and after each administration of the peptidomimetic macrocycle to the subject. In some embodiments a biological sample for pharmacokinetic analysis is collected before the first administration of the peptidomimetic macrocycle to the subject and at one or more time points after each administration of the peptidomimetic macrocycles to the subject. The biological sample collected before the administration of the peptidomimetic macrocycle to the subject can be done within 0-24 hour before the start of administration of the peptidomimetic macrocycle to the subject. For example, the biological sample can be collected within 24 h, within 23 h, within 22 h, within 21 h, within 20 h, within 19 h, within 18 h, within 17 h, within 16 h, within 15 h, within 14 h, within 13 h, within 12 h, within 11 h, within 10 h, within 9 h, within 8 h, within 7 h, within 6 h, within 5 h, within 4 h, within 3 h, within 2 h, within 1 h, within 30 min, within 15 min, or immediately before the administration of the peptidomimetic macrocycle to the subject. One or more biological samples collected after the administration of the peptidomimetic macrocycle to the subject can be collected, for example after 0 min, 5 min, 10 min, 20 min, 30 min, 45 min, 60 min, 1.25 h, 1.5 h, 1.75 h, 2.0 h, 2.25 h, 2.5 h, 2.75 h, 3.0 h, 3.25 h, 3.5 h, 3.75 h, 4.0 h, 4.25 h, 4.5 h, 4.75 h, 5.0 h, 5.25 h, 5.5 h, 5.75 h, 6.0 h, 6.25 h, 6.5 h, 6.75 h, 7.0 h, 7.25 h, 7.5 h, 7.75 h, 8.0 h, 8.25 h, 8.5 h, 8.75 h, 9.0 h, 9.25 h, 9.5 h, 9.75 h, 10.0 h, 10.25 h, 10.5 h, 10.75 h, 11.0 h, 11.25 h, 11.5 h, 11.75 h, 12.0 h, 20 h, 24 h, 28 h, 32 h, 36 h, 40 h, 44 h, 48 h, 52 h, 56 h, 60 h, 64 h, 68 h, 72 h, or 0-72 h after the administration of the peptidomimetic macrocycle to the subject. In some embodiments, the peptidomimetic macrocycle is administered on day 1, day 8, day 15 of a 28 day cycle and biological sample is collected before administration on day 1, after the administration on day 1 (multiple biological samples can be collected, for example after about 0 min, about 30 min, about 1 h, about 2 h, about 4 h, about 8 h, about 24 h, and 48 hour after administration), before administration on day 8, after administration on day 8 (multiple biological samples can be collected, for example after about 0 min, about 30 min, about 1 h, about 2 h, and about 4 h after administration), before administration on day 15 and after administration on day 15 (multiple biological samples can be collected, for example after about 0 min, about 30 min, about 1 h, about 2 h, about 4 h, about 8 h, and about 24 h after administration). In some embodiments, the peptidomimetic macrocycle is administered on day 1, day 8, day 11 of a 21 day cycle and biological sample is collected before administration on day 1, after the administration on day 1 (multiple biological samples can be collected, for example after about 0 min, about 30 min, about 1 h, about 2 h, about 4 h, about 8 h, about 24 h, and 48 hour after administration), before administration on day 8, after administration on day 8 (multiple biological samples can be collected, for example after about 0 min, about 30 min, about 1 h, about 2 h, and about 4 h after administration), before administration on day 11 and after administration on day 11 (multiple biological samples can be collected, for example after about 0 min, about 30 min, about 1 h, about 2 h, about 4 h, about 8 h, and about 24 h after administration).

The method of the disclosure can optionally include pharmacodynamic analysis of the peptidomimetic macrocycles disclosed herein. Accordingly, the methods can comprise collecting one or more biological samples from the subject at one or more specific time points for pharmacodynamic analysis. Pharmacodynamic analysis can include analyzing the levels of biomarkers including MIC-1, p53, MDM2, MDMX, p21 and/or cases in the biological sample. Detection of biomarkers in a biological sample can be performed by, for example, direct measurement, immunohistochemistry, immunoblotting, immunoflourescense, immunoabsorbence, immunoprecipitations, protein array, flourescence in situ hybridization, FACS analysis, hybridization, in situ hybridization, Northern blots, Southern blots, Western blots, ELISA, radioimmunoassay, gene array/chip, PCR, RT-PCR, or cytogenetic analysis. The biological sample for pharmacodynamic analysis can be a blood sample or a cancer cell specimen from the subject, for example, a biological sample for pharmacodynamic analysis can be a blood sample or a cancer cell specimen from the human subject. The biological samples for pharmacodynamic analysis of the peptidomimetic macrocycles can be collected any time before, during, or after the administration of the peptidomimetic macrocycle to the subject. In some embodiments a blood sample for pharmacokinetic analysis is collected before the first administration of the peptidomimetic macrocycle to the subject and at one or more time points after each administration of the peptidomimetic macrocycles to the subject. The blood sample collected before the administration of the peptidomimetic macrocycle to the subject can be done within 0-24 hour before the start of administration of the peptidomimetic macrocycle to the subject. For example, the biological sample can be collected within 24 h, within 23 h, within 22 h, within 21 h, within 20 h, within 19 h, within 18 h, within 17 h, within 16 h, within 15 h, within 14 h, within 13 h, within 12 h, within 11 h, within 10 h, within 9 h, within 8 h, within 7 h, within 6 h, within 5 h, within 4 h, within 3 h, within 2 h, within 1 h, within 30 min, within 15 min of, or immediately before the administration of the peptidomimetic macrocycle to the subject. One or more blood samples for pharmacodynamic analysis collected after the administration of the peptidomimetic macrocycle to the subject can be collected from 0-about 72 h, for example after about 12 h, after about 24 h, after about 36 h or after about 48 h after the administration of the peptidomimetic macrocycle to the subject. In some embodiments, the peptidomimetic macrocycle is administered on day 1, day 8, day 15 of a 28 day cycle and blood samples for pharmacodynamic analysis are collected before administration on day 1, after the administration on day 1 (multiple samples can be collected, for example after about 24 h and 48 hour after administration), before administration on day 8, after administration on day 8 (multiple samples can be collected, for example with about 1 h administration), before administration on day 15 and after administration on day 15 (multiple samples can be collected, for example with about 1 h and about 48 h after administration), and day 22. Biological samples for pharmacodynamic analysis can be collected at any time before, after or during the administration of the peptidomimetic macrocycle to the subject. For example the peptidomimetic macrocycle can be administered on day 1, day 8, day 15 of a 28 day cycle and cancer cell samples for pharmacodynamic analysis are collected before administration on day 1 and between day 14-day 18, for example of day 16. In some embodiments, the peptidomimetic macrocycle is administered on day 1, day 8, day 11, of a 21 day cycle and blood samples for pharmacodynamic analysis are collected before administration on day 1, after the administration on day 1 (multiple samples can be collected, for example after about 24 h and 48 hour after administration), before administration on day 8, after administration on day 8 (multiple samples can be collected, for example with about 1 h administration), before administration on day 11 and after administration on day 11 (multiple samples can be collected, for example with about 1 h and about 48 h after administration), and day 22. Biological samples for pharmacodynamic analysis can be collected at any time before, after or during the administration of the peptidomimetic macrocycle to the subject. For example the peptidomimetic macrocycle can be administered on day 1, day 8, day 11 of a 21 day cycle and cancer cell samples for pharmacodynamic analysis are collected before administration on day 1 and between day 10-day 14, for example of day 12.

The method of the disclosure can optionally include clinical activity analysis of the peptidomimetic macrocycles disclosed herein. Accordingly, the methods can comprise analyzing one or more biological samples collected from the subject at one or more specific time points. Any appropriate analytical procedure can be used for the analysis of the biological samples. For example, imaging techniques like radiographs, ultrasound, CT scan, PET scan, MRI scan, chest x-ray, laparoscopy, complete blood count (CBC) test, bone scanning and fecal occult blood test can be used. Further analytical procedures that can be used include blood chemistry analysis, chromosomal translocation analysis, needle biopsy, tissue biopsy, fluorescence in situ hybridization, laboratory biomarker analysis, immunohistochemistry staining method, flow cytometry, or a combination thereof. The method can further comprise tabulating and/or plotting results of the analytical procedure.

For example, pharmacodynamics can be assessed by laboratory-based evaluation of several biomarkers of p53 activation, including levels of p21, caspase and MDM2 in cancer cell tissue, and where available in CTC, as well as MIC-1 in blood, before and after treatment with the peptidomimetic macrocycles.

Results available from previous genetic and biomarker tests and additional tests of the blood and cancer cell samples for biomarkers relevant to the safety and efficacy of the peptidomimetic macrocycles can be investigated for possible correlation with patient outcome.

For example, clinical activity or response can be evaluated by standard imaging assessments, such as computed tomography (CT), magnetic resonance imaging (MRI), and bone scans. In addition, [¹⁸]-fluorodeoxyglucose and [¹⁸]-fluorothymidine positron emission tomography (FDG-PET and FLT-PET, respectively), or other techniques considered clinically appropriate for the patient's specific disease type can be used. CT-imaging can be performed, for example, at the end of Cycle 2, and every 2 cycles (e.g., Cycles 4 and 6) thereafter for DR-A and after the last infusion in Cycle 3 and every 3 cycles (e.g., Cycles 6 and 9) thereafter in DR-B. Anti-cancer cell activity can be assessed using IWG (2014) (Appendix H) criteria for patients with lymphomas. Additionally, for patients with an FDG-avid lymphoma, FDG-PET imaging can be performed at baseline and post-baseline as outlined in IWG 2014. FLT-PET imaging can be performed at baseline for patients with cancer cell commonly showing sufficient uptake of FLT tracer, e.g., patients with lymphoma. For example, DR-A assigned patients who demonstrate a standard uptake value (SUV) of >5 at baseline can have a repeat FLT image one day after their last infusion of study medication in Cycle 1, i.e., Day 16. For example, DR-B patients who demonstrate a standard uptake value (SUV) of >5 at baseline can have a repeat FLT image one day after their last infusion of study medication in Cycle 1, i.e., Day 12.

### Biological Samples

As used in the present application, “biological sample” means any fluid or other material derived from the body of a normal or diseased subject, such as blood, serum, plasma, lymph, urine, saliva, tears, cerebrospinal fluid, milk, amniotic fluid, bile, ascites fluid, pus, and the like. Also included within the meaning of the term “biological sample” is an organ or tissue extract and culture fluid in which any cells or tissue preparation from a subject has been incubated. The biological samples can be any samples from which genetic material can be obtained. Biological samples can also include solid or liquid cancer cell samples or specimens. The cancer cell sample can be a cancer cell tissue sample. In some embodiments, the cancer cell tissue sample can obtained from surgically excised tissue. Exemplary sources of biological samples include fine needle aspiration, core needle biopsy, vacuum assisted biopsy, incisional biopsy, excisional biopsy, punch biopsy, shave biopsy or skin biopsy. In some cases, the biological samples comprise fine needle aspiration samples. In some embodiments, the biological samples comprise tissue samples, including, for example, excisional biopsy, incisional biopsy, or other biopsy. The biological samples can comprise a mixture of two or more sources; for example, fine needle aspirates and tissue samples. Tissue samples and cellular samples can also be obtained without invasive surgery, for example by punctuating the chest wall or the abdominal wall or from masses of breast, thyroid or other sites with a fine needle and withdrawing cellular material (fine needle aspiration biopsy). In some embodiments, a biological sample is a bone marrow aspirate sample. A biological sample can be obtained by methods known in the art such as the biopsy methods provided herein, swabbing, scraping, phlebotomy, or any other suitable method.

The biological samples obtained can be used in fresh, frozen, or fixed (e.g., formaldehyde fixed-paraffin embedded) form, depending on the nature of the sample, the assay used, and the convenience of the practitioner. Although fresh, frozen and fixed materials are suitable for various RNA and protein assays, generally, fresh tissues can be preferred for ex vivo measurements of activity.

Fixed tissue samples can also be employed. Tissue obtained by biopsy is often fixed, usually by formalin, formaldehyde, or gluteraldehyde, for example, or by alcohol immersion. Fixed biological samples are often dehydrated and embedded in paraffin or other solid supports. See the reference Plenat et al., 2001, Ann. Pathol. 21:29-47. Non-embedded, fixed tissue, as well as fixed and embedded tissue, can be used in the present methods. Solid supports for embedding fixed tissue can be removed with organic solvents to enable subsequent rehydration of preserved tissue.

In some cases, the assay includes a step of cell or tissue culture. For example, cells from a biopsy can be disaggregated using enzymes (such as collagenase and hyaluronidase) and or physical disruption (e.g., repeated passage through a 25-gauge needle) to dissociate the cells, collected by centrifugation, and resuspended in desired buffer or culture medium for culture, immediate analysis, or further processing.

### Subject/Patient Population

In some embodiments, a subject treated for cancer in accordance with the methods provided herein is a human, who has or is diagnosed with a cancer. In other embodiments, a subject treated for cancer in accordance with the methods provided herein is a human, predisposed or susceptible to a cancer. In some embodiments, a subject treated for cancer in accordance with the methods provided herein is a human, at risk of developing a cancer.

In some embodiments, a subject treated for cancer in accordance with the methods provided herein is a human, who has or is diagnosed with a cancer, determined to lack a p53 deactivating mutation and/or expressing wild type p53. In other embodiments, a subject treated for cancer in accordance with the methods provided herein is a human, predisposed or susceptible to a cancer, determined to lack a p53 deactivating mutation and/or expressing wild type p53. In some embodiments, a subject treated for cancer in accordance with the methods provided herein is a human, at risk of developing a cancer, determined to lack a p53 deactivating mutation and/or expressing wild type p53. A p53 deactivating mutation, as used herein is any mutation that leads to loss of (or a decrease in) the in vitro apoptotic activity of p53. Non limiting examples of p53 deactivating mutations are included in Table 1. Accordingly, in some embodiments, a subject with a cancer in accordance with the composition as provided herein is a human who has or is diagnosed with a cancer that is determined to lack a p53 deactivation mutation, such as those shown in Table 7.

In some embodiments, a subject treated for cancer in accordance with the methods provided herein is a human, who has or is diagnosed with a cancer, determined to lack a dominant p53 deactivating mutation. Dominant p53 deactivating mutation or dominant negative mutation, as used herein, is a mutation wherein the mutated p53 inhibits or disrupt the activity of the wild type p53 gene.

TABLE 7
Examples of p53 deactivating mutations

| Mutation at position | Amino acid change |
|---|---|
| 62 | E62_W91del |
| 122 | V122X |
| 135 | C135S |
| 143 | V143A |
| 144 | Q144P |
| 146 | W146X |
| 157 | V157F |
| 158 | R158H |
| 163 | Y163N |
| 168 | H168Y |
| 173 | V173L |
| 175 | R175H |
| 175 | R175P |
| 175 | R175Q |
| 175 | R175S |
| 219 | P219H |
| 234 | Y234C |
| 234 | Y234H |
| 237 | M237I |
| 240 | S240R |
| 245 | G245C |
| 245 | G245S |
| 246 | M246I |
| 248 | R248Q |
| 248 | R248W |
| 249 | R249S |
| 272 | V272M |
| 273 | R273H |
| 274 | V274F |
| 279 | G279E |
| 280 | R280K |
| 281 | D281H |
| 282 | R282W |
| 306 | R306P |
| 308 | P300_L308del |
| 327 | P300_Y327del |
| 332 | D324_I332del |
| 337 | R337C |
| 344 | L344P |

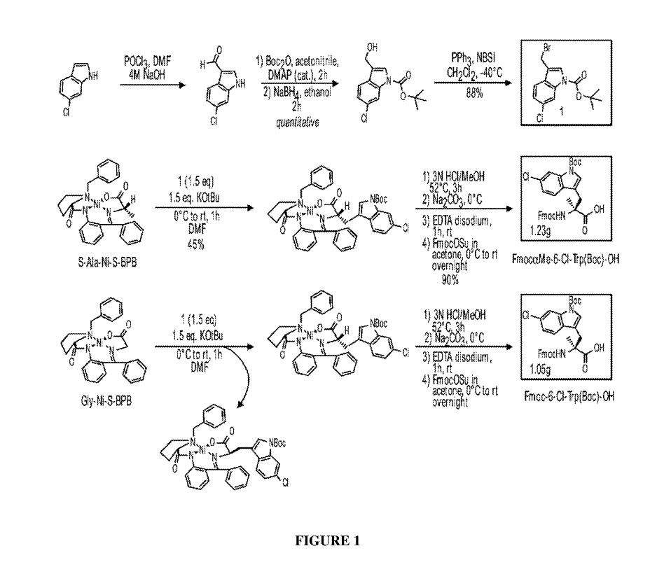

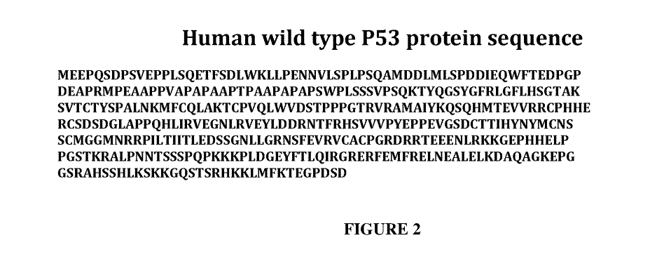

Table 7 refers to the sequence of the wild type human TP53 tumor protein p53 shown in FIG. 1. Amino acid changes are reported as: the amino acid being substituted followed by the position of the amino acid being substituted in the wild type p53 sequence, followed by the amino acid used for substitution. For example L344P, indicates that the leucine residue (L) at the 344 position in the wild type sequence is replaced by a proline residue (P).

In some embodiments, a subject treated for cancer in accordance with the methods provided herein is a refractory patient. In a certain embodiment, a refractory patient is a patient refractory to a standard therapy (e.g., surgery, radiation, anti-androgen therapy and/or drug therapy such as chemotherapy). In certain embodiments, a patient with the cancer is refractory to a therapy when the cancer has not significantly been eradicated and/or the one or more symptoms have not been significantly alleviated. The determination of whether a patient is refractory can be made either in vivo or in vitro by any method known in the art for assaying the effectiveness of a treatment of cancer. In various embodiments, a patient with cancer is refractory when the number of CTCs or MNBCs associated with the cancer has not decreased or has increased. In various embodiments, a patient with cancer is refractory when one or more cancer cells metastasize and/or spread to another organ.

In some embodiments, a subject treated for cancer accordance with the methods provided herein is a human that has proven refractory to therapies other than treatment with the peptidomimetic macrocycles of the disclosure, but is no longer on these therapies. In certain embodiments, a subject treated for cancer in accordance with the methods provided herein is a human already receiving one or more conventional anti-cancer therapies, such as surgery, drug therapy such as chemotherapy, anti-androgen therapy or radiation. Among these patients are refractory patients, patients who are too young for conventional therapies, and patients with recurring cancer cells despite treatment with existing therapies.

In some embodiments, the subject is a human who has had at least one unsuccessful prior treatment and/or therapy of the cancer.

Methods of Detecting Wild Type p53 and/or p53 Mutations

In some embodiments, a subject lacking p53-deactivating mutations is a candidate for cancer treatment with a compound of the invention. Cancer cells from patient groups should be assayed in order to determine p53-deactivating mutations and/or expression of wild type p53 prior to treatment with a compound of the invention.

The activity of the p53 pathway can be determined by the mutational status of genes involved in the p53 pathways, including, for example, AKT1, AKT2, AKT3, ALK, BRAF, CDK4, CDKN2A, DDR2, EGFR, ERBB2 (HER2), FGFR1, FGFR3, GNA11, GNQ, GNAS, KDR, KIT, KRAS, MAP2K1 (MEK1), MET, HRAS, NOTCH1, NRAS, NTRK2, PIK3CA, NF1, PTEN, RAC1, RB1, NTRK3, STK11, PIK3R1, TSC1, TSC2, RET, TP53, and VHL. Genes that modulate the activity of p53 can also be assessed, including, for example, kinases: ABL1, JAK1, JAAK2, JAK3; receptor tyrosine kinases: FLT3 and KIT; receptors: CSF3R, IL7R, MPL, and NOTCH1; transcription factors: BCOR, CEBPA, CREBBP, ETV6, GATA1, GATA2. MLL, KZF1, PAXS, RUNX1, STAT3, WT1, and TP53; epigenetic factors: ASXL1, DNMT3A, EZH2, KDM6A (UTX), SUZ12, TET2, PTPN11, SF3B1, SRSF2, U2AF35, ZRSR2; RAS proteins: HRAS, KRAS, and NRAS; adaptors CBL and CBL-B; FBXW7, IDH1, IDH2, and NPM1.

Cancer cell samples can be obtained, for example, from solid or liquid tumors via primary or metastatic tumor resection (e.g. pneumonectomy, lobetomy, wedge resection, and craniotomy) primary or metastatic disease biopsy (e.g. transbronchial or needle core), pleural or ascites fluid (e.g. FFPE cell pellet), bone marrow aspirate, bone marrow clot, and bone marrow biopsy, or macro-dissection of tumor rich areas (solid tumors).

To detect the p53 wild type gene and/or lack of p53 deactivation mutation in a tissue, cancerous tissue can be isolated from surrounding normal tissues. For example, the tissue can be isolated from paraffin or cryostat sections. Cancer cells can also be separated from normal cells by flow cytometry. If the cancer cells tissue is highly contaminated with normal cells, detection of mutations can be more difficult.

Various methods and assays for analyzing wild type p53 and/or p53 mutations are suitable for use in the invention. Non-limiting examples of assays include polymerase chain reaction (PCR), restriction fragment length polymorphism (RFLP), microarray, Southern Blot, Northern Blot, Western Blot, Eastern Blot, H&E staining, microscopic assessment of tumors, next-generation DNA sequencing (NGS) (e.g. extraction, purification, quantitiation, and amplification of DNA, library preparation), immunohistochemistry, and fluorescent in situ hybridization (FISH).

A microarray allows a researcher to investigate multiple DNA sequences attached to a surface, for example, a DNA chip made of glass or silicon, or a polymeric bead or resin. The DNA sequences are hybridized with fluorescent or luminescent probes. The microarray can indicate the presence of oligonucleotide sequences in a sample based on hybridization of sample sequences to the probes, followed by washing and subsequent detection of the probes. Quantification of the fluorescent or luminescent signal indicates the presence of known oligonucleotide sequences in the sample.

PCR allows amplification of DNA oligomers rapidly, and can be used to identify an oligonucleotide sequence in a sample. PCR experiments involve contacting an oligonucleotide sample with a PCR mixture containing primers complementary to a target sequence, one or more DNA polymerase enzymes, deoxnucleotide triphosphate (dNTP) building blocks, including dATP, dGTP, dTTP, and dCTP, and suitable buffers, salts, and additives. If a sample contains an oligonucleotide sequence complementary to a pair of primers, the experiment amplifies the sample sequence, which can be collected and identified.

In some embodiments, an assay comprises amplifying a biomolecule from the cancer sample. The biomolecule can be a nucleic acid molecule, such as DNA or RNA. In some embodiments, the assay comprises circularization of a nucleic acid molecule, followed by digestion of the circularized nucleic acid molecule.

In some embodiments, the assay comprises contacting an organism, or a biochemical sample collected from an organism, such as a nucleic acid sample, with a library of oligonucleotides, such as PCR primers. The library can contain any number of oligonucleotide molecules. The oligonucleotide molecules can bind individual DNA or RNA motifs, or any combination of motifs described herein. The motifs can be any distance apart, and the distance can be known or unknown. In some embodiments, two or more oligonucleotides in the same library bind motifs a known distance apart in a parent nucleic acid sequence. Binding of the primers to the parent sequence can take place based on the complementarity of the primers to the parent sequence. Binding can take place, for example, under annealing, or under stringent conditions.

In some embodiments, the results of an assay are used to design a new oligonucleotide sequence for future use. In some embodiments, the results of an assay are used to design a new oligonucleotide library for future use. In some embodiments, the results of an assay are used to revise, refine, or update an existing oligonucleotide library for future use. For example, an assay can reveal that a previously-undocumented nucleic acid sequence is associated with the presence of a target material. This information can be used to design or redesign nucleic acid molecules and libraries.

In some embodiments, one or more nucleic acid molecules in a library comprise a barcode tag. In some embodiments, one or more of the nucleic acid molecules in a library comprise type I or type II restriction sites suitable for circularization and cutting an amplified sample nucleic acid sequence. Such primers can be used to circularize a PCR product and cut the PCR product to provide a product nucleic acid sequence with a sequence that is organized differently from the nucleic acid sequence native to the sample organism.

After a PCR experiment, the presence of an amplified sequence can be verified. Non-limiting examples of methods for finding an amplified sequence include DNA sequencing, whole transcriptome shotgun sequencing (WTSS, or RNA-seq), mass spectrometry (MS), microarray, pyrosequencing, column purification analysis, polyacrylamide gel electrophoresis, and index tag sequencing of a PCR product generated from an index-tagged primer.

In some embodiments, more than one nucleic acid sequence in the sample organism is amplified. Non-limiting examples of methods of separating different nucleic acid sequences in a PCR product mixture include column purification, high performance liquid chromatography (HPLC), HPLC/MS, polyacrylamide gel electrophoresis, size exclusion chromatography.

The amplified nucleic acid molecules can be identified by sequencing. Nucleic acid sequencing can be done on automated instrumentation. Sequencing experiments can be done in parallel to analyze tens, hundreds, or thousands of sequences simultaneously. Non-limiting examples of sequencing techniques follow.

In pyrosequencing, DNA is amplified within a water droplet containing a single DNA template bound to a primer-coated bead in an oil solution. Nucleotides are added to a growing sequence, and the addition of each base is evidenced by visual light.

Ion semiconductor sequencing detects the addition of a nucleic acid residue as an electrical signal associated with a hydrogen ion liberated during synthesis. A reaction well containing a template is flooded with the four types of nucleotide building blocks, one at a time. The timing of the electrical signal identifies which building block was added, and identifies the corresponding residue in the template.

DNA nanoball uses rolling circle replication to amplify DNA into nanoballs. Unchained sequencing by ligation of the nanoballs reveals the DNA sequence.

In a reversible dyes approach, nucleic acid molecules are annealed to primers on a slide and amplified. Four types of fluorescent dye residues, each complementary to a native nucleobase, are added, the residue complementary to the next base in the nucleic acid sequence is added, and unincorporated dyes are rinsed from the slide. Four types of reversible terminator bases (RT-bases) are added, and non-incorporated nucleotides are washed away. Fluorescence indicates the addition of a dye residue, thus identifying the complementary base in the template sequence. The dye residue is chemically removed, and the cycle repeats.

Detection of point mutations can be accomplished by molecular cloning of the p53 allele(s) present in the cancer cell tissue and sequencing that allele(s). Alternatively, the polymerase chain reaction can be used to amplify p53 gene sequences directly from a genomic DNA preparation from the cancer cell tissue. The DNA sequence of the amplified sequences can then be determined. See e.g., Saiki et al., Science, Vol. 239, p. 487, 1988; U.S. Pat. No. 4,683,202; and U.S. Pat. No. 4,683,195. Specific deletions of p53 genes can also be detected. For example, restriction fragment length polymorphism (RFLP) probes for the p53 gene or surrounding marker genes can be used to score loss of a p53 allele.

Loss of wild type p53 genes can also be detected on the basis of the loss of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR).

Alternatively, mismatch detection can be used to detect point mutations in the p53 gene or the mRNA product. The method can involve the use of a labeled riboprobe that is complementary to the human wild type p53 gene. The riboprobe and either mRNA or DNA isolated from the cancer cell tissue are annealed (hybridized) together and subsequently digested with the enzyme RNase A which is able to detect some mismatches in a duplex RNA structure. If a mismatch is detected by RNase A, the enzyme cleaves at the site of the mismatch. Thus, when the annealed RNA preparation is separated on an electrophoretic gel matrix, if a mismatch has been detected and cleaved by RNase A, an RNA product is seen that is smaller than the full-length duplex RNA for the riboprobe and the p53 mRNA or DNA. The riboprobe need not be the full length of the p53 mRNA or gene but can be a segment of either. If the riboprobe comprises only a segment of the p53 mRNA or gene it will be desirable to use a number of these probes to screen the whole mRNA sequence for mismatches.

In similar fashion, DNA probes can be used to detect mismatches, through enzymatic or chemical cleavage. See, e.g., Cotton et al., Proc. Natl. Acad. Sci. USA, vol. 85, 4397, 1988; and Shenk et al., Proc. Natl. Acad. Sci. USA, vol. 72, p. 989, 1975. Alternatively, mismatches can be detected by shifts in the electrophoretic mobility of mismatched duplexes relative to matched duplexes. See, e.g., Cariello, Human Genetics, vol. 42, p. 726, 1988. With either riboprobes or DNA probes, the cellular mRNA or DNA which might contain a mutation can be amplified using PCR (see below) before hybridization.

DNA sequences of the p53 gene from the cancer cell tissue which have been amplified by use of polymerase chain reaction can also be screened using allele-specific probes. These probes are nucleic acid oligomers, each of which contains a region of the p53 gene sequence harboring a known mutation. For example, one oligomer can be about 30 nucleotides in length, corresponding to a portion of the p53 gene sequence. At the position coding for the 175th codon of p53 gene the oligomer encodes an alanine, rather than the wild type codon valine. By use of a battery of such allele-specific probes, the PCR amplification products can be screened to identify the presence of a previously identified mutation in the p53 gene. Hybridization of allele-specific probes with amplified p53 sequences can be performed, for example, on a nylon filter. Hybridization to a particular probe indicates the presence of the same mutation in the cancer cell tissue as in the allele-specific probe.

The identification of p53 gene structural changes in cancer cells can be facilitated through the application of a diverse series of high resolution, high throughput microarray platforms. Essentially two types of array include those that carry PCR products from cloned nucleic acids (e.g. cDNA, BACs, cosmids) and those that use oligonucleotides. The methods can provide a way to survey genome wide DNA copy number abnormalities and expression levels to allow correlations between losses, gains and amplifications in cancer cells with genes that are over- and under-expressed in the same samples. The gene expression arrays that provide estimates of mRNA levels in cancer cells have given rise to exon-specific arrays that can identify both gene expression levels, alternative splicing events and mRNA processing alterations.

Oligonucleotide arrays can be used to interrogate single nucleotide polymorphisms (SNPs) throughout the genome for linkage and association studies and these have been adapted to quantify copy number abnormalities and loss of heterozygosity events. DNA sequencing arrays can allow resequencing of chromosome regions and whole genomes.

SNP-based arrays or other gene arrays or chips can determine the presence of wild type p53 allele and the structure of mutations. A single nucleotide polymorphism (SNP), a variation at a single site in DNA, is the most frequent type of variation in the genome. For example, there are an estimated 5-10 million SNPs in the human genome. SNPs can be synonymous or nonsynonymous substitutions. Synonymous SNP substitutions do not result in a change of amino acid in the protein due to the degeneracy of the genetic code, but can affect function in other ways. For example, a seemingly silent mutation in gene that codes for a membrane transport protein can slow down translation, allowing the peptide chain to misfold, and produce a less functional mutant membrane transport protein. Nonsynonymous SNP substitutions can be missense substitutions or nonsense substitutions. Missense substitutions occur when a single base change results in change in amino acid sequence of the protein and malfunction thereof leads to disease. Nonsense substitutions occur when a point mutation results in a premature stop codon, or a nonsense codon in the transcribed mRNA, which results in a truncated and usually, nonfunctional, protein product. As SNPs are highly conserved throughout evolution and within a population, the map of SNPs serves as an excellent genotypic marker for research. SNP array is a useful tool to study the whole genome.

In addition, SNP array can be used for studying the Loss Of Heterozygosity (LOH). LOH is a form of allelic imbalance that can result from the complete loss of an allele or from an increase in copy number of one allele relative to the other. While other chip-based methods (e.g., comparative genomic hybridization can detect only genomic gains or deletions), SNP array has the additional advantage of detecting copy number neutral LOH due to uniparental disomy (UPD). In UPD, one allele or whole chromosome from one parent are missing leading to reduplication of the other parental allele (uni-parental=from one parent, disomy=duplicated). In a disease setting this occurrence can be pathologic when the wild type allele (e.g., from the mother) is missing and instead two copies of the heterozygous allele (e.g., from the father) are present. This usage of SNP array has a huge potential in cancer diagnostics as LOH is a prominent characteristic of most human cancers. SNP array technology have shown that cancers (e.g. gastric cancer, liver cancer, etc.) and hematologic malignancies (ALL, MDS, CML etc) have a high rate of LOH due to genomic deletions or UPD and genomic gains. In the present disclosure, using high density SNP array to detect LOH allows identification of pattern of allelic imbalance to determine the presence of wild type p53 allele (Lips et al., 2005; Lai et al., 2007).

Examples of p53 gene sequence and single nucleotide polymorphism arrays include p53 Gene Chip (Affymetrix, Santa Clara, Calif.), Roche p53 Ampli-Chip (Roche Molecular Systems, Pleasanton, Calif.), GeneChip Mapping arrays (Affymetrix, Santa Clara, Calif.), SNP Array 6.0 (Affymetrix, Santa Clara, Calif.), BeadArrays (Illumina, San Diego, Calif.), etc.

Mutations of wild type p53 genes can also be detected on the basis of the mutation of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR). A panel of monoclonal antibodies could be used in which each of the epitopes involved in p53 functions are represented by a monoclonal antibody. Loss or perturbation of binding of a monoclonal antibody in the panel can indicate mutational alteration of the p53 protein and thus of the p53 gene itself. Mutant p53 genes or gene products can also be detected in body samples, including, for example, serum, stool, urine, and sputum. The same techniques discussed above for detection of mutant p53 genes or gene products in tissues can be applied to other body samples.

Loss of wild type p53 genes can also be detected by screening for loss of wild type p53 protein function. Although all of the functions which the p53 protein undoubtedly possesses have yet to be elucidated, at least two specific functions are known. Protein p53 binds to the SV40 large T antigen as well as to the adenovirus E1B antigen. Loss of the ability of the p53 protein to bind to either or both of these antigens indicates a mutational alteration in the protein which reflects a mutational alteration of the gene itself. Alternatively, a panel of monoclonal antibodies could be used in which each of the epitopes involved in p53 functions are represented by a monoclonal antibody. Loss or perturbation of binding of a monoclonal antibody in the panel would indicate mutational alteration of the p53 protein and thus of the p53 gene itself. Any method for detecting an altered p53 protein can be used to detect loss of wild type p53 genes.

Determination of the lack of p53 deactivating mutation and/or expression of wild type p53 in the subject with cancer can be performed before, during, or after the administration of the peptidomimetic macrocycles. In some embodiments, the determination of the lack of a p53 deactivating mutation and/or expression of wild type p53 is performed before the first administration of the peptidomimetic macrocycle to the subject, for example about 5 years-about 1 month, about 4 years-about 1 month, about 3 years-1 month, about 2 years-about 1 month, about 1 years-about 1 month, about 5 years-about 1 week, about 4 years-about 1 week, about 3 years-about 1 month, about 2 years-about 1 week, about 1 year-about 1 week, about 5 years-about 1 day, about 4 years-about 1 day, about 3 years-about 1 day, about 2 years-about 1 day, about 1 year-about 1 day, about 15 months-about 1 month, about 15 months-about 1 week, about 15 months-about 1 day, about 12 months-about 1 month, about 12 months-about 1 week, about 12 months-about 1 day, about 6 months-1 about month, about 6 months-about 1 week, about 6 months-about 1 day, about 3 months-1 about month, about 3 months-about 1 week, or about 3 months-about 1 day prior to the first administration of the peptidomimetic macrocycle to the subject. In some examples, the confirmation of the lack of the p53 deactivating mutation and/or expression of wild type p53 is performed up to 6 years, 5 years, 4 years, 3 years, 24 months, 23 months, 22 months, 21 months, 20 months, 19 months, 18 months, 17 months, 16 months, 15 months, 14 months, 13 months, 12 months, 11 months, 10 months, 9 months, 8 months, 7 months, 6 months, 5 months, 4 months, 3 months, 2 months, 1 months, 4 weeks (28 days), 3 weeks (21 days), 2 weeks (14 days), 1 week (7 days), 6 days, 5 days, 4 days, 3 days, 2 days or 1 day before the first administration of the peptidomimetic macrocycle to the subject. In some examples the confirmation of the lack of the p53 deactivating mutation is performed within 1 month of the first administration of the peptidomimetic macrocycle to the subject. In some examples the confirmation of the lack of the p53 deactivating mutation is performed within 21 days of the first administration of the peptidomimetic macrocycle to the subject.

### Cancers

Solid cancers that can be treated by the instant methods include, but are not limited to, bone tumors (e.g. osteosarcoma, chondroblastoma, chondrosarcoma, Ewing sarcoma), germ cell tumors, renal tumors (e.g. Wilms tumor, malignant rhabdoid tumor), liver tumors (e.g. hepatoblastoma and hepatocellular carcinoma), neuroblastoma, melanoma, adrenocortical carcinoma, nasopharyngeal carcinoma, thyroid carcinoma, retinoblastoma, sarcomas and soft tissue tumors (e.g., rhabdomyosarcoma, desmoid tumor, fibrosarcoma, liposarcoma, malignant fibrous histiocytoma, and peripheral nerve sheath tumor (neurofibrosarcoma).

Liquid cancers that can be treated by the instant methods include, but are not limited to, lymphomas, leukemias, and myelomas. Examples of lymphomas and leukemias that can be treated in accordance with the methods described include, but are not limited to, chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma.

Examples of cancers that can be treated by the methods of the disclosure include cancers involving hyperplastic/neoplastic cells of hematopoietic origin, e.g., arising from myeloid, lymphoid or erythroid lineages, or precursor cells thereof. Examples of disorders include: acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), multiple mylenoma, hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. For example, cancers include, but are not limited to, acute lymphocytic leukemia (ALL); T-cell acute lymphocytic leukemia (T-ALL); anaplastic large cell lymphoma (ALCL); chronic myelogenous leukemia (CML); acute myeloid leukemia (AML); B-cell chronic lymphocytic leukemia (B-CLL); diffuse large B-cell lymphomas (DLBCL); hyper eosinophilia/chronic eosinophilia; and Burkitt's lymphoma.

In some embodiments, the cancer treated by the methods of the disclosure is an acute lymphoblastic leukemia; acute myeloid leukemia; AIDS-related cancers; AIDS-related lymphoma; chronic lymphocytic leukemia; chronic myelogenous leukemia; chronic myeloproliferative disorders; cutaneous T-cell lymphoma; Hodgkin lymphoma; multiple myeloma; multiple myeloma/plasma cell neoplasm; Non-Hodgkin lymphoma; primary central nervous system (CNS) lymphoma; or T-cell lymphoma; In various embodiments, the cancer can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma.

In some embodiments cancers treated by the methods disclosed herein exclude cancers that are known to be associated with HPV (human papillomavirus).

The effectiveness and/or response of cancer treatment by the methods disclosed herein can be determined by any suitable method. The response can be a complete response, and which can be an objective response, a clinical response, or a pathological response to treatment. For example, the response can be determined based upon the techniques for evaluating response to treatment of cancers as described in or by Revised International Working Group Response Criteria for lymphoma patients (IWG 2014), which is hereby incorporated by reference in its entirety. The response can be a duration of survival (or probability of such duration) or progression-free interval. The timing or duration of such events can be determined from about the time of diagnosis, or from about the time treatment is initiated or from about the time treatment is finished (like the final administration of the peptidomimetic macrocycle). Alternatively, the response can be based upon a reduction in the number of cancer cells, the number of cancer cells per unit volume, or cancer cell metabolism, or based upon cancer cell burden, or based upon levels of serum markers especially where elevated in the disease state.

The response in individual patients can be characterized as a complete response, a partial response, stable disease, and progressive disease. In some embodiments, the response is complete response (CR). Complete response, in some examples can be defined as disappearance of all circulating tumor cells (CTC) or a mononuclear blood cells (MNBC) i.e. any pathological lymph nodes (whether target or non-target) must have reduction in short axis to <10 mm. In certain embodiments, the response is a partial response (PR). Partial response can be defined to mean at least 30% decrease in the sum of diameters of circulating tumor cells (CTC) or a mononuclear blood cells (MNBC), taking as reference the baseline sum diameters. In some embodiments, the response is progressive disease (PD). Progressive disease can be defined as at least a 20% increase in the number of circulating tumor cells (CTC) or a mononuclear blood cells (MNBC), taking as reference the smallest number on study (this includes the baseline number if that is the smallest) and an absolute increase of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, or at least 100 or more circulating tumor cells (CTC) or a mononuclear blood cells (MNBC). The appearance of one or more new lesions can also be considered as progression. In some embodiments, the disease can be stable disease (SD). Stable disease can be characterized by neither sufficient decrease in cancer cell number to qualify for PR nor sufficient increase to qualify for PD, taking as reference the smallest number of CTCs and/or MNBCs while on study. In certain embodiments, the response is a pathological complete response. A pathological complete response, e.g., as determined by a pathologist following examination of tissue removed at the time of surgery or biopsy, generally refers to an absence of histological evidence of invasiveand/or non-invasive cancer cells in the surgical specimen.

### Combination Treatment

Also provided herein are combination therapies for the treatment of a cancer which involve the administration of the peptidomimetic macrocycles disclosed herein in combination with one or more additional therapies to a subject with cancer determined to lack a p53 deactivating mutation and/or express wild type p53. In a specific embodiment, presented herein are combination therapies for the treatment of cancer which involve the administration of an effective amount of the peptidomimetic macrocycles in combination with an effective amount of another therapy to a subject with a cancer determined to lack a p53 deactivating mutation and/or with a cancer expressing wild type p53.

As used herein, the term “in combination,” refers, in the context of the administration of the peptidomimetic macrocycles, to the administration of the peptidomimetic macrocycles prior to, concurrently with, or subsequent to the administration of one or more additional therapies (e.g., agents, surgery, or radiation) for use in treating cancer. The use of the term “in combination” does not restrict the order in which the peptidomimetic macrocycles and one or more additional therapies are administered to a subject. In specific embodiments, the interval of time between the administration of the peptidomimetic macrocycles and the administration of one or more additional therapies can be about 1-about 5 minutes, about 1-about 30 minutes, about 30 minutes to about 60 minutes, about 1 hour, about 1-about 2 hours, about 2-about 6 hours, about 2-about 12 hours, about 12-about 24 hours, about 1-about 2 days, about 2 days, about 3 days, about 4 days, about 5 days, about 6 days, about 7 days, about 1 week, about 2 weeks, about 3 weeks, about 4 weeks, about 5 weeks, about 6 weeks, about 7 weeks, about 8 weeks, about 9 weeks, about 10 weeks, about 15 weeks, about 20 weeks, about 26 weeks, about 52 weeks, about 11-about 15 weeks, about 15-about 20 weeks, about 20-about 30 weeks, about 30-about 40 weeks, about 40-about 50 weeks, about 1 month, about 2 months, about 3 months, about 4 months, about 5 months, about 6 months, about 7 months, about 8 months, about 9 months, about 10 months, about 11 months, about 12 months, about 1 year, about 2 years, or any period of time in between. In certain embodiments, the peptidomimetic macrocycles and one or more additional therapies are administered less than 1 day, less than 1 week, less than 2 weeks, less than 3 weeks, less than 4 weeks, less than one month, less than 2 months, less than 3 months, less than 6 months, less than 1 year, less than 2 years, or less than 5 years apart.

In some embodiments, the combination therapies provided herein involve administering of the peptidomimetic macrocycles 1-2 times a week, once every week, once every 2 weeks, once every 3 weeks, once every 4 weeks, once every 5 weeks, once every 6 weeks, once every 7 weeks or once every 8 weeks and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain embodiments, the peptidomimetic macrocycles and one or more additional therapies are cyclically administered to a subject. Cycling therapy involves the administration of the peptidomimetic macrocycles compounds for a period of time, followed by the administration of one or more additional therapies for a period of time, and repeating this sequential administration. In certain embodiments, cycling therapy can also include a period of rest where the peptidomimetic macrocycles or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an embodiment, the number of cycles administered is from 1 to 12 cycles, from 2 to 10 cycles, or from 2 to 8 cycles.

In some embodiments, the methods for treating cancer provided herein comprise administering the peptidomimetic macrocycles as a single agent for a period of time prior to administering the peptidomimetic macrocycles in combination with an additional therapy. In certain embodiments, the methods for treating cancer provided herein comprise administering an additional therapy alone for a period of time prior to administering the peptidomimetic macrocycles in combination with the additional therapy.

In some embodiments, the administration of the peptidomimetic macrocycles and one or more additional therapies in accordance with the methods presented herein have an additive effect relative the administration of the peptidomimetic macrocycles or said one or more additional therapies alone. In some embodiments, the administration of the peptidomimetic macrocycles and one or more additional therapies in accordance with the methods presented herein have a synergistic effect relative to the administration of the peptidomimetic macrocycles or said one or more additional therapies alone.

As used herein, the term “synergistic,” refers to the effect of the administration of the peptidomimetic macrocycles in combination with one or more additional therapies (e.g., agents), which combination is more effective than the additive effects of any two or more single therapies (e.g., agents). In a specific embodiment, a synergistic effect of a combination therapy permits the use of lower dosages (e.g., sub-optimal doses) of the peptidomimetic macrocycles or an additional therapy and/or less frequent administration of the peptidomimetic macrocycles or an additional therapy to a subject. In certain embodiments, the ability to utilize lower dosages of the peptidomimetic macrocycles or of an additional therapy and/or to administer the peptidomimetic macrocycles or said additional therapy less frequently reduces the toxicity associated with the administration of the peptidomimetic macrocycles or of said additional therapy, respectively, to a subject without reducing the efficacy of the peptidomimetic macrocycles or of said additional therapy, respectively, in the treatment of cancer. In some embodiments, a synergistic effect results in improved efficacy of the peptidomimetic macrocycles and each of said additional therapies in treating cancer. In some embodiments, a synergistic effect of a combination of the peptidomimetic macrocycles and one or more additional therapies avoids or reduces adverse or unwanted side effects associated with the use of any single therapy.

The combination of the peptidomimetic macrocycles and one or more additional therapies can be administered to a subject in the same pharmaceutical composition. Alternatively, the peptidomimetic macrocycles and one or more additional therapies can be administered concurrently to a subject in separate pharmaceutical compositions. The peptidomimetic macrocycles and one or more additional therapies can be administered sequentially to a subject in separate pharmaceutical compositions. The peptidomimetic macrocycles compounds and one or more additional therapies can also be administered to a subject by the same or different routes of administration.

The combination therapies provided herein involve administering to a subject to in need thereof the peptidomimetic macrocycles in combination with conventional, or known, therapies for treating cancer. Other therapies for cancer or a condition associated therewith are aimed at controlling or relieving one or more symptoms. Accordingly, in some embodiments, the combination therapies provided herein involve administering to a subject to in need thereof a pain reliever, or other therapies aimed at alleviating or controlling one or more symptoms associated with or a condition associated therewith.

Non-limiting specific examples of anti-cancer agents that can be used in combination with the peptidomimetic macrocycles include: a hormonal agent (e.g., aromatase inhibitor, selective estrogen receptor modulator (SERM), and estrogen receptor antagonist), chemotherapeutic agent (e.g., microtubule disassembly blocker, antimetabolite, topoisomerase inhibitor, and DNA crosslinker or damaging agent), anti-antigenic agent (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that can be used in combination with the peptidomimetic macrocycles include aromatase inhibitors, SERMs, and estrogen receptor antagonists. Hormonal agents that are aromatase inhibitors can be steroidal or no steroidal. Non-limiting examples of no steroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that can be used in combination with of peptidomimetic macrocycles include microtubule disassembly blocker, antimetabolite, topoisomerase inhibitor, and DNA crosslinker or damaging agent. Chemotherapeutic agents that are microtubule disassembly blockers include, but are not limited to, taxanes (e.g., paclitaxel (branded/marketed as TAXOL®), docetaxel, abraxane, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapeutic agents that are antimetabolites include, but are not limited to, folate anitmetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (GEMZAR®), cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, Ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

Non-limiting examples of other therapies that can be administered to a subject in combination with the peptidomimetic macrocycles include: (1) a statin such as lovostatin (e.g., branded/marketed as MEVACOR®); (2) an mTOR inhibitor such as sirolimus which is also known as Rapamycin (e.g., branded/marketed as RAPAMUNE®), temsirolimus (e.g., branded/marketed as TORISEL®), evorolimus (e.g., branded/marketed as AFINITOR®), and deforolimus; (3) a farnesyltransferase inhibitor agent such as tipifarnib; (4) an antifibrotic agent such as pirfenidone; (5) a pegylated interferon such as PEG-interferon alfa-2b; (6) a CNS stimulant such as methylphenidate (branded/marketed as RITALIN®); (7) a HER-2 antagonist such as anti-HER-2 antibody (e.g., trastuzumab) and kinase inhibitor (e.g., lapatinib); (8) an IGF-1 antagonist such as an anti-IGF-1 antibody (e.g., AVE1642 and IMC-A11) or an IGF-1 kinase inhibitor; (9) EGFR/HER-1 antagonist such as an anti-EGFR antibody (e.g., cetuximab, panitumamab) or EGFR kinase inhibitor (e.g., erlotinib; gefitinib); (10) SRC antagonist such as bosutinib; (11) cyclin dependent kinase (CDK) inhibitor such as seliciclib; (12) Janus kinase 2 inhibitor such as lestaurtinib; (13) proteasome inhibitor such as bortezomib; (14) phosphodiesterase inhibitor such as anagrelide; (15) inosine monophosphate dehydrogenase inhibitor such as tiazofurine; (16) lipoxygenase inhibitor such as masoprocol; (17) endothelin antagonist; (18) retinoid receptor antagonist such as tretinoin or alitretinoin; (19) immune modulator such as lenalidomide, pomalidomide, or thalidomide; (20) kinase (e.g., tyrosine kinase) inhibitor such as imatinib, dasatinib, erlotinib, nilotinib, gefitinib, sorafenib, sunitinib, lapatinib, or TG100801; (21) non-steroidal anti-inflammatory agent such as celecoxib (branded/marketed as CELEBREX®); (22) human granulocyte colony-stimulating factor (G-CSF) such as filgrastim (branded/marketed as NEUPOGEN®); (23) folinic acid or leucovorin calcium; (24) integrin antagonist such as an integrin α5β1-antagonist (e.g., JSM6427); (25) nuclear factor kappa beta (NF-κβ) antagonist such as OT-551, which is also an anti-oxidant. (26) hedgehog inhibitor such as CUR61414, cyclopamine, GDC-0449, and anti-hedgehog antibody; (27) histone deacetylase (HDAC) inhibitor such as SAHA (also known as vorinostat (branded/marketed as ZOLINZA)), PCI-24781, SB939, CHR-3996, CRA-024781, ITF2357, JNJ-26481585, or PCI-24781; (28) retinoid such as isotretinoin (e.g., branded/marketed as ACCUTANE®); (29) hepatocyte growth factor/scatter factor (HGF/SF) antagonist such as HGF/SF monoclonal antibody (e.g., AMG 102); (30) synthetic chemical such as antineoplaston; (31) anti-diabetic such as rosaiglitazone (e.g., branded/marketed as AVANDIA®); (32) antimalarial and amebicidal drug such as chloroquine (e.g., branded/marketed as ARALEN®); (33) synthetic bradykinin such as RMP-7; (34) platelet-derived growth factor receptor inhibitor such as SU-101; (35) receptor tyrosine kinase inhibitorsof Flk-1/KDR/VEGFR2, FGFR1 and PDGFR beta such as SU5416 and SU6668; (36) anti-inflammatory agent such as sulfasalazine (e.g., branded/marketed as AZULFIDINE®); and (37) TGF-beta antisense therapy.

In some embodiments the peptidomimetic macrocycles disclosed herein can inhibit one or more transporter enzymes (e.g., OATP1B1, OATP1B3, BSEP) at concentrations that can be clinically relevant. Therefore the peptidomimetic macrocycles disclosed herein can interact with medications that are predominantly cleared by hepatobiliary transporters. In particular, methotrexate and statins (e.g., atorvastatin, fluvastatin lovastatin, pitavastatin pravastatin, rosuvastatin and simvastatin) can not be dosed within 48 h, 36 h, 24 h, or 12 h ((for example within 24 h) of the administration of the peptidomimetic macrocycles disclosed herein. Examples of medications that can be affected by co-administration with peptidomimetic macrocycles disclosed herein are listed below. In various embodiments one or more of the medications selected from Table 8 is not dosed within 48 h, 36 h, 24 h, or 12 h (for example within 24 h) of the administration of the peptidomimetic macrocycles disclosed herein.

Example medications that can be affected by co-administration with peptidomimetic macrocycles disclosed in Table 8.

TABLE 8

| Medication | Therapeutic Area |
|---|---|
| Irinotecan | Oncology |
| Bosentan | Pulmonary artery hypertension |
| Caspofungin | Antifungal |
| Methotrexate | Oncology & rheumatology |
| Repaglinide | Diabetes mellitus |
| Atorvastatin | Hypercholesterolemia |
| Cerivastatin | Hypercholesterolemia |
| Fluvastatin | Hypercholesterolemia |
| Lovastatin | Hypercholesterolemia |
| Pitavastatin | Hypercholesterolemia |
| Pravastatin | Hypercholesterolemia |
| Rosuvastatin | Hypercholesterolemia |
| Simvastatin | Hypercholesterolemia |

### EXAMPLES

### Example 1. Synthesis of 6-Chlorotryptophan Fmoc Amino Acids

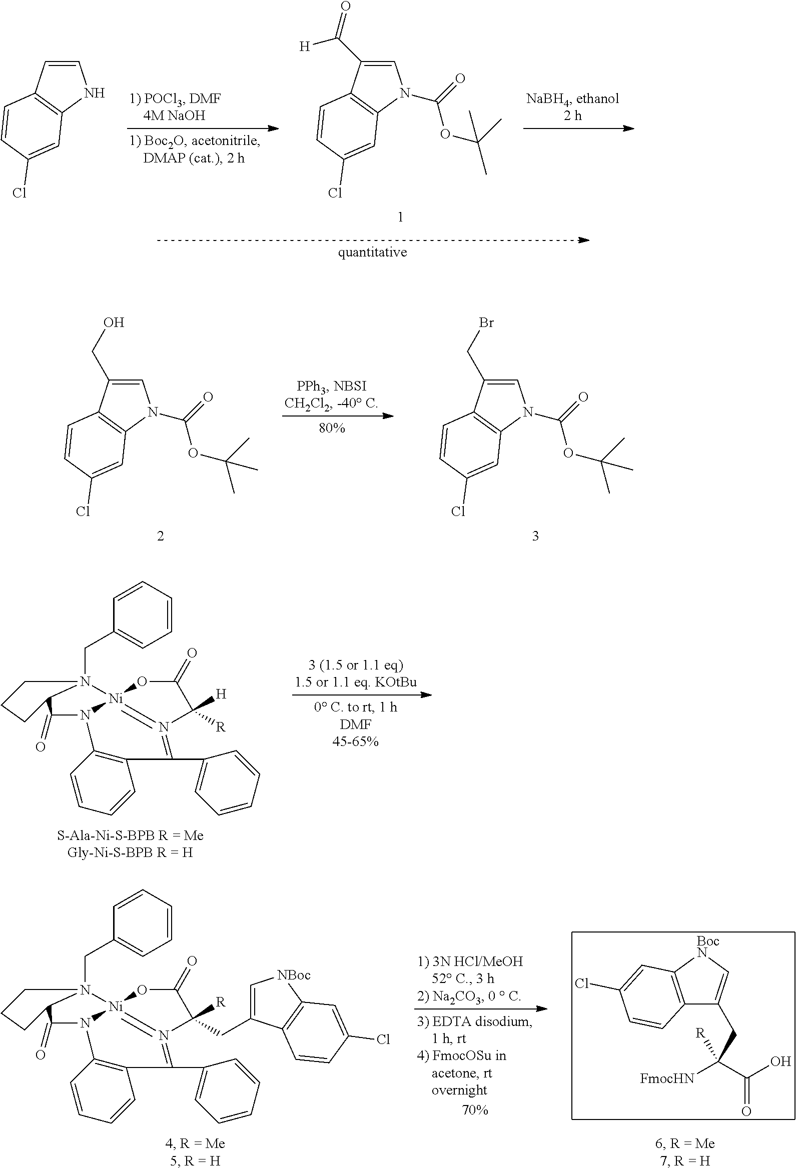

Tert-butyl 6-chloro-3-formyl-1H-indole-1-carboxylate, 1. To a stirred solution of dry DMF (12 mL) was added dropwise POCl₃ (3.92 mL, 43 mmol, 1.3 equiv) at 0° C. under Argon. The solution was stirred at the same temperature for 20 min before a solution of 6-chloroindole (5.0 g, 33 mmol, 1 eq.) in dry DMF (30 mL) was added dropwise. The resulting mixture was allowed to warm to room temperature and stirred for an additional 2.5 h. Water (50 mL) was added and the solution was neutralized with 4M aqueous NaOH (pH˜8). The resulting solid was filtered off, washed with water and dried under vacuum. This material was directly used in the next step without additional purification. To a stirred solution of the crude formyl indole (33 mmol, 1 eq.) in THF (150 mL) was added successively Boc₂O (7.91 g, 36.3 mmol, 1.1 equiv) and DMAP (0.4 g, 3.3 mmol, 0.1 equiv) at room temperature under N₂. The resulting mixture was stirred at room temperature for 1.5 h and the solvent was evaporated under reduced pressure. The residue was taken up in EtOAc and washed with 1N HCl, dried and concentrated to give the formyl indole 1 (9 g, 98% over 2 steps) as a white solid. ¹H NMR (CDCl₃) δ: 1.70 (s, Boc, 9H); 7.35 (dd, 1H); 8.21 (m, 3H); 10.07 (s, 1H).

Tert-butyl 6-chloro-3-(hydroxymethyl)-1H-indole-1-carboxylate, 2. To a solution of compound 1 (8.86 g, 32 mmol, 1 eq.) in ethanol (150 mL) was added NaBH₄ (2.4 g, 63 mmol, 2 eq.). The reaction was stirred for 3 h at room temperature. The reaction mixture was concentrated and the residue was poured into diethyl ether and water. The organic layer was separated, dried over magnesium sulfate and concentrated to give a white solid (8.7 g, 98%). This material was directly used in the next step without additional purification. ¹H NMR (CDCl₃) δ: 1.65 (s, Boc, 9H); 4.80 (s, 2H, CH₂); 7.21 (dd, 1H); 7.53 (m, 2H); 8.16 (bs, 1H).

Tert-butyl 3-(bromomethyl)-6-chloro-1H-indole-1-carboxylate, 3. To a solution of compound 2 (4.1 g, 14.6 mmol, 1 eq.) in dichloromethane (50 mL) under argon was added a solution of triphenylphosphine (4.59 g, 17.5 mmol, 1.2 eq.) in dichloromethane (50 mL) at −40° C. The reaction solution was stirred an additional 30 min at 40° C. Then NBS (3.38 g, 19 mmol, 1.3 eq.) was added. The resulting mixture was allowed to warm to room temperature and stirred overnight. Dichloromethane was evaporated, Carbon Tetrachloride (100 mL) was added and the mixture was stirred for 1h and filtrated. The filtrate was concentrated, loaded in a silica plug and quickly eluted with 25% EtOAc in Hexanes. The solution was concentrated to give a white foam (3.84 g, 77%). ¹H NMR (CDCl₃) δ: 1.66 (s, Boc, 9H); 4.63 (s, 2H, CH₂); 7.28 (dd, 1H); 7.57 (d, 1H); 7.64 (bs, 1H); 8.18 (bs, 1H).

αMe-6Cl-Trp(Boc)-Ni—S-BPB, 4. To S-A1α-Ni—S-BPB (2.66 g, 5.2 mmol, 1 eq.) and KO-tBu (0.87 g, 7.8 mmol, 1.5 eq.) was added 50 mL of DMF under argon. The bromide derivative compound 3 (2.68 g, 7.8 mmol, 1.5 eq.) in solution of DMF (5.0 mL) was added via syringe. The reaction mixture was stirred at ambient temperature for 1 h. The solution was then quenched with 5% aqueous acetic acid and diluted with water. The desired product was extracted in dichloromethane, dried and concentrated. The oily product 4 was purified by flash chromatography (solid loading) on normal phase using EtOAc and Hexanes as eluents to give a red solid (1.78 g, 45% yield). αMe-6Cl-Trp(Boc)-Ni—S-BPB, 4: M+H calc. 775.21, M+H obs. 775.26; ¹H NMR (CDCl₃) δ: 1.23 (s, 3H, αMe); 1.56 (m, 11H, Boc+CH₂); 1.82-2.20 (m, 4H, 2CH₂); 3.03 (m, 1H, CH_{α}); 3.24 (m, 2H, CH₂); 3.57 and 4.29 (AB system, 2H, CH₂ (benzyl), J=12.8 Hz); 6.62 (d, 2H); 6.98 (d, 1H); 7.14 (m, 2H); 7.23 (m, 1H); 7.32-7.36 (m, 5H); 7.50 (m, 2H); 7.67 (bs, 1H); 7.98 (d, 2H); 8.27 (m, 2H).

Fmoc-αMe-6Cl-Trp(Boc)-OH, 6. To a solution of 3 N HCl/MeOH (1/3, 15 mL) at 50° C. was added a solution of compound 4 (1.75 g, 2.3 mmol, 1 eq.) in MeOH (5 ml) dropwise. The starting material disappeared within 3-4 h. The acidic solution was then cooled to 0° C. with an ice bath and quenched with an aqueous solution of Na₂CO₃ (1.21 g, 11.5 mmol, 5 eq.). Methanol was removed and 8 more equivalents of Na₂CO₃ (1.95 g, 18.4 mmol) were added to the suspension. The Nickel scavenging EDTA disodium salt dihydrate (1.68 g, 4.5 mmol, 2 eq.) was then added and the suspension was stirred for 2 h. A solution of Fmoc-OSu (0.84 g, 2.5 mmol, 1.1 eq.) in acetone (50 mL) was added and the reaction was stirred overnight. Afterwards, the reaction was diluted with diethyl ether and 1N HCl. The organic layer was then dried over magnesium sulfate and concentrated in vacuo. The desired product 6 was purified on normal phase using acetone and dichloromethane as eluents to give a white foam (0.9 g, 70% yield). Fmoc-αMe-6Cl-Trp(Boc)-OH, 6: M+H calc. 575.19, M+H obs. 575.37; ¹H NMR (CDCl₃) 1.59 (s, 9H, Boc); 1.68 (s, 3H, Me); 3.48 (bs, 2H, CH₂); 4.22 (m, 1H, CH); 4.39 (bs, 2H, CH₂); 5.47 (s, 1H, NH); 7.10 (m, 1H); 7.18 (m, 2H); 7.27 (m, 2H); 7.39 (m, 2H); 7.50 (m, 2H); 7.75 (d, 2H); 8.12 (bs, 1H).

6Cl-Trp(Boc)-Ni—S-BPB, 5. To Gly-Ni—S-BPB (4.6 g, 9.2 mmol, 1 eq.) and KO-tBu (1.14 g, 10.1 mmol, 1.1 eq.) was added 95 mL of DMF under argon. The bromide derivative compound 3 (3.5 g, 4.6 mmol, 1.1 eq.) in solution of DMF (10 mL) was added via syringe. The reaction mixture was stirred at ambient temperature for 1 h. The solution was then quenched with 5% aqueous acetic acid and diluted with water. The desired product was extracted in dichloromethane, dried and concentrated. The oily product 5 was purified by flash chromatography (solid loading) on normal phase using EtOAc and Hexanes as eluents to give a red solid (5 g, 71% yield). 6Cl-Trp(Boc)-Ni—S-BPB, 5: M+H calc. 761.20, M+H obs. 761.34; ¹H NMR (CDCl₃) δ: 1.58 (m, 11H, Boc+CH₂); 1.84 (m, 1H); 1.96 (m, 1H); 2.24 (m, 2H, CH₂); 3.00 (m, 1H, CHₐ); 3.22 (m, 2H, CH₂); 3.45 and 4.25 (AB system, 2H, CH₂ (benzyl), J=12.8 Hz); 4.27 (m, 1H, CHₐ); 6.65 (d, 2H); 6.88 (d, 1H); 7.07 (m, 2H); 7.14 (m, 2H); 7.28 (m, 3H); 7.35-7.39 (m, 2H); 7.52 (m, 2H); 7.96 (d, 2H); 8.28 (m, 2H).

Fmoc-6Cl-Trp(Boc)-OH, 7. To a solution of 3N HCl/MeOH (1/3, 44 mL) at 50° C. was added a solution of compound 5 (5 g, 6.6 mmol, 1 eq.) in MeOH (10 ml) dropwise. The starting material disappeared within 3-4 h. The acidic solution was then cooled to 0° C. with an ice bath and quenched with an aqueous solution of Na₂CO₃ (3.48 g, 33 mmol, 5 eq.). Methanol was removed and 8 more equivalents of Na₂CO₃ (5.57 g, 52 mmol) were added to the suspension. The Nickel scavenging EDTA disodium salt dihydrate (4.89 g, 13.1 mmol, 2 eq.) and the suspension was stirred for 2 h. A solution of Fmoc-OSu (2.21 g, 6.55 mmol, 1.1 eq.) in acetone (100 mL) was added and the reaction was stirred overnight. Afterwards, the reaction was diluted with diethyl ether and 1N HCl. The organic layer was then dried over magnesium sulfate and concentrated in vacuo. The desired product 7 was purified on normal phase using acetone and dichloromethane as eluents to give a white foam (2.6 g, 69% yield). Fmoc-6Cl-Trp(Boc)-OH, 7: M+H calc. 561.17, M+H obs. 561.37; ¹H NMR (CDCl₃) 1.63 (s, 9H, Boc); 3.26 (m, 2H, CH₂); 4.19 (m, 1H, CH); 4.39 (m, 2H, CH₂); 4.76 (m, 1H); 5.35 (d, 1H, NH); 7.18 (m, 2H); 7.28 (m, 2H); 7.39 (m, 3H); 7.50 (m, 2H); 7.75 (d, 2H); 8.14 (bs, 1H).

Reactions from Example 1 are shown in FIG. 1.

### Example 2. Peptidomimetic Macrocycles of the Invention

Peptidomimetic macrocycles were synthesized, purified and analyzed as previously described and as described below (Schafmeister et al., J. Am. Chem. Soc. 122:5891-5892 (2000); Schafmeister & Verdin, J. Am. Chem. Soc. 122:5891 (2005); Walensky et al., Science 305:1466-1470 (2004); and U.S. Pat. No. 7,192,713). Peptidomimetic macrocycles were designed by replacing two or more naturally occurring amino acids with the corresponding synthetic amino acids. Substitutions were made at i and i+4, and i and i+7 positions. Peptide synthesis was performed either manually or on an automated peptide synthesizer (Applied Biosystems, model 433 Å), using solid phase conditions, rink amide AM resin (Novabiochem), and Fmoc main-chain protecting group chemistry. For the coupling of natural Fmoc-protected amino acids (Novabiochem), 10 equivalents of amino acid and a 1:1:2 molar ratio of coupling reagents HBTU/HOBt (Novabiochem)/DIEA were employed. Non-natural amino acids (4 equiv) were coupled with a 1:1:2 molar ratio of HATU (Applied Biosystems)/HOBt/DIEA. The N-termini of the synthetic peptides were acetylated, while the C-termini were amidated.

The fully protected resin-bound peptides are synthesized on a Rink amide MBHA resin (loading 0.62 mmol/g) on a 0.1 mmol scale. Deprotection of the temporary Fmoc group is achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate preactivated Fmoc-amino acid derivative. All protected amino acids (1 mmol) were dissolved in NMP and activated with HCTU (1 mmol) and DIEA (1 mmol) prior to transfer of the coupling solution to the deprotected resin-bound peptide. After coupling was completed, the resin was extensively flow washed in preparation for the next deprotection/coupling cycle. Acetylation of the amino terminus was carried out in the presence of acetic anhydride/DIEA in NMP/NMM. The LC-MS analysis of a cleaved and deprotected sample obtained from an aliquot of the fully assembled resin-bound peptide was accomplished in order to verifying the completion of each coupling.

Purification of cross-linked compounds was achieved by high performance liquid chromatography (HPLC) (Varian ProStar) on a reverse phase C18 column (Varian) to yield the pure compounds. Chemical composition of the pure products was confirmed by LC/MS mass spectrometry (Micromass LCT interfaced with Agilent 1100 HPLC system) and amino acid analysis (Applied Biosystems, model 420 Å).

The following protocol was used in the synthesis of dialkyne-crosslinked peptidomimetic macrocycles, including SP662, SP663 and SP664. Fully protected resin-bound peptides were synthesized on a PEG-PS resin (loading 0.45 mmol/g) on a 0.2 mmol scale. Deprotection of the temporary Fmoc group was achieved by 3×10 min treatments of the resin bound peptide with 20% (v/v) piperidine in DMF. After washing with NMP (3×), dichloromethane (3×) and NMP (3×), coupling of each successive amino acid was achieved with 1×60 min incubation with the appropriate preactivated Fmoc-amino acid derivative. All protected amino acids (0.4 mmol) were dissolved in NMP and activated with HCTU (0.4 mmol) and DIEA (0.8 mmol) prior to transfer of the coupling solution to the deprotected resin-bound peptide. After coupling was completed, the resin was washed in preparation for the next deprotection/coupling cycle. Acetylation of the amino terminus was carried out in the presence of acetic anhydride/DIEA in NMP. The LC-MS analysis of a cleaved and deprotected sample obtained from an aliquot of the fully assembled resin-bound peptide was accomplished in order to verifying the completion of each coupling. In a typical example, tetrahydrofuran (4 ml) and triethylamine (2 ml) were added to the peptide resin (0.2 mmol) in a 40 ml glass vial and shaken for 10 minutes. Pd(PPh₃)₂Cl₂ (0.014 g, 0.02 mmol) and copper iodide (0.008 g, 0.04 mmol) were then added and the resulting reaction mixture was mechanically shaken 16 hours while open to atmosphere. The diyne-cyclized resin-bound peptides were deprotected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC.

In a typical example, a peptide resin (0.1 mmol) was washed with DCM. Deprotection of the temporary Mmt group was achieved by 3×3 min treatments of the resin bound peptide with 2% TFA/DCM 5% TIPS, then 30 min treatments until no orange color is observed in the filtrate. In between treatments the resin was extensively flow washed with DCM. After complete removal of Mmt, the resin was washed with 5% DIEA/NMP solution 3× and considered ready for bisthioether coupling. Resin was loaded into a reaction vial. DCM/DMF 1/1 was added to the reaction vessel, followed by DIEA (2.4 eq). After mixing well for 5 minutes, 4,4′-Bis(bromomethyl)biphenyl (1.05 eq) (TCI America B1921) was added. The reaction was then mechanically agitated at room temperature overnight. Where needed, the reaction was allowed additional time to reach completion. A similar procedure may be used in the preparation of five-methylene, six-methylene or seven-methylene crosslinkers (“% c7”, “% c6”, or “% c5”).

The bisthioether resin-bound peptides were deprotected and cleaved from the solid support by treatment with TFA/H₂O/TIS (94/3/3 v/v) for 3 h at room temperature. After filtration of the resin the TFA solution was precipitated in cold diethyl ether and centrifuged to yield the desired product as a solid. The crude product was purified by preparative HPLC. Table 20 shows a list of peptidomimetic macrocycles.

The following protocol was used in the synthesis of single alkyne-crosslinked peptidomimetic macrocycles, including SP665. Fully protected resin-bound peptides were synthesized on a Rink amide MBHA resin (loading 0.62 mmol/g) on a 0.1 mmol scale. Deprotection of the temporary Fmoc group was achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate preactivated Fmoc-amino acid derivative. All protected amino acids (1 mmol) were dissolved in NMP and activated with HCTU (1 mmol) and DIEA (1 mmol) prior to transfer of the coupling solution to the deprotected resin-bound peptide. After coupling was completed, the resin was extensively flow washed in preparation for the next deprotection/coupling cycle. Acetylation of the amino terminus was carried out in the presence of acetic anhydride/DIEA in NMP/NMM. The LC-MS analysis of a cleaved and deprotected sample obtained from an aliquot of the fully assembled resin-bound peptide was accomplished in order to verifying the completion of each coupling. In a typical example, the peptide resin (0.1 mmol) was washed with DCM. Resin was loaded into a microwave vial. The vessel was evacuated and purged with nitrogen. Molybdenumhexacarbonyl (0.01 eq, Sigma Aldrich 199959) was added. Anhydrous chlorobenzene was added to the reaction vessel. Then 2-fluorophenol (1 eq, Sigma Aldrich F12804) was added. The reaction was then loaded into the microwave and held at 130° C. for 10 minutes. Reaction may need to be pushed a subsequent time for completion. The alkyne metathesized resin-bound peptides were deprotected and cleaved from the solid support by treatment with TFA/H₂O/TIS (94/3/3 v/v) for 3 h at room temperature. After filtration of the resin the TFA solution was precipitated in cold diethyl ether and centrifuged to yield the desired product as a solid. The crude product was purified by preparative HPLC.

Table 9 shows a list of peptidomimetic macrocycles of the invention prepared.

TABLE 9

| | SEQ | | Exact | | Observed |
|---|---|---|---|---|---|
| Number | ID NO: | Sequence | Mass | M + 2 | mass (m/e) |
| 1 | 42 | Ac-LSQETF$r8DLWKLL$EN-NH2 | 2068.13 | 1035.07 | 1035.36 |
| 2 | 43 | Ac-LSQETF$r8NLWKLL$QN-NH2 | 2066.16 | 1034.08 | 1034.31 |
| 3 | 44 | Ac-LSQQTF$r8NLWRLL$QN-NH2 | 2093.18 | 1047.59 | 1047.73 |
| 4 | 45 | Ac-QSQQTF$r8NLWKLL$QN-NH2 | 2080.15 | 1041.08 | 1041.31 |
| 5 | 46 | Ac-QSQQTF$r8NLWRLL$QN-NH2 | 2108.15 | 1055.08 | 1055.32 |
| 6 | 47 | Ac-QSQQTA$r8NLWRLL$QN-NH2 | 2032.12 | 1017.06 | 1017.24 |
| 7 | 48 | Ac-QAibQQTF$r8NLWRLL$QN-NH2 | 2106.17 | 1054.09 | 1054.34 |
| 8 | 49 | Ac-QSQQTFSNLWRLLPQN-NH2 | 2000.02 | 1001.01 | 1001.26 |
| 9 | 50 | Ac-QSQQTF$/r8NLWRLL$/QN-NH2 | 2136.18 | 1069.09 | 1069.37 |
| 10 | 51 | Ac-QSQAibTF$r8NLWRLL$QN-NH2 | 2065.15 | 1033.58 | 1033.71 |
| 11 | 52 | Ac-QSQQTF$r8NLWRLL$AN-NH2 | 2051.13 | 1026.57 | 1026.70 |
| 12 | 53 | Ac-ASQQTF$r8NLWRLL$QN-NH2 | 2051.13 | 1026.57 | 1026.90 |
| 13 | 54 | Ac-QSQQTF$r8ALWRLL$QN-NH2 | 2065.15 | 1033.58 | 1033.41 |
| 14 | 55 | Ac-QSQETF$r8NLWRLL$QN-NH2 | 2109.14 | 1055.57 | 1055.70 |
| 15 | 56 | Ac-RSQQTF$r8NLWRLL$QN-NH2 | 2136.20 | 1069.10 | 1069.17 |
| 16 | 57 | Ac-RSQQTF$r8NLWRLL$EN-NH2 | 2137.18 | 1069.59 | 1069.75 |
| 17 | 58 | Ac-LSQETFSDLWKLLPEN-NH2 | 1959.99 | 981.00 | 981.24 |
| 18 | 59 | Ac-QSQ$TFS$LWRLLPQN-NH2 | 2008.09 | 1005.05 | 1004.97 |
| 19 | 60 | Ac-QSQQ$FSN$WRLLPQN-NH2 | 2036.06 | 1019.03 | 1018.86 |
| 20 | 61 | Ac-QSQQT$SNL$RLLPQN-NH2 | 1917.04 | 959.52 | 959.32 |
| 21 | 62 | Ac-QSQQTF$NLW$LLPQN-NH2 | 2007.06 | 1004.53 | 1004.97 |
| 22 | 63 | Ac-RTQATF$r8NQWAibANle$TNAibTR-NH2 | 2310.26 | 1156.13 | 1156.52 |
| 23 | 64 | Ac-QSQQTF$r8NLWRLL$RN-NH2 | 2136.20 | 1069.10 | 1068.94 |
| 24 | 65 | Ac-QSQRTF$r8NLWRLL$QN-NH2 | 2136.20 | 1069.10 | 1068.94 |
| 25 | 66 | Ac-QSQQTF$r8NNleWRLL$QN-NH2 | 2108.15 | 1055.08 | 1055.44 |
| 26 | 67 | Ac-QSQQTF$r8NLWRNleL$QN-NH2 | 2108.15 | 1055.08 | 1055.84 |
| 27 | 68 | Ac-QSQQTF$r8NLWRLNle$QN-NH2 | 2108.15 | 1055.08 | 1055.12 |
| 28 | 69 | Ac-QSQQTY$r8NLWRLL$QN-NH2 | 2124.15 | 1063.08 | 1062.92 |
| 29 | 70 | Ac-RAibQQTF$r8NLWRLL$QN-NH2 | 2134.22 | 1068.11 | 1068.65 |
| 30 | 71 | Ac-MPRFMDYWEGLN-NH2 | 1598.70 | 800.35 | 800.45 |
| 31 | 72 | Ac-RSQQRF$r8NLWRLL$QN-NH2 | 2191.25 | 1096.63 | 1096.83 |
| 32 | 73 | Ac-QSQQRF$r8NLWRLL$QN-NH2 | 2163.21 | 1082.61 | 1082.87 |
| 33 | 74 | Ac-RAibQQRF$r8NLWRLL$QN-NH2 | 2189.27 | 1095.64 | 1096.37 |
| 34 | 75 | Ac-RSQQRF$r8NFWRLL$QN-NH2 | 2225.23 | 1113.62 | 1114.37 |
| 35 | 76 | Ac-RSQQRF$r8NYWRLL$QN-NH2 | 2241.23 | 1121.62 | 1122.37 |
| 36 | 77 | Ac-RSQQTF$r8NLWQLL$QN-NH2 | 2108.15 | 1055.08 | 1055.29 |
| 37 | 78 | Ac-QSQQTF$r8NLWQAmlL$QN-NH2 | 2094.13 | 1048.07 | 1048.32 |
| 38 | 79 | Ac-QSQQTF$r8NAmlWRLL$QN-NH2 | 2122.17 | 1062.09 | 1062.35 |
| 39 | 80 | Ac-NlePRF$r8DYWEGL$QN-NH2 | 1869.98 | 935.99 | 936.20 |
| 40 | 81 | Ac-NlePRF$r8NYWRLL$QN-NH2 | 1952.12 | 977.06 | 977.35 |
| 41 | 82 | Ac-RF$r8NLWRLL$Q-NH2 | 1577.96 | 789.98 | 790.18 |
| 42 | 83 | Ac-QSQQTF$r8N2ffWRLL$QN-NH2 | 2160.13 | 1081.07 | 1081.40 |
| 43 | 84 | Ac-QSQQTF$r8N3ffWRLL$QN-NH2 | 2160.13 | 1081.07 | 1081.34 |
| 44 | 85 | Ac-QSQQTF#r8NLWRLL#QN-NH2 | 2080.12 | 1041.06 | 1041.34 |
| 45 | 86 | Ac-RSQQTA$r8NLWRLL$QN-NH2 | 2060.16 | 1031.08 | 1031.38 |
| 46 | 87 | Ac-QSQQTF%r8NLWRLL%QN-NH2 | 2110.17 | 1056.09 | 1056.55 |
| 47 | 88 | HepQSQ$TFSNLWRLLPQN-NH2 | 2051.10 | 1026.55 | 1026.82 |
| 48 | 89 | HepQSQ$TF$r8NLWRLL$QN-NH2 | 2159.23 | 1080.62 | 1080.89 |
| 49 | 90 | Ac-QSQQTF$r8NL6clWRLL$QN-NH2 | 2142.11 | 1072.06 | 1072.35 |
| 50 | 91 | Ac-QSQQTF$r8NLMe6clwRLL$QN-NH2 | 2156.13 | 1079.07 | 1079.27 |
| 51 | 92 | Ac-LTFEHYWAQLTS-NH2 | 1535.74 | 768.87 | 768.91 |
| 52 | 93 | Ac-LTF$HYW$QLTS-NH2 | 1585.83 | 793.92 | 794.17 |
| 53 | 94 | Ac-LTFE$YWA$LTS-NH2 | 1520.79 | 761.40 | 761.67 |
| 54 | 95 | Ac-LTF$zr8HYWAQL$zS-NH2 | 1597.87 | 799.94 | 800.06 |
| 55 | 96 | Ac-LTF$r8HYWRQL$S-NH2 | 1682.93 | 842.47 | 842.72 |
| 56 | 97 | Ac-QS$QTFStNLWRLL$s8QN-NH2 | 2145.21 | 1073.61 | 1073.90 |
| 57 | 98 | Ac-QSQQTASNLWRLLPQN-NH2 | 1923.99 | 963.00 | 963.26 |
| 58 | 99 | Ac-QSQQTA$/r8NLWRLL$/QN-NH2 | 2060.15 | 1031.08 | 1031.24 |
| 59 | 100 | Ac-ASQQTF$/r8NLWRLL$/QN-NH2 | 2079.16 | 1040.58 | 1040.89 |
| 60 | 101 | Ac-$SQQ$FSNLWRLLAibQN-NH2 | 2009.09 | 1005.55 | 1005.86 |
| 61 | 102 | Ac-QS$QTF$NLWRLLAibQN-NH2 | 2023.10 | 1012.55 | 1012.79 |
| 62 | 103 | Ac-QSQQ$FSN$WRLLAibQN-NH2 | 2024.06 | 1013.03 | 1013.31 |
| 63 | 104 | Ac-QSQQTF$NLW$LLAibQN-NH2 | 1995.06 | 998.53 | 998.87 |
| 64 | 105 | Ac-QSQQTFS$LWR$LAibQN-NH2 | 2011.06 | 1006.53 | 1006.83 |
| 65 | 106 | Ac-QSQQTFSNLW$LLA$N-NH2 | 1940.02 | 971.01 | 971.29 |
| 66 | 107 | Ac-$/SQQ$/FSNLWRLLAibQN-NH2 | 2037.12 | 1019.56 | 1019.78 |
| 67 | 108 | Ac-QS$/QTF$/NLWRLLAibQN-NH2 | 2051.13 | 1026.57 | 1026.90 |
| 68 | 109 | Ac-QSQQ$/FSN$/WRLLAibQN-NH2 | 2052.09 | 1027.05 | 1027.36 |
| 69 | 110 | Ac-QSQQTF$/NLW$/LLAibQN-NH2 | 2023.09 | 1012.55 | 1013.82 |
| 70 | 111 | Ac-QSQ$TFS$LWRLLAibQN-NH2 | 1996.09 | 999.05 | 999.39 |
| 71 | 112 | Ac-QSQ$/TFS$/LWRLLAibQN-NH2 | 2024.12 | 1013.06 | 1013.37 |
| 72 | 113 | Ac-QS$/QTFSt//NLWRLL$/s8QN-NH2 | 2201.27 | 1101.64 | 1102.00 |
| 73 | 114 | Ac-$r8SQQTFS$LWRLLAibQN-NH2 | 2038.14 | 1020.07 | 1020.23 |
| 74 | 115 | Ac-QSQ$r8TFSNLW$LLAibQN-NH2 | 1996.08 | 999.04 | 999.32 |
| 75 | 116 | Ac-QSQQTFS$r8LWRLLA$N-NH2 | 2024.12 | 1013.06 | 1013.37 |
| 76 | 117 | Ac-QS$r5QTFStNLW$LLAibQN-NH2 | 2032.12 | 1017.06 | 1017.39 |
| 77 | 118 | Ac-$/r8SQQTFS$/LWRLLAibQN-NH2 | 2066.17 | 1034.09 | 1034.80 |
| 78 | 119 | Ac-QSQ$/r8TFSNLW$/LLAibQN-NH2 | 2024.11 | 1013.06 | 1014.34 |
| 79 | 120 | Ac-QSQQTFS$/r8LWRLLA$/N-NH2 | 2052.15 | 1027.08 | 1027.16 |
| 80 | 121 | Ac-QS$/r5QTFSt//NLW$/LLAibQN-NH2 | 2088.18 | 1045.09 | 1047.10 |
| 81 | 122 | Ac-QSQQTFSNLWRLLAibQN-NH2 | 1988.02 | 995.01 | 995.31 |
| 82 | 123 | Hep/QSQ$/TF$/r8NLWRLL$/QN-NH2 | 2215.29 | 1108.65 | 1108.93 |
| 83 | 124 | Ac-ASQQTF$r8NLRWLL$QN-NH2 | 2051.13 | 1026.57 | 1026.90 |
| 84 | 125 | Ac-QSQQTF$/r8NLWRLL$/Q-NH2 | 2022.14 | 1012.07 | 1012.66 |
| 85 | 126 | Ac-QSQQTF$r8NLWRLL$Q-NH2 | 1994.11 | 998.06 | 998.42 |
| 86 | 127 | Ac-AAARAA$r8AAARAA$AA-NH2 | 1515.90 | 758.95 | 759.21 |
| 87 | 128 | Ac-LTFEHYWAQLTSA-NH2 | 1606.78 | 804.39 | 804.59 |
| 88 | 129 | Ac-LTF$r8HYWAQL$SA-NH2 | 1668.90 | 835.45 | 835.67 |
| 89 | 130 | Ac-ASQQTFSNLWRLLPQN-NH2 | 1943.00 | 972.50 | 973.27 |
| 90 | 131 | Ac-QS$QTFStNLW$r5LLAibQN-NH2 | 2032.12 | 1017.06 | 1017.30 |
| 91 | 132 | Ac-QSQQTFAibNLWRLLAibQN-NH2 | 1986.04 | 994.02 | 994.19 |
| 92 | 133 | Ac-QSQQTFNleNLWRLLNleQN-NH2 | 2042.11 | 1022.06 | 1022.23 |
| 93 | 134 | Ac-QSQQTF$/r8NLWRLLAibQN-NH2 | 2082.14 | 1042.07 | 1042.23 |
| 94 | 135 | Ac-QSQQTF$/r8NLWRLLNleQN-NH2 | 2110.17 | 1056.09 | 1056.29 |
| 95 | 136 | Ac-QSQQTFAibNLWRLL$/QN-NH2 | 2040.09 | 1021.05 | 1021.25 |
| 96 | 137 | Ac-QSQQTFNleNLWRLL$/QN-NH2 | 2068.12 | 1035.06 | 1035.31 |
| 97 | 138 | Ac-QSQQTF%r8NL6clWRNleL%QN-NH2 | 2144.13 | 1073.07 | 1073.32 |
| 98 | 139 | Ac-QSQQTF%r8NLMe6clWRLL%QN-NH2 | 2158.15 | 1080.08 | 1080.31 |
| 101 | 140 | Ac-FNle$YWE$L-NH2 | 1160.63 | — | 1161.70 |
| 102 | 141 | Ac-F$r8AYWELL$A-NH2 | 1344.75 | — | 1345.90 |
| 103 | 142 | Ac-F$r8AYWQLL$A-NH2 | 1343.76 | — | 1344.83 |
| 104 | 143 | Ac-NlePRF$r8NYWELL$QN-NH2 | 1925.06 | 963.53 | 963.69 |
| 105 | 144 | Ac-NlePRF$r8DYWRLL$QN-NH2 | 1953.10 | 977.55 | 977.68 |
| 106 | 145 | Ac-NlePRF$r8NYWRLL$Q-NH2 | 1838.07 | 920.04 | 920.18 |
| 107 | 146 | Ac-NlePRF$r8NYWRLL$-NH2 | 1710.01 | 856.01 | 856.13 |
| 108 | 147 | Ac-QSQQTF$r8DLWRLL$QN-NH2 | 2109.14 | 1055.57 | 1055.64 |
| 109 | 148 | Ac-QSQQTF$r8NLWRLL$EN-NH2 | 2109.14 | 1055.57 | 1055.70 |
| 110 | 149 | Ac-QSQQTF$r8NLWRLL$QD-NH2 | 2109.14 | 1055.57 | 1055.64 |
| 111 | 150 | Ac-QSQQTF$r8NLWRLL$S-NH2 | 1953.08 | 977.54 | 977.60 |
| 112 | 151 | Ac-ESQQTF$r8NLWRLL$QN-NH2 | 2109.14 | 1055.57 | 1055.70 |
| 113 | 152 | Ac-LTF$r8NLWRNleL$Q-NH2 | 1635.99 | 819.00 | 819.10 |
| 114 | 153 | Ac-LRF$r8NLWRNleL$Q-NH2 | 1691.04 | 846.52 | 846.68 |
| 115 | 154 | Ac-QSQQTF$r8NWWRNleL$QN-NH2 | 2181.15 | 1091.58 | 1091.64 |
| 116 | 155 | Ac-QSQQTF$r8NLWRNleL$Q-NH2 | 1994.11 | 998.06 | 998.07 |
| 117 | 156 | Ac-QTF$r8NLWRNleL$QN-NH2 | 1765.00 | 883.50 | 883.59 |
| 118 | 157 | Ac-NlePRF$r8NWWRLL$QN-NH2 | 1975.13 | 988.57 | 988.75 |
| 119 | 158 | Ac-NlePRF$r8NWWRLL$A-NH2 | 1804.07 | 903.04 | 903.08 |
| 120 | 159 | Ac-TSFAEYWNLLNH2 | 1467.70 | 734.85 | 734.90 |
| 121 | 160 | Ac-QTF$r8HWWSQL$S-NH2 | 1651.85 | 826.93 | 827.12 |
| 122 | 161 | Ac-FM$YWE$L-NH2 | 1178.58 | — | 1179.64 |
| 123 | 162 | Ac-QTFEHWWSQLLS-NH2 | 1601.76 | 801.88 | 801.94 |
| 124 | 163 | Ac-QSQQTF$r8NLAmwRLNle$QN-NH2 | 2122.17 | 1062.09 | 1062.24 |
| 125 | 164 | Ac-FMAibY6clWEAc3cL-NH2 | 1130.47 | — | 1131.53 |
| 126 | 165 | Ac-FNle$Y6clWE$L-NH2 | 1194.59 | — | 1195.64 |
| 127 | 166 | Ac-F$zr8AY6clWEAc3cL$z-NH2 | 1277.63 | 639.82 | 1278.71 |
| 128 | 167 | Ac-F$r8AY6clWEAc3cL$A-NH2 | 1348.66 | — | 1350.72 |
| 129 | 168 | Ac-NlePRF$r8NY6clWRLL$QN-NH2 | 1986.08 | 994.04 | 994.64 |
| 130 | 169 | Ac-AF$r8AAWALA$A-NH2 | 1223.71 | — | 1224.71 |
| 131 | 170 | Ac-TF$r8AAWRLA$Q-NH2 | 1395.80 | 698.90 | 399.04 |
| 132 | 171 | Pr-TF$r8AAWRLA$Q-NH2 | 1409.82 | 705.91 | 706.04 |
| 133 | 172 | Ac-QSQQTF%r8NLWRNleL%QN-NH2 | 2110.17 | 1056.09 | 1056.22 |
| 134 | 173 | Ac-LTF%r8HYWAQL%SA-NH2 | 1670.92 | 836.46 | 836.58 |
| 135 | 174 | Ac-NlePRF%r8NYWRLL%QN-NH2 | 1954.13 | 978.07 | 978.19 |
| 136 | 175 | Ac-NlePRF%r8NY6clWRLL%QN-NH2 | 1988.09 | 995.05 | 995.68 |
| 137 | 176 | Ac-LTF%r8HY6clWAQL%S-NH2 | 1633.84 | 817.92 | 817.93 |
| 138 | 177 | Ac-QS%QTF%StNLWRLL%s8QN-NH2 | 2149.24 | 1075.62 | 1075.65 |
| 139 | 178 | Ac-LTF%r8HY6clWRQL%S-NH2 | 1718.91 | 860.46 | 860.54 |
| 140 | 179 | Ac-QSQQTF%r8NL6clWRLL%QN-NH2 | 2144.13 | 1073.07 | 1073.64 |
| 141 | 180 | Ac-%r8SQQTFS%LWRLLAibQN-NH2 | 2040.15 | 1021.08 | 1021.13 |
| 142 | 181 | Ac-LTF%r8HYWAQL%S-NH2 | 1599.88 | 800.94 | 801.09 |
| 143 | 182 | Ac-TSF%r8QYWNLL%P-NH2 | 1602.88 | 802.44 | 802.58 |
| 147 | 183 | Ac-LTFEHYWAQLTS-NH2 | 1535.74 | 768.87 | 769.5 |
| 152 | 184 | Ac-F$er8AY6clWEAc3cL$e-NH2 | 1277.63 | 639.82 | 1278.71 |
| 153 | 185 | Ac-AF$r8AAWALA$A-NH2 | 1277.63 | 639.82 | 1277.84 |
| 154 | 186 | Ac-TF$r8AAWRLA$Q-NH2 | 1395.80 | 698.90 | 699.04 |
| 155 | 187 | Pr-TF$r8AAWRLA$Q-NH2 | 1409.82 | 705.91 | 706.04 |
| 156 | 188 | Ac-LTF$er8HYWAQL$eS-NH2 | 1597.87 | 799.94 | 800.44 |
| 159 | 189 | Ac-CCPGCCBaQSQQTF$r8NLWRLL$QN-NH2 | 2745.30 | 1373.65 | 1372.99 |
| 160 | 190 | Ac-CCPGCCBaQSQQTA$r8NLWRLL$QN-NH2 | 2669.27 | 1335.64 | 1336.09 |
| 161 | 191 | Ac-CCPGCCBaNlePRF$r8NYWRLL$QN-NH2 | 2589.26 | 1295.63 | 1296.2 |
| 162 | 192 | Ac-LTF$/r8HYWAQL$/S-NH2 | 1625.90 | 813.95 | 814.18 |
| 163 | 193 | Ac-F%r8HY6clWRAc3cL%-NH2 | 1372.72 | 687.36 | 687.59 |
| 164 | 194 | Ac-QTF%r8HWWSQL%S-NH2 | 1653.87 | 827.94 | 827.94 |
| 165 | 195 | Ac-LTA$r8HYWRQL$S-NH2 | 1606.90 | 804.45 | 804.66 |
| 166 | 196 | Ac-Q$r8QQTFSN$WRLLAibQN-NH2 | 2080.12 | 1041.06 | 1041.61 |
| 167 | 197 | Ac-QSQQ$r8FSNLWR$LAibQN-NH2 | 2066.11 | 1034.06 | 1034.58 |
| 168 | 198 | Ac-F$r8AYWEAc3cL$A-NH2 | 1314.70 | 658.35 | 1315.88 |
| 169 | 199 | Ac-F$r8AYWEAc3cL$S-NH2 | 1330.70 | 666.35 | 1331.87 |
| 170 | 200 | Ac-F$r8AYWEAc3cL$Q-NH2 | 1371.72 | 686.86 | 1372.72 |
| 171 | 201 | Ac-F$r8AYWEAibL$S-NH2 | 1332.71 | 667.36 | 1334.83 |
| 172 | 202 | Ac-F$r8AYWEAL$S-NH2 | 1318.70 | 660.35 | 1319.73 |
| 173 | 203 | Ac-F$r8AYWEQL$S-NH2 | 1375.72 | 688.86 | 1377.53 |
| 174 | 204 | Ac-F$r8HYWEQL$S-NH2 | 1441.74 | 721.87 | 1443.48 |
| 175 | 205 | Ac-F$r8HYWAQL$S-NH2 | 1383.73 | 692.87 | 1385.38 |
| 176 | 206 | Ac-F$r8HYWAAc3cL$S-NH2 | 1338.71 | 670.36 | 1340.82 |
| 177 | 207 | Ac-F$r8HYWRAc3cL$S-NH2 | 1423.78 | 712.89 | 713.04 |
| 178 | 208 | Ac-F$r8AYWEAc3cL#A-NH2 | 1300.69 | 651.35 | 1302.78 |
| 179 | 209 | Ac-NlePTF%r8NYWRLL%QN-NH2 | 1899.08 | 950.54 | 950.56 |
| 180 | 210 | Ac-TF$r8AAWRAL$Q-NH2 | 1395.80 | 698.90 | 699.13 |
| 181 | 211 | Ac-TSF%r8HYWAQL%S-NH2 | 1573.83 | 787.92 | 787.98 |
| 184 | 212 | Ac-F%r8AY6clWEAc3cL%A-NH2 | 1350.68 | 676.34 | 676.91 |
| 185 | 213 | Ac-LTF$r8HYWAQI$S-NH2 | 1597.87 | 799.94 | 800.07 |
| 186 | 214 | Ac-LTF$r8HYWAQNle$S-NH2 | 1597.87 | 799.94 | 800.07 |
| 187 | 215 | Ac-LTF$r8HYWAQL$A-NH2 | 1581.87 | 791.94 | 792.45 |
| 188 | 216 | Ac-LTF$r8HYWAQL$Abu-NH2 | 1595.89 | 798.95 | 799.03 |
| 189 | 217 | Ac-LTF$r8HYWAbuQL$S-NH2 | 1611.88 | 806.94 | 807.47 |
| 190 | 218 | Ac-LTF$er8AYWAQL$eS-NH2 | 1531.84 | 766.92 | 766.96 |
| 191 | 219 | Ac-LAF$r8HYWAQL$S-NH2 | 1567.86 | 784.93 | 785.49 |
| 192 | 220 | Ac-LAF$r8AYWAQL$S-NH2 | 1501.83 | 751.92 | 752.01 |
| 193 | 221 | Ac-LTF$er8AYWAQL$eA-NH2 | 1515.85 | 758.93 | 758.97 |
| 194 | 222 | Ac-LAF$r8AYWAQL$A-NH2 | 1485.84 | 743.92 | 744.05 |
| 195 | 223 | Ac-LTF$r8NLWANleL$Q-NH2 | 1550.92 | 776.46 | 776.61 |
| 196 | 224 | Ac-LTF$r8NLWANleL$A-NH2 | 1493.90 | 747.95 | 1495.6 |
| 197 | 225 | Ac-LTF$r8ALWANleL$Q-NH2 | 1507.92 | 754.96 | 755 |
| 198 | 226 | Ac-LAF$r8NLWANleL$Q-NH2 | 1520.91 | 761.46 | 761.96 |
| 199 | 227 | Ac-LAF$r8ALWANleL$A-NH2 | 1420.89 | 711.45 | 1421.74 |
| 200 | 228 | Ac-A$r8AYWEAc3cL$A-NH2 | 1238.67 | 620.34 | 1239.65 |
| 201 | 229 | Ac-F$r8AYWEAc3cL$AA-NH2 | 1385.74 | 693.87 | 1386.64 |
| 202 | 230 | Ac-F$r8AYWEAc3cL$Abu-NH2 | 1328.72 | 665.36 | 1330.17 |
| 203 | 231 | Ac-F$r8AYWEAc3cL$Nle-NH2 | 1356.75 | 679.38 | 1358.22 |
| 204 | 232 | Ac-F$r5AYWEAc3cL$s8A-NH2 | 1314.70 | 658.35 | 1315.51 |
| 205 | 233 | Ac-F$AYWEAc3cL$r8A-NH2 | 1314.70 | 658.35 | 1315.66 |
| 206 | 234 | Ac-F$r8AYWEAc3cI$A-NH2 | 1314.70 | 658.35 | 1316.18 |
| 207 | 235 | Ac-F$r8AYWEAc3cNle$A-NH2 | 1314.70 | 658.35 | 1315.66 |
| 208 | 236 | Ac-F$r8AYWEAmlL$A-NH2 | 1358.76 | 680.38 | 1360.21 |
| 209 | 237 | Ac-F$r8AYWENleL$A-NH2 | 1344.75 | 673.38 | 1345.71 |
| 210 | 238 | Ac-F$r8AYWQAc3cL$A-NH2 | 1313.72 | 657.86 | 1314.7 |
| 211 | 239 | Ac-F$r8AYWAAc3cL$A-NH2 | 1256.70 | 629.35 | 1257.56 |
| 212 | 240 | Ac-F$r8AYWAbuAc3cL$A-NH2 | 1270.71 | 636.36 | 1272.14 |
| 213 | 241 | Ac-F$r8AYWNleAc3cL$A-NH2 | 1298.74 | 650.37 | 1299.67 |
| 214 | 242 | Ac-F$r8AbuYWEAc3cL$A-NH2 | 1328.72 | 665.36 | 1329.65 |
| 215 | 243 | Ac-F$r8NleYWEAc3cL$A-NH2 | 1356.75 | 679.38 | 1358.66 |
| 216 | 244 | 5-FAM-BaLTFEHYWAQLTS-NH2 | 1922.82 | 962.41 | 962.87 |
| 217 | 245 | 5-FAM-BaLTF%r8HYWAQL%S-NH2 | 1986.96 | 994.48 | 994.97 |
| 218 | 246 | Ac-LTF$r8HYWAQhL$S-NH2 | 1611.88 | 806.94 | 807 |
| 219 | 247 | Ac-LTF$r8HYWAQTle$S-NH2 | 1597.87 | 799.94 | 799.97 |
| 220 | 248 | Ac-LTF$r8HYWAQAdm$S-NH2 | 1675.91 | 838.96 | 839.09 |
| 221 | 249 | Ac-LTF$r8HYWAQhCha$S-NH2 | 1651.91 | 826.96 | 826.98 |
| 222 | 250 | Ac-LTF$r8HYWAQCha$S-NH2 | 1637.90 | 819.95 | 820.02 |
| 223 | 251 | Ac-LTF$r8HYWAc6cQL$S-NH2 | 1651.91 | 826.96 | 826.98 |
| 224 | 252 | Ac-LTF$r8HYWAc5cQL$S-NH2 | 1637.90 | 819.95 | 820.02 |
| 225 | 253 | Ac-LThF$r8HYWAQL$S-NH2 | 1611.88 | 806.94 | 807 |
| 226 | 254 | Ac-LTIgl$r8HYWAQL$S-NH2 | 1625.90 | 813.95 | 812.99 |
| 227 | 255 | Ac-LTF$r8HYWAQChg$S-NH2 | 1623.88 | 812.94 | 812.99 |
| 228 | 256 | Ac-LTF$r8HYWAQF$S-NH2 | 1631.85 | 816.93 | 816.99 |
| 229 | 257 | Ac-LTF$r8HYWAQIgl$S-NH2 | 1659.88 | 830.94 | 829.94 |
| 230 | 258 | Ac-LTF$r8HYWAQCba$S-NH2 | 1609.87 | 805.94 | 805.96 |
| 231 | 259 | Ac-LTF$r8HYWAQCpg$S-NH2 | 1609.87 | 805.94 | 805.96 |
| 232 | 260 | Ac-LTF$r8HhYWAQL$S-NH2 | 1611.88 | 806.94 | 807 |
| 233 | 261 | Ac-F$r8AYWEAc3chL$A-NH2 | 1328.72 | 665.36 | 665.43 |
| 234 | 262 | Ac-F$r8AYWEAc3cTle$A-NH2 | 1314.70 | 658.35 | 1315.62 |
| 235 | 263 | Ac-F$r8AYWEAc3cAdm$A-NH2 | 1392.75 | 697.38 | 697.47 |
| 236 | 264 | Ac-F$r8AYWEAc3chCha$A-NH2 | 1368.75 | 685.38 | 685.34 |
| 237 | 265 | Ac-F$r8AYWEAc3cCha$A-NH2 | 1354.73 | 678.37 | 678.38 |
| 238 | 266 | Ac-F$r8AYWEAc6cL$A-NH2 | 1356.75 | 679.38 | 679.42 |
| 239 | 267 | Ac-F$r8AYWEAc5cL$A-NH2 | 1342.73 | 672.37 | 672.46 |
| 240 | 268 | Ac-hF$r8AYWEAc3cL$A-NH2 | 1328.72 | 665.36 | 665.43 |
| 241 | 269 | Ac-Igl$r8AYWEAc3cL$A-NH2 | 1342.73 | 672.37 | 671.5 |
| 243 | 270 | Ac-F$r8AYWEAc3cF$A-NH2 | 1348.69 | 675.35 | 675.35 |
| 244 | 271 | Ac-F$r8AYWEAc3cIgl$A-NH2 | 1376.72 | 689.36 | 688.37 |
| 245 | 272 | Ac-F$r8AYWEAc3cCba$A-NH2 | 1326.70 | 664.35 | 664.47 |
| 246 | 273 | Ac-F$r8AYWEAc3cCpg$A-NH2 | 1326.70 | 664.35 | 664.39 |
| 247 | 274 | Ac-F$r8AhYWEAc3cL$A-NH2 | 1328.72 | 665.36 | 665.43 |
| 248 | 275 | Ac-F$r8AYWEAc3cL$Q-NH2 | 1371.72 | 686.86 | 1372.87 |
| 249 | 276 | Ac-F$r8AYWEAibL$A-NH2 | 1316.72 | 659.36 | 1318.18 |
| 250 | 277 | Ac-F$r8AYWEAL$A-NH2 | 1302.70 | 652.35 | 1303.75 |
| 251 | 278 | Ac-LAF$r8AYWAAL$A-NH2 | 1428.82 | 715.41 | 715.49 |
| 252 | 279 | Ac-LTF$r8HYWAAc3cL$S-NH2 | 1552.84 | 777.42 | 777.5 |
| 253 | 280 | Ac-NleTF$r8HYWAQL$S-NH2 | 1597.87 | 799.94 | 800.04 |
| 254 | 281 | Ac-VTF$r8HYWAQL$S-NH2 | 1583.85 | 792.93 | 793.04 |
| 255 | 282 | Ac-FTF$r8HYWAQL$S-NH2 | 1631.85 | 816.93 | 817.02 |
| 256 | 283 | Ac-WTF$r8HYWAQL$S-NH2 | 1670.86 | 836.43 | 836.85 |
| 257 | 284 | Ac-RTF$r8HYWAQL$S-NH2 | 1640.88 | 821.44 | 821.9 |
| 258 | 285 | Ac-KTF$r8HYWAQL$S-NH2 | 1612.88 | 807.44 | 807.91 |
| 259 | 286 | Ac-LNleF$r8HYWAQL$S-NH2 | 1609.90 | 805.95 | 806.43 |
| 260 | 287 | Ac-LVF$r8HYWAQL$S-NH2 | 1595.89 | 798.95 | 798.93 |
| 261 | 288 | Ac-LFF$r8HYWAQL$S-NH2 | 1643.89 | 822.95 | 823.38 |
| 262 | 289 | Ac-LWF$r8HYWAQL$S-NH2 | 1682.90 | 842.45 | 842.55 |
| 263 | 290 | Ac-LRF$r8HYWAQL$S-NH2 | 1652.92 | 827.46 | 827.52 |
| 264 | 291 | Ac-LKF$r8HYWAQL$S-NH2 | 1624.91 | 813.46 | 813.51 |
| 265 | 292 | Ac-LTF$r8NleYWAQL$S-NH2 | 1573.89 | 787.95 | 788.05 |
| 266 | 293 | Ac-LTF$r8VYWAQL$S-NH2 | 1559.88 | 780.94 | 780.98 |
| 267 | 294 | Ac-LTF$r8FYWAQL$S-NH2 | 1607.88 | 804.94 | 805.32 |
| 268 | 295 | Ac-LTF$r8WYWAQL$S-NH2 | 1646.89 | 824.45 | 824.86 |
| 269 | 296 | Ac-LTF$r8RYWAQL$S-NH2 | 1616.91 | 809.46 | 809.51 |
| 270 | 297 | Ac-LTF$r8KYWAQL$S-NH2 | 1588.90 | 795.45 | 795.48 |
| 271 | 298 | Ac-LTF$r8HNleWAQL$S-NH2 | 1547.89 | 774.95 | 774.98 |
| 272 | 299 | Ac-LTF$r8HVWAQL$S-NH2 | 1533.87 | 767.94 | 767.95 |
| 273 | 300 | Ac-LTF$r8HFWAQL$S-NH2 | 1581.87 | 791.94 | 792.3 |
| 274 | 301 | Ac-LTF$r8HWWAQL$S-NH2 | 1620.88 | 811.44 | 811.54 |
| 275 | 302 | Ac-LTF$r8HRWAQL$S-NH2 | 1590.90 | 796.45 | 796.52 |
| 276 | 303 | Ac-LTF$r8HKWAQL$S-NH2 | 1562.90 | 782.45 | 782.53 |
| 277 | 304 | Ac-LTF$r8HYWNleQL$S-NH2 | 1639.91 | 820.96 | 820.98 |
| 278 | 305 | Ac-LTF$r8HYWVQL$S-NH2 | 1625.90 | 813.95 | 814.03 |
| 279 | 306 | Ac-LTF$r8HYWFQL$S-NH2 | 1673.90 | 837.95 | 838.03 |
| 280 | 307 | Ac-LTF$r8HYWWQL$S-NH2 | 1712.91 | 857.46 | 857.5 |
| 281 | 308 | Ac-LTF$r8HYWKQL$S-NH2 | 1654.92 | 828.46 | 828.49 |
| 282 | 309 | Ac-LTF$r8HYWANleL$S-NH2 | 1582.89 | 792.45 | 792.52 |
| 283 | 310 | Ac-LTF$r8HYWAVL$S-NH2 | 1568.88 | 785.44 | 785.49 |
| 284 | 311 | Ac-LTF$r8HYWAFL$S-NH2 | 1616.88 | 809.44 | 809.47 |
| 285 | 312 | Ac-LTF$r8HYWAWL$S-NH2 | 1655.89 | 828.95 | 829 |
| 286 | 313 | Ac-LTF$r8HYWARL$S-NH2 | 1625.91 | 813.96 | 813.98 |
| 287 | 314 | Ac-LTF$r8HYWAQL$Nle-NH2 | 1623.92 | 812.96 | 813.39 |
| 288 | 315 | Ac-LTF$r8HYWAQL$V-NH2 | 1609.90 | 805.95 | 805.99 |
| 289 | 316 | Ac-LTF$r8HYWAQL$F-NH2 | 1657.90 | 829.95 | 830.26 |
| 290 | 317 | Ac-LTF$r8HYWAQL$W-NH2 | 1696.91 | 849.46 | 849.5 |
| 291 | 318 | Ac-LTF$r8HYWAQL$R-NH2 | 1666.94 | 834.47 | 834.56 |
| 292 | 319 | Ac-LTF$r8HYWAQL$K-NH2 | 1638.93 | 820.47 | 820.49 |
| 293 | 320 | Ac-Q$r8QQTFSN$WRLLAibQN-NH2 | 2080.12 | 1041.06 | 1041.54 |
| 294 | 321 | Ac-QSQQ$r8FSNLWR$LAibQN-NH2 | 2066.11 | 1034.06 | 1034.58 |
| 295 | 322 | Ac-LT2Pal$r8HYWAQL$S-NH2 | 1598.86 | 800.43 | 800.49 |
| 296 | 323 | Ac-LT3Pal$r8HYWAQL$S-NH2 | 1598.86 | 800.43 | 800.49 |
| 297 | 324 | Ac-LT4Pal$r8HYWAQL$S-NH2 | 1598.86 | 800.43 | 800.49 |
| 298 | 325 | Ac-LTF2CF3$r8HYWAQL$S-NH2 | 1665.85 | 833.93 | 834.01 |
| 299 | 326 | Ac-LTF2CN$r8HYWAQL$S-NH2 | 1622.86 | 812.43 | 812.47 |
| 300 | 327 | Ac-LTF2Me$r8HYWAQL$S-NH2 | 1611.88 | 806.94 | 807 |
| 301 | 328 | Ac-LTF3Cl$r8HYWAQL$S-NH2 | 1631.83 | 816.92 | 816.99 |
| 302 | 329 | Ac-LTF4CF3$r8HYWAQL$S-NH2 | 1665.85 | 833.93 | 833.94 |
| 303 | 330 | Ac-LTF4tBu$r8HYWAQL$S-NH2 | 1653.93 | 827.97 | 828.02 |
| 304 | 331 | Ac-LTF5F$r8HYWAQL$S-NH2 | 1687.82 | 844.91 | 844.96 |
| 305 | 332 | Ac-LTF$r8HY3BthAAQL$S-NH2 | 1614.83 | 808.42 | 808.48 |
| 306 | 333 | Ac-LTF2Br$r8HYWAQL$S-NH2 | 1675.78 | 838.89 | 838.97 |
| 307 | 334 | Ac-LTF4Br$r8HYWAQL$S-NH2 | 1675.78 | 838.89 | 839.86 |
| 308 | 335 | Ac-LTF2Cl$r8HYWAQL$S-NH2 | 1631.83 | 816.92 | 816.99 |
| 309 | 336 | Ac-LTF4Cl$r8HYWAQL$S-NH2 | 1631.83 | 816.92 | 817.36 |
| 310 | 337 | Ac-LTF3CN$r8HYWAQL$S-NH2 | 1622.86 | 812.43 | 812.47 |
| 311 | 338 | Ac-LTF4CN$r8HYWAQL$S-NH2 | 1622.86 | 812.43 | 812.47 |
| 312 | 339 | Ac-LTF34Cl2$r8HYWAQL$S-NH2 | 1665.79 | 833.90 | 833.94 |
| 313 | 340 | Ac-LTF34F2$r8HYWAQL$S-NH2 | 1633.85 | 817.93 | 817.95 |
| 314 | 341 | Ac-LTF35F2$r8HYWAQL$S-NH2 | 1633.85 | 817.93 | 817.95 |
| 315 | 342 | Ac-LTDip$r8HYWAQL$S-NH2 | 1673.90 | 837.95 | 838.01 |
| 316 | 343 | Ac-LTF2F$r8HYWAQL$S-NH2 | 1615.86 | 808.93 | 809 |
| 317 | 344 | Ac-LTF3F$r8HYWAQL$S-NH2 | 1615.86 | 808.93 | 809 |
| 318 | 345 | Ac-LTF4F$r8HYWAQL$S-NH2 | 1615.86 | 808.93 | 809 |
| 319 | 346 | Ac-LTF4I$r8HYWAQL$S-NH2 | 1723.76 | 862.88 | 862.94 |
| 320 | 347 | Ac-LTF3Me$r8HYWAQL$S-NH2 | 1611.88 | 806.94 | 807.07 |
| 321 | 348 | Ac-LTF4Me$r8HYWAQL$S-NH2 | 1611.88 | 806.94 | 807 |
| 322 | 349 | Ac-LT1Nal$r8HYWAQL$S-NH2 | 1647.88 | 824.94 | 824.98 |
| 323 | 350 | Ac-LT2Nal$r8HYWAQL$S-NH2 | 1647.88 | 824.94 | 825.06 |
| 324 | 351 | Ac-LTF3CF3$r8HYWAQL$S-NH2 | 1665.85 | 833.93 | 834.01 |
| 325 | 352 | Ac-LTF4NO2$r8HYWAQL$S-NH2 | 1642.85 | 822.43 | 822.46 |
| 326 | 353 | Ac-LTF3NO2$r8HYWAQL$S-NH2 | 1642.85 | 822.43 | 822.46 |
| 327 | 354 | Ac-LTF$r82ThiYWAQL$S-NH2 | 1613.83 | 807.92 | 807.96 |
| 328 | 355 | Ac-LTF$r8HBipWAQL$S-NH2 | 1657.90 | 829.95 | 830.01 |
| 329 | 356 | Ac-LTF$r8HF4tBuWAQL$S-NH2 | 1637.93 | 819.97 | 820.02 |
| 330 | 357 | Ac-LTF$r8HF4CF3WAQL$S-NH2 | 1649.86 | 825.93 | 826.02 |
| 331 | 358 | Ac-LTF$r8HF4ClWAQL$S-NH2 | 1615.83 | 808.92 | 809.37 |
| 332 | 359 | Ac-LTF$r8HF4MeWAQL$S-NH2 | 1595.89 | 798.95 | 799.01 |
| 333 | 360 | Ac-LTF$r8HF4BrWAQL$S-NH2 | 1659.78 | 830.89 | 830.98 |
| 334 | 361 | Ac-LTF$r8HF4CNWAQL$S-NH2 | 1606.87 | 804.44 | 804.56 |
| 335 | 362 | Ac-LTF$r8HF4NO2WAQL$S-NH2 | 1626.86 | 814.43 | 814.55 |
| 336 | 363 | Ac-LTF$r8H1NalWAQL$S-NH2 | 1631.89 | 816.95 | 817.06 |
| 337 | 364 | Ac-LTF$r8H2NalWAQL$S-NH2 | 1631.89 | 816.95 | 816.99 |
| 338 | 365 | Ac-LTF$r8HWAQL$S-NH2 | 1434.80 | 718.40 | 718.49 |
| 339 | 366 | Ac-LTF$r8HY1NalAQL$S-NH2 | 1608.87 | 805.44 | 805.52 |
| 340 | 367 | Ac-LTF$r8HY2NalAQL$S-NH2 | 1608.87 | 805.44 | 805.52 |
| 341 | 368 | Ac-LTF$r8HYWAQI$S-NH2 | 1597.87 | 799.94 | 800.07 |
| 342 | 369 | Ac-LTF$r8HYWAQNle$S-NH2 | 1597.87 | 799.94 | 800.44 |
| 343 | 370 | Ac-LTF$er8HYWAQL$eA-NH2 | 1581.87 | 791.94 | 791.98 |
| 344 | 371 | Ac-LTF$r8HYWAQL$Abu-NH2 | 1595.89 | 798.95 | 799.03 |
| 345 | 372 | Ac-LTF$r8HYWAbuQL$S-NH2 | 1611.88 | 806.94 | 804.47 |
| 346 | 373 | Ac-LAF$r8HYWAQL$S-NH2 | 1567.86 | 784.93 | 785.49 |
| 347 | 374 | Ac-LTF$r8NLWANleL$Q-NH2 | 1550.92 | 776.46 | 777.5 |
| 348 | 375 | Ac-LTF$r8ALWANleL$Q-NH2 | 1507.92 | 754.96 | 755.52 |
| 349 | 376 | Ac-LAF$r8NLWANleL$Q-NH2 | 1520.91 | 761.46 | 762.48 |
| 350 | 377 | Ac-F$r8AYWAAc3cL$A-NH2 | 1256.70 | 629.35 | 1257.56 |
| 351 | 378 | Ac-LTF$r8AYWAAL$S-NH2 | 1474.82 | 738.41 | 738.55 |
| 352 | 379 | Ac-LVF$r8AYWAQL$S-NH2 | 1529.87 | 765.94 | 766 |
| 353 | 380 | Ac-LTF$r8AYWAbuQL$S-NH2 | 1545.86 | 773.93 | 773.92 |
| 354 | 381 | Ac-LTF$r8AYWNleQL$S-NH2 | 1573.89 | 787.95 | 788.17 |
| 355 | 382 | Ac-LTF$r8AbuYWAQL$S-NH2 | 1545.86 | 773.93 | 773.99 |
| 356 | 383 | Ac-LTF$r8AYWHQL$S-NH2 | 1597.87 | 799.94 | 799.97 |
| 357 | 384 | Ac-LTF$r8AYWKQL$S-NH2 | 1588.90 | 795.45 | 795.53 |
| 358 | 385 | Ac-LTF$r8AYWOQL$S-NH2 | 1574.89 | 788.45 | 788.5 |
| 359 | 386 | Ac-LTF$r8AYWRQL$S-NH2 | 1616.91 | 809.46 | 809.51 |
| 360 | 387 | Ac-LTF$r8AYWSQL$S-NH2 | 1547.84 | 774.92 | 774.96 |
| 361 | 388 | Ac-LTF$r8AYWRAL$S-NH2 | 1559.89 | 780.95 | 780.95 |
| 362 | 389 | Ac-LTF$r8AYWRQL$A-NH2 | 1600.91 | 801.46 | 801.52 |
| 363 | 390 | Ac-LTF$r8AYWRAL$A-NH2 | 1543.89 | 772.95 | 773.03 |
| 364 | 391 | Ac-LTF$r5HYWAQL$s8S-NH2 | 1597.87 | 799.94 | 799.97 |
| 365 | 392 | Ac-LTF$HYWAQL$r8S-NH2 | 1597.87 | 799.94 | 799.97 |
| 366 | 393 | Ac-LTF$r8HYWAAL$S-NH2 | 1540.84 | 771.42 | 771.48 |
| 367 | 394 | Ac-LTF$r8HYWAAbuL$S-NH2 | 1554.86 | 778.43 | 778.51 |
| 368 | 395 | Ac-LTF$r8HYWALL$S-NH2 | 1582.89 | 792.45 | 792.49 |
| 369 | 396 | Ac-F$r8AYWHAL$A-NH2 | 1310.72 | 656.36 | 656.4 |
| 370 | 397 | Ac-F$r8AYWAAL$A-NH2 | 1244.70 | 623.35 | 1245.61 |
| 371 | 398 | Ac-F$r8AYWSAL$A-NH2 | 1260.69 | 631.35 | 1261.6 |
| 372 | 399 | Ac-F$r8AYWRAL$A-NH2 | 1329.76 | 665.88 | 1330.72 |
| 373 | 400 | Ac-F$r8AYWKAL$A-NH2 | 1301.75 | 651.88 | 1302.67 |
| 374 | 401 | Ac-F$r8AYWOAL$A-NH2 | 1287.74 | 644.87 | 1289.13 |
| 375 | 402 | Ac-F$r8VYWEAc3cL$A-NH2 | 1342.73 | 672.37 | 1343.67 |
| 376 | 403 | Ac-F$r8FYWEAc3cL$A-NH2 | 1390.73 | 696.37 | 1392.14 |
| 377 | 404 | Ac-F$r8WYWEAc3cL$A-NH2 | 1429.74 | 715.87 | 1431.44 |
| 378 | 405 | Ac-F$r8RYWEAc3cL$A-NH2 | 1399.77 | 700.89 | 700.95 |
| 379 | 406 | Ac-F$r8KYWEAc3cL$A-NH2 | 1371.76 | 686.88 | 686.97 |
| 380 | 407 | Ac-F$r8ANleWEAc3cL$A-NH2 | 1264.72 | 633.36 | 1265.59 |
| 381 | 408 | Ac-F$r8AVWEAc3cL$A-NH2 | 1250.71 | 626.36 | 1252.2 |
| 382 | 409 | Ac-F$r8AFWEAc3cL$A-NH2 | 1298.71 | 650.36 | 1299.64 |
| 383 | 410 | Ac-F$r8AWWEAc3cL$A-NH2 | 1337.72 | 669.86 | 1338.64 |
| 384 | 411 | Ac-F$r8ARWEAc3cL$A-NH2 | 1307.74 | 654.87 | 655 |
| 385 | 412 | Ac-F$r8AKWEAc3cL$A-NH2 | 1279.73 | 640.87 | 641.01 |
| 386 | 413 | Ac-F$r8AYWVAc3cL$A-NH2 | 1284.73 | 643.37 | 643.38 |
| 387 | 414 | Ac-F$r8AYWFAc3cL$A-NH2 | 1332.73 | 667.37 | 667.43 |
| 388 | 415 | Ac-F$r8AYWWAc3cL$A-NH2 | 1371.74 | 686.87 | 686.97 |
| 389 | 416 | Ac-F$r8AYWRAc3cL$A-NH2 | 1341.76 | 671.88 | 671.94 |
| 390 | 417 | Ac-F$r8AYWKAc3cL$A-NH2 | 1313.75 | 657.88 | 657.88 |
| 391 | 418 | Ac-F$r8AYWEVL$A-NH2 | 1330.73 | 666.37 | 666.47 |
| 392 | 419 | Ac-F$r8AYWEFL$A-NH2 | 1378.73 | 690.37 | 690.44 |
| 393 | 420 | Ac-F$r8AYWEWL$A-NH2 | 1417.74 | 709.87 | 709.91 |
| 394 | 421 | Ac-F$r8AYWERL$A-NH2 | 1387.77 | 694.89 | 1388.66 |
| 395 | 422 | Ac-F$r8AYWEKL$A-NH2 | 1359.76 | 680.88 | 1361.21 |
| 396 | 423 | Ac-F$r8AYWEAc3cL$V-NH2 | 1342.73 | 672.37 | 1343.59 |
| 397 | 424 | Ac-F$r8AYWEAc3cL$F-NH2 | 1390.73 | 696.37 | 1392.58 |
| 398 | 425 | Ac-F$r8AYWEAc3cL$W-NH2 | 1429.74 | 715.87 | 1431.29 |
| 399 | 426 | Ac-F$r8AYWEAc3cL$R-NH2 | 1399.77 | 700.89 | 700.95 |
| 400 | 427 | Ac-F$r8AYWEAc3cL$K-NH2 | 1371.76 | 686.88 | 686.97 |
| 401 | 428 | Ac-F$r8AYWEAc3cL$AV-NH2 | 1413.77 | 707.89 | 707.91 |
| 402 | 429 | Ac-F$r8AYWEAc3cL$AF-NH2 | 1461.77 | 731.89 | 731.96 |
| 403 | 430 | Ac-F$r8AYWEAc3cL$AW-NH2 | 1500.78 | 751.39 | 751.5 |
| 404 | 431 | Ac-F$r8AYWEAc3cL$AR-NH2 | 1470.80 | 736.40 | 736.47 |
| 405 | 432 | Ac-F$r8AYWEAc3cL$AK-NH2 | 1442.80 | 722.40 | 722.41 |
| 406 | 433 | Ac-F$r8AYWEAc3cL$AH-NH2 | 1451.76 | 726.88 | 726.93 |
| 407 | 434 | Ac-LTF2NO2$r8HYWAQL$S-NH2 | 1642.85 | 822.43 | 822.54 |
| 408 | 435 | Ac-LTA$r8HYAAQL$S-NH2 | 1406.79 | 704.40 | 704.5 |
| 409 | 436 | Ac-LTF$r8HYAAQL$S-NH2 | 1482.82 | 742.41 | 742.47 |
| 410 | 437 | Ac-QSQQTF$r8NLWALL$AN-NH2 | 1966.07 | 984.04 | 984.38 |
| 411 | 438 | Ac-QAibQQTF$r8NLWALL$AN-NH2 | 1964.09 | 983.05 | 983.42 |
| 412 | 439 | Ac-QAibQQTF$r8ALWALL$AN-NH2 | 1921.08 | 961.54 | 961.59 |
| 413 | 440 | Ac-AAAATF$r8AAWAAL$AA-NH2 | 1608.90 | 805.45 | 805.52 |
| 414 | 441 | Ac-F$r8AAWRAL$Q-NH2 | 1294.76 | 648.38 | 648.48 |
| 415 | 442 | Ac-TF$r8AAWAAL$Q-NH2 | 1310.74 | 656.37 | 1311.62 |
| 416 | 443 | Ac-TF$r8AAWRAL$A-NH2 | 1338.78 | 670.39 | 670.46 |
| 417 | 444 | Ac-VF$r8AAWRAL$Q-NH2 | 1393.82 | 697.91 | 697.99 |
| 418 | 445 | Ac-AF$r8AAWAAL$A-NH2 | 1223.71 | 612.86 | 1224.67 |
| 420 | 446 | Ac-TF$r8AAWKAL$Q-NH2 | 1367.80 | 684.90 | 684.97 |
| 421 | 447 | Ac-TF$r8AAWOAL$Q-NH2 | 1353.78 | 677.89 | 678.01 |
| 422 | 448 | Ac-TF$r8AAWSAL$Q-NH2 | 1326.73 | 664.37 | 664.47 |
| 423 | 449 | Ac-LTF$r8AAWRAL$Q-NH2 | 1508.89 | 755.45 | 755.49 |
| 424 | 450 | Ac-F$r8AYWAQL$A-NH2 | 1301.72 | 651.86 | 651.96 |
| 425 | 451 | Ac-F$r8AWWAAL$A-NH2 | 1267.71 | 634.86 | 634.87 |
| 426 | 452 | Ac-F$r8AWWAQL$A-NH2 | 1324.73 | 663.37 | 663.43 |
| 427 | 453 | Ac-F$r8AYWEAL$-NH2 | 1231.66 | 616.83 | 1232.93 |
| 428 | 454 | Ac-F$r8AYWAAL$-NH2 | 1173.66 | 587.83 | 1175.09 |
| 429 | 455 | Ac-F$r8AYWKAL$-NH2 | 1230.72 | 616.36 | 616.44 |
| 430 | 456 | Ac-F$r8AYWOAL$-NH2 | 1216.70 | 609.35 | 609.48 |
| 431 | 457 | Ac-F$r8AYWQAL$-NH2 | 1230.68 | 616.34 | 616.44 |
| 432 | 458 | Ac-F$r8AYWAQL$-NH2 | 1230.68 | 616.34 | 616.37 |
| 433 | 459 | Ac-F$r8HYWDQL$S-NH2 | 1427.72 | 714.86 | 714.86 |
| 434 | 460 | Ac-F$r8HFWEQL$S-NH2 | 1425.74 | 713.87 | 713.98 |
| 435 | 461 | Ac-F$r8AYWHQL$S-NH2 | 1383.73 | 692.87 | 692.96 |
| 436 | 462 | Ac-F$r8AYWKQL$S-NH2 | 1374.77 | 688.39 | 688.45 |
| 437 | 463 | Ac-F$r8AYWOQL$S-NH2 | 1360.75 | 681.38 | 681.49 |
| 438 | 464 | Ac-F$r8HYWSQL$S-NH2 | 1399.73 | 700.87 | 700.95 |
| 439 | 465 | Ac-F$r8HWWEQL$S-NH2 | 1464.76 | 733.38 | 733.44 |
| 440 | 466 | Ac-F$r8HWWAQL$S-NH2 | 1406.75 | 704.38 | 704.43 |
| 441 | 467 | Ac-F$r8AWWHQL$S-NH2 | 1406.75 | 704.38 | 704.43 |
| 442 | 468 | Ac-F$r8AWWKQL$S-NH2 | 1397.79 | 699.90 | 699.92 |
| 443 | 469 | Ac-F$r8AWWOQL$S-NH2 | 1383.77 | 692.89 | 692.96 |
| 444 | 470 | Ac-F$r8HWWSQL$S-NH2 | 1422.75 | 712.38 | 712.42 |
| 445 | 471 | Ac-LTF$r8NYWANleL$Q-NH2 | 1600.90 | 801.45 | 801.52 |
| 446 | 472 | Ac-LTF$r8NLWAQL$Q-NH2 | 1565.90 | 783.95 | 784.06 |
| 447 | 473 | Ac-LTF$r8NYWANleL$A-NH2 | 1543.88 | 772.94 | 773.03 |
| 448 | 474 | Ac-LTF$r8NLWAQL$A-NH2 | 1508.88 | 755.44 | 755.49 |
| 449 | 475 | Ac-LTF$r8AYWANleL$Q-NH2 | 1557.90 | 779.95 | 780.06 |
| 450 | 476 | Ac-LTF$r8ALWAQL$Q-NH2 | 1522.89 | 762.45 | 762.45 |
| 451 | 477 | Ac-LAF$r8NYWANleL$Q-NH2 | 1570.89 | 786.45 | 786.5 |
| 452 | 478 | Ac-LAF$r8NLWAQL$Q-NH2 | 1535.89 | 768.95 | 769.03 |
| 453 | 479 | Ac-LAF$r8AYWANleL$A-NH2 | 1470.86 | 736.43 | 736.47 |
| 454 | 480 | Ac-LAF$r8ALWAQL$A-NH2 | 1435.86 | 718.93 | 719.01 |
| 455 | 481 | Ac-LAF$r8AYWAAL$A-NH2 | 1428.82 | 715.41 | 715.41 |
| 456 | 482 | Ac-F$r8AYWEAc3cL$AAib-NH2 | 1399.75 | 700.88 | 700.95 |
| 457 | 483 | Ac-F$r8AYWAQL$AA-NH2 | 1372.75 | 687.38 | 687.78 |
| 458 | 484 | Ac-F$r8AYWAAc3cL$AA-NH2 | 1327.73 | 664.87 | 664.84 |
| 459 | 485 | Ac-F$r8AYWSAc3cL$AA-NH2 | 1343.73 | 672.87 | 672.9 |
| 460 | 486 | Ac-F$r8AYWEAc3cL$AS-NH2 | 1401.73 | 701.87 | 701.84 |
| 461 | 487 | Ac-F$r8AYWEAc3cL$AT-NH2 | 1415.75 | 708.88 | 708.87 |
| 462 | 488 | Ac-F$r8AYWEAc3cL$AL-NH2 | 1427.79 | 714.90 | 714.94 |
| 463 | 489 | Ac-F$r8AYWEAc3cL$AQ-NH2 | 1442.76 | 722.38 | 722.41 |
| 464 | 490 | Ac-F$r8AFWEAc3cL$AA-NH2 | 1369.74 | 685.87 | 685.93 |
| 465 | 491 | Ac-F$r8AWWEAc3cL$AA-NH2 | 1408.75 | 705.38 | 705.39 |
| 466 | 492 | Ac-F$r8AYWEAc3cL$SA-NH2 | 1401.73 | 701.87 | 701.99 |
| 467 | 493 | Ac-F$r8AYWEAL$AA-NH2 | 1373.74 | 687.87 | 687.93 |
| 468 | 494 | Ac-F$r8AYWENleL$AA-NH2 | 1415.79 | 708.90 | 708.94 |
| 469 | 495 | Ac-F$r8AYWEAc3cL$AbuA-NH2 | 1399.75 | 700.88 | 700.95 |
| 470 | 496 | Ac-F$r8AYWEAc3cL$NleA-NH2 | 1427.79 | 714.90 | 714.86 |
| 471 | 497 | Ac-F$r8AYWEAibL$NleA-NH2 | 1429.80 | 715.90 | 715.97 |
| 472 | 498 | Ac-F$r8AYWEAL$NleA-NH2 | 1415.79 | 708.90 | 708.94 |
| 473 | 499 | Ac-F$r8AYWENleL$NleA-NH2 | 1457.83 | 729.92 | 729.96 |
| 474 | 500 | Ac-F$r8AYWEAibL$Abu-NH2 | 1330.73 | 666.37 | 666.39 |
| 475 | 501 | Ac-F$r8AYWENleL$Abu-NH2 | 1358.76 | 680.38 | 680.39 |
| 476 | 502 | Ac-F$r8AYWEAL$Abu-NH2 | 1316.72 | 659.36 | 659.36 |
| 477 | 503 | Ac-LTF$r8AFWAQL$S-NH2 | 1515.85 | 758.93 | 759.12 |
| 478 | 504 | Ac-LTF$r8AWWAQL$S-NH2 | 1554.86 | 778.43 | 778.51 |
| 479 | 505 | Ac-LTF$r8AYWAQI$S-NH2 | 1531.84 | 766.92 | 766.96 |
| 480 | 506 | Ac-LTF$r8AYWAQNle$S-NH2 | 1531.84 | 766.92 | 766.96 |
| 481 | 507 | Ac-LTF$r8AYWAQL$SA-NH2 | 1602.88 | 802.44 | 802.48 |
| 482 | 508 | Ac-LTF$r8AWWAQL$A-NH2 | 1538.87 | 770.44 | 770.89 |
| 483 | 509 | Ac-LTF$r8AYWAQI$A-NH2 | 1515.85 | 758.93 | 759.42 |
| 484 | 510 | Ac-LTF$r8AYWAQNle$A-NH2 | 1515.85 | 758.93 | 759.42 |
| 485 | 511 | Ac-LTF$r8AYWAQL$AA-NH2 | 1586.89 | 794.45 | 794.94 |
| 486 | 512 | Ac-LTF$r8HWWAQL$S-NH2 | 1620.88 | 811.44 | 811.47 |
| 487 | 513 | Ac-LTF$r8HRWAQL$S-NH2 | 1590.90 | 796.45 | 796.52 |
| 488 | 514 | Ac-LTF$r8HKWAQL$S-NH2 | 1562.90 | 782.45 | 782.53 |
| 489 | 515 | Ac-LTF$r8HYWAQL$W-NH2 | 1696.91 | 849.46 | 849.5 |
| 491 | 516 | Ac-F$r8AYWAbuAL$A-NH2 | 1258.71 | 630.36 | 630.5 |
| 492 | 517 | Ac-F$r8AbuYWEAL$A-NH2 | 1316.72 | 659.36 | 659.51 |
| 493 | 518 | Ac-NlePRF%r8NYWRLL%QN-NH2 | 1954.13 | 978.07 | 978.54 |
| 494 | 519 | Ac-TSF%r8HYWAQL%S-NH2 | 1573.83 | 787.92 | 787.98 |
| 495 | 520 | Ac-LTF%r8AYWAQL%S-NH2 | 1533.86 | 767.93 | 768 |
| 496 | 521 | Ac-HTF$r8HYWAQL$S-NH2 | 1621.84 | 811.92 | 811.96 |
| 497 | 522 | Ac-LHF$r8HYWAQL$S-NH2 | 1633.88 | 817.94 | 818.02 |
| 498 | 523 | Ac-LTF$r8HHWAQL$S-NH2 | 1571.86 | 786.93 | 786.94 |
| 499 | 524 | Ac-LTF$r8HYWHQL$S-NH2 | 1663.89 | 832.95 | 832.38 |
| 500 | 525 | Ac-LTF$r8HYWAHL$S-NH2 | 1606.87 | 804.44 | 804.48 |
| 501 | 526 | Ac-LTF$r8HYWAQL$H-NH2 | 1647.89 | 824.95 | 824.98 |
| 502 | 527 | Ac-LTF$r8HYWAQL$S-NHPr | 1639.91 | 820.96 | 820.98 |
| 503 | 528 | Ac-LTF$r8HYWAQL$S-NHsBu | 1653.93 | 827.97 | 828.02 |
| 504 | 529 | Ac-LTF$r8HYWAQL$S-NHiBu | 1653.93 | 827.97 | 828.02 |
| 505 | 530 | Ac-LTF$r8HYWAQL$S-NHBn | 1687.91 | 844.96 | 844.44 |
| 506 | 531 | Ac-LTF$r8HYWAQL$S-NHPe | 1700.92 | 851.46 | 851.99 |
| 507 | 532 | Ac-LTF$r8HYWAQL$S-NHChx | 1679.94 | 840.97 | 841.04 |
| 508 | 533 | Ac-ETF$r8AYWAQL$S-NH2 | 1547.80 | 774.90 | 774.96 |
| 509 | 534 | Ac-STF$r8AYWAQL$S-NH2 | 1505.79 | 753.90 | 753.94 |
| 510 | 535 | Ac-LEF$r8AYWAQL$S-NH2 | 1559.84 | 780.92 | 781.25 |
| 511 | 536 | Ac-LSF$r8AYWAQL$S-NH2 | 1517.83 | 759.92 | 759.93 |
| 512 | 537 | Ac-LTF$r8EYWAQL$S-NH2 | 1589.85 | 795.93 | 795.97 |
| 513 | 538 | Ac-LTF$r8SYWAQL$S-NH2 | 1547.84 | 774.92 | 774.96 |
| 514 | 539 | Ac-LTF$r8AYWEQL$S-NH2 | 1589.85 | 795.93 | 795.9 |
| 515 | 540 | Ac-LTF$r8AYWAEL$S-NH2 | 1532.83 | 767.42 | 766.96 |
| 516 | 541 | Ac-LTF$r8AYWASL$S-NH2 | 1490.82 | 746.41 | 746.46 |
| 517 | 542 | Ac-LTF$r8AYWAQL$E-NH2 | 1573.85 | 787.93 | 787.98 |
| 518 | 543 | Ac-LTF2CN$r8HYWAQL$S-NH2 | 1622.86 | 812.43 | 812.47 |
| 519 | 544 | Ac-LTF3Cl$r8HYWAQL$S-NH2 | 1631.83 | 816.92 | 816.99 |
| 520 | 545 | Ac-LTDip$r8HYWAQL$S-NH2 | 1673.90 | 837.95 | 838.01 |
| 521 | 546 | Ac-LTF$r8HYWAQTle$S-NH2 | 1597.87 | 799.94 | 800.04 |
| 522 | 547 | Ac-F$r8AY6clWEAL$A-NH2 | 1336.66 | 669.33 | 1338.56 |
| 523 | 548 | Ac-F$r8AYdl6brWEAL$A-NH2 | 1380.61 | 691.31 | 692.2 |
| 524 | 549 | Ac-F$r8AYdl6fWEAL$A-NH2 | 1320.69 | 661.35 | 1321.61 |
| 525 | 550 | Ac-F$r8AYdl4mWEAL$A-NH2 | 1316.72 | 659.36 | 659.36 |
| 526 | 551 | Ac-F$r8AYdl5clWEAL$A-NH2 | 1336.66 | 669.33 | 669.35 |
| 527 | 552 | Ac-F$r8AYdl7mWEAL$A-NH2 | 1316.72 | 659.36 | 659.36 |
| 528 | 553 | Ac-LTF%r8HYWAQL%A-NH2 | 1583.89 | 792.95 | 793.01 |
| 529 | 554 | Ac-LTF$r8HCouWAQL$S-NH2 | 1679.87 | 840.94 | 841.38 |
| 530 | 555 | Ac-LTFEHCouWAQLTS-NH2 | 1617.75 | 809.88 | 809.96 |
| 531 | 556 | Ac-LTA$r8HCouWAQL$S-NH2 | 1603.84 | 802.92 | 803.36 |
| 532 | 557 | Ac-F$r8AYWEAL$AbuA-NH2 | 1387.75 | 694.88 | 694.88 |
| 533 | 558 | Ac-F$r8AYWEAI$AA-NH2 | 1373.74 | 687.87 | 687.93 |
| 534 | 559 | Ac-F$r8AYWEANle$AA-NH2 | 1373.74 | 687.87 | 687.93 |
| 535 | 560 | Ac-F$r8AYWEAmlL$AA-NH2 | 1429.80 | 715.90 | 715.97 |
| 536 | 561 | Ac-F$r8AYWQAL$AA-NH2 | 1372.75 | 687.38 | 687.48 |
| 537 | 562 | Ac-F$r8AYWAAL$AA-NH2 | 1315.73 | 658.87 | 658.92 |
| 538 | 563 | Ac-F$r8AYWAbuAL$AA-NH2 | 1329.75 | 665.88 | 665.95 |
| 539 | 564 | Ac-F$r8AYWNleAL$AA-NH2 | 1357.78 | 679.89 | 679.94 |
| 540 | 565 | Ac-F$r8AbuYWEAL$AA-NH2 | 1387.75 | 694.88 | 694.96 |
| 541 | 566 | Ac-F$r8NleYWEAL$AA-NH2 | 1415.79 | 708.90 | 708.94 |
| 542 | 567 | Ac-F$r8FYWEAL$AA-NH2 | 1449.77 | 725.89 | 725.97 |
| 543 | 568 | Ac-LTF$r8HYWAQhL$S-NH2 | 1611.88 | 806.94 | 807 |
| 544 | 569 | Ac-LTF$r8HYWAQAdm$S-NH2 | 1675.91 | 838.96 | 839.04 |
| 545 | 570 | Ac-LTF$r8HYWAQIgl$S-NH2 | 1659.88 | 830.94 | 829.94 |
| 546 | 571 | Ac-F$r8AYWAQL$AA-NH2 | 1372.75 | 687.38 | 687.48 |
| 547 | 572 | Ac-LTF$r8ALWAQL$Q-NH2 | 1522.89 | 762.45 | 762.52 |
| 548 | 573 | Ac-F$r8AYWEAL$AA-NH2 | 1373.74 | 687.87 | 687.93 |
| 549 | 574 | Ac-F$r8AYWENleL$AA-NH2 | 1415.79 | 708.90 | 708.94 |
| 550 | 575 | Ac-F$r8AYWEAibL$Abu-NH2 | 1330.73 | 666.37 | 666.39 |
| 551 | 576 | Ac-F$r8AYWENleL$Abu-NH2 | 1358.76 | 680.38 | 680.38 |
| 552 | 577 | Ac-F$r8AYWEAL$Abu-NH2 | 1316.72 | 659.36 | 659.36 |
| 553 | 578 | Ac-F$r8AYWEAc3cL$AbuA-NH2 | 1399.75 | 700.88 | 700.95 |
| 554 | 579 | Ac-F$r8AYWEAc3cL$NleA-NH2 | 1427.79 | 714.90 | 715.01 |
| 555 | 580 | H-LTF$r8AYWAQL$S-NH2 | 1489.83 | 745.92 | 745.95 |
| 556 | 581 | mdPEG3-LTF$r8AYWAQL$S-NH2 | 1679.92 | 840.96 | 840.97 |
| 557 | 582 | mdPEG7-LTF$r8AYWAQL$S-NH2 | 1856.02 | 929.01 | 929.03 |
| 558 | 583 | Ac-F$r8ApmpEt6clWEAL$A-NH2 | 1470.71 | 736.36 | 788.17 |
| 559 | 584 | Ac-LTF3Cl$r8AYWAQL$S-NH2 | 1565.81 | 783.91 | 809.18 |
| 560 | 585 | Ac-LTF3Cl$r8HYWAQL$A-NH2 | 1615.83 | 808.92 | 875.24 |
| 561 | 586 | Ac-LTF3Cl$r8HYWWQL$S-NH2 | 1746.87 | 874.44 | 841.65 |
| 562 | 587 | Ac-LTF3Cl$r8AYWWQL$S-NH2 | 1680.85 | 841.43 | 824.63 |
| 563 | 588 | Ac-LTF$r8AYWWQL$S-NH2 | 1646.89 | 824.45 | 849.98 |
| 564 | 589 | Ac-LTF$r8HYWWQL$A-NH2 | 1696.91 | 849.46 | 816.67 |
| 565 | 590 | Ac-LTF$r8AYWWQL$A-NH2 | 1630.89 | 816.45 | 776.15 |
| 566 | 591 | Ac-LTF4F$r8AYWAQL$S-NH2 | 1549.83 | 775.92 | 776.15 |
| 567 | 592 | Ac-LTF2F$r8AYWAQL$S-NH2 | 1549.83 | 775.92 | 776.15 |
| 568 | 593 | Ac-LTF3F$r8AYWAQL$S-NH2 | 1549.83 | 775.92 | 785.12 |
| 569 | 594 | Ac-LTF34F2$r8AYWAQL$S-NH2 | 1567.83 | 784.92 | 785.12 |
| 570 | 595 | Ac-LTF35F2$r8AYWAQL$S-NH2 | 1567.83 | 784.92 | 1338.74 |
| 571 | 596 | Ac-F3Cl$r8AYWEAL$A-NH2 | 1336.66 | 669.33 | 705.28 |
| 572 | 597 | Ac-F3Cl$r8AYWEAL$AA-NH2 | 1407.70 | 704.85 | 680.11 |
| 573 | 598 | Ac-F$r8AY6clWEAL$AA-NH2 | 1407.70 | 704.85 | 736.83 |
| 574 | 599 | Ac-F$r8AY6clWEAL$-NH2 | 1265.63 | 633.82 | 784.1 |
| 575 | 600 | Ac-LTF$r8HYWAQLSt/S-NH2 | 16.03 | 9.02 | 826.98 |
| 576 | 601 | Ac-LTF$r8HYWAQL$S-NHsBu | 1653.93 | 827.97 | 828.02 |
| 577 | 602 | Ac-STF$r8AYWAQL$S-NH2 | 1505.79 | 753.90 | 753.94 |
| 578 | 603 | Ac-LTF$r8AYWAEL$S-NH2 | 1532.83 | 767.42 | 767.41 |
| 579 | 604 | Ac-LTF$r8AYWAQL$E-NH2 | 1573.85 | 787.93 | 787.98 |
| 580 | 605 | mdPEG3-LTF$r8AYWAQL$S-NH2 | 1679.92 | 840.96 | 840.97 |
| 581 | 606 | Ac-LTF$r8AYWAQhL$S-NH2 | 1545.86 | 773.93 | 774.31 |
| 583 | 607 | Ac-LTF$r8AYWAQCha$S-NH2 | 1571.88 | 786.94 | 787.3 |
| 584 | 608 | Ac-LTF$r8AYWAQChg$S-NH2 | 1557.86 | 779.93 | 780.4 |
| 585 | 609 | Ac-LTF$r8AYWAQCba$S-NH2 | 1543.84 | 772.92 | 780.13 |
| 586 | 610 | Ac-LTF$r8AYWAQF$S-NH2 | 1565.83 | 783.92 | 784.2 |
| 587 | 611 | Ac-LTF4F$r8HYWAQhL$S-NH2 | 1629.87 | 815.94 | 815.36 |
| 588 | 612 | Ac-LTF4F$r8HYWAQCha$S-NH2 | 1655.89 | 828.95 | 828.39 |
| 589 | 613 | Ac-LTF4F$r8HYWAQChg$S-NH2 | 1641.87 | 821.94 | 821.35 |
| 590 | 614 | Ac-LTF4F$r8HYWAQCba$S-NH2 | 1627.86 | 814.93 | 814.32 |
| 591 | 615 | Ac-LTF4F$r8AYWAQhL$S-NH2 | 1563.85 | 782.93 | 782.36 |
| 592 | 616 | Ac-LTF4F$r8AYWAQCha$S-NH2 | 1589.87 | 795.94 | 795.38 |
| 593 | 617 | Ac-LTF4F$r8AYWAQChg$S-NH2 | 1575.85 | 788.93 | 788.35 |
| 594 | 618 | Ac-LTF4F$r8AYWAQCba$S-NH2 | 1561.83 | 781.92 | 781.39 |
| 595 | 619 | Ac-LTF3Cl$r8AYWAQhL$S-NH2 | 1579.82 | 790.91 | 790.35 |
| 596 | 620 | Ac-LTF3Cl$r8AYWAQCha$S-NH2 | 1605.84 | 803.92 | 803.67 |
| 597 | 621 | Ac-LTF3Cl$r8AYWAQChg$S-NH2 | 1591.82 | 796.91 | 796.34 |
| 598 | 622 | Ac-LTF3Cl$r8AYWAQCba$S-NH2 | 1577.81 | 789.91 | 789.39 |
| 599 | 623 | Ac-LTF$r8AYWAQhF$S-NH2 | 1579.84 | 790.92 | 791.14 |
| 600 | 624 | Ac-LTF$r8AYWAQF3CF3$S-NH2 | 1633.82 | 817.91 | 818.15 |
| 601 | 625 | Ac-LTF$r8AYWAQF3Me$S-NH2 | 1581.86 | 791.93 | 791.32 |
| 602 | 626 | Ac-LTF$r8AYWAQ1Nal$S-NH2 | 1615.84 | 808.92 | 809.18 |
| 603 | 627 | Ac-LTF$r8AYWAQBip$S-NH2 | 1641.86 | 821.93 | 822.13 |
| 604 | 628 | Ac-LTF$r8FYWAQL$A-NH2 | 1591.88 | 796.94 | 797.33 |
| 605 | 629 | Ac-LTF$r8HYWAQL$S-NHAm | 1667.94 | 834.97 | 835.92 |
| 606 | 630 | Ac-LTF$r8HYWAQL$S-NHiAm | 1667.94 | 834.97 | 835.55 |
| 607 | 631 | Ac-LTF$r8HYWAQL$S-NHnPr3Ph | 1715.94 | 858.97 | 859.79 |
| 608 | 632 | Ac-LTF$r8HYWAQL$S-NHnBu3,3Me | 1681.96 | 841.98 | 842.49 |
| 610 | 633 | Ac-LTF$r8HYWAQL$S-NHnPr | 1639.91 | 820.96 | 821.58 |
| 611 | 634 | Ac-LTF$r8HYWAQL$S-NHnEt2Ch | 1707.98 | 854.99 | 855.35 |
| 612 | 635 | Ac-LTF$r8HYWAQL$S-NHHex | 1681.96 | 841.98 | 842.4 |
| 613 | 636 | Ac-LTF$r8AYWAQL$S-NHmdPeg2 | 1633.91 | 817.96 | 818.35 |
| 614 | 637 | Ac-LTF$r8AYWAQL$A-NHmdPeg2 | 1617.92 | 809.96 | 810.3 |
| 615 | 638 | Ac-LTF$r8AYWAQL$A-NHmdPeg4 | 1705.97 | 853.99 | 854.33 |
| 616 | 639 | Ac-F$r8AYdl4mWEAL$A-NH2 | 1316.72 | 659.36 | 659.44 |
| 617 | 640 | Ac-F$r8AYdl5clWEAL$A-NH2 | 1336.66 | 669.33 | 669.43 |
| 618 | 641 | Ac-LThF$r8AYWAQL$S-NH2 | 1545.86 | 773.93 | 774.11 |
| 619 | 642 | Ac-LT2Nal$r8AYWAQL$S-NH2 | 1581.86 | 791.93 | 792.43 |
| 620 | 643 | Ac-LTA$r8AYWAQL$S-NH2 | 1455.81 | 728.91 | 729.15 |
| 621 | 644 | Ac-LTF$r8AYWVQL$S-NH2 | 1559.88 | 780.94 | 781.24 |
| 622 | 645 | Ac-LTF$r8HYWAAL$A-NH2 | 1524.85 | 763.43 | 763.86 |
| 623 | 646 | Ac-LTF$r8VYWAQL$A-NH2 | 1543.88 | 772.94 | 773.37 |
| 624 | 647 | Ac-LTF$r8IYWAQL$S-NH2 | 1573.89 | 787.95 | 788.17 |
| 625 | 648 | Ac-FTF$r8VYWSQL$S-NH2 | 1609.85 | 805.93 | 806.22 |
| 626 | 649 | Ac-ITF$r8FYWAQL$S-NH2 | 1607.88 | 804.94 | 805.2 |
| 627 | 650 | Ac-2NalTF$r8VYWSQL$S-NH2 | 1659.87 | 830.94 | 831.2 |
| 628 | 651 | Ac-ITF$r8LYWSQL$S-NH2 | 1589.89 | 795.95 | 796.13 |
| 629 | 652 | Ac-FTF$r8FYWAQL$S-NH2 | 1641.86 | 821.93 | 822.13 |
| 630 | 653 | Ac-WTF$r8VYWAQL$S-NH2 | 1632.87 | 817.44 | 817.69 |
| 631 | 654 | Ac-WTF$r8WYWAQL$S-NH2 | 1719.88 | 860.94 | 861.36 |
| 632 | 655 | Ac-VTF$r8AYWSQL$S-NH2 | 1533.82 | 767.91 | 768.19 |
| 633 | 656 | Ac-WTF$r8FYWSQL$S-NH2 | 1696.87 | 849.44 | 849.7 |
| 634 | 657 | Ac-FTF$r8IYWAQL$S-NH2 | 1607.88 | 804.94 | 805.2 |
| 635 | 658 | Ac-WTF$r8VYWSQL$S-NH2 | 1648.87 | 825.44 | 824.8 |
| 636 | 659 | Ac-FTF$r8LYWSQL$S-NH2 | 1623.87 | 812.94 | 812.8 |
| 637 | 660 | Ac-YTF$r8FYWSQL$S-NH2 | 1673.85 | 837.93 | 837.8 |
| 638 | 661 | Ac-LTF$r8AY6clWEAL$A-NH2 | 1550.79 | 776.40 | 776.14 |
| 639 | 662 | Ac-LTF$r8AY6clWSQL$S-NH2 | 1581.80 | 791.90 | 791.68 |
| 640 | 663 | Ac-F$r8AY6clWSAL$A-NH2 | 1294.65 | 648.33 | 647.67 |
| 641 | 664 | Ac-F$r8AY6clWQAL$AA-NH2 | 1406.72 | 704.36 | 703.84 |
| 642 | 665 | Ac-LHF$r8AYWAQL$S-NH2 | 1567.86 | 784.93 | 785.21 |
| 643 | 666 | Ac-LTF$r8AYWAQL$S-NH2 | 1531.84 | 766.92 | 767.17 |
| 644 | 667 | Ac-LTF$r8AHWAQL$S-NH2 | 1505.84 | 753.92 | 754.13 |
| 645 | 668 | Ac-LTF$r8AYWAHL$S-NH2 | 1540.84 | 771.42 | 771.61 |
| 646 | 669 | Ac-LTF$r8AYWAQL$H-NH2 | 1581.87 | 791.94 | 792.15 |
| 647 | 670 | H-LTF$r8AYWAQL$A-NH2 | 1473.84 | 737.92 | 737.29 |
| 648 | 671 | Ac-HHF$r8AYWAQL$S-NH2 | 1591.83 | 796.92 | 797.35 |
| 649 | 672 | Ac-aAibWTF$r8VYWSQL$S-NH2 | 1804.96 | 903.48 | 903.64 |
| 650 | 673 | Ac-AibWTF$r8HYWAQL$S-NH2 | 1755.91 | 878.96 | 879.4 |
| 651 | 674 | Ac-AibAWTF$r8HYWAQL$S-NH2 | 1826.95 | 914.48 | 914.7 |
| 652 | 675 | Ac-fWTF$r8HYWAQL$S-NH2 | 1817.93 | 909.97 | 910.1 |
| 653 | 676 | Ac-AibWWTF$r8HYWAQL$S-NH2 | 1941.99 | 972.00 | 972.2 |
| 654 | 677 | Ac-WTF$r8LYWSQL$S-NH2 | 1662.88 | 832.44 | 832.8 |
| 655 | 678 | Ac-WTF$r8NleYWSQL$S-NH2 | 1662.88 | 832.44 | 832.6 |
| 656 | 679 | Ac-LTF$r8AYWSQL$a-NH2 | 1531.84 | 766.92 | 767.2 |
| 657 | 680 | Ac-LTF$r8EYWARL$A-NH2 | 1601.90 | 801.95 | 802.1 |
| 658 | 681 | Ac-LTF$r8EYWAHL$A-NH2 | 1582.86 | 792.43 | 792.6 |
| 659 | 682 | Ac-aTF$r8AYWAQL$S-NH2 | 1489.80 | 745.90 | 746.08 |
| 660 | 683 | Ac-AibTF$r8AYWAQL$S-NH2 | 1503.81 | 752.91 | 753.11 |
| 661 | 684 | Ac-AmfTF$r8AYWAQL$S-NH2 | 1579.84 | 790.92 | 791.14 |
| 662 | 685 | Ac-AmwTF$r8AYWAQL$S-NH2 | 1618.86 | 810.43 | 810.66 |
| 663 | 686 | Ac-NmLTF$r8AYWAQL$S-NH2 | 1545.86 | 773.93 | 774.11 |
| 664 | 687 | Ac-LNmTF$r8AYWAQL$S-NH2 | 1545.86 | 773.93 | 774.11 |
| 665 | 688 | Ac-LSarF$r8AYWAQL$S-NH2 | 1501.83 | 751.92 | 752.18 |
| 667 | 689 | Ac-LGF$r8AYWAQL$S-NH2 | 1487.82 | 744.91 | 745.15 |
| 668 | 690 | Ac-LTNmF$r8AYWAQL$S-NH2 | 1545.86 | 773.93 | 774.2 |
| 669 | 691 | Ac-TF$r8AYWAQL$S-NH2 | 1418.76 | 710.38 | 710.64 |
| 670 | 692 | Ac-ETF$r8AYWAQL$A-NH2 | 1531.81 | 766.91 | 767.2 |
| 671 | 693 | Ac-LTF$r8EYWAQL$A-NH2 | 1573.85 | 787.93 | 788.1 |
| 672 | 694 | Ac-LT2Nal$r8AYWSQL$S-NH2 | 1597.85 | 799.93 | 800.4 |
| 673 | 695 | Ac-LTF$r8AYWAAL$S-NH2 | 1474.82 | 738.41 | 738.68 |
| 674 | 696 | Ac-LTF$r8AYWAQhCha$S-NH2 | 1585.89 | 793.95 | 794.19 |
| 675 | 697 | Ac-LTF$r8AYWAQChg$S-NH2 | 1557.86 | 779.93 | 780.97 |
| 676 | 698 | Ac-LTF$r8AYWAQCba$S-NH2 | 1543.84 | 772.92 | 773.19 |
| 677 | 699 | Ac-LTF$r8AYWAQF3CF3$S-NH2 | 1633.82 | 817.91 | 818.15 |
| 678 | 700 | Ac-LTF$r8AYWAQ1Nal$S-NH2 | 1615.84 | 808.92 | 809.18 |
| 679 | 701 | Ac-LTF$r8AYWAQBip$S-NH2 | 1641.86 | 821.93 | 822.32 |
| 680 | 702 | Ac-LT2Nal$r8AYWAQL$S-NH2 | 1581.86 | 791.93 | 792.15 |
| 681 | 703 | Ac-LTF$r8AYWVQL$S-NH2 | 1559.88 | 780.94 | 781.62 |
| 682 | 704 | Ac-LTF$r8AWWAQL$S-NH2 | 1554.86 | 778.43 | 778.65 |
| 683 | 705 | Ac-FTF$r8VYWSQL$S-NH2 | 1609.85 | 805.93 | 806.12 |
| 684 | 706 | Ac-ITF$r8FYWAQL$S-NH2 | 1607.88 | 804.94 | 805.2 |
| 685 | 707 | Ac-ITF$r8LYWSQL$S-NH2 | 1589.89 | 795.95 | 796.22 |
| 686 | 708 | Ac-FTF$r8FYWAQL$S-NH2 | 1641.86 | 821.93 | 822.41 |
| 687 | 709 | Ac-VTF$r8AYWSQL$S-NH2 | 1533.82 | 767.91 | 768.19 |
| 688 | 710 | Ac-LTF$r8AHWAQL$S-NH2 | 1505.84 | 753.92 | 754.31 |
| 689 | 711 | Ac-LTF$r8AYWAQL$H-NH2 | 1581.87 | 791.94 | 791.94 |
| 690 | 712 | Ac-LTF$r8AYWAHL$S-NH2 | 1540.84 | 771.42 | 771.61 |
| 691 | 713 | Ac-aAibWTF$r8VYWSQL$S-NH2 | 1804.96 | 903.48 | 903.9 |
| 692 | 714 | Ac-AibWTF$r8HYWAQL$S-NH2 | 1755.91 | 878.96 | 879.5 |
| 693 | 715 | Ac-AibAWTF$r8HYWAQL$S-NH2 | 1826.95 | 914.48 | 914.7 |
| 694 | 716 | Ac-fWTF$r8HYWAQL$S-NH2 | 1817.93 | 909.97 | 910.2 |
| 695 | 717 | Ac-AibWWTF$r8HYWAQL$S-NH2 | 1941.99 | 972.00 | 972.7 |
| 696 | 718 | Ac-WTF$r8LYWSQL$S-NH2 | 1662.88 | 832.44 | 832.7 |
| 697 | 719 | Ac-WTF$r8NleYWSQL$S-NH2 | 1662.88 | 832.44 | 832.7 |
| 698 | 720 | Ac-LTF$r8AYWSQL$a-NH2 | 1531.84 | 766.92 | 767.2 |
| 699 | 721 | Ac-LTF$r8EYWARL$A-NH2 | 1601.90 | 801.95 | 802.2 |
| 700 | 722 | Ac-LTF$r8EYWAHL$A-NH2 | 1582.86 | 792.43 | 792.6 |
| 701 | 723 | Ac-aTF$r8AYWAQL$S-NH2 | 1489.80 | 745.90 | 746.1 |
| 702 | 724 | Ac-AibTF$r8AYWAQL$S-NH2 | 1503.81 | 752.91 | 753.2 |
| 703 | 725 | Ac-AmfTF$r8AYWAQL$S-NH2 | 1579.84 | 790.92 | 791.2 |
| 704 | 726 | Ac-AmwTF$r8AYWAQL$S-NH2 | 1618.86 | 810.43 | 810.7 |
| 705 | 727 | Ac-NmLTF$r8AYWAQL$S-NH2 | 1545.86 | 773.93 | 774.1 |
| 706 | 728 | Ac-LNmTF$r8AYWAQL$S-NH2 | 1545.86 | 773.93 | 774.4 |
| 707 | 729 | Ac-LSarF$r8AYWAQL$S-NH2 | 1501.83 | 751.92 | 752.1 |
| 708 | 730 | Ac-TF$r8AYWAQL$S-NH2 | 1418.76 | 710.38 | 710.8 |
| 709 | 731 | Ac-ETF$r8AYWAQL$A-NH2 | 1531.81 | 766.91 | 767.4 |
| 710 | 732 | Ac-LTF$r8EYWAQL$A-NH2 | 1573.85 | 787.93 | 788.2 |
| 711 | 733 | Ac-WTF$r8VYWSQL$S-NH2 | 1648.87 | 825.44 | 825.2 |
| 713 | 734 | Ac-YTF$r8FYWSQL$S-NH2 | 1673.85 | 837.93 | 837.3 |
| 714 | 735 | Ac-F$r8AY6clWSAL$A-NH2 | 1294.65 | 648.33 | 647.74 |
| 715 | 736 | Ac-ETF$r8EYWVQL$S-NH2 | 1633.84 | 817.92 | 817.36 |
| 716 | 737 | Ac-ETF$r8EHWAQL$A-NH2 | 1563.81 | 782.91 | 782.36 |
| 717 | 738 | Ac-ITF$r8EYWAQL$S-NH2 | 1589.85 | 795.93 | 795.38 |
| 718 | 739 | Ac-ITF$r8EHWVQL$A-NH2 | 1575.88 | 788.94 | 788.42 |
| 719 | 740 | Ac-ITF$r8EHWAQL$S-NH2 | 1563.85 | 782.93 | 782.43 |
| 720 | 741 | Ac-LTF4F$r8AYWAQCba$S-NH2 | 1561.83 | 781.92 | 781.32 |
| 721 | 742 | Ac-LTF3Cl$r8AYWAQhL$S-NH2 | 1579.82 | 790.91 | 790.64 |
| 722 | 743 | Ac-LTF3Cl$r8AYWAQCha$S-NH2 | 1605.84 | 803.92 | 803.37 |
| 723 | 744 | Ac-LTF3Cl$r8AYWAQChg$S-NH2 | 1591.82 | 796.91 | 796.27 |
| 724 | 745 | Ac-LTF3Cl$r8AYWAQCba$S-NH2 | 1577.81 | 789.91 | 789.83 |
| 725 | 746 | Ac-LTF$r8AY6clWSQL$S-NH2 | 1581.80 | 791.90 | 791.75 |
| 726 | 747 | Ac-LTF4F$r8HYWAQhL$S-NH2 | 1629.87 | 815.94 | 815.36 |
| 727 | 748 | Ac-LTF4F$r8HYWAQCba$S-NH2 | 1627.86 | 814.93 | 814.32 |
| 728 | 749 | Ac-LTF4F$r8AYWAQhL$S-NH2 | 1563.85 | 782.93 | 782.36 |
| 729 | 750 | Ac-LTF4F$r8AYWAQChg$S-NH2 | 1575.85 | 788.93 | 788.35 |
| 730 | 751 | Ac-ETF$r8EYWVAL$S-NH2 | 1576.82 | 789.41 | 788.79 |
| 731 | 752 | Ac-ETF$r8EHWAAL$A-NH2 | 1506.79 | 754.40 | 754.8 |
| 732 | 753 | Ac-ITF$r8EYWAAL$S-NH2 | 1532.83 | 767.42 | 767.75 |
| 733 | 754 | Ac-ITF$r8EHWVAL$A-NH2 | 1518.86 | 760.43 | 760.81 |
| 734 | 755 | Ac-ITF$r8EHWAAL$S-NH2 | 1506.82 | 754.41 | 754.8 |
| 735 | 756 | Pam-LTF$r8EYWAQL$S-NH2 | 1786.07 | 894.04 | 894.48 |
| 736 | 757 | Pam-ETF$r8EYWAQL$S-NH2 | 1802.03 | 902.02 | 902.34 |
| 737 | 758 | Ac-LTF$r8AYWLQL$S-NH2 | 1573.89 | 787.95 | 787.39 |
| 738 | 759 | Ac-LTF$r8EYWLQL$S-NH2 | 1631.90 | 816.95 | 817.33 |
| 739 | 760 | Ac-LTF$r8EHWLQL$S-NH2 | 1605.89 | 803.95 | 804.29 |
| 740 | 761 | Ac-LTF$r8VYWAQL$S-NH2 | 1559.88 | 780.94 | 781.34 |
| 741 | 762 | Ac-LTF$r8AYWSQL$S-NH2 | 1547.84 | 774.92 | 775.33 |
| 742 | 763 | Ac-ETF$r8AYWAQL$S-NH2 | 1547.80 | 774.90 | 775.7 |
| 743 | 764 | Ac-LTF$r8EYWAQL$S-NH2 | 1589.85 | 795.93 | 796.33 |
| 744 | 765 | Ac-LTF$r8HYWAQL$S-NHAm | 1667.94 | 834.97 | 835.37 |
| 745 | 766 | Ac-LTF$r8HYWAQL$S-NHiAm | 1667.94 | 834.97 | 835.27 |
| 746 | 767 | Ac-LTF$r8HYWAQL$S-NHnPr3Ph | 1715.94 | 858.97 | 859.42 |
| 747 | 768 | Ac-LTF$r8HYWAQL$S-NHnBu3,3Me | 1681.96 | 841.98 | 842.67 |
| 748 | 769 | Ac-LTF$r8HYWAQL$S-NHnBu | 1653.93 | 827.97 | 828.24 |
| 749 | 770 | Ac-LTF$r8HYWAQL$S-NHnPr | 1639.91 | 820.96 | 821.31 |
| 750 | 771 | Ac-LTF$r8HYWAQL$S-NHnEt2Ch | 1707.98 | 854.99 | 855.35 |
| 751 | 772 | Ac-LTF$r8HYWAQL$S-NHHex | 1681.96 | 841.98 | 842.4 |
| 752 | 773 | Ac-LTF$r8AYWAQL$S-NHmdPeg2 | 1633.91 | 817.96 | 855.35 |
| 753 | 774 | Ac-LTF$r8AYWAQL$A-NHmdPeg2 | 1617.92 | 809.96 | 810.58 |
| 754 | 775 | Ac-LTF$r5AYWAAL$s8S-NH2 | 1474.82 | 738.41 | 738.79 |
| 755 | 776 | Ac-LTF$r8AYWCouQL$S-NH2 | 1705.88 | 853.94 | 854.61 |
| 756 | 777 | Ac-LTF$r8CouYWAQL$S-NH2 | 1705.88 | 853.94 | 854.7 |
| 757 | 778 | Ac-CouTF$r8AYWAQL$S-NH2 | 1663.83 | 832.92 | 833.33 |
| 758 | 779 | H-LTF$r8AYWAQL$A-NH2 | 1473.84 | 737.92 | 737.29 |
| 759 | 780 | Ac-HHF$r8AYWAQL$S-NH2 | 1591.83 | 796.92 | 797.72 |
| 760 | 781 | Ac-LT2Nal$r8AYWSQL$S-NH2 | 1597.85 | 799.93 | 800.68 |
| 761 | 782 | Ac-LTF$r8HCouWAQL$S-NH2 | 1679.87 | 840.94 | 841.38 |
| 762 | 783 | Ac-LTF$r8AYWCou2QL$S-NH2 | 1789.94 | 895.97 | 896.51 |
| 763 | 784 | Ac-LTF$r8Cou2YWAQL$S-NH2 | 1789.94 | 895.97 | 896.5 |
| 764 | 785 | Ac-Cou2TF$r8AYWAQL$S-NH2 | 1747.90 | 874.95 | 875.42 |
| 765 | 786 | Ac-LTF$r8ACou2WAQL$S-NH2 | 1697.92 | 849.96 | 850.82 |
| 766 | 787 | Dmaac-LTF$r8AYWAQL$S-NH2 | 1574.89 | 788.45 | 788.82 |
| 767 | 788 | Hexac-LTF$r8AYWAQL$S-NH2 | 1587.91 | 794.96 | 795.11 |
| 768 | 789 | Napac-LTF$r8AYWAQL$S-NH2 | 1657.89 | 829.95 | 830.36 |
| 769 | 790 | Pam-LTF$r8AYWAQL$S-NH2 | 1728.06 | 865.03 | 865.45 |
| 770 | 791 | Ac-LT2Nal$r8HYAAQL$S-NH2 | 1532.84 | 767.42 | 767.61 |
| 771 | 792 | Ac-LT2Nal$/r8HYWAQL$/S-NH2 | 1675.91 | 838.96 | 839.1 |
| 772 | 793 | Ac-LT2Nal$r8HYFAQL$S-NH2 | 1608.87 | 805.44 | 805.9 |
| 773 | 794 | Ac-LT2Nal$r8HWAAQL$S-NH2 | 1555.86 | 778.93 | 779.08 |
| 774 | 795 | Ac-LT2Nal$r8HYAWQL$S-NH2 | 1647.88 | 824.94 | 825.04 |
| 775 | 796 | Ac-LT2Nal$r8HYAAQW$S-NH2 | 1605.83 | 803.92 | 804.05 |
| 776 | 797 | Ac-LTW$r8HYWAQL$S-NH2 | 1636.88 | 819.44 | 819.95 |
| 777 | 798 | Ac-LT1Nal$r8HYWAQL$S-NH2 | 1647.88 | 824.94 | 825.41 |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon i to i+4 crosslinker comprising one double bond. Amino acids represented as “$r5” are alpha-Me R5-pentenyl-alanine olefin amino acids connected by an all-carbon i to i+4 crosslinker comprising one double bond. Amino acids represented as “$s8” are alpha-Me S8-octenyl-alanine olefin amino acids connected by an all-carbon i to i+7 crosslinker comprising one double bond. Amino acids represented as “$r8” are alpha-Me R8-octenyl-alanine olefin amino acids connected by an all-carbon i to i+7 crosslinker comprising one double bond. “Ahx” represents an aminocyclohexyl linker. The crosslinkers are linear all-carbon crosslinker comprising eight or eleven carbon atoms between the alpha carbons of each amino acid. Amino acids represented as “$/” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker. Amino acids represented as “Amw” are alpha-Me tryptophan amino acids. Amino acids represented as “Aml” are alpha-Me leucine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “SW” are amino acids comprising two pentenyl-alanine olefin side chains that are not crosslinked. Amino acids represented as “% St” are amino acids comprising two pentenyl-alanine olefin side chains, each of which is crosslinked to another amino acid as indicated via fully saturated hydrocarbon crosslinks.

For example, the compounds represented as SP-72, SP-56 and SP-138 have the following structures (SEQ ID NOS 113, 97, 177, 87, 181, 307, 167, 229, 277, 370, 221, 217, 391 and 188, respectively, in order of appearance):

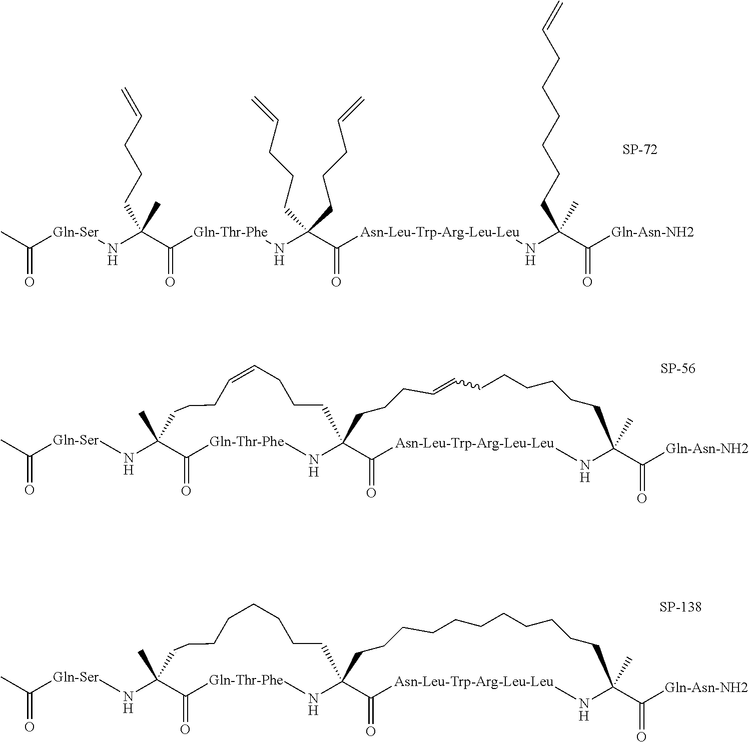

For example, additional compounds have the following structures:

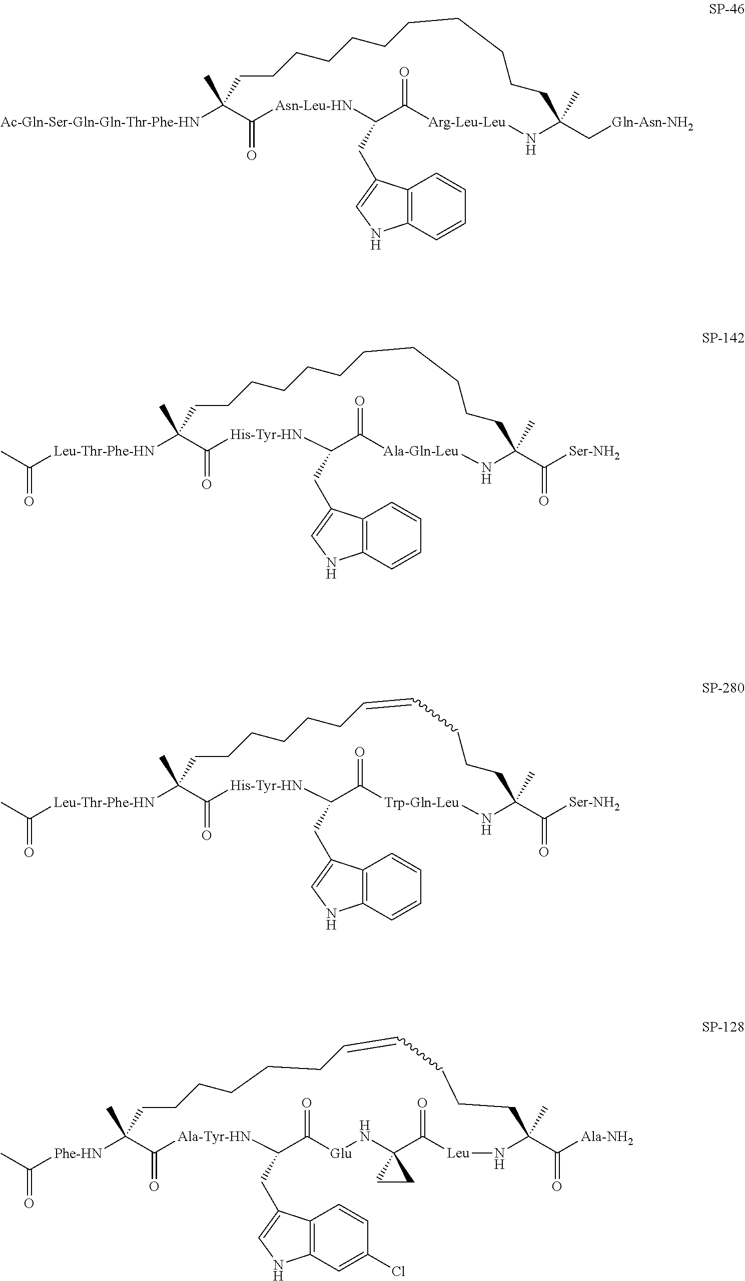

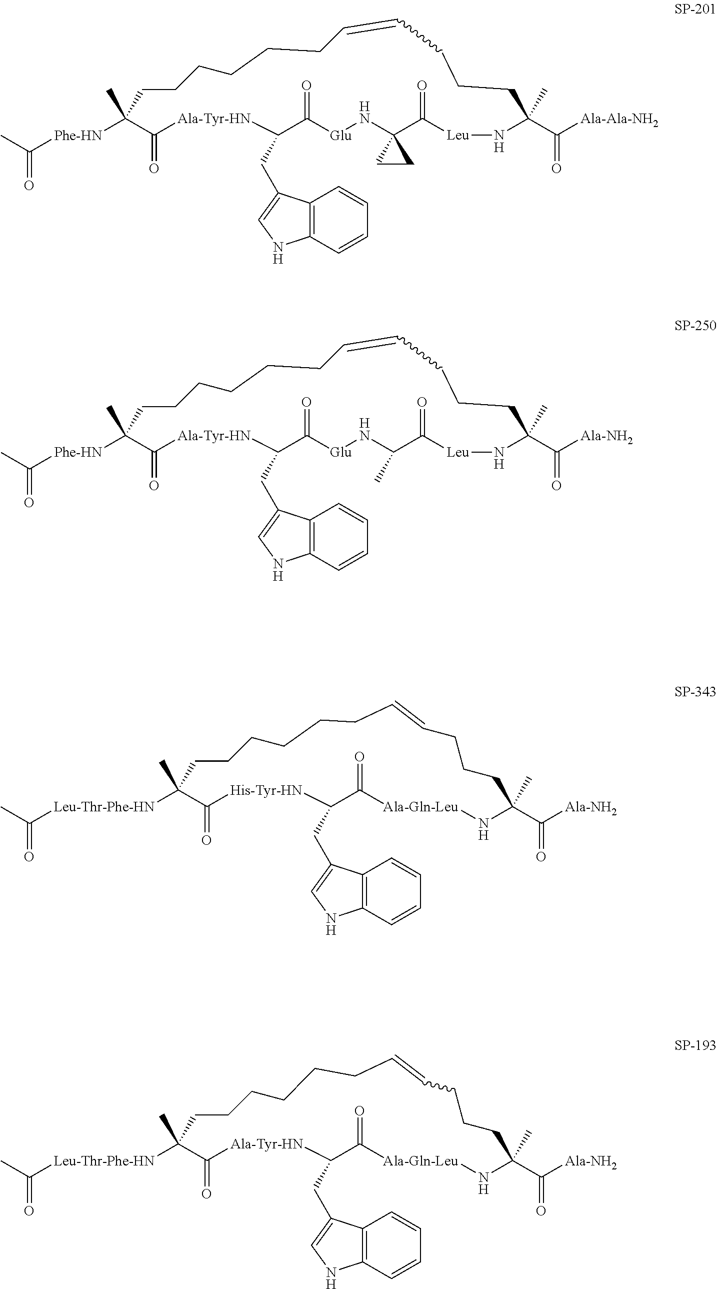

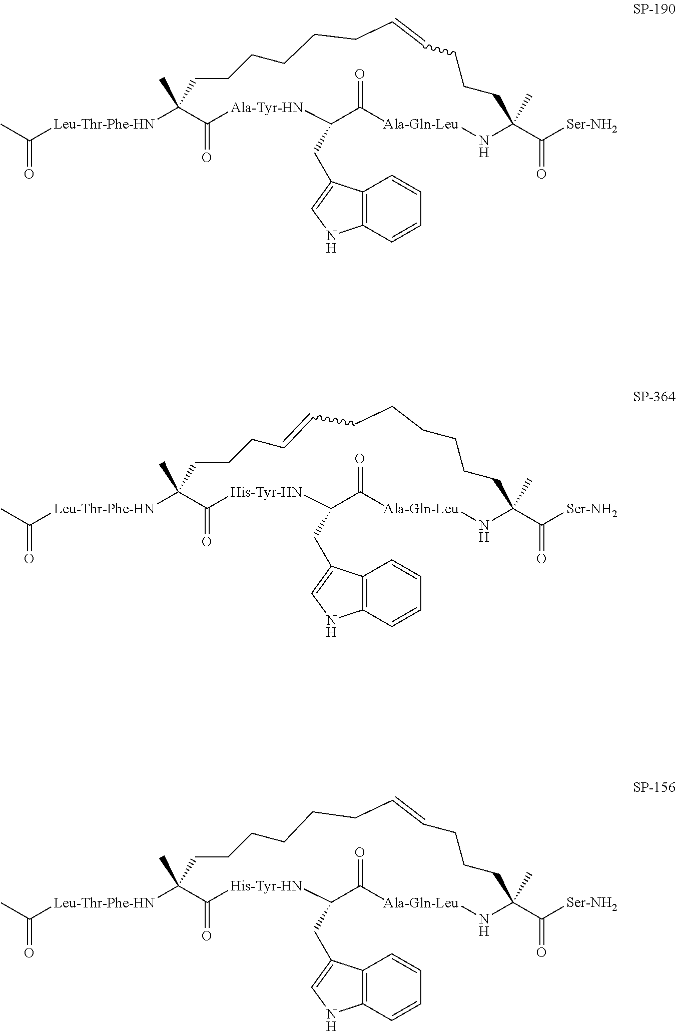

Tables 10-13 show a selection of peptidomimetic macrocycles.

TABLE 10

| | SEQ | | | Exact | Found | Calc | Calc | Calc |
|---|---|---|---|---|---|---|---|---|
| SP | ID NO: | Sequence | Isomer | Mass | Mass | (M + 1)/1 | (M + 2)/2 | (M + 3)/3 |
| SP1 | 799 | Ac-F$r8AYWEAc3cL$AAA-NH2 | | 1456.78 | 729.44 | 1457.79 | 729.4 | 486.6 |
| SP2 | 800 | Ac-F$r8AYWEAc3cL$AAibA-NH2 | | 1470.79 | 736.4 | 1471.8 | 736.4 | 491.27 |
| SP3 | 801 | Ac-LTF$r8AYWAQL$SANle-NH2 | | 1715.97 | 859.02 | 1716.98 | 858.99 | 573 |
| SP4 | 802 | Ac-LTF$r8AYWAQL$SAL-NH2 | | 1715.97 | 859.02 | 1716.98 | 858.99 | 573 |
| SP5 | 803 | Ac-LTF$r8AYWAQL$SAM-NH2 | | 1733.92 | 868.48 | 1734.93 | 867.97 | 578.98 |
| SP6 | 804 | Ac-LTF$r8AYWAQL$SAhL-NH2 | | 1729.98 | 865.98 | 1730.99 | 866 | 577.67 |
| SP7 | 805 | Ac-LTF$r8AYWAQL$SAF-NH2 | | 1749.95 | 876.36 | 1750.96 | 875.98 | 584.32 |
| SP8 | 806 | Ac-LTF$r8AYWAQL$SAI-NH2 | | 1715.97 | 859.02 | 1716.98 | 858.99 | 573 |
| SP9 | 807 | Ac-LTF$r8AYWAQL$SAChg-NH2 | | 1741.98 | 871.98 | 1742.99 | 872 | 581.67 |
| SP10 | 808 | Ac-LTF$r8AYWAQL$SAAib-NH2 | | 1687.93 | 845.36 | 1688.94 | 844.97 | 563.65 |
| SP11 | 809 | Ac-LTF$r8AYWAQL$SAA-NH2 | | 1673.92 | 838.01 | 1674.93 | 837.97 | 558.98 |
| SP12 | 810 | Ac-LTF$r8AYWA$L$S$Nle-NH2 | | 1767.04 | 884.77 | 1768.05 | 884.53 | 590.02 |
| SP13 | 811 | Ac-LTF$r8AYWA$L$S$A-NH2 | | 1724.99 | 864.23 | 1726 | 863.5 | 576 |
| SP14 | 812 | Ac-F$r8AYWEAc3cL$AANle-NH2 | | 1498.82 | 750.46 | 1499.83 | 750.42 | 500.61 |
| SP15 | 813 | Ac-F$r8AYWEAc3cL$AAL-NH2 | | 1498.82 | 750.46 | 1499.83 | 750.42 | 500.61 |
| SP16 | 814 | Ac-F$r8AYWEAc3cL$AAM-NH2 | | 1516.78 | 759.41 | 1517.79 | 759.4 | 506.6 |
| SP17 | 815 | Ac-F$r8AYWEAc3cL$AAhL-NH2 | | 1512.84 | 757.49 | 1513.85 | 757.43 | 505.29 |
| SP18 | 816 | Ac-F$r8AYWEAc3cL$AAF-NH2 | | 1532.81 | 767.48 | 1533.82 | 767.41 | 511.94 |
| SP19 | 817 | Ac-F$r8AYWEAc3cL$AAI-NH2 | | 1498.82 | 750.39 | 1499.83 | 750.42 | 500.61 |
| SP20 | 818 | Ac-F$r8AYWEAc3cL$AAChg-NH2 | | 1524.84 | 763.48 | 1525.85 | 763.43 | 509.29 |
| SP21 | 819 | Ac-F$r8AYWEAc3cL$AACha-NH2 | | 1538.85 | 770.44 | 1539.86 | 770.43 | 513.96 |
| SP22 | 820 | Ac-F$r8AYWEAc3cL$AAAib-NH2 | | 1470.79 | 736.84 | 1471.8 | 736.4 | 491.27 |
| SP23 | 821 | Ac-LTF$r8AYWAQL$AAAibV-NH2 | | 1771.01 | 885.81 | 1772.02 | 886.51 | 591.34 |
| SP24 | 822 | Ac-LTF$r8AYWAQL$AAAibV-NH2 | iso2 | 1771.01 | 886.26 | 1772.02 | 886.51 | 591.34 |
| SP25 | 823 | Ac-LTF$r8AYWAQL$SAibAA-NH2 | | 1758.97 | 879.89 | 1759.98 | 880.49 | 587.33 |
| SP26 | 824 | Ac-LTF$r8AYWAQL$SAibAA-NH2 | iso2 | 1758.97 | 880.34 | 1759.98 | 880.49 | 587.33 |
| SP27 | 825 | Ac-HLTF$r8HHWHQL$AANleNle-NH2 | | 2056.15 | 1028.86 | 2057.16 | 1029.08 | 686.39 |
| SP28 | 826 | Ac-DLTF$r8HHWHQL$RRLV-NH2 | | 2190.23 | 731.15 | 2191.24 | 1096.12 | 731.08 |
| SP29 | 827 | Ac-HHTF$r8HHWHQL$AAML-NH2 | | 2098.08 | 700.43 | 2099.09 | 1050.05 | 700.37 |
| SP30 | 828 | Ac-F$r8HHWHQL$RRDCha-NH2 | | 1917.06 | 959.96 | 1918.07 | 959.54 | 640.03 |
| SP31 | 829 | Ac-F$r8HHWHQL$HRFV-NH2 | | 1876.02 | 938.65 | 1877.03 | 939.02 | 626.35 |
| SP32 | 830 | Ac-HLTF$r8HHWHQL$AAhLA-NH2 | | 2028.12 | 677.2 | 2029.13 | 1015.07 | 677.05 |
| SP33 | 831 | Ac-DLTF$r8HHWHQL$RRChgl-NH2 | | 2230.26 | 1115.89 | 2231.27 | 1116.14 | 744.43 |
| SP34 | 832 | Ac-DLTF$r8HHWHQL$RRChgl-NH2 | iso2 | 2230.26 | 1115.96 | 2231.27 | 1116.14 | 744.43 |
| SP35 | 833 | Ac-HHTF$r8HHWHQL$AAChav-NH2 | | 2106.14 | 1053.95 | 2107.15 | 1054.08 | 703.05 |
| SP36 | 834 | Ac-F$r8HHWHQL$RRDa-NH2 | | 1834.99 | 918.3 | 1836 | 918.5 | 612.67 |
| SP37 | 835 | Ac-F$r8HHWHQL$HRAibG-NH2 | | 1771.95 | 886.77 | 1772.96 | 886.98 | 591.66 |
| SP38 | 836 | Ac-F$r8AYWAQL$HHNleL-NH2 | | 1730.97 | 866.57 | 1731.98 | 866.49 | 578 |
| SP39 | 837 | Ac-F$r8AYWSAL$HQANle-NH2 | | 1638.89 | 820.54 | 1639.9 | 820.45 | 547.3 |
| SP40 | 838 | Ac-F$r8AYWVQL$QHChgl-NH2 | | 1776.01 | 889.44 | 1777.02 | 889.01 | 593.01 |
| SP41 | 839 | Ac-F$r8AYWTAL$QQNlev-NH2 | | 1671.94 | 836.97 | 1672.95 | 836.98 | 558.32 |
| SP42 | 840 | Ac-F$r8AYWYQL$HAibAa-NH2 | | 1686.89 | 844.52 | 1687.9 | 844.45 | 563.3 |
| SP43 | 841 | Ac-LTF$r8AYWAQL$HHLa-NH2 | | 1903.05 | 952.27 | 1904.06 | 952.53 | 635.36 |
| SP44 | 842 | Ac-LTF$r8AYWAQL$HHLa-NH2 | iso2 | 1903.05 | 952.27 | 1904.06 | 952.53 | 635.36 |
| SP45 | 843 | Ac-LTF$r8AYWAQL$HQNlev-NH2 | | 1922.08 | 962.48 | 1923.09 | 962.05 | 641.7 |
| SP46 | 844 | Ac-LTF$r8AYWAQL$HQNlev-NH2 | iso2 | 1922.08 | 962.4 | 1923.09 | 962.05 | 641.7 |
| SP47 | 845 | Ac-LTF$r8AYWAQL$QQMl-NH2 | | 1945.05 | 973.95 | 1946.06 | 973.53 | 649.36 |
| SP48 | 846 | Ac-LTF$r8AYWAQL$QQMl-NH2 | iso2 | 1945.05 | 973.88 | 1946.06 | 973.53 | 649.36 |
| SP49 | 847 | Ac-LTF$r8AYWAQL$HAibhLV-NH2 | | 1893.09 | 948.31 | 1894.1 | 947.55 | 632.04 |
| SP50 | 848 | Ac-LTF$r8AYWAQL$AHFA-NH2 | | 1871.01 | 937.4 | 1872.02 | 936.51 | 624.68 |
| SP51 | 849 | Ac-HLTF$r8HHWHQL$AANlel-NH2 | | 2056.15 | 1028.79 | 2057.16 | 1029.08 | 686.39 |
| SP52 | 850 | Ac-DLTF$r8HHWHQL$RRLa-NH2 | | 2162.2 | 721.82 | 2163.21 | 1082.11 | 721.74 |
| SP53 | 851 | Ac-HHTF$r8HHWHQL$AAMv-NH2 | | 2084.07 | 1042.92 | 2085.08 | 1043.04 | 695.7 |
| SP54 | 852 | Ac-F$r8HHWHQL$RRDA-NH2 | | 1834.99 | 612.74 | 1836 | 918.5 | 612.67 |
| SP55 | 853 | Ac-F$r8HHWHQL$HRFCha-NH2 | | 1930.06 | 966.47 | 1931.07 | 966.04 | 644.36 |
| SP56 | 854 | Ac-F$r8AYWEAL$AA-NHAm | | 1443.82 | 1445.71 | 1444.83 | 722.92 | 482.28 |
| SP57 | 855 | Ac-F$r8AYWEAL$AA-NHiAm | | 1443.82 | 723.13 | 1444.83 | 722.92 | 482.28 |
| SP58 | 856 | Ac-F$r8AYWEAL$AA-NHnPr3Ph | | 1491.82 | 747.3 | 1492.83 | 746.92 | 498.28 |
| SP59 | 857 | Ac-F$r8AYWEAL$AA-NHnBu33Me | | 1457.83 | 1458.94 | 1458.84 | 729.92 | 486.95 |
| SP60 | 858 | Ac-F$r8AYWEAL$AA-NHnPr | | 1415.79 | 709.28 | 1416.8 | 708.9 | 472.94 |
| SP61 | 859 | Ac-F$r8AYWEAL$AA-NHnEt2Ch | | 1483.85 | 1485.77 | 1484.86 | 742.93 | 495.62 |
| SP62 | 860 | Ac-F$r8AYWEAL$AA-NHnEt2Cp | | 1469.83 | 1470.78 | 1470.84 | 735.92 | 490.95 |
| SP63 | 861 | Ac-F$r8AYWEAL$AA-NHHex | | 1457.83 | 730.19 | 1458.84 | 729.92 | 486.95 |
| SP64 | 862 | Ac-LTF$r8AYWAQL$AAIA-NH2 | | 1771.01 | 885.81 | 1772.02 | 886.51 | 591.34 |
| SP65 | 863 | Ac-LTF$r8AYWAQL$AAIA-NH2 | iso2 | 1771.01 | 866.8 | 1772.02 | 886.51 | 591.34 |
| SP66 | 864 | Ac-LTF$r8AYWAAL$AAMA-NH2 | | 1731.94 | 867.08 | 1732.95 | 866.98 | 578.32 |
| SP67 | 865 | Ac-LTF$r8AYWAAL$AAMA-NH2 | iso2 | 1731.94 | 867.28 | 1732.95 | 866.98 | 578.32 |
| SP68 | 866 | Ac-LTF$r8AYWAQL$AANleA-NH2 | | 1771.01 | 867.1 | 1772.02 | 886.51 | 591.34 |
| SP69 | 867 | Ac-LTF$r8AYWAQL$AANleA-NH2 | iso2 | 1771.01 | 886.89 | 1772.02 | 886.51 | 591.34 |
| SP70 | 868 | Ac-LTF$r8AYWAQL$AAIa-NH2 | | 1771.01 | 886.8 | 1772.02 | 886.51 | 591.34 |
| SP71 | 869 | Ac-LTF$r8AYWAQL$AAIa-NH2 | iso2 | 1771.01 | 887.09 | 1772.02 | 886.51 | 591.34 |
| SP72 | 870 | Ac-LTF$r8AYWAAL$AAMa-NH2 | | 1731.94 | 867.17 | 1732.95 | 866.98 | 578.32 |
| SP73 | 871 | Ac-LTF$r8AYWAAL$AAMa-NH2 | iso2 | 1731.94 | 867.37 | 1732.95 | 866.98 | 578.32 |
| SP74 | 872 | Ac-LTF$r8AYWAQL$AANlea-NH2 | | 1771.01 | 887.08 | 1772.02 | 886.51 | 591.34 |
| SP75 | 873 | Ac-LTF$r8AYWAQL$AANlea-NH2 | iso2 | 1771.01 | 887.08 | 1772.02 | 886.51 | 591.34 |
| SP76 | 874 | Ac-LTF$r8AYWAAL$AAIv-NH2 | | 1742.02 | 872.37 | 1743.03 | 872.02 | 581.68 |
| SP77 | 875 | Ac-LTF$r8AYWAAL$AAIv-NH2 | iso2 | 1742.02 | 872.74 | 1743.03 | 872.02 | 581.68 |
| SP78 | 876 | Ac-LTF$r8AYWAQL$AAMv-NH2 | | 1817 | 910.02 | 1818.01 | 909.51 | 606.67 |
| SP79 | 877 | Ac-LTF$r8AYWAAL$AANlev-NH2 | | 1742.02 | 872.37 | 1743.03 | 872.02 | 581.68 |
| SP80 | 878 | Ac-LTF$r8AYWAAL$AANlev-NH2 | iso2 | 1742.02 | 872.28 | 1743.03 | 872.02 | 581.68 |
| SP81 | 879 | Ac-LTF$r8AYWAQL$AAIl-NH2 | | 1813.05 | 907.81 | 1814.06 | 907.53 | 605.36 |
| SP82 | 880 | Ac-LTF$r8AYWAQL$AAIl-NH2 | iso2 | 1813.05 | 907.81 | 1814.06 | 907.53 | 605.36 |
| SP83 | 881 | Ac-LTF$r8AYWAAL$AAMl-NH2 | | 1773.99 | 887.37 | 1775 | 888 | 592.34 |
| SP84 | 882 | Ac-LTF$r8AYWAQL$AANlel-NH2 | | 1813.05 | 907.61 | 1814.06 | 907.53 | 605.36 |
| SP85 | 883 | Ac-LTF$r8AYWAQL$AANlel-NH2 | iso2 | 1813.05 | 907.71 | 1814.06 | 907.53 | 605.36 |
| SP86 | 884 | Ac-F$r8AYWEAL$AAMA-NH2 | | 1575.82 | 789.02 | 1576.83 | 788.92 | 526.28 |
| SP87 | 885 | Ac-F$r8AYWEAL$AANleA-NH2 | | 1557.86 | 780.14 | 1558.87 | 779.94 | 520.29 |
| SP88 | 886 | Ac-F$r8AYWEAL$AAIa-NH2 | | 1557.86 | 780.33 | 1558.87 | 779.94 | 520.29 |
| SP89 | 887 | Ac-F$r8AYWEAL$AAMa-NH2 | | 1575.82 | 789.3 | 1576.83 | 788.92 | 526.28 |
| SP90 | 888 | Ac-F$r8AYWEAL$AANlea-NH2 | | 1557.86 | 779.4 | 1558.87 | 779.94 | 520.29 |
| SP91 | 889 | Ac-F$r8AYWEAL$AAIv-NH2 | | 1585.89 | 794.29 | 1586.9 | 793.95 | 529.64 |
| SP92 | 890 | Ac-F$r8AYWEAL$AAMv-NH2 | | 1603.85 | 803.08 | 1604.86 | 802.93 | 535.62 |
| SP93 | 891 | Ac-F$r8AYWEAL$AANlev-NH2 | | 1585.89 | 793.46 | 1586.9 | 793.95 | 529.64 |
| SP94 | 892 | Ac-F$r8AYWEAL$AAIl-NH2 | | 1599.91 | 800.49 | 1600.92 | 800.96 | 534.31 |
| SP95 | 893 | Ac-F$r8AYWEAL$AAMl-NH2 | | 1617.86 | 809.44 | 1618.87 | 809.94 | 540.29 |
| SP96 | 894 | Ac-F$r8AYWEAL$AANlel-NH2 | | 1599.91 | 801.7 | 1600.92 | 800.96 | 534.31 |
| SP97 | 895 | Ac-F$r8AYWEAL$AANlel-NH2 | iso2 | 1599.91 | 801.42 | 1600.92 | 800.96 | 534.31 |
| SP98 | 896 | Ac-LTF$r8AY6clWAQL$SAA-NH2 | | 1707.88 | 855.72 | 1708.89 | 854.95 | 570.3 |
| SP99 | 897 | Ac-LTF$r8AY6clWAQL$SAA-NH2 | iso2 | 1707.88 | 855.35 | 1708.89 | 854.95 | 570.3 |
| SP100 | 898 | Ac-WTF$r8FYWSQL$AVAa-NH2 | | 1922.01 | 962.21 | 1923.02 | 962.01 | 641.68 |
| SP101 | 899 | Ac-WTF$r8FYWSQL$AVAa-NH2 | iso2 | 1922.01 | 962.49 | 1923.02 | 962.01 | 641.68 |
| SP102 | 900 | Ac-WTF$r8VYWSQL$AVA-NH2 | | 1802.98 | 902.72 | 1803.99 | 902.5 | 602 |
| SP103 | 901 | Ac-WTF$r8VYWSQL$AVA-NH2 | iso2 | 1802.98 | 903 | 1803.99 | 902.5 | 602 |
| SP104 | 902 | Ac-WTF$r8FYWSQL$SAAa-NH2 | | 1909.98 | 956.47 | 1910.99 | 956 | 637.67 |
| SP105 | 903 | Ac-WTF$r8FYWSQL$SAAa-NH2 | iso2 | 1909.98 | 956.47 | 1910.99 | 956 | 637.67 |
| SP106 | 904 | Ac-WTF$r8VYWSQL$AVAaa-NH2 | | 1945.05 | 974.15 | 1946.06 | 973.53 | 649.36 |
| SP107 | 905 | Ac-WTF$r8VYWSQL$AVAaa-NH2 | iso2 | 1945.05 | 973.78 | 1946.06 | 973.53 | 649.36 |
| SP108 | 906 | Ac-LTF$r8AYWAQL$AVG-NH2 | | 1671.94 | 837.52 | 1672.95 | 836.98 | 558.32 |
| SP109 | 907 | Ac-LTF$r8AYWAQL$AVG-NH2 | iso2 | 1671.94 | 837.21 | 1672.95 | 836.98 | 558.32 |
| SP110 | 908 | Ac-LTF$r8AYWAQL$AVQ-NH2 | | 1742.98 | 872.74 | 1743.99 | 872.5 | 582 |
| SP111 | 909 | Ac-LTF$r8AYWAQL$AVQ-NH2 | iso2 | 1742.98 | 872.74 | 1743.99 | 872.5 | 582 |
| SP112 | 910 | Ac-LTF$r8AYWAQL$SAa-NH2 | | 1673.92 | 838.23 | 1674.93 | 837.97 | 558.98 |
| SP113 | 911 | Ac-LTF$r8AYWAQL$SAa-NH2 | iso2 | 1673.92 | 838.32 | 1674.93 | 837.97 | 558.98 |
| SP114 | 912 | Ac-LTF$r8AYWAQhL$SAA-NH2 | | 1687.93 | 844.37 | 1688.94 | 844.97 | 563.65 |
| SP115 | 913 | Ac-LTF$r8AYWAQhL$SAA-NH2 | iso2 | 1687.93 | 844.81 | 1688.94 | 844.97 | 563.65 |
| SP116 | 914 | Ac-LTF$r8AYWEQLStSA$-NH2 | | 1826 | 905.27 | 1827.01 | 914.01 | 609.67 |
| SP117 | 915 | Ac-LTF$r8AYWAQL$SLA-NH2 | | 1715.97 | 858.48 | 1716.98 | 858.99 | 573 |
| SP118 | 916 | Ac-LTF$r8AYWAQL$SLA-NH2 | iso2 | 1715.97 | 858.87 | 1716.98 | 858.99 | 573 |
| SP119 | 917 | Ac-LTF$r8AYWAQL$SWA-NH2 | | 1788.96 | 895.21 | 1789.97 | 895.49 | 597.33 |
| SP120 | 918 | Ac-LTF$r8AYWAQL$SWA-NH2 | iso2 | 1788.96 | 895.28 | 1789.97 | 895.49 | 597.33 |
| SP121 | 919 | Ac-LTF$r8AYWAQL$SVS-NH2 | | 1717.94 | 859.84 | 1718.95 | 859.98 | 573.65 |
| SP122 | 920 | Ac-LTF$r8AYWAQL$SAS-NH2 | | 1689.91 | 845.85 | 1690.92 | 845.96 | 564.31 |
| SP123 | 921 | Ac-LTF$r8AYWAQL$SVG-NH2 | | 1687.93 | 844.81 | 1688.94 | 844.97 | 563.65 |
| SP124 | 922 | Ac-ETF$r8VYWAQL$SAa-NH2 | | 1717.91 | 859.76 | 1718.92 | 859.96 | 573.64 |
| SP125 | 923 | Ac-ETF$r8VYWAQL$SAA-NH2 | | 1717.91 | 859.84 | 1718.92 | 859.96 | 573.64 |
| SP126 | 924 | Ac-ETF$r8VYWAQL$SVA-NH2 | | 1745.94 | 873.82 | 1746.95 | 873.98 | 582.99 |
| SP127 | 925 | Ac-ETF$r8VYWAQL$SLA-NH2 | | 1759.96 | 880.85 | 1760.97 | 880.99 | 587.66 |
| SP128 | 926 | Ac-ETF$r8VYWAQL$SWA-NH2 | | 1832.95 | 917.34 | 1833.96 | 917.48 | 611.99 |
| SP129 | 927 | Ac-ETF$r8KYWAQL$SWA-NH2 | | 1861.98 | 931.92 | 1862.99 | 932 | 621.67 |
| SP130 | 928 | Ac-ETF$r8VYWAQL$SVS-NH2 | | 1761.93 | 881.89 | 1762.94 | 881.97 | 588.32 |
| SP131 | 929 | Ac-ETF$r8VYWAQL$SAS-NH2 | | 1733.9 | 867.83 | 1734.91 | 867.96 | 578.97 |
| SP132 | 930 | Ac-ETF$r8VYWAQL$SVG-NH2 | | 1731.92 | 866.87 | 1732.93 | 866.97 | 578.31 |
| SP133 | 931 | Ac-LTF$r8VYWAQL$SSa-NH2 | | 1717.94 | 859.47 | 1718.95 | 859.98 | 573.65 |
| SP134 | 932 | Ac-ETF$r8VYWAQL$SSa-NH2 | | 1733.9 | 867.83 | 1734.91 | 867.96 | 578.97 |
| SP135 | 933 | Ac-LTF$r8VYWAQL$SNa-NH2 | | 1744.96 | 873.38 | 1745.97 | 873.49 | 582.66 |
| SP136 | 934 | Ac-ETF$r8VYWAQL$SNa-NH2 | | 1760.91 | 881.3 | 1761.92 | 881.46 | 587.98 |
| SP137 | 935 | Ac-LTF$r8VYWAQL$SAa-NH2 | | 1701.95 | 851.84 | 1702.96 | 851.98 | 568.32 |
| SP138 | 936 | Ac-LTF$r8VYWAQL$SVA-NH2 | | 1729.98 | 865.53 | 1730.99 | 866 | 577.67 |
| SP139 | 937 | Ac-LTF$r8VYWAQL$SVA-NH2 | iso2 | 1729.98 | 865.9 | 1730.99 | 866 | 577.67 |
| SP140 | 938 | Ac-LTF$r8VYWAQL$SWA-NH2 | | 1816.99 | 909.42 | 1818 | 909.5 | 606.67 |
| SP141 | 939 | Ac-LTF$r8VYWAQL$SVS-NH2 | | 1745.98 | 873.9 | 1746.99 | 874 | 583 |
| SP142 | 940 | Ac-LTF$r8VYWAQL$SVS-NH2 | iso2 | 1745.98 | 873.9 | 1746.99 | 874 | 583 |
| SP143 | 941 | Ac-LTF$r8VYWAQL$SAS-NH2 | | 1717.94 | 859.84 | 1718.95 | 859.98 | 573.65 |
| SP144 | 942 | Ac-LTF$r8VYWAQL$SAS-NH2 | iso2 | 1717.94 | 859.91 | 1718.95 | 859.98 | 573.65 |
| SP145 | 943 | Ac-LTF$r8VYWAQL$SVG-NH2 | | 1715.97 | 858.87 | 1716.98 | 858.99 | 573 |
| SP146 | 944 | Ac-LTF$r8VYWAQL$SVG-NH2 | iso2 | 1715.97 | 858.87 | 1716.98 | 858.99 | 573 |
| SP147 | 945 | Ac-LTF$r8EYWAQCha$SAA-NH2 | | 1771.96 | 886.85 | 1772.97 | 886.99 | 591.66 |
| SP148 | 946 | Ac-LTF$r8EYWAQCha$SAA-NH2 | iso2 | 1771.96 | 886.85 | 1772.97 | 886.99 | 591.66 |
| SP149 | 947 | Ac-LTF$r8EYWAQCpg$SAA-NH2 | | 1743.92 | 872.86 | 1744.93 | 872.97 | 582.31 |
| SP150 | 948 | Ac-LTF$r8EYWAQCpg$SAA-NH2 | iso2 | 1743.92 | 872.86 | 1744.93 | 872.97 | 582.31 |
| SP151 | 949 | Ac-LTF$r8EYWAQF$SAA-NH2 | | 1765.91 | 883.44 | 1766.92 | 883.96 | 589.64 |
| SP152 | 950 | Ac-LTF$r8EYWAQF$SAA-NH2 | iso2 | 1765.91 | 883.89 | 1766.92 | 883.96 | 589.64 |
| SP153 | 951 | Ac-LTF$r8EYWAQCba$SAA-NH2 | | 1743.92 | 872.42 | 1744.93 | 872.97 | 582.31 |
| SP154 | 952 | Ac-LTF$r8EYWAQCba$SAA-NH2 | iso2 | 1743.92 | 873.39 | 1744.93 | 872.97 | 582.31 |
| SP155 | 953 | Ac-LTF3Cl$r8EYWAQL$SAA-NH2 | | 1765.89 | 883.89 | 1766.9 | 883.95 | 589.64 |
| SP156 | 954 | Ac-LTF3Cl$r8EYWAQL$SAA-NH2 | iso2 | 1765.89 | 883.96 | 1766.9 | 883.95 | 589.64 |
| SP157 | 955 | Ac-LTF34F2$r8EYWAQL$SAA-NH2 | | 1767.91 | 884.48 | 1768.92 | 884.96 | 590.31 |
| SP158 | 956 | Ac-LTF34F2$r8EYWAQL$SAA-NH2 | iso2 | 1767.91 | 884.48 | 1768.92 | 884.96 | 590.31 |
| SP159 | 957 | Ac-LTF34F2$r8EYWAQhL$SAA-NH2 | | 1781.92 | 891.44 | 1782.93 | 891.97 | 594.98 |
| SP160 | 958 | Ac-LTF34F2$r8EYWAQhL$SAA-NH2 | iso2 | 1781.92 | 891.88 | 1782.93 | 891.97 | 594.98 |
| SP161 | 959 | Ac-ETF$r8EYWAQL$SAA-NH2 | | 1747.88 | 874.34 | 1748.89 | 874.95 | 583.63 |
| SP162 | 960 | Ac-LTF$r8AYWVQL$SAA-NH2 | | 1701.95 | 851.4 | 1702.96 | 851.98 | 568.32 |
| SP163 | 961 | Ac-LTF$r8AHWAQL$SAA-NH2 | | 1647.91 | 824.83 | 1648.92 | 824.96 | 550.31 |
| SP164 | 962 | Ac-LTF$r8AEWAQL$SAA-NH2 | | 1639.9 | 820.39 | 1640.91 | 820.96 | 547.64 |
| SP165 | 963 | Ac-LTF$r8ASWAQL$SAA-NH2 | | 1597.89 | 799.38 | 1598.9 | 799.95 | 533.64 |
| SP166 | 964 | Ac-LTF$r8AEWAQL$SAA-NH2 | iso2 | 1639.9 | 820.39 | 1640.91 | 820.96 | 547.64 |
| SP167 | 965 | Ac-LTF$r8ASWAQL$SAA-NH2 | iso2 | 1597.89 | 800.31 | 1598.9 | 799.95 | 533.64 |
| SP168 | 966 | Ac-LTF$r8AF4coohWAQL$SAA-NH2 | | 1701.91 | 851.4 | 1702.92 | 851.96 | 568.31 |
| SP169 | 967 | Ac-LTF$r8AF4coohWAQL$SAA-NH2 | iso2 | 1701.91 | 851.4 | 1702.92 | 851.96 | 568.31 |
| SP170 | 968 | Ac-LTF$r8AHWAQL$AAIa-NH2 | | 1745 | 874.13 | 1746.01 | 873.51 | 582.67 |
| SP171 | 969 | Ac-ITF$r8FYWAQL$AAIa-NH2 | | 1847.04 | 923.92 | 1848.05 | 924.53 | 616.69 |
| SP172 | 970 | Ac-ITF$r8EHWAQL$AAIa-NH2 | | 1803.01 | 903.17 | 1804.02 | 902.51 | 602.01 |
| SP173 | 971 | Ac-ITF$r8EHWAQL$AAIa-NH2 | iso2 | 1803.01 | 903.17 | 1804.02 | 902.51 | 602.01 |
| SP174 | 972 | Ac-ETF$r8EHWAQL$AAIa-NH2 | | 1818.97 | 910.76 | 1819.98 | 910.49 | 607.33 |
| SP175 | 973 | Ac-ETF$r8EHWAQL$AAIa-NH2 | iso2 | 1818.97 | 910.85 | 1819.98 | 910.49 | 607.33 |
| SP176 | 974 | Ac-LTF$r8AHWVQL$AAIa-NH2 | | 1773.03 | 888.09 | 1774.04 | 887.52 | 592.02 |
| SP177 | 975 | Ac-ITF$r8FYWVQL$AAIa-NH2 | | 1875.07 | 939.16 | 1876.08 | 938.54 | 626.03 |
| SP178 | 976 | Ac-ITF$r8EYWVQL$AAIa-NH2 | | 1857.04 | 929.83 | 1858.05 | 929.53 | 620.02 |
| SP179 | 977 | Ac-ITF$r8EHWVQL$AAIa-NH2 | | 1831.04 | 916.86 | 1832.05 | 916.53 | 611.35 |
| SP180 | 978 | Ac-LTF$r8AEWAQL$AAIa-NH2 | | 1736.99 | 869.87 | 1738 | 869.5 | 580 |
| SP181 | 979 | Ac-LTF$r8AF4coohWAQL$AAIa-NH2 | | 1799 | 900.17 | 1800.01 | 900.51 | 600.67 |
| SP182 | 980 | Ac-LTF$r8AF4coohWAQL$AAIa-NH2 | iso2 | 1799 | 900.24 | 1800.01 | 900.51 | 600.67 |
| SP183 | 981 | Ac-LTF$r8AHWAQL$AHFA-NH2 | | 1845.01 | 923.89 | 1846.02 | 923.51 | 616.01 |
| SP184 | 982 | Ac-ITF$r8FYWAQL$AHFA-NH2 | | 1947.05 | 975.05 | 1948.06 | 974.53 | 650.02 |
| SP185 | 983 | Ac-ITF$r8FYWAQL$AHFA-NH2 | iso2 | 1947.05 | 976.07 | 1948.06 | 974.53 | 650.02 |
| SP186 | 984 | Ac-ITF$r8FHWAQL$AEFA-NH2 | | 1913.02 | 958.12 | 1914.03 | 957.52 | 638.68 |
| SP187 | 985 | Ac-ITF$r8FHWAQL$AEFA-NH2 | iso2 | 1913.02 | 957.86 | 1914.03 | 957.52 | 638.68 |
| SP188 | 986 | Ac-ITF$r8EHWAQL$AHFA-NH2 | | 1903.01 | 952.94 | 1904.02 | 952.51 | 635.34 |
| SP189 | 987 | Ac-ITF$r8EHWAQL$AHFA-NH2 | iso2 | 1903.01 | 953.87 | 1904.02 | 952.51 | 635.34 |
| SP190 | 988 | Ac-LTF$r8AHWVQL$AHFA-NH2 | | 1873.04 | 937.86 | 1874.05 | 937.53 | 625.35 |
| SP191 | 989 | Ac-ITF$r8FYWVQL$AHFA-NH2 | | 1975.08 | 988.83 | 1976.09 | 988.55 | 659.37 |
| SP192 | 990 | Ac-ITF$r8EYWVQL$AHFA-NH2 | | 1957.05 | 979.35 | 1958.06 | 979.53 | 653.36 |
| SP193 | 991 | Ac-ITF$r8EHWVQL$AHFA-NH2 | | 1931.05 | 967 | 1932.06 | 966.53 | 644.69 |
| SP194 | 992 | Ac-ITF$r8EHWVQL$AHFA-NH2 | iso2 | 1931.05 | 967.93 | 1932.06 | 966.53 | 644.69 |
| SP195 | 993 | Ac-ETF$r8EYWAAL$SAA-NH2 | | 1690.86 | 845.85 | 1691.87 | 846.44 | 564.63 |
| SP196 | 994 | Ac-LTF$r8AYWVAL$SAA-NH2 | | 1644.93 | 824.08 | 1645.94 | 823.47 | 549.32 |
| SP197 | 995 | Ac-LTF$r8AHWAAL$SAA-NH2 | | 1590.89 | 796.88 | 1591.9 | 796.45 | 531.3 |
| SP198 | 996 | Ac-LTF$r8AEWAAL$SAA-NH2 | | 1582.88 | 791.9 | 1583.89 | 792.45 | 528.63 |
| SP199 | 997 | Ac-LTF$r8AEWAAL$SAA-NH2 | iso2 | 1582.88 | 791.9 | 1583.89 | 792.45 | 528.63 |
| SP200 | 998 | Ac-LTF$r8ASWAAL$SAA-NH2 | | 1540.87 | 770.74 | 1541.88 | 771.44 | 514.63 |
| SP201 | 999 | Ac-LTF$r8ASWAAL$SAA-NH2 | iso2 | 1540.87 | 770.88 | 1541.88 | 771.44 | 514.63 |
| SP202 | 1000 | Ac-LTF$r8AYWAAL$AAIa-NH2 | | 1713.99 | 857.39 | 1715 | 858 | 572.34 |
| SP203 | 1001 | Ac-LTF$r8AYWAAL$AAIa-NH2 | iso2 | 1713.99 | 857.84 | 1715 | 858 | 572.34 |
| SP204 | 1002 | Ac-LTF$r8AYWAAL$AHFA-NH2 | | 1813.99 | 907.86 | 1815 | 908 | 605.67 |
| SP205 | 1003 | Ac-LTF$r8EHWAQL$AHIa-NH2 | | 1869.03 | 936.1 | 1870.04 | 935.52 | 624.02 |
| SP206 | 1004 | Ac-LTF$r8EHWAQL$AHIa-NH2 | iso2 | 1869.03 | 937.03 | 1870.04 | 935.52 | 624.02 |
| SP207 | 1005 | Ac-LTF$r8AHWAQL$AHIa-NH2 | | 1811.03 | 906.87 | 1812.04 | 906.52 | 604.68 |
| SP208 | 1006 | Ac-LTF$r8EYWAQL$AHIa-NH2 | | 1895.04 | 949.15 | 1896.05 | 948.53 | 632.69 |
| SP209 | 1007 | Ac-LTF$r8AYWAQL$AAFa-NH2 | | 1804.99 | 903.2 | 1806 | 903.5 | 602.67 |
| SP210 | 1008 | Ac-LTF$r8AYWAQL$AAFa-NH2 | iso2 | 1804.99 | 903.28 | 1806 | 903.5 | 602.67 |
| SP211 | 1009 | Ac-LTF$r8AYWAQL$AAWa-NH2 | | 1844 | 922.81 | 1845.01 | 923.01 | 615.67 |
| SP212 | 1010 | Ac-LTF$r8AYWAQL$AAVa-NH2 | | 1756.99 | 878.86 | 1758 | 879.5 | 586.67 |
| SP213 | 1011 | Ac-LTF$r8AYWAQL$AAVa-NH2 | iso2 | 1756.99 | 879.3 | 1758 | 879.5 | 586.67 |
| SP214 | 1012 | Ac-LTF$r8AYWAQL$AALa-NH2 | | 1771.01 | 886.26 | 1772.02 | 886.51 | 591.34 |
| SP215 | 1013 | Ac-LTF$r8AYWAQL$AALa-NH2 | iso2 | 1771.01 | 886.33 | 1772.02 | 886.51 | 591.34 |
| SP216 | 1014 | Ac-LTF$r8EYWAQL$AAIa-NH2 | | 1829.01 | 914.89 | 1830.02 | 915.51 | 610.68 |
| SP217 | 1015 | Ac-LTF$r8EYWAQL$AAIa-NH2 | iso2 | 1829.01 | 915.34 | 1830.02 | 915.51 | 610.68 |
| SP218 | 1016 | Ac-LTF$r8EYWAQL$AAFa-NH2 | | 1863 | 932.87 | 1864.01 | 932.51 | 622.01 |
| SP219 | 1017 | Ac-LTF$r8EYWAQL$AAFa-NH2 | iso2 | 1863 | 932.87 | 1864.01 | 932.51 | 622.01 |
| SP220 | 1018 | Ac-LTF$r8EYWAQL$AAVa-NH2 | | 1815 | 908.23 | 1816.01 | 908.51 | 606.01 |
| SP221 | 1019 | Ac-LTF$r8EYWAQL$AAVa-NH2 | iso2 | 1815 | 908.31 | 1816.01 | 908.51 | 606.01 |
| SP222 | 1020 | Ac-LTF$r8EHWAQL$AAIa-NH2 | | 1803.01 | 903.17 | 1804.02 | 902.51 | 602.01 |
| SP223 | 1021 | Ac-LTF$r8EHWAQL$AAIa-NH2 | iso2 | 1803.01 | 902.8 | 1804.02 | 902.51 | 602.01 |
| SP224 | 1022 | Ac-LTF$r8EHWAQL$AAWa-NH2 | | 1876 | 939.34 | 1877.01 | 939.01 | 626.34 |
| SP225 | 1023 | Ac-LTF$r8EHWAQL$AAWa-NH2 | iso2 | 1876 | 939.62 | 1877.01 | 939.01 | 626.34 |
| SP226 | 1024 | Ac-LTF$r8EHWAQL$AALa-NH2 | | 1803.01 | 902.8 | 1804.02 | 902.51 | 602.01 |
| SP227 | 1025 | Ac-LTF$r8EHWAQL$AALa-NH2 | iso2 | 1803.01 | 902.9 | 1804.02 | 902.51 | 602.01 |
| SP228 | 1026 | Ac-ETF$r8EHWVQL$AALa-NH2 | | 1847 | 924.82 | 1848.01 | 924.51 | 616.67 |
| SP229 | 1027 | Ac-LTF$r8AYWAQL$AAAa-NH2 | | 1728.96 | 865.89 | 1729.97 | 865.49 | 577.33 |
| SP230 | 1028 | Ac-LTF$r8AYWAQL$AAAa-NH2 | iso2 | 1728.96 | 865.89 | 1729.97 | 865.49 | 577.33 |
| SP231 | 1029 | Ac-LTF$r8AYWAQL$AAAibA-NH2 | | 1742.98 | 872.83 | 1743.99 | 872.5 | 582 |
| SP232 | 1030 | Ac-LTF$r8AYWAQL$AAAibA-NH2 | iso2 | 1742.98 | 872.92 | 1743.99 | 872.5 | 582 |
| SP233 | 1031 | Ac-LTF$r8AYWAQL$AAAAa-NH2 | | 1800 | 901.42 | 1801.01 | 901.01 | 601.01 |
| SP234 | 1032 | Ac-LTF$r5AYWAQL$s8AAIa-NH2 | | 1771.01 | 887.17 | 1772.02 | 886.51 | 591.34 |
| SP235 | 1033 | Ac-LTF$r5AYWAQL$s8SAA-NH2 | | 1673.92 | 838.33 | 1674.93 | 837.97 | 558.98 |
| SP236 | 1034 | Ac-LTF$r8AYWAQCba$AANleA-NH2 | | 1783.01 | 892.64 | 1784.02 | 892.51 | 595.34 |
| SP237 | 1035 | Ac-ETF$r8AYWAQCba$AANleA-NH2 | | 1798.97 | 900.59 | 1799.98 | 900.49 | 600.66 |
| SP238 | 1036 | Ac-LTF$r8EYWAQCba$AANleA-NH2 | | 1841.01 | 922.05 | 1842.02 | 921.51 | 614.68 |
| SP239 | 1037 | Ac-LTF$r8AYWAQCba$AWNleA-NH2 | | 1898.05 | 950.46 | 1899.06 | 950.03 | 633.69 |
| SP240 | 1038 | Ac-ETF$r8AYWAQCba$AWNleA-NH2 | | 1914.01 | 958.11 | 1915.02 | 958.01 | 639.01 |
| SP241 | 1039 | Ac-LTF$r8EYWAQCba$AWNleA-NH2 | | 1956.06 | 950.62 | 1957.07 | 979.04 | 653.03 |
| SP242 | 1040 | Ac-LTF$r8EYWAQCba$SAFA-NH2 | | 1890.99 | 946.55 | 1892 | 946.5 | 631.34 |
| SP243 | 1041 | Ac-LTF34F2$r8EYWAQCba$SANleA-NH2 | | 1892.99 | 947.57 | 1894 | 947.5 | 632 |
| SP244 | 1042 | Ac-LTF$r8EF4coohWAQCba$SANleA-NH2 | | 1885 | 943.59 | 1886.01 | 943.51 | 629.34 |
| SP245 | 1043 | Ac-LTF$r8EYWSQCba$SANleA-NH2 | | 1873 | 937.58 | 1874.01 | 937.51 | 625.34 |
| SP246 | 1044 | Ac-LTF$r8EYWWQCba$SANleA-NH2 | | 1972.05 | 987.61 | 1973.06 | 987.03 | 658.36 |
| SP247 | 1045 | Ac-LTF$r8EYWAQCba$AAIa-NH2 | | 1841.01 | 922.05 | 1842.02 | 921.51 | 614.68 |
| SP248 | 1046 | Ac-LTF34F2$r8EYWAQCba$AAIa-NH2 | | 1876.99 | 939.99 | 1878 | 939.5 | 626.67 |
| SP249 | 1047 | Ac-LTF$r8EF4coohWAQCba$AAIa-NH2 | | 1869.01 | 935.64 | 1870.02 | 935.51 | 624.01 |
| SP250 | 1048 | Pam-ETF$r8EYWAQCba$SAA-NH2 | | 1956.1 | 979.57 | 1957.11 | 979.06 | 653.04 |
| SP251 | 1049 | Ac-LThF$r8EFWAQCba$SAA-NH2 | | 1741.94 | 872.11 | 1742.95 | 871.98 | 581.65 |
| SP252 | 1050 | Ac-LTA$r8EYWAQCba$SAA-NH2 | | 1667.89 | 835.4 | 1668.9 | 834.95 | 556.97 |
| SP253 | 1051 | Ac-LTF$r8EYAAQCba$SAA-NH2 | | 1628.88 | 815.61 | 1629.89 | 815.45 | 543.97 |
| SP254 | 1052 | Ac-LTF$r8EY2NalAQCba$SAA-NH2 | | 1754.93 | 879.04 | 1755.94 | 878.47 | 585.98 |
| SP255 | 1053 | Ac-LTF$r8AYWAQCba$SAA-NH2 | | 1685.92 | 844.71 | 1686.93 | 843.97 | 562.98 |
| SP256 | 1054 | Ac-LTF$r8EYWAQCba$SAF-NH2 | | 1819.96 | 911.41 | 1820.97 | 910.99 | 607.66 |
| SP257 | 1055 | Ac-LTF$r8EYWAQCba$SAFa-NH2 | | 1890.99 | 947.41 | 1892 | 946.5 | 631.34 |
| SP258 | 1056 | Ac-LTF$r8AYWAQCba$SAF-NH2 | | 1761.95 | 882.73 | 1762.96 | 881.98 | 588.32 |
| SP259 | 1057 | Ac-LTF34F2$r8AYWAQCba$SAF-NH2 | | 1797.93 | 900.87 | 1798.94 | 899.97 | 600.32 |
| SP260 | 1058 | Ac-LTF$r8AF4coohWAQCba$SAF-NH2 | | 1789.94 | 896.43 | 1790.95 | 895.98 | 597.65 |
| SP261 | 1059 | Ac-LTF$r8EY6clWAQCba$SAF-NH2 | | 1853.92 | 929.27 | 1854.93 | 927.97 | 618.98 |
| SP262 | 1060 | Ac-LTF$r8AYWSQCba$SAF-NH2 | | 1777.94 | 890.87 | 1778.95 | 889.98 | 593.65 |
| SP263 | 1061 | Ac-LTF$r8AYWWQCba$SAF-NH2 | | 1876.99 | 939.91 | 1878 | 939.5 | 626.67 |
| SP264 | 1062 | Ac-LTF$r8AYWAQCba$AAIa-NH2 | | 1783.01 | 893.19 | 1784.02 | 892.51 | 595.34 |
| SP265 | 1063 | Ac-LTF34F2$r8AYWAQCba$AAIa-NH2 | | 1818.99 | 911.23 | 1820 | 910.5 | 607.34 |
| SP266 | 1064 | Ac-LTF$r8AY6clWAQCba$AAIa-NH2 | | 1816.97 | 909.84 | 1817.98 | 909.49 | 606.66 |
| SP267 | 1065 | Ac-LTF$r8AF4coohWAQCba$AAIa-NH2 | | 1811 | 906.88 | 1812.01 | 906.51 | 604.67 |
| SP268 | 1066 | Ac-LTF$r8EYWAQCba$AAFa-NH2 | | 1875 | 938.6 | 1876.01 | 938.51 | 626.01 |
| SP269 | 1067 | Ac-LTF$r8EYWAQCba$AAFa-NH2 | iso2 | 1875 | 938.6 | 1876.01 | 938.51 | 626.01 |
| SP270 | 1068 | Ac-ETF$r8AYWAQCba$AWNlea-NH2 | | 1914.01 | 958.42 | 1915.02 | 958.01 | 639.01 |
| SP271 | 1069 | Ac-LTF$r8EYWAQCba$AWNlea-NH2 | | 1956.06 | 979.42 | 1957.07 | 979.04 | 653.03 |
| SP272 | 1070 | Ac-ETF$r8EYWAQCba$AWNlea-NH2 | | 1972.01 | 987.06 | 1973.02 | 987.01 | 658.34 |
| SP273 | 1071 | Ac-ETF$r8EYWAQCba$AWNlea-NH2 | iso2 | 1972.01 | 987.06 | 1973.02 | 987.01 | 658.34 |
| SP274 | 1072 | Ac-LTF$r8AYWAQCba$SAFa-NH2 | | 1832.99 | 917.89 | 1834 | 917.5 | 612 |
| SP275 | 1073 | Ac-LTF$r8AYWAQCba$SAFa-NH2 | iso2 | 1832.99 | 918.07 | 1834 | 917.5 | 612 |
| SP276 | 1074 | Ac-ETF$r8AYWAQL$AWNlea-NH2 | | 1902.01 | 952.22 | 1903.02 | 952.01 | 635.01 |
| SP277 | 1075 | Ac-LTF$r8EYWAQL$AWNlea-NH2 | | 1944.06 | 973.5 | 1945.07 | 973.04 | 649.03 |
| SP278 | 1076 | Ac-ETF$r8EYWAQL$AWNlea-NH2 | | 1960.01 | 981.46 | 1961.02 | 981.01 | 654.34 |
| SP279 | 1077 | Dmaac-LTF$r8EYWAQhL$SAA-NH2 | | 1788.98 | 896.06 | 1789.99 | 895.5 | 597.33 |
| SP280 | 1078 | Hexac-LTF$r8EYWAQhL$SAA-NH2 | | 1802 | 902.9 | 1803.01 | 902.01 | 601.67 |
| SP281 | 1079 | Napac-LTF$r8EYWAQhL$SAA-NH2 | | 1871.99 | 937.58 | 1873 | 937 | 625 |
| SP282 | 1080 | Decac-LTF$r8EYWAQhL$SAA-NH2 | | 1858.06 | 930.55 | 1859.07 | 930.04 | 620.36 |
| SP283 | 1081 | Admac-LTF$r8EYWAQhL$SAA-NH2 | | 1866.03 | 934.07 | 1867.04 | 934.02 | 623.02 |
| SP284 | 1082 | Tmac-LTF$r8EYWAQhL$SAA-NH2 | | 1787.99 | 895.41 | 1789 | 895 | 597 |
| SP285 | 1083 | Pam-LTF$r8EYWAQhL$SAA-NH2 | | 1942.16 | 972.08 | 1943.17 | 972.09 | 648.39 |
| SP286 | 1084 | Ac-LTF$r8AYWAQCba$AANleA-NH2 | iso2 | 1783.01 | 892.64 | 1784.02 | 892.51 | 595.34 |
| SP287 | 1085 | Ac-LTF34F2$r8EYWAQCba$AAIa-NH2 | iso2 | 1876.99 | 939.62 | 1878 | 939.5 | 626.67 |
| SP288 | 1086 | Ac-LTF34F2$r8EYWAQCba$SAA-NH2 | | 1779.91 | 892.07 | 1780.92 | 890.96 | 594.31 |
| SP289 | 1087 | Ac-LTF34F2$r8EYWAQCba$SAA-NH2 | iso2 | 1779.91 | 891.61 | 1780.92 | 890.96 | 594.31 |
| SP290 | 1088 | Ac-LTF$r8EF4coohWAQCba$SAA-NH2 | | 1771.92 | 887.54 | 1772.93 | 886.97 | 591.65 |
| SP291 | 1089 | Ac-LTF$r8EF4coohWAQCba$SAA-NH2 | iso2 | 1771.92 | 887.63 | 1772.93 | 886.97 | 591.65 |
| SP292 | 1090 | Ac-LTF$r8EYWSQCba$SAA-NH2 | | 1759.92 | 881.9 | 1760.93 | 880.97 | 587.65 |
| SP293 | 1091 | Ac-LTF$r8EYWSQCba$SAA-NH2 | iso2 | 1759.92 | 881.9 | 1760.93 | 880.97 | 587.65 |
| SP294 | 1092 | Ac-LTF$r8EYWAQhL$SAA-NH2 | | 1745.94 | 875.05 | 1746.95 | 873.98 | 582.99 |
| SP295 | 1093 | Ac-LTF$r8AYWAQhL$SAF-NH2 | | 1763.97 | 884.02 | 1764.98 | 882.99 | 589 |
| SP296 | 1094 | Ac-LTF$r8AYWAQhL$SAF-NH2 | iso2 | 1763.97 | 883.56 | 1764.98 | 882.99 | 589 |
| SP297 | 1095 | Ac-LTF34F2$r8AYWAQhL$SAA-NH2 | | 1723.92 | 863.67 | 1724.93 | 862.97 | 575.65 |
| SP298 | 1096 | Ac-LTF34F2$r8AYWAQhL$SAA-NH2 | iso2 | 1723.92 | 864.04 | 1724.93 | 862.97 | 575.65 |
| SP299 | 1097 | Ac-LTF$r8AF4coohWAQhL$SAA-NH2 | | 1715.93 | 859.44 | 1716.94 | 858.97 | 572.98 |
| SP300 | 1098 | Ac-LTF$r8AF4coohWAQhL$SAA-NH2 | iso2 | 1715.93 | 859.6 | 1716.94 | 858.97 | 572.98 |
| SP301 | 1099 | Ac-LTF$r8AYWSQhL$SAA-NH2 | | 1703.93 | 853.96 | 1704.94 | 852.97 | 568.98 |
| SP302 | 1100 | Ac-LTF$r8AYWSQhL$SAA-NH2 | iso2 | 1703.93 | 853.59 | 1704.94 | 852.97 | 568.98 |
| SP303 | 1101 | Ac-LTF$r8EYWAQL$AANleA-NH2 | | 1829.01 | 915.45 | 1830.02 | 915.51 | 610.68 |
| SP304 | 1102 | Ac-LTF34F2$r8AYWAQL$AANleA-NH2 | | 1806.99 | 904.58 | 1808 | 904.5 | 603.34 |
| SP305 | 1103 | Ac-LTF$r8AF4coohWAQL$AANleA-NH2 | | 1799 | 901.6 | 1800.01 | 900.51 | 600.67 |
| SP306 | 1104 | Ac-LTF$r8AYWSQL$AANleA-NH2 | | 1787 | 894.75 | 1788.01 | 894.51 | 596.67 |
| SP307 | 1105 | Ac-LTF34F2$r8AYWAQhL$AANleA-NH2 | | 1821 | 911.79 | 1822.01 | 911.51 | 608.01 |
| SP308 | 1106 | Ac-LTF34F2$r8AYWAQhL$AANleA-NH2 | iso2 | 1821 | 912.61 | 1822.01 | 911.51 | 608.01 |
| SP309 | 1107 | Ac-LTF$r8AF4coohWAQhL$AANleA-NH2 | | 1813.02 | 907.95 | 1814.03 | 907.52 | 605.35 |
| SP310 | 1108 | Ac-LTF$r8AF4coohWAQhL$AANleA-NH2 | iso2 | 1813.02 | 908.54 | 1814.03 | 907.52 | 605.35 |
| SP311 | 1109 | Ac-LTF$r8AYWSQhL$AANleA-NH2 | | 1801.02 | 901.84 | 1802.03 | 901.52 | 601.35 |
| SP312 | 1110 | Ac-LTF$r8AYWSQhL$AANleA-NH2 | iso2 | 1801.02 | 902.62 | 1802.03 | 901.52 | 601.35 |
| SP313 | 1111 | Ac-LTF$r8AYWAQhL$AAAAa-NH2 | | 1814.01 | 908.63 | 1815.02 | 908.01 | 605.68 |
| SP314 | 1112 | Ac-LTF$r8AYWAQhL$AAAAa-NH2 | iso2 | 1814.01 | 908.34 | 1815.02 | 908.01 | 605.68 |
| SP315 | 1113 | Ac-LTF$r8AYWAQL$AAAAAa-NH2 | | 1871.04 | 936.94 | 1872.05 | 936.53 | 624.69 |
| SP316 | 1114 | Ac-LTF$r8AYWAQL$AAAAAAa-NH2 | iso2 | 1942.07 | 972.5 | 1943.08 | 972.04 | 648.37 |
| SP317 | 1115 | Ac-LTF$r8AYWAQL$AAAAAAa-NH2 | iso1 | 1942.07 | 972.5 | 1943.08 | 972.04 | 648.37 |
| SP318 | 1116 | Ac-LTF$r8EYWAQhL$AANleA-NH2 | | 1843.03 | 922.54 | 1844.04 | 922.52 | 615.35 |
| SP319 | 1117 | Ac-AATF$r8AYWAQL$AANleA-NH2 | | 1800 | 901.39 | 1801.01 | 901.01 | 601.01 |
| SP320 | 1118 | Ac-LTF$r8AYWAQL$AANleAA-NH2 | | 1842.04 | 922.45 | 1843.05 | 922.03 | 615.02 |
| SP321 | 1119 | Ac-ALTF$r8AYWAQL$AANleAA-NH2 | | 1913.08 | 957.94 | 1914.09 | 957.55 | 638.7 |
| SP322 | 1120 | Ac-LTF$r8AYWAQCba$AANleAA-NH2 | | 1854.04 | 928.43 | 1855.05 | 928.03 | 619.02 |
| SP323 | 1121 | Ac-LTF$r8AYWAQhL$AANleAA-NH2 | | 1856.06 | 929.4 | 1857.07 | 929.04 | 619.69 |
| SP324 | 1122 | Ac-LTF$r8EYWAQCba$SAAA-NH2 | | 1814.96 | 909.37 | 1815.97 | 908.49 | 605.99 |
| SP325 | 1123 | Ac-LTF$r8EYWAQCba$SAAA-NH2 | iso2 | 1814.96 | 909.37 | 1815.97 | 908.49 | 605.99 |
| SP326 | 1124 | Ac-LTF$r8EYWAQCba$SAAAA-NH2 | | 1886 | 944.61 | 1887.01 | 944.01 | 629.67 |
| SP327 | 1125 | Ac-LTF$r8EYWAQCba$SAAAA-NH2 | iso2 | 1886 | 944.61 | 1887.01 | 944.01 | 629.67 |
| SP328 | 1126 | Ac-ALTF$r8EYWAQCba$SAA-NH2 | | 1814.96 | 909.09 | 1815.97 | 908.49 | 605.99 |
| SP329 | 1127 | Ac-ALTF$r8EYWAQCba$SAAA-NH2 | | 1886 | 944.61 | 1887.01 | 944.01 | 629.67 |
| SP330 | 1128 | Ac-ALTF$r8EYWAQCba$SAA-NH2 | iso2 | 1814.96 | 909.09 | 1815.97 | 908.49 | 605.99 |
| SP331 | 1129 | Ac-LTF$r8EYWAQL$AAAAAa-NH2 | iso2 | 1929.04 | 966.08 | 1930.05 | 965.53 | 644.02 |
| SP332 | 1130 | Ac-LTF$r8EY6clWAQCba$SAA-NH2 | | 1777.89 | 890.78 | 1778.9 | 889.95 | 593.64 |
| SP333 | 1131 | Ac-LTF$r8EF4cooh6clWAQCba$SANleA-NH2 | | 1918.96 | 961.27 | 1919.97 | 960.49 | 640.66 |
| SP334 | 1132 | Ac-LTF$r8EF4cooh6clWAQCba$SANleA-NH2 | iso2 | 1918.96 | 961.27 | 1919.97 | 960.49 | 640.66 |
| SP335 | 1133 | Ac-LTF$r8EF4cooh6clWAQCba$AAIa-NH2 | | 1902.97 | 953.03 | 1903.98 | 952.49 | 635.33 |
| SP336 | 1134 | Ac-LTF$r8EF4cooh6clWAQCba$AAIa-NH2 | iso2 | 1902.97 | 953.13 | 1903.98 | 952.49 | 635.33 |
| SP337 | 1135 | Ac-LTF$r8AY6clWAQL$AAAAAa-NH2 | | 1905 | 954.61 | 1906.01 | 953.51 | 636.01 |
| SP338 | 1136 | Ac-LTF$r8AY6clWAQL$AAAAAa-NH2 | iso2 | 1905 | 954.9 | 1906.01 | 953.51 | 636.01 |
| SP339 | 1137 | Ac-F$r8AY6clWEAL$AAAAAAa-NH2 | | 1762.89 | 883.01 | 1763.9 | 882.45 | 588.64 |
| SP340 | 1138 | Ac-ETF$r8EYWAQL$AAAAAa-NH2 | | 1945 | 974.31 | 1946.01 | 973.51 | 649.34 |
| SP341 | 1139 | Ac-ETF$r8EYWAQL$AAAAAa-NH2 | iso2 | 1945 | 974.49 | 1946.01 | 973.51 | 649.34 |
| SP342 | 1140 | Ac-LTF$r8EYWAQL$AAAAAAa-NH2 | | 2000.08 | 1001.6 | 2001.09 | 1001.05 | 667.7 |
| SP343 | 1141 | Ac-LTF$r8EYWAQL$AAAAAAa-NH2 | iso2 | 2000.08 | 1001.6 | 2001.09 | 1001.05 | 667.7 |
| SP344 | 1142 | Ac-LTF$r8AYWAQL$AANleAAa-NH2 | | 1913.08 | 958.58 | 1914.09 | 957.55 | 638.7 |
| SP345 | 1143 | Ac-LTF$r8AYWAQL$AANleAAa-NH2 | iso2 | 1913.08 | 958.58 | 1914.09 | 957.55 | 638.7 |
| SP346 | 1144 | Ac-LTF$r8EYWAQCba$AAAAAa-NH2 | | 1941.04 | 972.55 | 1942.05 | 971.53 | 648.02 |
| SP347 | 1145 | Ac-LTF$r8EYWAQCba$AAAAAa-NH2 | iso2 | 1941.04 | 972.55 | 1942.05 | 971.53 | 648.02 |
| SP348 | 1146 | Ac-LTF$r8EF4coohWAQCba$AAAAAa-NH2 | | 1969.04 | 986.33 | 1970.05 | 985.53 | 657.35 |
| SP349 | 1147 | Ac-LTF$r8EF4coohWAQCba$AAAAAa-NH2 | iso2 | 1969.04 | 986.06 | 1970.05 | 985.53 | 657.35 |
| SP350 | 1148 | Ac-LTF$r8EYWSQCba$AAAAAa-NH2 | | 1957.04 | 980.04 | 1958.05 | 979.53 | 653.35 |
| SP351 | 1149 | Ac-LTF$r8EYWSQCba$AAAAAa-NH2 | iso2 | 1957.04 | 980.04 | 1958.05 | 979.53 | 653.35 |
| SP352 | 1150 | Ac-LTF$r8EYWAQCba$SAAa-NH2 | | 1814.96 | 909 | 1815.97 | 908.49 | 605.99 |
| SP353 | 1151 | Ac-LTF$r8EYWAQCba$SAAa-NH2 | iso2 | 1814.96 | 909 | 1815.97 | 908.49 | 605.99 |
| SP354 | 1152 | Ac-ALTF$r8EYWAQCba$SAAa-NH2 | | 1886 | 944.52 | 1887.01 | 944.01 | 629.67 |
| SP355 | 1153 | Ac-ALTF$r8EYWAQCba$SAAa-NH2 | iso2 | 1886 | 944.98 | 1887.01 | 944.01 | 629.67 |
| SP356 | 1154 | Ac-ALTF$r8EYWAQCba$SAAAa-NH2 | | 1957.04 | 980.04 | 1958.05 | 979.53 | 653.35 |
| SP357 | 1155 | Ac-ALTF$r8EYWAQCba$SAAAa-NH2 | iso2 | 1957.04 | 980.04 | 1958.05 | 979.53 | 653.35 |
| SP358 | 1156 | Ac-AALTF$r8EYWAQCba$SAAAa-NH2 | | 2028.07 | 1016.1 | 2029.08 | 1015.04 | 677.03 |
| SP359 | 1157 | Ac-AALTF$r8EYWAQCba$SAAAa-NH2 | iso2 | 2028.07 | 1015.57 | 2029.08 | 1015.04 | 677.03 |
| SP360 | 1158 | Ac-RTF$r8EYWAQCba$SAA-NH2 | | 1786.94 | 895.03 | 1787.95 | 894.48 | 596.65 |
| SP361 | 1159 | Ac-LRF$r8EYWAQCba$SAA-NH2 | | 1798.98 | 901.51 | 1799.99 | 900.5 | 600.67 |
| SP362 | 1160 | Ac-LTF$r8EYWRQCba$SAA-NH2 | | 1828.99 | 916.4 | 1830 | 915.5 | 610.67 |
| SP363 | 1161 | Ac-LTF$r8EYWARCba$SAA-NH2 | | 1771.97 | 887.63 | 1772.98 | 886.99 | 591.66 |
| SP364 | 1162 | Ac-LTF$r8EYWAQCba$RAA-NH2 | | 1812.99 | 908.08 | 1814 | 907.5 | 605.34 |
| SP365 | 1163 | Ac-LTF$r8EYWAQCba$SRA-NH2 | | 1828.99 | 916.12 | 1830 | 915.5 | 610.67 |
| SP366 | 1164 | Ac-LTF$r8EYWAQCba$SAR-NH2 | | 1828.99 | 916.12 | 1830 | 915.5 | 610.67 |
| SP367 | 1165 | 5-FAM-BaLTF$r8EYWAQCba$SAA-NH2 | | 2131 | 1067.09 | 2132.01 | 1066.51 | 711.34 |
| SP368 | 1166 | 5-FAM-BaLTF$r8AYWAQL$AANleA-NH2 | | 2158.08 | 1080.6 | 2159.09 | 1080.05 | 720.37 |
| SP369 | 1167 | Ac-LAF$r8EYWAQL$AANleA-NH2 | | 1799 | 901.05 | 1800.01 | 900.51 | 600.67 |
| SP370 | 1168 | Ac-ATF$r8EYWAQL$AANleA-NH2 | | 1786.97 | 895.03 | 1787.98 | 894.49 | 596.66 |
| SP371 | 1169 | Ac-AAF$r8EYWAQL$AANleA-NH2 | | 1756.96 | 880.05 | 1757.97 | 879.49 | 586.66 |
| SP372 | 1170 | Ac-AAAF$r8EYWAQL$AANleA-NH2 | | 1827.99 | 915.57 | 1829 | 915 | 610.34 |
| SP373 | 1171 | Ac-AAAAF$r8EYWAQL$AANleA-NH2 | | 1899.03 | 951.09 | 1900.04 | 950.52 | 634.02 |
| SP374 | 1172 | Ac-AATF$r8EYWAQL$AANleA-NH2 | | 1858 | 930.92 | 1859.01 | 930.01 | 620.34 |
| SP375 | 1173 | Ac-AALTF$r8EYWAQL$AANleA-NH2 | | 1971.09 | 987.17 | 1972.1 | 986.55 | 658.04 |
| SP376 | 1174 | Ac-AAALTF$r8EYWAQL$AANleA-NH2 | | 2042.12 | 1023.15 | 2043.13 | 1022.07 | 681.71 |
| SP377 | 1175 | Ac-LTF$r8EYWAQL$AANleAA-NH2 | | 1900.05 | 952.02 | 1901.06 | 951.03 | 634.36 |
| SP378 | 1176 | Ac-ALTF$r8EYWAQL$AANleAA-NH2 | | 1971.09 | 987.63 | 1972.1 | 986.55 | 658.04 |
| SP379 | 1177 | Ac-AALTF$r8EYWAQL$AANleAA-NH2 | | 2042.12 | 1022.69 | 2043.13 | 1022.07 | 681.71 |
| SP380 | 1178 | Ac-LTF$r8EYWAQCba$AANleAA-NH2 | | 1912.05 | 958.03 | 1913.06 | 957.03 | 638.36 |
| SP381 | 1179 | Ac-LTF$r8EYWAQhL$AANleAA-NH2 | | 1914.07 | 958.68 | 1915.08 | 958.04 | 639.03 |
| SP382 | 1180 | Ac-ALTF$r8EYWAQhL$AANleAA-NH2 | | 1985.1 | 994.1 | 1986.11 | 993.56 | 662.71 |
| SP383 | 1181 | Ac-LTF$r8ANmYWAQL$AANleA-NH2 | | 1785.02 | 894.11 | 1786.03 | 893.52 | 596.01 |
| SP384 | 1182 | Ac-LTF$r8ANmYWAQL$AANleA-NH2 | iso2 | 1785.02 | 894.11 | 1786.03 | 893.52 | 596.01 |
| SP385 | 1183 | Ac-LTF$r8AYNmWAQL$AANleA-NH2 | | 1785.02 | 894.11 | 1786.03 | 893.52 | 596.01 |
| SP386 | 1184 | Ac-LTF$r8AYNmWAQL$AANleA-NH2 | iso2 | 1785.02 | 894.11 | 1786.03 | 893.52 | 596.01 |
| SP387 | 1185 | Ac-LTF$r8AYAmwAQL$AANleA-NH2 | | 1785.02 | 894.01 | 1786.03 | 893.52 | 596.01 |
| SP388 | 1186 | Ac-LTF$r8AYAmwAQL$AANleA-NH2 | iso2 | 1785.02 | 894.01 | 1786.03 | 893.52 | 596.01 |
| SP389 | 1187 | Ac-LTF$r8AYWAibQL$AANleA-NH2 | | 1785.02 | 894.01 | 1786.03 | 893.52 | 596.01 |
| SP390 | 1188 | Ac-LTF$r8AYWAibQL$AANleA-NH2 | iso2 | 1785.02 | 894.01 | 1786.03 | 893.52 | 596.01 |
| SP391 | 1189 | Ac-LTF$r8AYWAQL$AAibNleA-NH2 | | 1785.02 | 894.38 | 1786.03 | 893.52 | 596.01 |
| SP392 | 1190 | Ac-LTF$r8AYWAQL$AAibNleA-NH2 | iso2 | 1785.02 | 894.38 | 1786.03 | 893.52 | 596.01 |
| SP393 | 1191 | Ac-LTF$r8AYWAQL$AaNleA-NH2 | | 1771.01 | 887.54 | 1772.02 | 886.51 | 591.34 |
| SP394 | 1192 | Ac-LTF$r8AYWAQL$AaNleA-NH2 | iso2 | 1771.01 | 887.54 | 1772.02 | 886.51 | 591.34 |
| SP395 | 1193 | Ac-LTF$r8AYWAQL$ASarNleA-NH2 | | 1771.01 | 887.35 | 1772.02 | 886.51 | 591.34 |
| SP396 | 1194 | Ac-LTF$r8AYWAQL$ASarNleA-NH2 | iso2 | 1771.01 | 887.35 | 1772.02 | 886.51 | 591.34 |
| SP397 | 1195 | Ac-LTF$r8AYWAQL$AANleAib-NH2 | | 1785.02 | 894.75 | 1786.03 | 893.52 | 596.01 |
| SP398 | 1196 | Ac-LTF$r8AYWAQL$AANleAib-NH2 | iso2 | 1785.02 | 894.75 | 1786.03 | 893.52 | 596.01 |
| SP399 | 1197 | Ac-LTF$r8AYWAQL$AANleNmA-NH2 | | 1785.02 | 894.6 | 1786.03 | 893.52 | 596.01 |
| SP400 | 1198 | Ac-LTF$r8AYWAQL$AANleNmA-NH2 | iso2 | 1785.02 | 894.6 | 1786.03 | 893.52 | 596.01 |
| SP401 | 1199 | Ac-LTF$r8AYWAQL$AANleSar-NH2 | | 1771.01 | 886.98 | 1772.02 | 886.51 | 591.34 |
| SP402 | 1200 | Ac-LTF$r8AYWAQL$AANleSar-NH2 | iso2 | 1771.01 | 886.98 | 1772.02 | 886.51 | 591.34 |
| SP403 | 1201 | Ac-LTF$r8AYWAQL$AANleAAib-NH2 | | 1856.06 | | 1857.07 | 929.04 | 619.69 |
| SP404 | 1202 | Ac-LTF$r8AYWAQL$AANleAAib-NH2 | iso2 | 1856.06 | | 1857.07 | 929.04 | 619.69 |
| SP405 | 1203 | Ac-LTF$r8AYWAQL$AANleANmA-NH2 | | 1856.06 | 930.37 | 1857.07 | 929.04 | 619.69 |
| SP406 | 1204 | Ac-LTF$r8AYWAQL$AANleANmA-NH2 | iso2 | 1856.06 | 930.37 | 1857.07 | 929.04 | 619.69 |
| SP407 | 1205 | Ac-LTF$r8AYWAQL$AANleAa-NH2 | | 1842.04 | 922.69 | 1843.05 | 922.03 | 615.02 |
| SP408 | 1206 | Ac-LTF$r8AYWAQL$AANleAa-NH2 | iso2 | 1842.04 | 922.69 | 1843.05 | 922.03 | 615.02 |
| SP409 | 1207 | Ac-LTF$r8AYWAQL$AANleASar-NH2 | | 1842.04 | 922.6 | 1843.05 | 922.03 | 615.02 |
| SP410 | 1208 | Ac-LTF$r8AYWAQL$AANleASar-NH2 | iso2 | 1842.04 | 922.6 | 1843.05 | 922.03 | 615.02 |
| SP411 | 1209 | Ac-LTF$/r8AYWAQL$/AANleA-NH2 | | 1799.04 | 901.14 | 1800.05 | 900.53 | 600.69 |
| SP412 | 1210 | Ac-LTFAibAYWAQLAibAANleA-NH2 | | 1648.9 | 826.02 | 1649.91 | 825.46 | 550.64 |
| SP413 | 1211 | Ac-LTF$r8Cou4YWAQL$AANleA-NH2 | | 1975.05 | 989.11 | 1976.06 | 988.53 | 659.36 |
| SP414 | 1212 | Ac-LTF$r8Cou4YWAQL$AANleA-NH2 | iso2 | 1975.05 | 989.11 | 1976.06 | 988.53 | 659.36 |
| SP415 | 1213 | Ac-LTF$r8AYWCou4QL$AANleA-NH2 | | 1975.05 | 989.11 | 1976.06 | 988.53 | 659.36 |
| SP416 | 1214 | Ac-LTF$r8AYWAQL$Cou4ANleA-NH2 | | 1975.05 | 989.57 | 1976.06 | 988.53 | 659.36 |
| SP417 | 1215 | Ac-LTF$r8AYWAQL$Cou4ANleA-NH2 | iso2 | 1975.05 | 989.57 | 1976.06 | 988.53 | 659.36 |
| SP418 | 1216 | Ac-LTF$r8AYWAQL$ACou4NleA-NH2 | | 1975.05 | 989.57 | 1976.06 | 988.53 | 659.36 |
| SP419 | 1217 | Ac-LTF$r8AYWAQL$ACou4NleA-NH2 | iso2 | 1975.05 | 989.57 | 1976.06 | 988.53 | 659.36 |
| SP420 | 1218 | Ac-LTF$r8AYWAQL$AANleA-OH | | 1771.99 | 887.63 | 1773 | 887 | 591.67 |
| SP421 | 1219 | Ac-LTF$r8AYWAQL$AANleA-OH | iso2 | 1771.99 | 887.63 | 1773 | 887 | 591.67 |
| SP422 | 1220 | Ac-LTF$r8AYWAQL$AANleA-NHnPr | | 1813.05 | 908.08 | 1814.06 | 907.53 | 605.36 |
| SP423 | 1221 | Ac-LTF$r8AYWAQL$AANleA-NHnPr | iso2 | 1813.05 | 908.08 | 1814.06 | 907.53 | 605.36 |
| SP424 | 1222 | Ac-LTF$r8AYWAQL$AANleA-NHnBu33Me | | 1855.1 | 929.17 | 1856.11 | 928.56 | 619.37 |
| SP425 | 1223 | Ac-LTF$r8AYWAQL$AANleA-NHnBu33Me | iso2 | 1855.1 | 929.17 | 1856.11 | 928.56 | 619.37 |
| SP426 | 1224 | Ac-LTF$r8AYWAQL$AANleA-NHHex | | 1855.1 | 929.17 | 1856.11 | 928.56 | 619.37 |
| SP427 | 1225 | Ac-LTF$r8AYWAQL$AANleA-NHHex | iso2 | 1855.1 | 929.17 | 1856.11 | 928.56 | 619.37 |
| SP428 | 1226 | Ac-LTA$r8AYWAQL$AANleA-NH2 | | 1694.98 | 849.33 | 1695.99 | 848.5 | 566 |
| SP429 | 1227 | Ac-LThL$r8AYWAQL$AANleA-NH2 | | 1751.04 | 877.09 | 1752.05 | 876.53 | 584.69 |
| SP430 | 1228 | Ac-LTF$r8AYAAQL$AANleA-NH2 | | 1655.97 | 829.54 | 1656.98 | 828.99 | 553 |
| SP431 | 1229 | Ac-LTF$r8AY2NalAQL$AANleA-NH2 | | 1782.01 | 892.63 | 1783.02 | 892.01 | 595.01 |
| SP432 | 1230 | Ac-LTF$r8EYWCou4QCba$SAA-NH2 | | 1947.97 | 975.8 | 1948.98 | 974.99 | 650.33 |
| SP433 | 1231 | Ac-LTF$r8EYWCou7QCba$SAA-NH2 | | 16.03 | 974.9 | 17.04 | 9.02 | 6.35 |
| SP434 | 1232 | Ac-LTF%r8EYWAQCba%SAA-NH2 | | 1745.94 | 874.8 | 1746.95 | 873.98 | 582.99 |
| SP435 | 1233 | Dmaac-LTF$r8EYWAQCba$SAA-NH2 | | 1786.97 | 894.8 | 1787.98 | 894.49 | 596.66 |
| SP436 | 1234 | Dmaac-LTF$r8AYWAQL$AAAAAa-NH2 | | 1914.08 | 958.2 | 1915.09 | 958.05 | 639.03 |
| SP437 | 1235 | Dmaac-LTF$r8AYWAQL$AAAAAa-NH2 | iso2 | 1914.08 | 958.2 | 1915.09 | 958.05 | 639.03 |
| SP438 | 1236 | Dmaac-LTF$r8EYWAQL$AAAAAa-NH2 | | 1972.08 | 987.3 | 1973.09 | 987.05 | 658.37 |
| SP439 | 1237 | Dmaac-LTF$r8EYWAQL$AAAAAa-NH2 | iso2 | 1972.08 | 987.3 | 1973.09 | 987.05 | 658.37 |
| SP440 | 1238 | Dmaac-LTF$r8EF4coohWAQCba$AAIa-NH2 | | 1912.05 | 957.4 | 1913.06 | 957.03 | 638.36 |
| SP441 | 1239 | Dmaac-LTF$r8EF4coohWAQCba$AAIa-NH2 | iso2 | 1912.05 | 957.4 | 1913.06 | 957.03 | 638.36 |
| SP442 | 1240 | Dmaac-LTF$r8AYWAQL$AANleA-NH2 | | 1814.05 | 908.3 | 1815.06 | 908.03 | 605.69 |
| SP443 | 1241 | Dmaac-LTF$r8AYWAQL$AANleA-NH2 | iso2 | 1814.05 | 908.3 | 1815.06 | 908.03 | 605.69 |
| SP444 | 1242 | Ac-LTF%r8AYWAQL%AANleA-NH2 | | 1773.02 | 888.37 | 1774.03 | 887.52 | 592.01 |
| SP445 | 1243 | Ac-LTF%r8EYWAQL%AAAAAa-NH2 | | 1931.06 | 966.4 | 1932.07 | 966.54 | 644.69 |
| SP446 | 1244 | Cou6BaLTF$r8EYWAQhL$SAA-NH2 | | 2018.05 | 1009.9 | 2019.06 | 1010.03 | 673.69 |
| SP447 | 1245 | Cou8BaLTF$r8EYWAQhL$SAA-NH2 | | 1962.96 | 982.34 | 1963.97 | 982.49 | 655.32 |
| SP448 | 1246 | Ac-LTF4I$r8EYWAQL$AAAAAa-NH2 | | 2054.93 | 1028.68 | 2055.94 | 1028.47 | 685.98 |
| SP449 | 1247 | Ac-LTF$r8EYWAQL$AAAAAa-NH2 | | 1929.04 | 966.17 | 1930.05 | 965.53 | 644.02 |
| SP550 | 1248 | Ac-LTF$r8EYWAQL$AAAAAa-OH | | 1930.02 | 966.54 | 1931.03 | 966.02 | 644.35 |
| SP551 | 1249 | Ac-LTF$r8EYWAQL$AAAAAa-OH | iso2 | 1930.02 | 965.89 | 1931.03 | 966.02 | 644.35 |
| SP552 | 1250 | Ac-LTF$r8EYWAEL$AAAAAa-NH2 | | 1930.02 | 966.82 | 1931.03 | 966.02 | 644.35 |
| SP553 | 1251 | Ac-LTF$r8EYWAEL$AAAAAa-NH2 | iso2 | 1930.02 | 966.91 | 1931.03 | 966.02 | 644.35 |
| SP554 | 1252 | Ac-LTF$r8EYWAEL$AAAAAa-OH | | 1931.01 | 967.28 | 1932.02 | 966.51 | 644.68 |
| SP555 | 1253 | Ac-LTF$r8EY6clWAQL$AAAAAa-NH2 | | 1963 | 983.28 | 1964.01 | 982.51 | 655.34 |
| SP556 | 1254 | Ac-LTF$r8EF4bOH2WAQL$AAAAAa-NH2 | | 1957.05 | 980.04 | 1958.06 | 979.53 | 653.36 |
| SP557 | 1255 | Ac-AAALTF$r8EYWAQL$AAAAAa-NH2 | | 2142.15 | 1072.83 | 2143.16 | 1072.08 | 715.06 |
| SP558 | 1256 | Ac-LTF34F2$r8EYWAQL$AAAAAa-NH2 | | 1965.02 | 984.3 | 1966.03 | 983.52 | 656.01 |
| SP559 | 1257 | Ac-RTF$r8EYWAQL$AAAAAa-NH2 | | 1972.06 | 987.81 | 1973.07 | 987.04 | 658.36 |
| SP560 | 1258 | Ac-LTA$r8EYWAQL$AAAAAa-NH2 | | 1853.01 | 928.33 | 1854.02 | 927.51 | 618.68 |
| SP561 | 1259 | Ac-LTF$r8EYWAibQL$AAAAAa-NH2 | | 1943.06 | 973.48 | 1944.07 | 972.54 | 648.69 |
| SP562 | 1260 | Ac-LTF$r8EYWAQL$AAibAAAa-NH2 | | 1943.06 | 973.11 | 1944.07 | 972.54 | 648.69 |
| SP563 | 1261 | Ac-LTF$r8EYWAQL$AAAibAAa-NH2 | | 1943.06 | 973.48 | 1944.07 | 972.54 | 648.69 |
| SP564 | 1262 | Ac-LTF$r8EYWAQL$AAAAibAa-NH2 | | 1943.06 | 973.48 | 1944.07 | 972.54 | 648.69 |
| SP565 | 1263 | Ac-LTF$r8EYWAQL$AAAAAiba-NH2 | | 1943.06 | 973.38 | 1944.07 | 972.54 | 648.69 |
| SP566 | 1264 | Ac-LTF$r8EYWAQL$AAAAAiba-NH2 | iso2 | 1943.06 | 973.38 | 1944.07 | 972.54 | 648.69 |
| SP567 | 1265 | Ac-LTF$r8EYWAQL$AAAAAAib-NH2 | | 1943.06 | 973.01 | 1944.07 | 972.54 | 648.69 |
| SP568 | 1266 | Ac-LTF$r8EYWAQL$AaAAAa-NH2 | | 1929.04 | 966.54 | 1930.05 | 965.53 | 644.02 |
| SP569 | 1267 | Ac-LTF$r8EYWAQL$AAaAAa-NH2 | | 1929.04 | 966.35 | 1930.05 | 965.53 | 644.02 |
| SP570 | 1268 | Ac-LTF$r8EYWAQL$AAAaAa-NH2 | | 1929.04 | 966.54 | 1930.05 | 965.53 | 644.02 |
| SP571 | 1269 | Ac-LTF$r8EYWAQL$AAAaAa-NH2 | iso2 | 1929.04 | 966.35 | 1930.05 | 965.53 | 644.02 |
| SP572 | 1270 | Ac-LTF$r8EYWAQL$AAAAaa-NH2 | | 1929.04 | 966.35 | 1930.05 | 965.53 | 644.02 |
| SP573 | 1271 | Ac-LTF$r8EYWAQL$AAAAAA-NH2 | | 1929.04 | 966.35 | 1930.05 | 965.53 | 644.02 |
| SP574 | 1272 | Ac-LTF$r8EYWAQL$ASarAAAa-NH2 | | 1929.04 | 966.54 | 1930.05 | 965.53 | 644.02 |
| SP575 | 1273 | Ac-LTF$r8EYWAQL$AASarAAa-NH2 | | 1929.04 | 966.35 | 1930.05 | 965.53 | 644.02 |
| SP576 | 1274 | Ac-LTF$r8EYWAQL$AAASarAa-NH2 | | 1929.04 | 966.35 | 1930.05 | 965.53 | 644.02 |
| SP577 | 1275 | Ac-LTF$r8EYWAQL$AAAASara-NH2 | | 1929.04 | 966.35 | 1930.05 | 965.53 | 644.02 |
| SP578 | 1276 | Ac-LTF$r8EYWAQL$AAAAASar-NH2 | | 1929.04 | 966.08 | 1930.05 | 965.53 | 644.02 |
| SP579 | 1277 | Ac-7LTF$r8EYWAQL$AAAAAa-NH2 | | 1918.07 | 951.99 | 1919.08 | 960.04 | 640.37 |
| SP581 | 1278 | Ac-TF$r8EYWAQL$AAAAAa-NH2 | | 1815.96 | 929.85 | 1816.97 | 908.99 | 606.33 |
| SP582 | 1279 | Ac-F$r8EYWAQL$AAAAAa-NH2 | | 1714.91 | 930.92 | 1715.92 | 858.46 | 572.64 |
| SP583 | 1280 | Ac-LVF$r8EYWAQL$AAAAAa-NH2 | | 1927.06 | 895.12 | 1928.07 | 964.54 | 643.36 |
| SP584 | 1281 | Ac-AAF$r8EYWAQL$AAAAAa-NH2 | | 1856.98 | 859.51 | 1857.99 | 929.5 | 620 |
| SP585 | 1282 | Ac-LTF$r8EYWAQL$AAAAa-NH2 | | 1858 | 824.08 | 1859.01 | 930.01 | 620.34 |
| SP586 | 1283 | Ac-LTF$r8EYWAQL$AAAa-NH2 | | 1786.97 | 788.56 | 1787.98 | 894.49 | 596.66 |
| SP587 | 1284 | Ac-LTF$r8EYWAQL$AAa-NH2 | | 1715.93 | 1138.57 | 1716.94 | 858.97 | 572.98 |
| SP588 | 1285 | Ac-LTF$r8EYWAQL$Aa-NH2 | | 1644.89 | 1144.98 | 1645.9 | 823.45 | 549.3 |
| SP589 | 1286 | Ac-LTF$r8EYWAQL$a-NH2 | | 1573.85 | 1113.71 | 1574.86 | 787.93 | 525.62 |
| SP590 | 1287 | Ac-LTF$r8EYWAQL$AAA-OH | | 1716.91 | 859.55 | 1717.92 | 859.46 | 573.31 |
| SP591 | 1288 | Ac-LTF$r8EYWAQL$A-OH | | 1574.84 | 975.14 | 1575.85 | 788.43 | 525.95 |
| SP592 | 1289 | Ac-LTF$r8EYWAQL$AAA-NH2 | | 1715.93 | 904.75 | 1716.94 | 858.97 | 572.98 |
| SP593 | 1290 | Ac-LTF$r8EYWAQCba$SAA-OH | | 1744.91 | 802.49 | 1745.92 | 873.46 | 582.64 |
| SP594 | 1291 | Ac-LTF$r8EYWAQCba$S-OH | | 1602.83 | 913.53 | 1603.84 | 802.42 | 535.28 |
| SP595 | 1292 | Ac-LTF$r8EYWAQCba$S-NH2 | | 1601.85 | 979.58 | 1602.86 | 801.93 | 534.96 |
| SP596 | 1293 | 4-FBzl-LTF$r8EYWAQL$AAAAAa-NH2 | | 2009.05 | 970.52 | 2010.06 | 1005.53 | 670.69 |
| SP597 | 1294 | 4-FBzl-LTF$r8EYWAQCba$SAA-NH2 | | 1823.93 | 965.8 | 1824.94 | 912.97 | 608.98 |
| SP598 | 1295 | Ac-LTF$r8RYWAQL$AAAAAa-NH2 | | 1956.1 | 988.28 | 1957.11 | 979.06 | 653.04 |
| SP599 | 1296 | Ac-LTF$r8HYWAQL$AAAAAa-NH2 | | 1937.06 | 1003.54 | 1938.07 | 969.54 | 646.69 |
| SP600 | 1297 | Ac-LTF$r8QYWAQL$AAAAAa-NH2 | | 1928.06 | 993.92 | 1929.07 | 965.04 | 643.69 |
| SP601 | 1298 | Ac-LTF$r8CitYWAQL$AAAAAa-NH2 | | 1957.08 | 987 | 1958.09 | 979.55 | 653.37 |
| SP602 | 1299 | Ac-LTF$r8GlaYWAQL$AAAAAa-NH2 | | 1973.03 | 983 | 1974.04 | 987.52 | 658.68 |
| SP603 | 1300 | Ac-LTF$r8F4gYWAQL$AAAAAa-NH2 | | 2004.1 | 937.86 | 2005.11 | 1003.06 | 669.04 |
| SP604 | 1301 | Ac-LTF$r82mRYWAQL$AAAAAa-NH2 | | 1984.13 | 958.58 | 1985.14 | 993.07 | 662.38 |
| SP605 | 1302 | Ac-LTF$r8ipKYWAQL$AAAAAa-NH2 | | 1970.14 | 944.52 | 1971.15 | 986.08 | 657.72 |
| SP606 | 1303 | Ac-LTF$r8F4NH2YWAQL$AAAAAa-NH2 | | 1962.08 | 946 | 1963.09 | 982.05 | 655.03 |
| SP607 | 1304 | Ac-LTF$r8EYWAAL$AAAAAa-NH2 | | 1872.02 | 959.32 | 1873.03 | 937.02 | 625.01 |
| SP608 | 1305 | Ac-LTF$r8EYWALL$AAAAAa-NH2 | | 1914.07 | 980.88 | 1915.08 | 958.04 | 639.03 |
| SP609 | 1306 | Ac-LTF$r8EYWAAibL$AAAAAa-NH2 | | 1886.03 | 970.61 | 1887.04 | 944.02 | 629.68 |
| SP610 | 1307 | Ac-LTF$r8EYWASL$AAAAAa-NH2 | | 1888.01 | 980.51 | 1889.02 | 945.01 | 630.34 |
| SP611 | 1308 | Ac-LTF$r8EYWANL$AAAAAa-NH2 | | 1915.02 | 1006.41 | 1916.03 | 958.52 | 639.35 |
| SP612 | 1309 | Ac-LTF$r8EYWACitL$AAAAAa-NH2 | | 1958.07 | | 1959.08 | 980.04 | 653.7 |
| SP613 | 1310 | Ac-LTF$r8EYWAHL$AAAAAa-NH2 | | 1938.04 | 966.24 | 1939.05 | 970.03 | 647.02 |
| SP614 | 1311 | Ac-LTF$r8EYWARL$AAAAAa-NH2 | | 1957.08 | | 1958.09 | 979.55 | 653.37 |
| SP615 | 1312 | Ac-LTF$r8EpYWAQL$AAAAAa-NH2 | | 2009.01 | | 2010.02 | 1005.51 | 670.68 |
| SP616 | 1313 | Cbm-LTF$r8EYWAQCba$SAA-NH2 | | 1590.85 | | 1591.86 | 796.43 | 531.29 |
| SP617 | 1314 | Cbm-LTF$r8EYWAQL$AAAAAa-NH2 | | 1930.04 | | 1931.05 | 966.03 | 644.35 |
| SP618 | 1315 | Ac-LTF$r8EYWAQL$SAAAAa-NH2 | | 1945.04 | 1005.11 | 1946.05 | 973.53 | 649.35 |
| SP619 | 1316 | Ac-LTF$r8EYWAQL$AAAASa-NH2 | | 1945.04 | 986.52 | 1946.05 | 973.53 | 649.35 |
| SP620 | 1317 | Ac-LTF$r8EYWAQL$SAAASa-NH2 | | 1961.03 | 993.27 | 1962.04 | 981.52 | 654.68 |
| SP621 | 1318 | Ac-LTF$r8EYWAQTba$AAAAAa-NH2 | | 1943.06 | 983.1 | 1944.07 | 972.54 | 648.69 |
| SP622 | 1319 | Ac-LTF$r8EYWAQAdm$AAAAAa-NH2 | | 2007.09 | 990.31 | 2008.1 | 1004.55 | 670.04 |
| SP623 | 1320 | Ac-LTF$r8EYWAQCha$AAAAAa-NH2 | | 1969.07 | 987.17 | 1970.08 | 985.54 | 657.36 |
| SP624 | 1321 | Ac-LTF$r8EYWAQhCha$AAAAAa-NH2 | | 1983.09 | 1026.11 | 1984.1 | 992.55 | 662.04 |
| SP625 | 1322 | Ac-LTF$r8EYWAQF$AAAAAa-NH2 | | 1963.02 | 957.01 | 1964.03 | 982.52 | 655.35 |
| SP626 | 1323 | Ac-LTF$r8EYWAQhF$AAAAAa-NH2 | | 1977.04 | 1087.81 | 1978.05 | 989.53 | 660.02 |
| SP627 | 1324 | Ac-LTF$r8EYWAQL$AANleAAa-NH2 | | 1971.09 | 933.45 | 1972.1 | 986.55 | 658.04 |
| SP628 | 1325 | Ac-LTF$r8EYWAQAdm$AANleAAa-NH2 | | 2049.13 | 1017.97 | 2050.14 | 1025.57 | 684.05 |
| SP629 | 1326 | 4-FBz-BaLTF$r8EYWAQL$AAAAAa-NH2 | | 2080.08 | | 2081.09 | 1041.05 | 694.37 |
| SP630 | 1327 | 4-FBz-BaLTF$r8EYWAQCba$SAA-NH2 | | 1894.97 | | 1895.98 | 948.49 | 632.66 |
| SP631 | 1328 | Ac-LTF$r5EYWAQL$s8AAAAAa-NH2 | | 1929.04 | 1072.68 | 1930.05 | 965.53 | 644.02 |
| SP632 | 1329 | Ac-LTF$r5EYWAQCba$s8SAA-NH2 | | 1743.92 | 1107.79 | 1744.93 | 872.97 | 582.31 |
| SP633 | 1330 | Ac-LTF$r8EYWAQL$AAhhLAAa-NH2 | | 1999.12 | | 2000.13 | 1000.57 | 667.38 |
| SP634 | 1331 | Ac-LTF$r8EYWAQL$AAAAAAAa-NH2 | | 2071.11 | | 2072.12 | 1036.56 | 691.38 |
| SP635 | 1332 | Ac-LTF$r8EYWAQL$AAAAAAAAa-NH2 | | 2142.15 | 778.1 | 2143.16 | 1072.08 | 715.06 |
| SP636 | 1333 | Ac-LTF$r8EYWAQL$AAAAAAAAAa-NH2 | | 2213.19 | 870.53 | 2214.2 | 1107.6 | 738.74 |
| SP637 | 1334 | Ac-LTA$r8EYAAQCba$SAA-NH2 | | 1552.85 | | 1553.86 | 777.43 | 518.62 |
| SP638 | 1335 | Ac-LTA$r8EYAAQL$AAAAAa-NH2 | | 1737.97 | 779.45 | 1738.98 | 869.99 | 580.33 |
| SP639 | 1336 | Ac-LTF$r8EPmpWAQL$AAAAAa-NH2 | | 2007.03 | 779.54 | 2008.04 | 1004.52 | 670.02 |
| SP640 | 1337 | Ac-LTF$r8EPmpWAQCba$SAA-NH2 | | 1821.91 | 838.04 | 1822.92 | 911.96 | 608.31 |
| SP641 | 1338 | Ac-ATF$r8HYWAQL$S-NH2 | | 1555.82 | 867.83 | 1556.83 | 778.92 | 519.61 |
| SP642 | 1339 | Ac-LTF$r8HAWAQL$S-NH2 | | 1505.84 | 877.91 | 1506.85 | 753.93 | 502.95 |
| SP643 | 1340 | Ac-LTF$r8HYWAQA$S-NH2 | | 1555.82 | 852.52 | 1556.83 | 778.92 | 519.61 |
| SP644 | 1341 | Ac-LTF$r8EYWAQCba$SA-NH2 | | 1672.89 | 887.18 | 1673.9 | 837.45 | 558.64 |
| SP645 | 1342 | Ac-LTF$r8EYWAQL$SAA-NH2 | | 1731.92 | 873.32 | 1732.93 | 866.97 | 578.31 |
| SP646 | 1343 | Ac-LTF$r8HYWAQCba$SAA-NH2 | | 1751.94 | 873.05 | 1752.95 | 876.98 | 584.99 |
| SP647 | 1344 | Ac-LTF$r8SYWAQCba$SAA-NH2 | | 1701.91 | 844.88 | 1702.92 | 851.96 | 568.31 |
| SP648 | 1345 | Ac-LTF$r8RYWAQCba$SAA-NH2 | | 1770.98 | 865.58 | 1771.99 | 886.5 | 591.33 |
| SP649 | 1346 | Ac-LTF$r8KYWAQCba$SAA-NH2 | | 1742.98 | 936.57 | 1743.99 | 872.5 | 582 |
| SP650 | 1347 | Ac-LTF$r8QYWAQCba$SAA-NH2 | | 1742.94 | 930.93 | 1743.95 | 872.48 | 581.99 |
| SP651 | 1348 | Ac-LTF$r8EYWAACba$SAA-NH2 | | 1686.9 | 1032.45 | 1687.91 | 844.46 | 563.31 |
| SP652 | 1349 | Ac-LTF$r8EYWAQCba$AAA-NH2 | | 1727.93 | 895.46 | 1728.94 | 864.97 | 576.98 |
| SP653 | 1350 | Ac-LTF$r8EYWAQL$AAAAA-OH | | 1858.99 | 824.54 | 1860 | 930.5 | 620.67 |
| SP654 | 1351 | Ac-LTF$r8EYWAQL$AAAA-OH | | 1787.95 | 894.48 | 1788.96 | 894.98 | 596.99 |
| SP655 | 1352 | Ac-LTF$r8EYWAQL$AA-OH | | 1645.88 | 856 | 1646.89 | 823.95 | 549.63 |
| SP656 | 1353 | Ac-LTF$r8AF4bOH2WAQL$AAAAAa-NH2 | | | | | | |
| SP657 | 1354 | Ac-LTF$r8AF4bOH2WAAL$AAAAAa-NH2 | | | | | | |
| SP658 | 1355 | Ac-LTF$r8EF4bOH2WAQCba$SAA-NH2 | | | | | | |
| SP659 | 1356 | Ac-LTF$r8ApYWAQL$AAAAAa-NH2 | | | | | | |
| SP660 | 1357 | Ac-LTF$r8ApYWAAL$AAAAAa-NH2 | | | | | | |
| SP661 | 1358 | Ac-LTF$r8EpYWAQCba$SAA-NH2 | | | | | | |
| SP662 | 1359 | Ac-LTF$rda6AYWAQL$da5AAAAAa-NH2 | | 1974.06 | 934.44 | | | |
| SP663 | 1360 | Ac-LTF$rda6EYWAQCba$da5SAA-NH2 | | 1846.95 | 870.52 | | 869.94 | |
| SP664 | 1361 | Ac-LTF$rda6EYWAQL$da5AAAAAa-NH2 | | | | | | |
| SP665 | 1362 | Ac-LTF$ra9EYWAQL$a6AAAAAa-NH2 | | | 936.57 | | 935.51 | |
| SP666 | 1363 | Ac-LTF$ra9EYWAQL$a6AAAAAa-NH2 | | | | | | |
| SP667 | 1364 | Ac-LTF$ra9EYWAQCba$a6SAA-NH2 | | | | | | |
| SP668 | 1365 | Ac-LTA$ra9EYWAQCba$a6SAA-NH2 | | | | | | |
| SP669 | 1366 | 5-FAM-BaLTF$ra9EYWAQCba$a6SAA-NH2 | | | | | | |
| SP670 | 1367 | 5-FAM-BaLTF$r8EYWAQL$AAAAAa-NH2 | | 2316.11 | | | | |
| SP671 | 1368 | 5-FAM-BaLTF$/r8EYWAQL$/AAAAAa-NH2 | | 2344.15 | | | | |
| SP672 | 1369 | 5-FAM-BaLTA$r8EYWAQL$AAAAAa-NH2 | | 2240.08 | | | | |
| SP673 | 1370 | 5-FAM-BaLTF$r8AYWAQL$AAAAAa-NH2 | | 2258.11 | | | | |
| SP674 | 1371 | 5-FAM-BaATF$r8EYWAQL$AAAAAa-NH2 | | 2274.07 | | | | |
| SP675 | 1372 | 5-FAM-BaLAF$r8EYWAQL$AAAAAa-NH2 | | 2286.1 | | | | |
| SP676 | 1373 | 5-FAM-BaLTF$r8EAWAQL$AAAAAa-NH2 | | 2224.09 | | | | |
| SP677 | 1374 | 5-FAM-BaLTF$r8EYAAQL$AAAAAa-NH2 | | 2201.07 | | | | |
| SP678 | 1375 | 5-FAM-BaLTA$r8EYAAQL$AAAAAa-NH2 | | 2125.04 | | | | |
| SP679 | 1376 | 5-FAM-BaLTF$r8EYWAAL$AAAAAa-NH2 | | 2259.09 | | | | |
| SP680 | 1377 | 5-FAM-BaLTF$r8EYWAQA$AAAAAa-NH2 | | 2274.07 | | | | |
| SP681 | 1378 | 5-FAM-BaLTF$/r8EYWAQCba$/SAA-NH2 | | 2159.03 | | | | |
| SP682 | 1379 | 5-FAM-BaLTA$r8EYWAQCba$SAA-NH2 | | 2054.97 | | | | |
| SP683 | 1380 | 5-FAM-BaLTF$r8EYAAQCba$SAA-NH2 | | 2015.96 | | | | |
| SP684 | 1381 | 5-FAM-BaLTA$r8EYAAQCba$SAA-NH2 | | 1939.92 | | | | |
| SP685 | 1382 | 5-FAM-BaQSQQTF$r8NLWRLL$QN-NH2 | | 2495.23 | | | | |
| SP686 | 1383 | 5-TAMRA-BaLTF$r8EYWAQCba$SAA-NH2 | | 2186.1 | | | | |
| SP687 | 1384 | 5-TAMRA-BaLTA$r8EYWAQCba$SAA-NH2 | | 2110.07 | | | | |
| SP688 | 1385 | 5-TAMRA-BaLTF$r8EYAAQCba$SAA-NH2 | | 2071.06 | | | | |
| SP689 | 1386 | 5-TAMRA-BaLTA$r8EYAAQCba$SAA-NH2 | | 1995.03 | | | | |
| SP690 | 1387 | 5-TAMRA-BaLTF$/r8EYWAQCba$/SAA-NH2 | | 2214.13 | | | | |
| SP691 | 1388 | 5-TAMRA-BaLTF$r8EYWAQL$AAAAAa-NH2 | | 2371.22 | | | | |
| SP692 | 1389 | 5-TAMRA-BaLTA$r8EYWAQL$AAAAAa-NH2 | | 2295.19 | | | | |
| SP693 | 1390 | 5-TAMRA-BaLTF$/r8EYWAQL$/AAAAAa-NH2 | | 2399.25 | | | | |
| SP694 | 1391 | Ac-LTF$r8EYWCou7QCba$SAA-OH | | 1947.93 | | | | |
| SP695 | 1392 | Ac-LTF$r8EYWCou7QCba$S-OH | | 1805.86 | | | | |
| SP696 | 1393 | Ac-LTA$r8EYWCou7QCba$SAA-NH2 | | 1870.91 | | | | |
| SP697 | 1394 | Ac-LTF$r8EYACou7QCba$SAA-NH2 | | 1831.9 | | | | |
| SP698 | 1395 | Ac-LTA$r8EYACou7QCba$SAA-NH2 | | 1755.87 | | | | |
| SP699 | 1396 | Ac-LTF$/r8EYWCou7QCba$/SAA-NH2 | | 1974.98 | | | | |
| SP700 | 1397 | Ac-LTF$r8EYWCou7QL$AAAAAa-NH2 | | 2132.06 | | | | |
| SP701 | 1398 | Ac-LTF$/r8EYWCou7QL$/AAAAAa-NH2 | | 2160.09 | | | | |
| SP702 | 1399 | Ac-LTF$r8EYWCou7QL$AAAAA-OH | | 2062.01 | | | | |
| SP703 | 1400 | Ac-LTF$r8EYWCou7QL$AAAA-OH | | 1990.97 | | | | |
| SP704 | 1401 | Ac-LTF$r8EYWCou7QL$AAA-OH | | 1919.94 | | | | |
| SP705 | 1402 | Ac-LTF$r8EYWCou7QL$AA-OH | | 1848.9 | | | | |
| SP706 | 1403 | Ac-LTF$r8EYWCou7QL$A-OH | | 1777.86 | | | | |
| SP707 | 1404 | Ac-LTF$r8EYWAQL$AAAASa-NH2 | iso2 | | 974.4 | | 973.53 | |
| SP708 | 1405 | Ac-LTF$r8AYWAAL$AAAAAa-NH2 | iso2 | 1814.01 | 908.82 | 1815.02 | 908.01 | 605.68 |
| SP709 | 1406 | Biotin-BaLTF$r8EYWAQL$AAAAAa-NH2 | | 2184.14 | 1093.64 | 2185.15 | 1093.08 | 729.05 |
| SP710 | 1407 | Ac-LTF$r8HAWAQL$S-NH2 | iso2 | 1505.84 | 754.43 | 1506.85 | 753.93 | 502.95 |
| SP711 | 1408 | Ac-LTF$r8EYWAQCba$SA-NH2 | iso2 | 1672.89 | 838.05 | 1673.9 | 837.45 | 558.64 |
| SP712 | 1409 | Ac-LTF$r8HYWAQCba$SAA-NH2 | iso2 | 1751.94 | 877.55 | 1752.95 | 876.98 | 584.99 |
| SP713 | 1410 | Ac-LTF$r8SYWAQCba$SAA-NH2 | iso2 | 1701.91 | 852.48 | 1702.92 | 851.96 | 568.31 |
| SP714 | 1411 | Ac-LTF$r8RYWAQCba$SAA-NH2 | iso2 | 1770.98 | 887.45 | 1771.99 | 886.5 | 591.33 |
| SP715 | 1412 | Ac-LTF$r8KYWAQCba$SAA-NH2 | iso2 | 1742.98 | 872.92 | 1743.99 | 872.5 | 582 |
| SP716 | 1413 | Ac-LTF$r8EYWAQCba$AAA-NH2 | iso2 | 1727.93 | 865.71 | 1728.94 | 864.97 | 576.98 |
| SP717 | 1414 | Ac-LTF$r8EYWAQL$AAAAAaBaC-NH2 | | 2103.09 | 1053.12 | 2104.1 | 1052.55 | 702.04 |
| SP718 | 1415 | Ac-LTF$r8EYWAQL$AAAAAadPeg4C-NH2 | | 2279.19 | 1141.46 | 2280.2 | 1140.6 | 760.74 |
| SP719 | 1416 | Ac-LTA$r8AYWAAL$AAAAAa-NH2 | | 1737.98 | 870.43 | 1738.99 | 870 | 580.33 |
| SP720 | 1417 | Ac-LTF$r8AYAAAL$AAAAAa-NH2 | | 1698.97 | 851 | 1699.98 | 850.49 | 567.33 |
| SP721 | 1418 | 5-FAM-BaLTF$r8AYWAAL$AAAAAa-NH2 | | 2201.09 | 1101.87 | 2202.1 | 1101.55 | 734.7 |
| SP722 | 1419 | Ac-LTA$r8AYWAQL$AAAAAa-NH2 | | 1795 | 898.92 | 1796.01 | 898.51 | 599.34 |
| SP723 | 1420 | Ac-LTF$r8AYAAQL$AAAAAa-NH2 | | 1755.99 | 879.49 | 1757 | 879 | 586.34 |
| SP724 | 1421 | Ac-LTF$rda6AYWAAL$da5AAAAAa-NH2 | | 1807.97 | | 1808.98 | 904.99 | 603.66 |
| SP725 | 1422 | FITC-BaLTF$r8EYWAQL$AAAAAa-NH2 | | 2347.1 | 1174.49 | 2348.11 | 1174.56 | 783.37 |
| SP726 | 1423 | FITC-BaLTF$r8EYWAQCba$SAA-NH2 | | 2161.99 | 1082.35 | 2163 | 1082 | 721.67 |
| SP733 | 1424 | Ac-LTF$r8EYWAQL$EAAAAa-NH2 | | 1987.05 | 995.03 | 1988.06 | 994.53 | 663.36 |
| SP734 | 1425 | Ac-LTF$r8AYWAQL$EAAAAa-NH2 | | 1929.04 | 966.35 | 1930.05 | 965.53 | 644.02 |
| SP735 | 1426 | Ac-LTF$r8EYWAQL$AAAAAaBaKbio-NH2 | | 2354.25 | 1178.47 | 2355.26 | 1178.13 | 785.76 |
| SP736 | 1427 | Ac-LTF$r8AYWAAL$AAAAAa-NH2 | | 1814.01 | 908.45 | 1815.02 | 908.01 | 605.68 |
| SP737 | 1428 | Ac-LTF$r8AYAAAL$AAAAAa-NH2 | iso2 | 1698.97 | 850.91 | 1699.98 | 850.49 | 567.33 |
| SP738 | 1429 | Ac-LTF$r8AYAAQL$AAAAAa-NH2 | iso2 | 1755.99 | 879.4 | 1757 | 879 | 586.34 |
| SP739 | 1430 | Ac-LTF$r8EYWAQL$EAAAAa-NH2 | iso2 | 1987.05 | 995.21 | 1988.06 | 994.53 | 663.36 |
| SP740 | 1431 | Ac-LTF$r8AYWAQL$EAAAAa-NH2 | iso2 | 1929.04 | 966.08 | 1930.05 | 965.53 | 644.02 |
| SP741 | 1432 | Ac-LTF$r8EYWAQCba$SAAAAa-NH2 | | 1957.04 | 980.04 | 1958.05 | 979.53 | 653.35 |
| SP742 | 1433 | Ac-LTF$r8EYWAQLStAAA$r5AA-NH2 | | 2023.12 | 1012.83 | 2024.13 | 1012.57 | 675.38 |
| SP743 | 1434 | Ac-LTF$r8EYWAQL$A$AAA$A-NH2 | | 2108.17 | 1055.44 | 2109.18 | 1055.09 | 703.73 |
| SP744 | 1435 | Ac-LTF$r8EYWAQL$AA$AAA$A-NH2 | | 2179.21 | 1090.77 | 2180.22 | 1090.61 | 727.41 |
| SP745 | 1436 | Ac-LTF$r8EYWAQL$AAA$AAA$A-NH2 | | 2250.25 | 1126.69 | 2251.26 | 1126.13 | 751.09 |
| SP746 | 1437 | Ac-AAALTF$r8EYWAQL$AAA-OH | | 1930.02 | | 1931.03 | 966.02 | 644.35 |
| SP747 | 1438 | Ac-AAALTF$r8EYWAQL$AAA-NH2 | | 1929.04 | 965.85 | 1930.05 | 965.53 | 644.02 |
| SP748 | 1439 | Ac-AAAALTF$r8EYWAQL$AAA-NH2 | | 2000.08 | 1001.4 | 2001.09 | 1001.05 | 667.7 |
| SP749 | 1440 | Ac-AAAAALTF$r8EYWAQL$AAA-NH2 | | 2071.11 | 1037.13 | 2072.12 | 1036.56 | 691.38 |
| SP750 | 1441 | Ac-AAAAAALTF$r8EYWAQL$AAA-NH2 | | 2142.15 | | 2143.16 | 1072.08 | 715.06 |
| SP751 | 1442 | Ac-LTF$rda6EYWAQCba$da6SAA-NH2 | iso2 | 1751.89 | 877.36 | 1752.9 | 876.95 | 584.97 |
| SP752 | 1443 | Ac-t$r5wya$r5f4CF3ekllr-NH2 | | | 844.25 | | | |
| SP753 | 1444 | Ac-tawy$r5nf4CF3e$r5llr-NH2 | | | 837.03 | | | |
| SP754 | 1445 | Ac-tawya$r5f4CF3ek$r5lr-NH2 | | | 822.97 | | | |
| SP755 | 1446 | Ac-tawyanf4CF3e$r5llr$r5a-NH2 | | | 908.35 | | | |
| SP756 | 1447 | Ac-t$s8wyanf4CF3e$r5llr-NH2 | | | 858.03 | | | |
| SP757 | 1448 | Ac-tawy$s8nf4CF3ekll$r5a-NH2 | | | 879.86 | | | |
| SP758 | 1449 | Ac-tawya$s8f4CF3ekllr$r5a-NH2 | | | 936.38 | | | |
| SP759 | 1450 | Ac-tawy$s8naekll$r5a-NH2 | | | 844.25 | | | |
| SP760 | 1451 | 5-FAM-Batawy$s8nf4CF3ekll$r5a-NH2 | | | | | | |
| SP761 | 1452 | 5-FAM-Batawy$s8naekll$r5a-NH2 | | | | | | |
| SP762 | 1453 | Ac-tawy$s8nf4CF3eall$r5a-NH2 | | | | | | |
| SP763 | 1454 | Ac-tawy$s8nf4CF3ekll$r5aaaaa-NH2 | | | | | | |
| SP764 | 1455 | Ac-tawy$s8nf4CF3eall$r5aaaaa-NH2 | | | | | | |

TABLE 11

| | SEQ | | | Exact | Found | Calc | Calc | Calc |
|---|---|---|---|---|---|---|---|---|
| SP | ID NO: | Sequence | Isomer | Mass | Mass | (M + 1)/1 | (M + 2)/2 | (M + 3)/3 |
| SP244 | 1456 | Ac-LTF$r8EF4coohWAQCba$SANleA-NH2 | | 1885 | 943.59 | 1886.01 | 943.51 | 629.34 |
| SP331 | 1457 | Ac-LTF$r8EYWAQL$AAAAAa-NH2 | iso2 | 1929.04 | 966.08 | 1930.05 | 965.53 | 644.02 |
| SP555 | 1458 | Ac-LTF$r8EY6clWAQL$AAAAAa-NH2 | | 1963 | 983.28 | 1964.01 | 982.51 | 655.34 |
| SP557 | 1459 | Ac-AAALTF$r8EYWAQL$AAAAAa-NH2 | | 2142.15 | 1072.83 | 2143.16 | 1072.08 | 715.06 |
| SP558 | 1460 | Ac-LTF34F2$r8EYWAQL$AAAAAa-NH2 | | 1965.02 | 984.3 | 1966.03 | 983.52 | 656.01 |
| SP562 | 1461 | Ac-LTF$r8EYWAQL$AAibAAAa-NH2 | | 1943.06 | 973.11 | 1944.07 | 972.54 | 648.69 |
| SP564 | 1462 | Ac-LTF$r8EYWAQL$AAAAibAa-NH2 | | 1943.06 | 973.48 | 1944.07 | 972.54 | 648.69 |
| SP566 | 1463 | Ac-LTF$r8EYWAQL$AAAAAiba-NH2 | iso2 | 1943.06 | 973.38 | 1944.07 | 972.54 | 648.69 |
| SP567 | 1464 | Ac-LTF$r8EYWAQL$AAAAAAib-NH2 | | 1943.06 | 973.01 | 1944.07 | 972.54 | 648.69 |
| SP572 | 1465 | Ac-LTF$r8EYWAQL$AAAAaa-NH2 | | 1929.04 | 966.35 | 1930.05 | 965.53 | 644.02 |
| SP573 | 1466 | Ac-LTF$r8EYWAQL$AAAAAA-NH2 | | 1929.04 | 966.35 | 1930.05 | 965.53 | 644.02 |
| SP578 | 1467 | Ac-LTF$r8EYWAQL$AAAAASar-NH2 | | 1929.04 | 966.08 | 1930.05 | 965.53 | 644.02 |
| SP551 | 1468 | Ac-LTF$r8EYWAQL$AAAAAa-OH | iso2 | 1930.02 | 965.89 | 1931.03 | 966.02 | 644.35 |
| SP662 | 1469 | Ac-LTF$rda6AYWAQL$da5AAAAAa-NH2 | | 1974.06 | 934.44 | | 933.49 | |
| SP367 | 1470 | 5-FAM-BaLTF$r8EYWAQCba$SAA-NH2 | | 2131 | 1067.09 | 2132.01 | 1066.51 | 711.34 |
| SP349 | 1471 | Ac-LTF$r8EF4coohWAQCba$AAAAAa-NH2 | iso2 | 1969.04 | 986.06 | 1970.05 | 985.53 | 657.35 |
| SP347 | 1472 | Ac-LTF$r8EYWAQCba$AAAAAa-NH2 | iso2 | 1941.04 | 972.55 | 1942.05 | 971.53 | 648.02 |

TABLE 12

| | | | | | | | | |
|---|---|---|---|---|---|---|---|---|
| | SEQ | | | Exact | Found | Calc | Calc | Calc |
| SP | ID NO: | Sequence | Isomer | Mass | Mass | (M + 1)/1 | (M + 2)/2 | (M + 3)/3 |

| | | | | | | | |
|---|---|---|---|---|---|---|---|
| SP581 | 1473 | Ac-TF$r8EYWAQL$AAAAAa-NH2 | 1815.96 | 929.85 | 1816.97 | 908.99 | 606.33 |
| SP582 | 1474 | Ac-F$r8EYWAQL$AAAAAa-NH2 | 1714.91 | 930.92 | 1715.92 | 858.46 | 572.64 |
| SP583 | 1475 | Ac-LVF$r8EYWAQL$AAAAAa-NH2 | 1927.06 | 895.12 | 1928.07 | 964.54 | 643.36 |
| SP584 | 1476 | Ac-AAF$r8EYWAQL$AAAAAa-NH2 | 1856.98 | 859.51 | 1857.99 | 929.5 | 620 |
| SP585 | 1477 | Ac-LTF$r8EYWAQL$AAAAa-NH2 | 1858 | 824.08 | 1859.01 | 930.01 | 620.34 |
| SP586 | 1478 | Ac-LTF$r8EYWAQL$AAAa-NH2 | 1786.97 | 788.56 | 1787.98 | 894.49 | 596.66 |
| SP587 | 1479 | Ac-LTF$r8EYWAQL$AAa-NH2 | 1715.93 | 1138.57 | 1716.94 | 858.97 | 572.98 |
| SP588 | 1480 | Ac-LTF$r8EYWAQL$Aa-NH2 | 1644.89 | 1144.98 | 1645.9 | 823.45 | 549.3 |
| SP589 | 1481 | Ac-LTF$r8EYWAQL$a-NH2 | 1573.85 | 1113.71 | 1574.86 | 787.93 | 525.62 |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon crosslinker comprising one double bond. Amino acids represented as “$r5” are alpha-Me R5-pentenyl-alanine olefin amino acids connected by an all-carbon comprising one double bond. Amino acids represented as “$s8” are alpha-Me S8-octenyl-alanine olefin amino acids connected by an all-carbon crosslinker comprising one double bond. Amino acids represented as “$r8” are alpha-Me R8-octenyl-alanine olefin amino acids connected by an all-carbon crosslinker comprising one double bond. “Ahx” represents an aminocyclohexyl linker. The crosslinkers are linear all-carbon crosslinker comprising eight or eleven carbon atoms between the alpha carbons of each amino acid. Amino acids represented as “$/” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker. Amino acids represented as “Amw” are alpha-Me tryptophan amino acids. Amino acids represented as “Aml” are alpha-Me leucine amino acids. Amino acids represented as “Amf” are alpha-Me phenylalanine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “St//” are amino acids comprising two pentenyl-alanine olefin side chains that are not crosslinked. Amino acids represented as “% St” are amino acids comprising two pentenyl-alanine olefin side chains, each of which is crosslinked to another amino acid as indicated via fully saturated hydrocarbon crosslinks. Amino acids represented as “Ba” are beta-alanine. The lower-case character “e” or “z” within the designation of a crosslinked amino acid (e.g. “$er8” or “$zr8”) represents the configuration of the double bond (E or Z, respectively). In other contexts, lower-case letters such as “a” or “f” represent D amino acids (e.g. D-alanine, or D-phenylalanine, respectively). Amino acids designated as “NmW” represent N-methyltryptophan. Amino acids designated as “NmY” represent N-methyltyrosine. Amino acids designated as “NmA” represent N-methylalanine. “Kbio” represents a biotin group attached to the side chain amino group of a lysine residue. Amino acids designated as “Sar” represent sarcosine. Amino acids designated as “Cha” represent cyclohexyl alanine. Amino acids designated as “Cpg” represent cyclopentyl glycine. Amino acids designated as “Chg” represent cyclohexyl glycine. Amino acids designated as “Cba” represent cyclobutyl alanine. Amino acids designated as “F4I” represent 4-iodo phenylalanine. “7L” represents N15 isotopic leucine. Amino acids designated as “F3Cl” represent 3-chloro phenylalanine. Amino acids designated as “F4cooh” represent 4-carboxy phenylalanine. Amino acids designated as “F34F2” represent 3,4-difluoro phenylalanine. Amino acids designated as “6clW” represent 6-chloro tryptophan. Amino acids designated as “$rda6” represent alpha-Me R6-hexynyl-alanine alkynyl amino acids, crosslinked via a dialkyne bond to a second alkynyl amino acid. Amino acids designated as “$da5” represent alpha-Me S5-pentynyl-alanine alkynyl amino acids, wherein the alkyne forms one half of a dialkyne bond with a second alkynyl amino acid. Amino acids designated as “$ra9” represent alpha-Me R9-nonynyl-alanine alkynyl amino acids, crosslinked via an alkyne metathesis reaction with a second alkynyl amino acid. Amino acids designated as “$a6” represent alpha-Me S6-hexynyl-alanine alkynyl amino acids, crosslinked via an alkyne metathesis reaction with a second alkynyl amino acid. The designation “iso1” or “iso2” indicates that the peptidomimetic macrocycle is a single isomer.

Amino acids designated as “Cit” represent citrulline. Amino acids designated as “Cou4”, “Cou6”, “Cou7” and “Cou8”, respectively, represent the following structures:

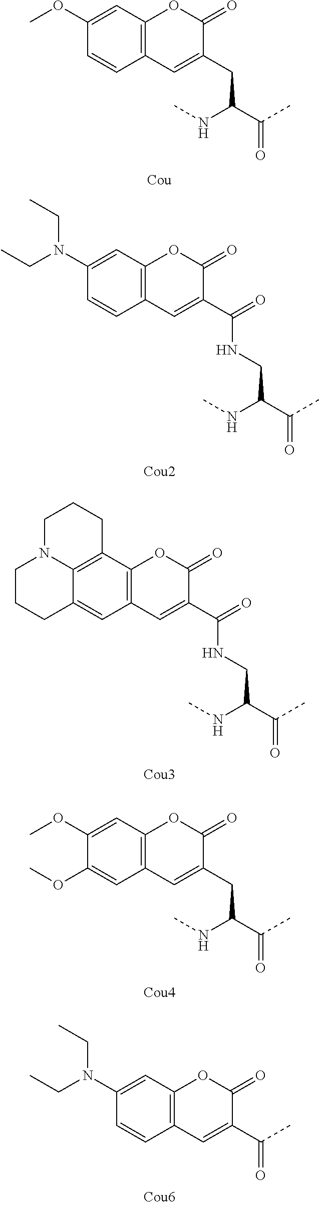

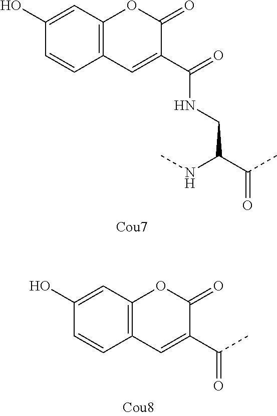

In some embodiments, a peptidomimetic macrocycle is obtained in more than one isomer, for example due to the configuration of a double bond within the structure of the crosslinker (E vs Z). Such isomers can or can not be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy, higher helicity, or improved cell permeability relative to its Z counterpart. In another embodiment, a Z crosslinker olefin isomer of a peptidomimetic macrocycle has better solubility, better target affinity, better in vivo or in vitro efficacy, higher helicity, or improved cell permeability relative to its E counterpart.

TABLE 13
(SEQ ID NOS 1482-1511, respectively, in order of appearance)
Structure

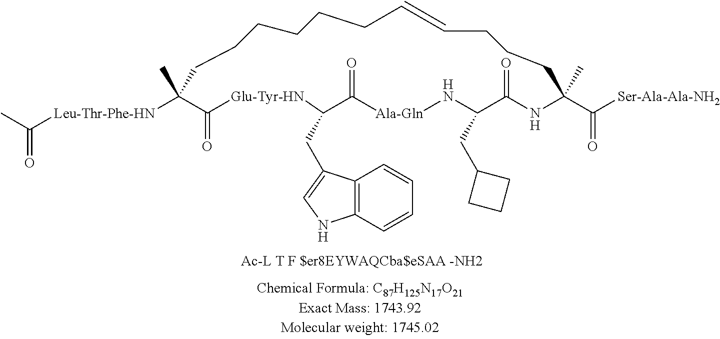

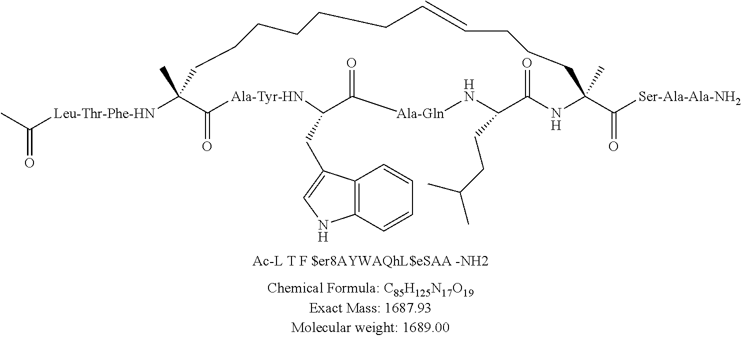

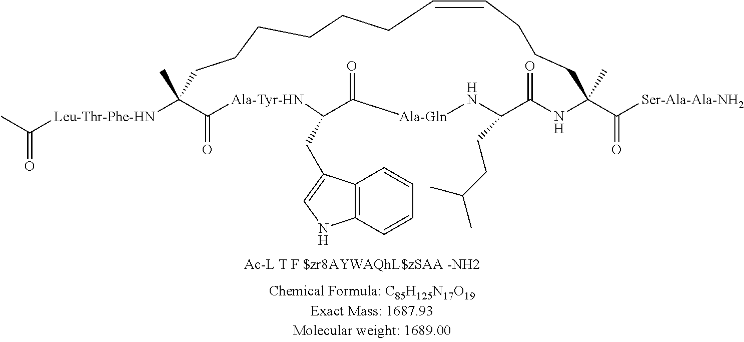

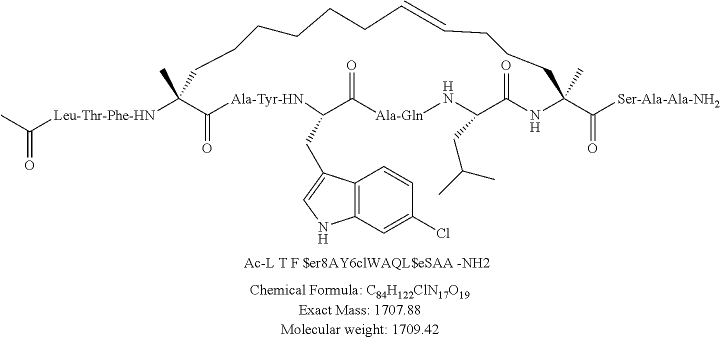

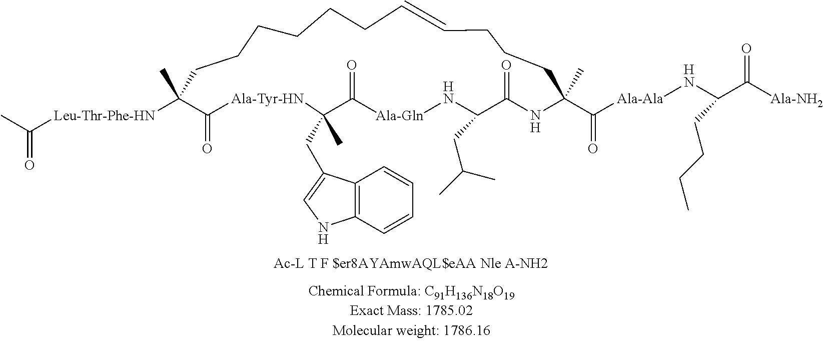

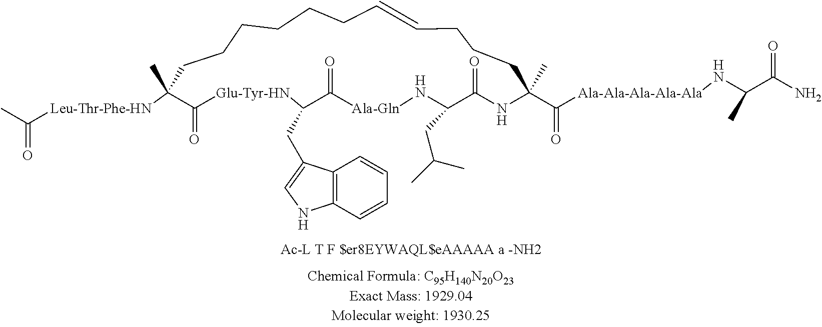

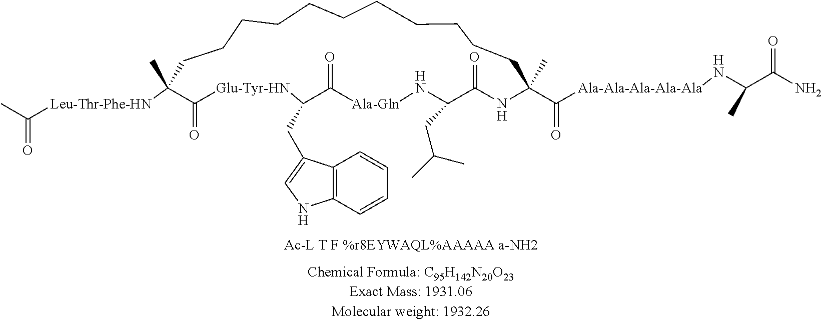

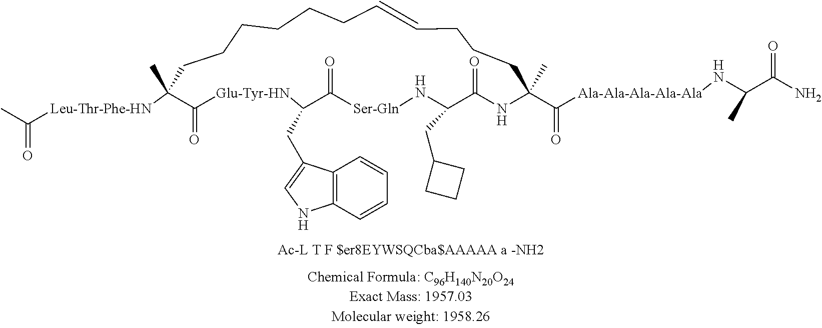

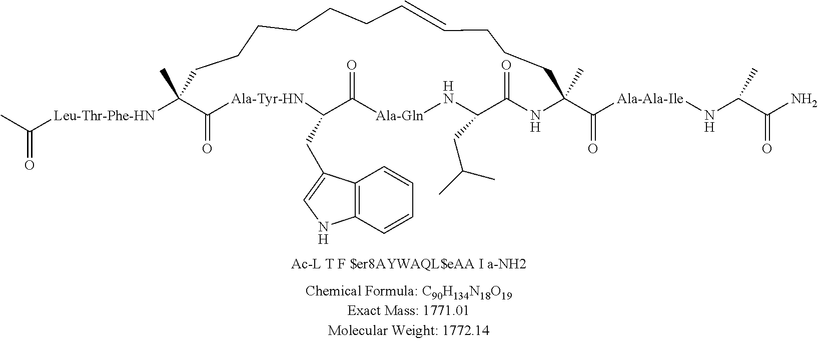

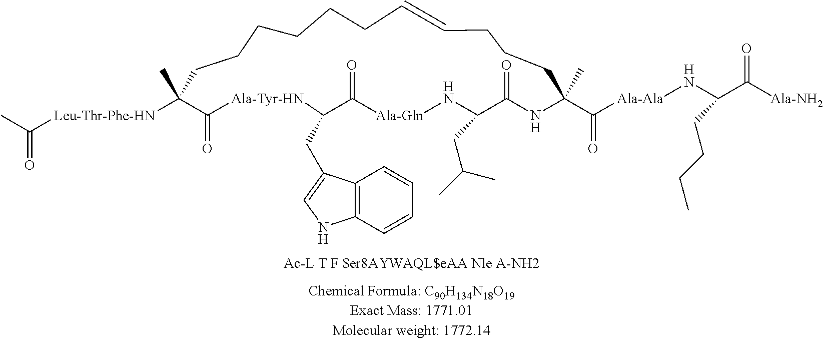

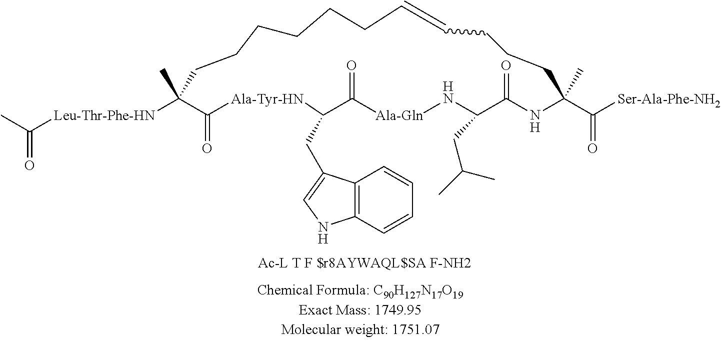

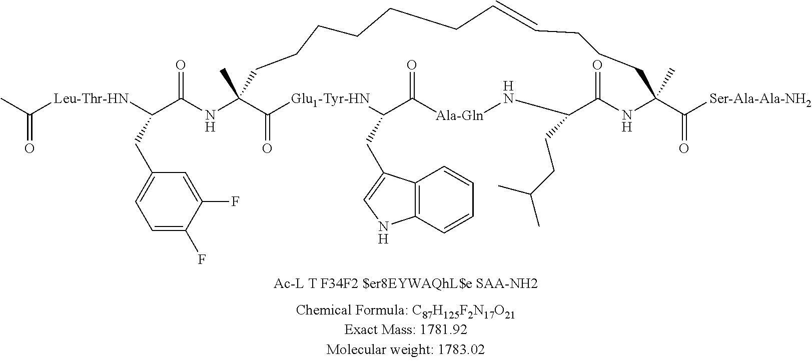

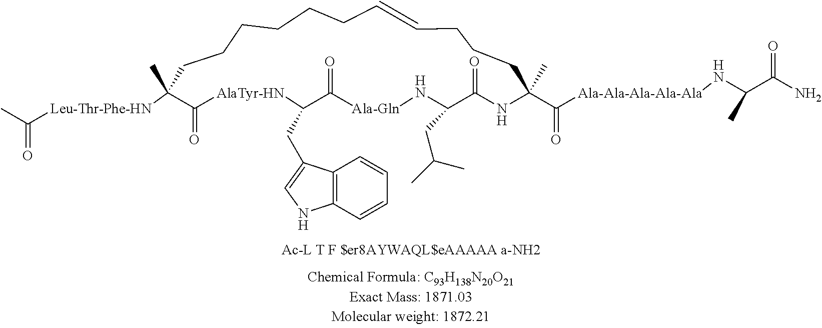

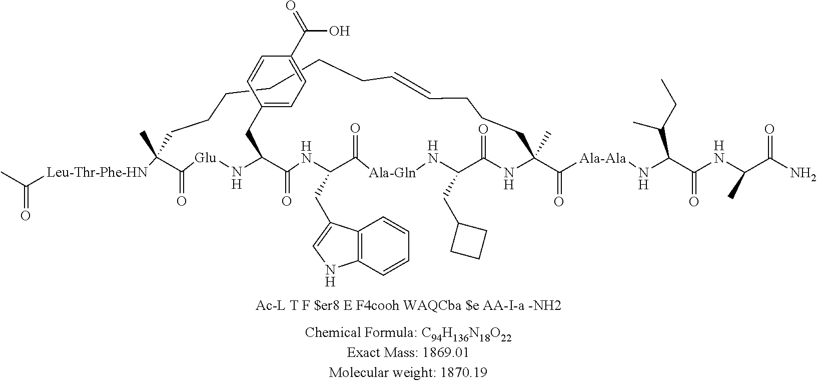

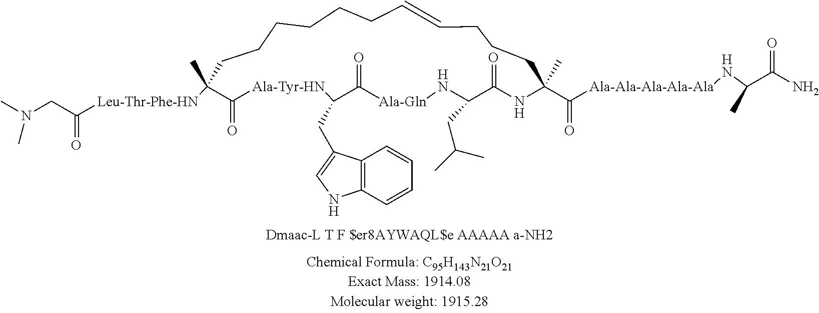

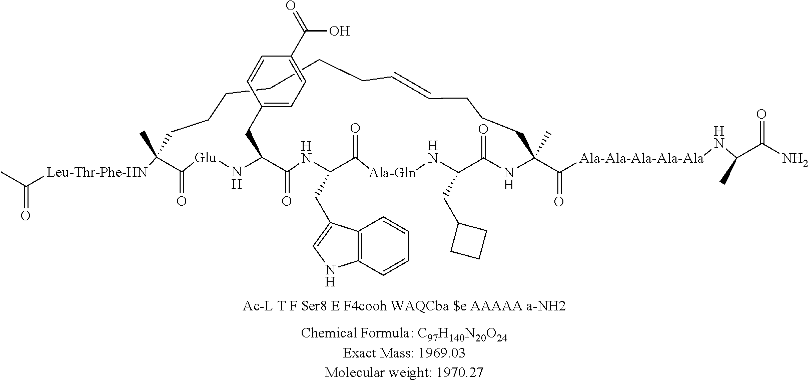

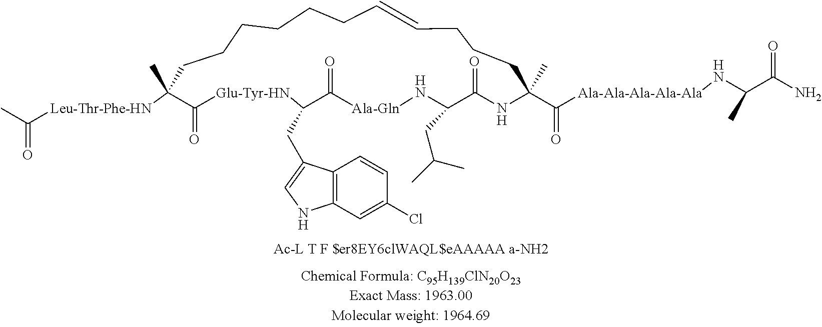

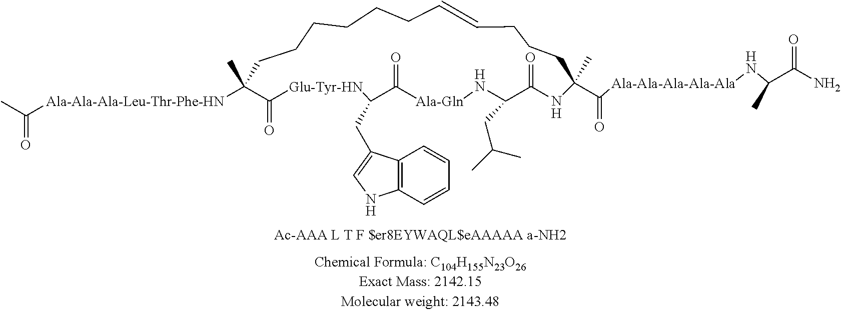

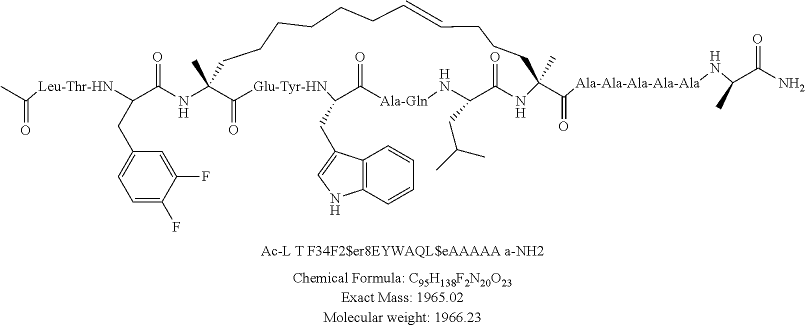

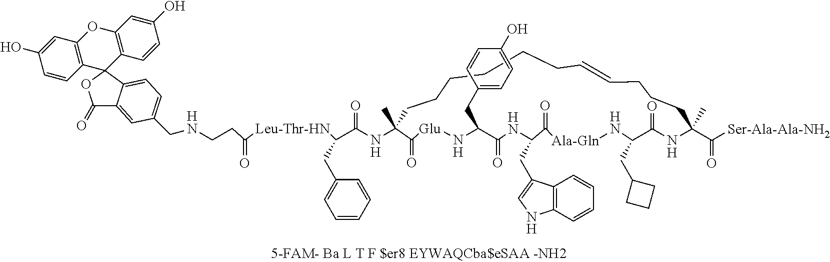

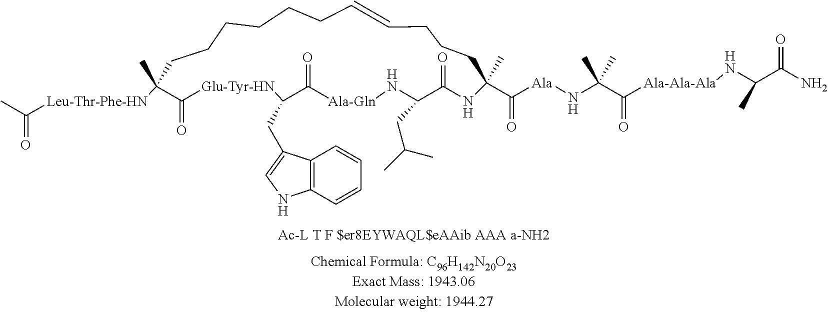

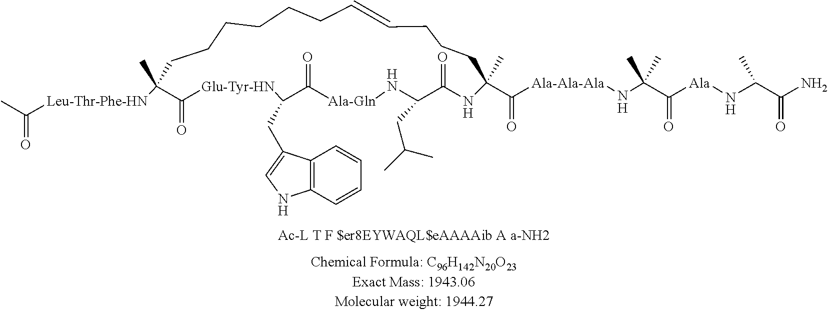

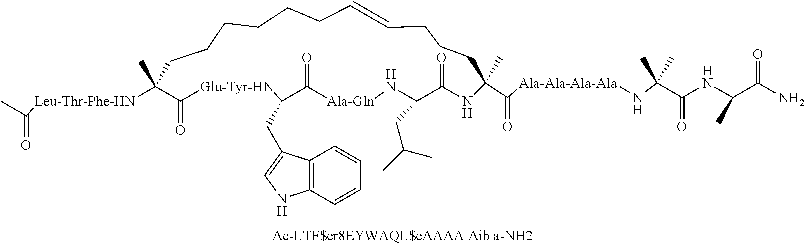

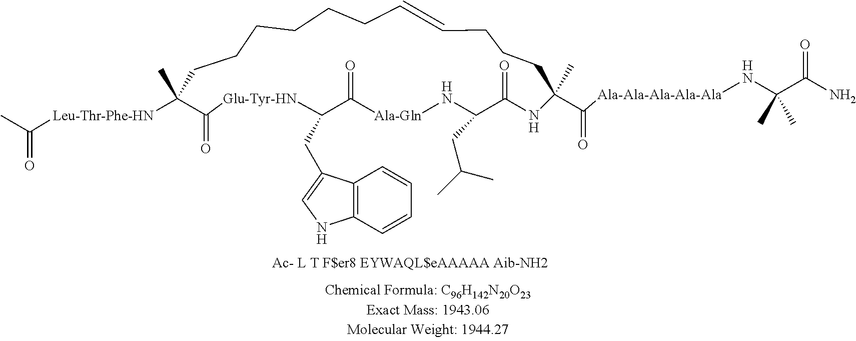

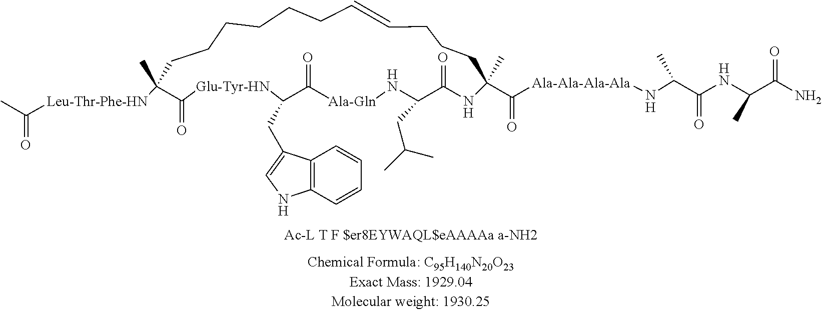

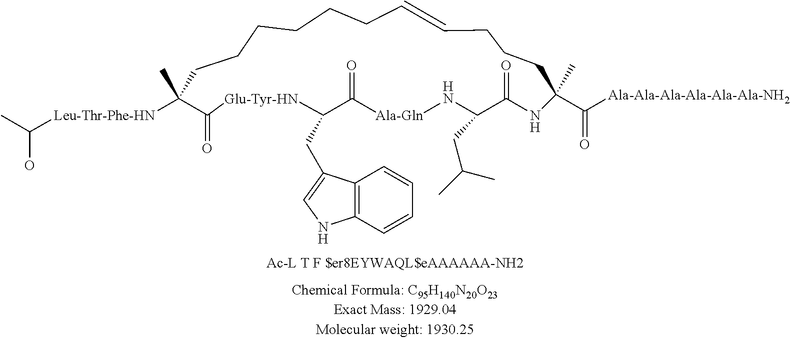

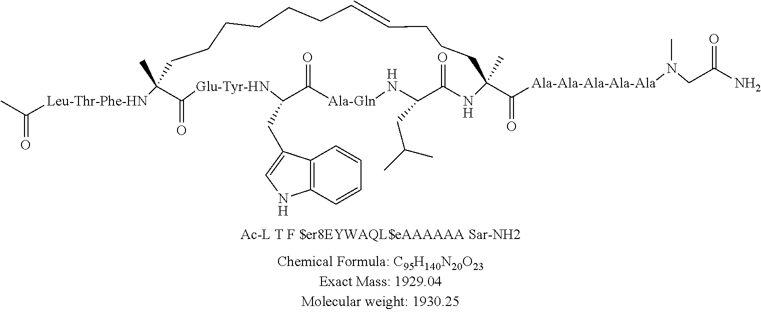

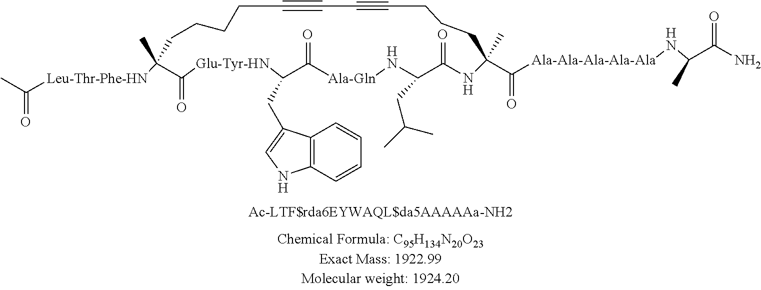

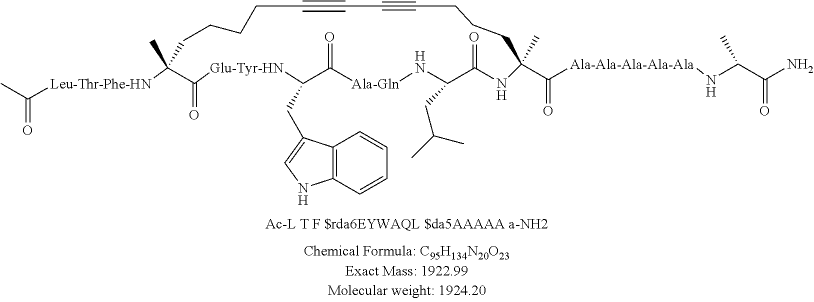

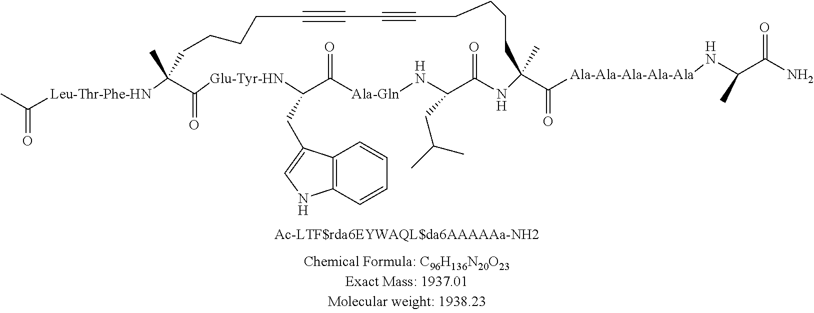

In some embodiments, peptidomimetic macrocycles exclude peptidomimetic macrocycles shown in Table 14. In some embodiments, peptidomimetic macrocycles do not comprise a peptidomimetic macrocycle structure as shown in Table 14.

TABLE 14

| Number | SEQ ID NO: | Sequence |
|---|---|---|
| 1 | 1512 | L$r5QETFSD$s8WKLLPEN |
| 2 | 1513 | LSQ$r5TFSDLW$s8LLPEN |
| 3 | 1514 | LSQE$r5FSDLWK$s8LPEN |
| 4 | 1515 | LSQET$r5SDLWKL$s8PEN |
| 5 | 1516 | LSQETF$r5DLWKLL$s8EN |
| 6 | 1517 | LXQETFS$r5LWKLLP$s8N |
| 7 | 1518 | LSQETFSD$r5WKLLPE$s8 |
| 8 | 1519 | LSQQTF$r5DLWKLL$s8EN |
| 9 | 1520 | LSQETF$r5DLWKLL$s8QN |
| 10 | 1521 | LSQQTF$r5DLWKLL$s8QN |
| 11 | 1522 | LSQETF$r5NLWKLL$s8QN |
| 12 | 1523 | LSQQTF$r5NLWKLL$s8QN |
| 13 | 1524 | LSQQTF$r5NLWRLL$s8QN |
| 14 | 1525 | QSQQTF$r5NLWKLL$s8QN |
| 15 | 1526 | QSQQTF$r5NLWRLL$s8QN |
| 16 | 1527 | QSQQTA$r5NLWRLL$s8QN |
| 17 | 1528 | L$r8QETFSD$WKLLPEN |
| 18 | 1529 | LSQ$r8TFSDLW$LLPEN |
| 19 | 1530 | LSQE$r8FSDLWK$LPEN |
| 20 | 1531 | LSQET$r8SDLWKL$PEN |
| 21 | 1532 | LSQETF$r8DLWKLL$EN |
| 22 | 1533 | LXQETFS$r8LWKLLP$N |
| 23 | 1534 | LSQETFSD$r8WKLLPE$ |
| 24 | 1535 | LSQQTF$r8DLWKLL$EN |
| 25 | 1536 | LSQETF$r8DLWKLL$QN |
| 26 | 1537 | LSQQTF$r8DLWKLL$QN |
| 27 | 1538 | LSQETF$r8NLWKLL$QN |
| 28 | 1539 | LSQQTF$r8NLWKLL$QN |
| 29 | 1540 | LSQQTF$r8NLWRLL$QN |
| 30 | 1541 | QSQQTF$r8NLWKLL$QN |
| 31 | 1542 | QSQQTF$r8NLWRLL$QN |
| 32 | 1543 | QSQQTA$r8NLWRLL$QN |
| 33 | 1544 | QSQQTF$r8NLWRKK$QN |
| 34 | 1545 | QQTF$r8DLWRLL$EN |
| 35 | 1546 | QQTF$r8DLWRLL$ |
| 36 | 1547 | LSQQTF$DLW$LL |
| 37 | 1548 | QQTF$DLW$LL |
| 38 | 1549 | QQTA$r8DLWRLL$EN |
| 39 | 1550 | QSQQTF$r5NLWRLL$s8QN |
| | | (dihydroxylated olefin) |
| 40 | 1551 | QSQQTA$r5NLWRLL$s8QN |
| | | (dihydroxylated olefin) |
| 41 | 1552 | QSQQTF$r8DLWRLL$QN |
| 42 | 1553 | QTF$r8NLWRLL$ |
| 43 | 1554 | QSQQTF$NLW$LLPQN |
| 44 | 1555 | QS$QTF$NLWRLLPQN |
| 45 | 1556 | $TFS$LWKLL |
| 46 | 1557 | ETF$DLW$LL |
| 47 | 1558 | QTF$NLW$LL |
| 48 | 1559 | $SQE$FSNLWKLL |

In Table 14, X represents S or any amino acid. Peptides shown can comprise an N-terminal capping group such as acetyl or an additional linker such as beta-alanine between the capping group and the start of the peptide sequence.

Table 15 shows examples of non-crosslinked polypeptides comprising D-amino acids.

TABLE 15

| | | | | | | | | |
|---|---|---|---|---|---|---|---|---|
| | SEQ | | | Exact | Found | Calc | Calc | Calc |
| SP | ID NO: | Sequence | Isomer | Mass | Mass | (M + 1)/1 | (M + 2)/2 | (M + 3)/3 |

| | | | |
|---|---|---|---|
| SP765 | 1560 | Ac-tawyanfekllr-NH2 | 777.46 |
| SP766 | 1561 | Ac-tawyanf4CF3ekllr-NH2 | 811.41 |

Peptidomimetic macrocycles are prepared as described herein and as in pending U.S. patent application Ser. No. 12/037,041, filed Feb. 25, 2008, which is hereby incorporated by reference in its entirety.

Generally, fully protected resin-bound peptides were synthesized on a PEG-PS resin (loading 0.45 mmol/g) on a 0.5 mmol scale. Deprotection of the temporary Fmoc group was achieved by 3×10 min treatments of the resin bound peptide with 20% (v/v) piperidine in DMF. After washing with NMP (3×), dichloromethane (3×) and NMP (3×), coupling of each successive amino acid was achieved with 1×60 min incubation with the appropriate preactivated Fmoc-amino acid derivative. All protected amino acids (1.0 mmol) were dissolved in NMP and activated with HCTU (1.0 mmol), Cl-HOBt (1.0 mmol) and DIEA (2.0 mmol) prior to transfer of the coupling solution to the deprotected resin-bound peptide. After coupling was completed, the resin was washed in preparation for the next deprotection/coupling cycle. Acetylation of the amino terminus was carried out in the presence of acetic anhydride/DIEA in NMP. The LC-MS analysis of a cleaved and deprotected sample obtained from an aliquot of the fully assembled resin-bound peptide was accomplished in order to verifying the completion of each coupling.

In a typical example for the preparation of a peptidomimetic macrocycle comprising a 1,4-triazole group (e.g. SP153), 20% (v/v) 2,6-lutidine in DMF was added to the peptide resin (0.5 mmol) in a 40 ml glass vial and shaken for 10 minutes. Sodium ascorbate (0.25 g, 1.25 mmol) and diisopropylethylamine (0.22 ml, 1.25 mmol) were then added, followed by copper(I) iodide (0.24 g, 1.25 mmol) and the resulting reaction mixture was mechanically shaken 16 hours at ambient temperature.

In a typical example for the preparation of a peptidomimetic macrocycle comprising a 1,5-triazole group (SP932, SP933), a peptide resin (0.25 mmol) was washed with anhydrous DCM. Resin was loaded into a microwave vial. Vessel was evacuated and purged with nitrogen. Chloro(penta-methylcyclopentadienyl) bis(triphenylphosphine)ruthenium(II), 10% loading, (Strem 44-0117) was added. Anhydrous toluene was added to the reaction vessel. The reaction was then loaded into the microwave and held at 90° C. for 10 minutes. Reaction may need to be pushed a subsequent time for completion. In other cases, chloro(1,5cyclooctadiene)(pentamethylcyclopenta-dienyl)ruthenium (“Cp*RuCl(cod)”) may be used, for example at at room temperature in a solvent comprising toluene.

In a typical example for the preparation of a peptidomimetic macrocycle comprising an iodo-substituted triazole group (e.g. SP457), THF (2 ml) was added to the peptide resin (0.05 mmol) in a 40 ml glass vial and shaken for 10 minutes. N-bromosuccimide (0.04 g, 0.25 mmol), copper(I) iodide (0.05 g, 0.25 mmol) and diisopropylethylamine (0.04 ml, 0.25 mmol) were then added and the resulting reaction mixture was mechanically shaken 16 hours at ambient temperature. Iodo-triazole crosslinkers may be further substituted by a coupling reaction, for example with boronic acids, to result in a peptidomimetic macrocycle such as SP465. In a typical example, DMF (3 ml) was added to the iodo-triazole peptide resin (0.1 mmol) in a 40 ml glass vial and shaken for 10 minutes. Phenyl boronic acid (0.04 g, 0.3 mmol), tetrakis(triphenylphosphine)palladium(0) (0.006 g, 0.005 mmol) and potassium carbonate (0.083 g, 0.6 mmol) were then added and the resulting reaction mixture was mechanically shaken 16 hours at 70° C. Iodo-triazole crosslinkers may also be further substituted by a coupling reaction, for example with a terminal alkyne (e.g. Sonogashira coupling), to result in a peptidomimetic macrocycle such as SP468. In a typical example, 2:1 THF:triethylamine (3 ml) was added to the iodo-triazole peptide resin (0.1 mmol) in a 40 ml glass vial and shaken for 10 minutes. N-BOC-4-pentyne-1-amine (0.04 g, 0.2 mmol) and bis(triphenylphosphine)palladiumchloride (0.014 g, 0.02 mmol) were added and shaken for 5 minutes. Copper(I) iodide (0.004 g, 0.02 mmol) was then added and the resulting reaction mixture was mechanically shaken 16 hours at 70° C.

The triazole-cyclized resin-bound peptides were deprotected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC.

Table 16 shows a list of peptidomimetic macrocycles.

TABLE 16

| | SEQ | |
|---|---|---|
| SP- | ID NO: | Sequence |
| 1 | 1562 | Ac-F$4rn6AYWEAc3cL$4a5AAA-NH2 |
| 2 | 1563 | Ac-F$4rn6AYWEAc3cL$4a5AAibA-NH2 |
| 3 | 1564 | Ac-LTF$4rn6AYWAQL$4a5SANle-NH2 |
| 4 | 1565 | Ac-LTF$4rn6AYWAQL$4a5SAL-NH2 |
| 5 | 1566 | Ac-LTF$4rn6AYWAQL$4a5SAM-NH2 |
| 6 | 1567 | Ac-LTF$4rn6AYWAQL$4a5SAhL-NH2 |
| 7 | 1568 | Ac-LTF$4rn6AYWAQL$4a5SAF-NH2 |
| 8 | 1569 | Ac-LTF$4rn6AYWAQL$4a5SAI-NH2 |
| 9 | 1570 | Ac-LTF$4rn6AYWAQL$4a5SAChg-NH2 |
| 10 | 1571 | Ac-LTF$4rn6AYWAQL$4a5SAAib-NH2 |
| 11 | 1572 | Ac-LTF$4rn6AYWAQL$4a5SAA-NH2 |
| 12 | 1573 | Ac-LTF$4rn6AYWA$4a5L$S$Nle-NH2 |
| 13 | 1574 | Ac-LTF$4rn6AYWA$4a5L$S$A-NH2 |
| 14 | 1575 | Ac-F$4rn6AYWEAc3cL$4a5AANle-NH2 |
| 15 | 1576 | Ac-F$4rn6AYWEAc3cL$4a5AAL-NH2 |
| 16 | 1577 | Ac-F$4rn6AYWEAc3cL$4a5AAM-NH2 |
| 17 | 1578 | Ac-F$4rn6AYWEAc3cL$4a5AAhL-NH2 |
| 18 | 1579 | Ac-F$4rn6AYWEAc3cL$4a5AAF-NH2 |
| 19 | 1580 | Ac-F$4rn6AYWEAc3cL$4a5AAI-NH2 |
| 20 | 1581 | Ac-F$4rn6AYWEAc3cL$4a5AAChg-NH2 |
| 21 | 1582 | Ac-F$4rn6AYWEAc3cL$4a5AACha-NH2 |
| 22 | 1583 | Ac-F$4rn6AYWEAc3cL$4a5AAAib-NH2 |
| 23 | 1584 | Ac-LTF$4rn6AYWAQL$4a5AAAibV-NH2 |
| 24 | 1585 | Ac-LTF$4rn6AYWAQL$4a5AAAibV-NH2 |
| 25 | 1586 | Ac-LTF$4rn6AYWAQL$4a5SAibAA-NH2 |
| 26 | 1587 | Ac-LTF$4rn6AYWAQL$4a5SAibAA-NH2 |
| 27 | 1588 | Ac-HLTF$4rn6HHWHQL$4a5AANleNle-NH2 |
| 28 | 1589 | Ac-DLTF$4rn6HHWHQL$4a5RRLV-NH2 |
| 29 | 1590 | Ac-HHTF$4rn6HHWHQL$4a5AAML-NH2 |
| 30 | 1591 | Ac-F$4rn6HHWHQL$4a5RRDCha-NH2 |
| 31 | 1592 | Ac-F$4rn6HHWHQL$4a5HRFV-NH2 |
| 32 | 1593 | Ac-HLTF$4rn6HHWHQL$4a5AAhLA-NH2 |
| 33 | 1594 | Ac-DLTF$4rn6HHWHQL$4a5RRChgl-NH2 |
| 34 | 1595 | Ac-DLTF$4rn6HHWHQL$4a5RRChgl-NH2 |
| 35 | 1596 | Ac-HHTF$4rn6HHWHQL$4a5AAChav-NH2 |
| 36 | 1597 | Ac-F$4rn6HHWHQL$4a5RRDa-NH2 |
| 37 | 1598 | Ac-F$4rn6HHWHQL$4a5HRAibG-NH2 |
| 38 | 1599 | Ac-F$4rn6AYWAQL$4a5HHNleL-NH2 |
| 39 | 1600 | Ac-F$4rn6AYWSAL$4a5HQANle-NH2 |
| 40 | 1601 | Ac-F$4rn6AYWVQL$4a5QHChgl-NH2 |
| 41 | 1602 | Ac-F$4rn6AYWTAL$4a5QQNlev-NH2 |
| 42 | 1603 | Ac-F$4rn6AYWYQL$4a5HAibAa-NH2 |
| 43 | 1604 | Ac-LTF$4rn6AYWAQL$4a5HHLa-NH2 |
| 44 | 1605 | Ac-LTF$4rn6AYWAQL$4a5HHLa-NH2 |
| 45 | 1606 | Ac-LTF$4rn6AYWAQL$4a5HQNlev-NH2 |
| 46 | 1607 | Ac-LTF$4rn6AYWAQL$4a5HQNlev-NH2 |
| 47 | 1608 | Ac-LTF$4rn6AYWAQL$4a5QQMl-NH2 |
| 48 | 1609 | Ac-LTF$4rn6AYWAQL$4a5QQMl-NH2 |
| 49 | 1610 | Ac-LTF$4rn6AYWAQL$4a5HAibhLV-NH2 |
| 50 | 1611 | Ac-LTF$4rn6AYWAQL$4a5AHFA-NH2 |
| 51 | 1612 | Ac-HLTF$4rn6HHWHQL$4a5AANlel-NH2 |
| 52 | 1613 | Ac-DLTF$4rn6HHWHQL$4a5RRLa-NH2 |
| 53 | 1614 | Ac-HHTF$4rn6HHWHQL$4a5AAMv-NH2 |
| 54 | 1615 | Ac-F$4rn6HHWHQL$4a5RRDA-NH2 |
| 55 | 1616 | Ac-F$4rn6HHWHQL$4a5HRFCha-NH2 |
| 56 | 1617 | Ac-F$4rn6AYWEAL$4a5AA-NHAm |
| 57 | 1618 | Ac-F$4rn6AYWEAL$4a5AA-NHiAm |
| 58 | 1619 | Ac-F$4rn6AYWEAL$4a5AA-NHnPr3Ph |
| 59 | 1620 | Ac-F$4rn6AYWEAL$4a5AA-NHnBu33Me |
| 60 | 1621 | Ac-F$4rn6AYWEAL$4a5AA-NHnPr |
| 61 | 1622 | Ac-F$4rn6AYWEAL$4a5AA-NHnEt2Ch |
| 62 | 1623 | Ac-F$4rn6AYWEAL$4a5AA-NHnEt2Cp |
| 63 | 1624 | Ac-F$4rn6AYWEAL$4a5AA-NHHex |
| 64 | 1625 | Ac-LTF$4rn6AYWAQL$4a5AAIA-NH2 |
| 65 | 1626 | Ac-LTF$4rn6AYWAQL$4a5AAIA-NH2 |
| 66 | 1627 | Ac-LTF$4rn6AYWAAL$4a5AAMA-NH2 |
| 67 | 1628 | Ac-LTF$4rn6AYWAAL$4a5AAMA-NH2 |
| 68 | 1629 | Ac-LTF$4rn6AYWAQL$4a5AANleA-NH2 |
| 69 | 1630 | Ac-LTF$4rn6AYWAQL$4a5AANleA-NH2 |
| 70 | 1631 | Ac-LTF$4rn6AYWAQL$4a5AAIa-NH2 |
| 71 | 1632 | Ac-LTF$4rn6AYWAQL$4a5AAIa-NH2 |
| 72 | 1633 | Ac-LTF$4rn6AYWAAL$4a5AAMa-NH2 |
| 73 | 1634 | Ac-LTF$4rn6AYWAAL$4a5AAMa-NH2 |
| 74 | 1635 | Ac-LTF$4rn6AYWAQL$4a5AANlea-NH2 |
| 75 | 1636 | Ac-LTF$4rn6AYWAQL$4a5AANlea-NH2 |
| 76 | 1637 | Ac-LTF$4rn6AYWAAL$4a5AAIv-NH2 |
| 77 | 1638 | Ac-LTF$4rn6AYWAAL$4a5AAIv-NH2 |
| 78 | 1639 | Ac-LTF$4rn6AYWAQL$4a5AAMv-NH2 |
| 79 | 1640 | Ac-LTF$4rn6AYWAAL$4a5AANlev-NH2 |
| 80 | 1641 | Ac-LTF$4rn6AYWAAL$4a5AANlev-NH2 |
| 81 | 1642 | Ac-LTF$4rn6AYWAQL$4a5AAIl-NH2 |
| 82 | 1643 | Ac-LTF$4rn6AYWAQL$4a5AAIl-NH2 |
| 83 | 1644 | Ac-LTF$4rn6AYWAAL$4a5AAMl-NH2 |
| 84 | 1645 | Ac-LTF$4rn6AYWAQL$4a5AANlel-NH2 |
| 85 | 1646 | Ac-LTF$4rn6AYWAQL$4a5AANlel-NH2 |
| 86 | 1647 | Ac-F$4rn6AYWEAL$4a5AAMA-NH2 |
| 87 | 1648 | Ac-F$4rn6AYWEAL$4a5AANleA-NH2 |
| 88 | 1649 | Ac-F$4rn6AYWEAL$4a5AAIa-NH2 |
| 89 | 1650 | Ac-F$4rn6AYWEAL$4a5AAMa-NH2 |
| 90 | 1651 | Ac-F$4rn6AYWEAL$4a5AANlea-NH2 |
| 91 | 1652 | Ac-F$4rn6AYWEAL$4a5AAIv-NH2 |
| 92 | 1653 | Ac-F$4rn6AYWEAL$4a5AAMv-NH2 |
| 93 | 1654 | Ac-F$4rn6AYWEAL$4a5AANlev-NH2 |
| 94 | 1655 | Ac-F$4rn6AYWEAL$4a5AAIl-NH2 |
| 95 | 1656 | Ac-F$4rn6AYWEAL$4a5AAMl-NH2 |
| 96 | 1657 | Ac-F$4rn6AYWEAL$4a5AANlel-NH2 |
| 97 | 1658 | Ac-F$4rn6AYWEAL$4a5AANlel-NH2 |
| 98 | 1659 | Ac-LTF$4rn6AY6clWAQL$4a5SAA-NH2 |
| 99 | 1660 | Ac-LTF$4rn6AY6clWAQL$4a5SAA-NH2 |
| 100 | 1661 | Ac-WTF$4rn6FYWSQL$4a5AVAa-NH2 |
| 101 | 1662 | Ac-WTF$4rn6FYWSQL$4a5AVAa-NH2 |
| 102 | 1663 | Ac-WTF$4rn6VYWSQL$4a5AVA-NH2 |
| 103 | 1664 | Ac-WTF$4rn6VYWSQL$4a5AVA-NH2 |
| 104 | 1665 | Ac-WTF$4rn6FYWSQL$4a5SAAa-NH2 |
| 105 | 1666 | Ac-WTF$4rn6FYWSQL$4a5SAAa-NH2 |
| 106 | 1667 | Ac-WTF$4rn6VYWSQL$4a5AVAaa-NH2 |
| 107 | 1668 | Ac-WTF$4rn6VYWSQL$4a5AVAaa-NH2 |
| 108 | 1669 | Ac-LTF$4rn6AYWAQL$4a5AVG-NH2 |
| 109 | 1670 | Ac-LTF$4rn6AYWAQL$4a5AVG-NH2 |
| 110 | 1671 | Ac-LTF$4rn6AYWAQL$4a5AVQ-NH2 |
| 111 | 1672 | Ac-LTF$4rn6AYWAQL$4a5AVQ-NH2 |
| 112 | 1673 | Ac-LTF$4rn6AYWAQL$4a5SAa-NH2 |
| 113 | 1674 | Ac-LTF$4rn6AYWAQL$4a5SAa-NH2 |
| 114 | 1675 | Ac-LTF$4rn6AYWAQhL$4a5SAA-NH2 |
| 115 | 1676 | Ac-LTF$4rn6AYWAQhL$4a5SAA-NH2 |
| 116 | 1677 | Ac-LTF$4rn6AYWEQLStSA$4a5-NH2 |
| 117 | 1678 | Ac-LTF$4rn6AYWAQL$4a5SLA-NH2 |
| 118 | 1679 | Ac-LTF$4rn6AYWAQL$4a5SLA-NH2 |
| 119 | 1680 | Ac-LTF$4rn6AYWAQL$4a5SWA-NH2 |
| 120 | 1681 | Ac-LTF$4rn6AYWAQL$4a5SWA-NH2 |
| 121 | 1682 | Ac-LTF$4rn6AYWAQL$4a5SVS-NH2 |
| 122 | 1683 | Ac-LTF$4rn6AYWAQL$4a5SAS-NH2 |
| 123 | 1684 | Ac-LTF$4rn6AYWAQL$4a5SVG-NH2 |
| 124 | 1685 | Ac-ETF$4rn6VYWAQL$4a5SAa-NH2 |
| 125 | 1686 | Ac-ETF$4rn6VYWAQL$4a5SAA-NH2 |
| 126 | 1687 | Ac-ETF$4rn6VYWAQL$4a5SVA-NH2 |
| 127 | 1688 | Ac-ETF$4rn6VYWAQL$4a5SLA-NH2 |
| 128 | 1689 | Ac-ETF$4rn6VYWAQL$4a5SWA-NH2 |
| 129 | 1690 | Ac-ETF$4rn6KYWAQL$4a5SWA-NH2 |
| 130 | 1691 | Ac-ETF$4rn6VYWAQL$4a5SVS-NH2 |
| 131 | 1692 | Ac-ETF$4rn6VYWAQL$4a5SAS-NH2 |
| 132 | 1693 | Ac-ETF$4rn6VYWAQL$4a5SVG-NH2 |
| 133 | 1694 | Ac-LTF$4rn6VYWAQL$4a5SSa-NH2 |
| 134 | 1695 | Ac-ETF$4rn6VYWAQL$4a5SSa-NH2 |
| 135 | 1696 | Ac-LTF$4rn6VYWAQL$4a5SNa-NH2 |
| 136 | 1697 | Ac-ETF$4rn6VYWAQL$4a5SNa-NH2 |
| 137 | 1698 | Ac-LTF$4rn6VYWAQL$4a5SAa-NH2 |
| 138 | 1699 | Ac-LTF$4rn6VYWAQL$4a5SVA-NH2 |
| 139 | 1700 | Ac-LTF$4rn6VYWAQL$4a5SVA-NH2 |
| 140 | 1701 | Ac-LTF$4rn6VYWAQL$4a5SWA-NH2 |
| 141 | 1702 | Ac-LTF$4rn6VYWAQL$4a5SVS-NH2 |
| 142 | 1703 | Ac-LTF$4rn6VYWAQL$4a5SVS-NH2 |
| 143 | 1704 | Ac-LTF$4rn6VYWAQL$4a5SAS-NH2 |
| 144 | 1705 | Ac-LTF$4rn6VYWAQL$4a5SAS-NH2 |
| 145 | 1706 | Ac-LTF$4rn6VYWAQL$4a5SVG-NH2 |
| 146 | 1707 | Ac-LTF$4rn6VYWAQL$4a5SVG-NH2 |
| 147 | 1708 | Ac-LTF$4rn6EYWAQCha$4a5SAA-NH2 |
| 148 | 1709 | Ac-LTF$4rn6EYWAQCha$4a5SAA-NH2 |
| 149 | 1710 | Ac-LTF$4rn6EYWAQCpg$4a5SAA-NH2 |
| 150 | 1711 | Ac-LTF$4rn6EYWAQCpg$4a5SAA-NH2 |
| 151 | 1712 | Ac-LTF$4rn6EYWAQF$4a5SAA-NH2 |
| 152 | 1713 | Ac-LTF$4rn6EYWAQF$4a5SAA-NH2 |
| 153 | 1714 | Ac-LTF$4rn6EYWAQCba$4a5SAA-NH2 |
| 154 | 1715 | Ac-LTF$4rn6EYWAQCba$4a5SAA-NH2 |
| 155 | 1716 | Ac-LTF3Cl$4rn6EYWAQL$4a5SAA-NH2 |
| 156 | 1717 | Ac-LTF3Cl$4rn6EYWAQL$4a5SAA-NH2 |
| 157 | 1718 | Ac-LTF34F2$4rn6EYWAQL$4a5SAA-NH2 |
| 158 | 1719 | Ac-LTF34F2$4rn6EYWAQL$4a5SAA-NH2 |
| 159 | 1720 | Ac-LTF34F2$4rn6EYWAQhL$4a5SAA-NH2 |
| 160 | 1721 | Ac-LTF34F2$4rn6EYWAQhL$4a5SAA-NH2 |
| 161 | 1722 | Ac-ETF$4rn6EYWAQL$4a5SAA-NH2 |
| 162 | 1723 | Ac-LTF$4rn6AYWVQL$4a5SAA-NH2 |
| 163 | 1724 | Ac-LTF$4rn6AHWAQL$4a5SAA-NH2 |
| 164 | 1725 | Ac-LTF$4rn6AEWAQL$4a5SAA-NH2 |
| 165 | 1726 | Ac-LTF$4rn6ASWAQL$4a5SAA-NH2 |
| 166 | 1727 | Ac-LTF$4rn6AEWAQL$4a5SAA-NH2 |
| 167 | 1728 | Ac-LTF$4rn6ASWAQL$4a5SAA-NH2 |
| 168 | 1729 | Ac-LTF$4rn6AF4coohWAQL$4a5SAA-NH2 |
| 169 | 1730 | Ac-LTF$4rn6AF4coohWAQL$4a5SAA-NH2 |
| 170 | 1731 | Ac-LTF$4rn6AHWAQL$4a5AAIa-NH2 |
| 171 | 1732 | Ac-ITF$4rn6FYWAQL$4a5AAIa-NH2 |
| 172 | 1733 | Ac-ITF$4rn6EHWAQL$4a5AAIa-NH2 |
| 173 | 1734 | Ac-ITF$4rn6EHWAQL$4a5AAIa-NH2 |
| 174 | 1735 | Ac-ETF$4rn6EHWAQL$4a5AAIa-NH2 |
| 175 | 1736 | Ac-ETF$4rn6EHWAQL$4a5AAIa-NH2 |
| 176 | 1737 | Ac-LTF$4rn6AHWVQL$4a5AAIa-NH2 |
| 177 | 1738 | Ac-ITF$4rn6FYWVQL$4a5AAIa-NH2 |
| 178 | 1739 | Ac-ITF$4rn6EYWVQL$4a5AAIa-NH2 |
| 179 | 1740 | Ac-ITF$4rn6EHWVQL$4a5AAIa-NH2 |
| 180 | 1741 | Ac-LTF$4rn6AEWAQL$4a5AAIa-NH2 |
| 181 | 1742 | Ac-LTF$4rn6AF4coohWAQL$4a5AAIa-NH2 |
| 182 | 1743 | Ac-LTF$4rn6AF4coohWAQL$4a5AAIa-NH2 |
| 183 | 1744 | Ac-LTF$4rn6AHWAQL$4a5AHFA-NH2 |
| 184 | 1745 | Ac-ITF$4rn6FYWAQL$4a5AHFA-NH2 |
| 185 | 1746 | Ac-ITF$4rn6FYWAQL$4a5AHFA-NH2 |
| 186 | 1747 | Ac-ITF$4rn6FHWAQL$4a5AEFA-NH2 |
| 187 | 1748 | Ac-ITF$4rn6FHWAQL$4a5AEFA-NH2 |
| 188 | 1749 | Ac-ITF$4rn6EHWAQL$4a5AHFA-NH2 |
| 189 | 1750 | Ac-ITF$4rn6EHWAQL$4a5AHFA-NH2 |
| 190 | 1751 | Ac-LTF$4rn6AHWVQL$4a5AHFA-NH2 |
| 191 | 1752 | Ac-ITF$4rn6FYWVQL$4a5AHFA-NH2 |
| 192 | 1753 | Ac-ITF$4rn6EYWVQL$4a5AHFA-NH2 |
| 193 | 1754 | Ac-ITF$4rn6EHWVQL$4a5AHFA-NH2 |
| 194 | 1755 | Ac-ITF$4rn6EHWVQL$4a5AHFA-NH2 |
| 195 | 1756 | Ac-ETF$4rn6EYWAAL$4a5SAA-NH2 |
| 196 | 1757 | Ac-LTF$4rn6AYWVAL$4a5SAA-NH2 |
| 197 | 1758 | Ac-LTF$4rn6AHWAAL$4a5SAA-NH2 |
| 198 | 1759 | Ac-LTF$4rn6AEWAAL$4a5SAA-NH2 |
| 199 | 1760 | Ac-LTF$4rn6AEWAAL$4a5SAA-NH2 |
| 200 | 1761 | Ac-LTF$4rn6ASWAAL$4a5SAA-NH2 |
| 201 | 1762 | Ac-LTF$4rn6ASWAAL$4a5SAA-NH2 |
| 202 | 1763 | Ac-LTF$4rn6AYWAAL$4a5AAIa-NH2 |
| 203 | 1764 | Ac-LTF$4rn6AYWAAL$4a5AAIa-NH2 |
| 204 | 1765 | Ac-LTF$4rn6AYWAAL$4a5AHFA-NH2 |
| 205 | 1766 | Ac-LTF$4rn6EHWAQL$4a5AHIa-NH2 |
| 206 | 1767 | Ac-LTF$4rn6EHWAQL$4a5AHIa-NH2 |
| 207 | 1768 | Ac-LTF$4rn6AHWAQL$4a5AHIa-NH2 |
| 208 | 1769 | Ac-LTF$4rn6EYWAQL$4a5AHIa-NH2 |
| 209 | 1770 | Ac-LTF$4rn6AYWAQL$4a5AAFa-NH2 |
| 210 | 1771 | Ac-LTF$4rn6AYWAQL$4a5AAFa-NH2 |
| 211 | 1772 | Ac-LTF$4rn6AYWAQL$4a5AAWa-NH2 |
| 212 | 1773 | Ac-LTF$4rn6AYWAQL$4a5AAVa-NH2 |
| 213 | 1774 | Ac-LTF$4rn6AYWAQL$4a5AAVa-NH2 |
| 214 | 1775 | Ac-LTF$4rn6AYWAQL$4a5AALa-NH2 |
| 215 | 1776 | Ac-LTF$4rn6AYWAQL$4a5AALa-NH2 |
| 216 | 1777 | Ac-LTF$4rn6EYWAQL$4a5AAIa-NH2 |
| 217 | 1778 | Ac-LTF$4rn6EYWAQL$4a5AAIa-NH2 |
| 218 | 1779 | Ac-LTF$4rn6EYWAQL$4a5AAFa-NH2 |
| 219 | 1780 | Ac-LTF$4rn6EYWAQL$4a5AAFa-NH2 |
| 220 | 1781 | Ac-LTF$4rn6EYWAQL$4a5AAVa-NH2 |
| 221 | 1782 | Ac-LTF$4rn6EYWAQL$4a5AAVa-NH2 |
| 222 | 1783 | Ac-LTF$4rn6EHWAQL$4a5AAIa-NH2 |
| 223 | 1784 | Ac-LTF$4rn6EHWAQL$4a5AAIa-NH2 |
| 224 | 1785 | Ac-LTF$4rn6EHWAQL$4a5AAWa-NH2 |
| 225 | 1786 | Ac-LTF$4rn6EHWAQL$4a5AAWa-NH2 |
| 226 | 1787 | Ac-LTF$4rn6EHWAQL$4a5AALa-NH2 |
| 227 | 1788 | Ac-LTF$4rn6EHWAQL$4a5AALa-NH2 |
| 228 | 1789 | Ac-ETF$4rn6EHWVQL$4a5AALa-NH2 |
| 229 | 1790 | Ac-LTF$4rn6AYWAQL$4a5AAAa-NH2 |
| 230 | 1791 | Ac-LTF$4rn6AYWAQL$4a5AAAa-NH2 |
| 231 | 1792 | Ac-LTF$4rn6AYWAQL$4a5AAAibA-NH2 |
| 232 | 1793 | Ac-LTF$4rn6AYWAQL$4a5AAAibA-NH2 |
| 233 | 1794 | Ac-LTF$4rn6AYWAQL$4a5AAAAa-NH2 |
| 234 | 1795 | Ac-LTF$r5AYWAQL$4a5s8AAIa-NH2 |
| 235 | 1796 | Ac-LTF$r5AYWAQL$4a5s8SAA-NH2 |
| 236 | 1797 | Ac-LTF$4rn6AYWAQCba$4a5AANleA-NH2 |
| 237 | 1798 | Ac-ETF$4rn6AYWAQCba$4a5AANleA-NH2 |
| 238 | 1799 | Ac-LTF$4rn6EYWAQCba$4a5AANleA-NH2 |
| 239 | 1800 | Ac-LTF$4rn6AYWAQCba$4a5AWNleA-NH2 |
| 240 | 1801 | Ac-ETF$4rn6AYWAQCba$4a5AWNleA-NH2 |
| 241 | 1802 | Ac-LTF$4rn6EYWAQCba$4a5AWNleA-NH2 |
| 242 | 1803 | Ac-LTF$4rn6EYWAQCba$4a5SAFA-NH2 |
| 243 | 1804 | Ac-LTF34F2$4rn6EYWAQCba$4a5SANleA-NH2 |
| 244 | 1805 | Ac-LTF$4rn6EF4coohWAQCba$4a5SANleA-NH2 |
| 245 | 1806 | Ac-LTF$4rn6EYWSQCba$4a5SANleA-NH2 |
| 246 | 1807 | Ac-LTF$4rn6EYWWQCba$4a5SANleA-NH2 |
| 247 | 1808 | Ac-LTF$4rn6EYWAQCba$4a5AAIa-NH2 |
| 248 | 1809 | Ac-LTF34F2$4rn6EYWAQCba$4a5AAIa-NH2 |
| 249 | 1810 | Ac-LTF$4rn6EF4coohWAQCba$4a5AAIa-NH2 |
| 250 | 1811 | Pam-ETF$4rn6EYWAQCba$4a5SAA-NH2 |
| 251 | 1812 | Ac-LThF$4rn6EFWAQCba$4a5SAA-NH2 |
| 252 | 1813 | Ac-LTA$4rn6EYWAQCba$4a5SAA-NH2 |
| 253 | 1814 | Ac-LTF$4rn6EYAAQCba$4a5SAA-NH2 |
| 254 | 1815 | Ac-LTF$4rn6EY2NalAQCba$4a5SAA-NH2 |
| 255 | 1816 | Ac-LTF$4rn6AYWAQCba$4a5SAA-NH2 |
| 256 | 1817 | Ac-LTF$4rn6EYWAQCba$4a5SAF-NH2 |
| 257 | 1818 | Ac-LTF$4rn6EYWAQCba$4a5SAFa-NH2 |
| 258 | 1819 | Ac-LTF$4rn6AYWAQCba$4a5SAF-NH2 |
| 259 | 1820 | Ac-LTF34F2$4rn6AYWAQCba$4a5SAF-NH2 |
| 260 | 1821 | Ac-LTF$4rn6AF4coohWAQCba$4a5SAF-NH2 |
| 261 | 1822 | Ac-LTF$4rn6EY6clWAQCba$4a5SAF-NH2 |
| 262 | 1823 | Ac-LTF$4rn6AYWSQCba$4a5SAF-NH2 |
| 263 | 1824 | Ac-LTF$4rn6AYWWQCba$4a5SAF-NH2 |
| 264 | 1825 | Ac-LTF$4rn6AYWAQCba$4a5AAIa-NH2 |
| 265 | 1826 | Ac-LTF34F2$4rn6AYWAQCba$4a5AAIa-NH2 |
| 266 | 1827 | Ac-LTF$4rn6AY6clWAQCba$4a5AAIa-NH2 |
| 267 | 1828 | Ac-LTF$4rn6AF4coohWAQCba$4a5AAIa-NH2 |
| 268 | 1829 | Ac-LTF$4rn6EYWAQCba$4a5AAFa-NH2 |
| 269 | 1830 | Ac-LTF$4rn6EYWAQCba$4a5AAFa-NH2 |
| 270 | 1831 | Ac-ETF$4rn6AYWAQCba$4a5AWNlea-NH2 |
| 271 | 1832 | Ac-LTF$4rn6EYWAQCba$4a5AWNlea-NH2 |
| 272 | 1833 | Ac-ETF$4rn6EYWAQCba$4a5AWNlea-NH2 |
| 273 | 1834 | Ac-ETF$4rn6EYWAQCba$4a5AWNlea-NH2 |
| 274 | 1835 | Ac-LTF$4rn6AYWAQCba$4a5SAFa-NH2 |
| 275 | 1836 | Ac-LTF$4rn6AYWAQCba$4a5SAFa-NH2 |
| 276 | 1837 | Ac-ETF$4rn6AYWAQL$4a5AWNlea-NH2 |
| 277 | 1838 | Ac-LTF$4rn6EYWAQL$4a5AWNlea-NH2 |
| 278 | 1839 | Ac-ETF$4rn6EYWAQL$4a5AWNlea-NH2 |
| 279 | 1840 | Dmaac-LTF$4rn6EYWAQhL$4a5SAA-NH2 |
| 280 | 1841 | Hexac-LTF$4rn6EYWAQhL$4a5SAA-NH2 |
| 281 | 1842 | Napac-LTF$4rn6EYWAQhL$4a5SAA-NH2 |
| 282 | 1843 | Decac-LTF$4rn6EYWAQhL$4a5SAA-NH2 |
| 283 | 1844 | Admac-LTF$4rn6EYWAQhL$4a5SAA-NH2 |
| 284 | 1845 | Tmac-LTF$4rn6EYWAQhL$4a5SAA-NH2 |
| 285 | 1846 | Pam-LTF$4rn6EYWAQhL$4a5SAA-NH2 |
| 286 | 1847 | Ac-LTF$4rn6AYWAQCba$4a5AANleA-NH2 |
| 287 | 1848 | Ac-LTF34F2$4rn6EYWAQCba$4a5AAIa-NH2 |
| 288 | 1849 | Ac-LTF34F2$4rn6EYWAQCba$4a5SAA-NH2 |
| 289 | 1850 | Ac-LTF34F2$4rn6EYWAQCba$4a5SAA-NH2 |
| 290 | 1851 | Ac-LTF$4rn6EF4coohWAQCba$4a5SAA-NH2 |
| 291 | 1852 | Ac-LTF$4rn6EF4coohWAQCba$4a5SAA-NH2 |
| 292 | 1853 | Ac-LTF$4rn6EYWSQCba$4a5SAA-NH2 |
| 293 | 1854 | Ac-LTF$4rn6EYWSQCba$4a5SAA-NH2 |
| 294 | 1855 | Ac-LTF$4rn6EYWAQhL$4a5SAA-NH2 |
| 295 | 1856 | Ac-LTF$4rn6AYWAQhL$4a5SAF-NH2 |
| 296 | 1857 | Ac-LTF$4rn6AYWAQhL$4a5SAF-NH2 |
| 297 | 1858 | Ac-LTF34F2$4rn6AYWAQhL$4a5SAA-NH2 |
| 298 | 1859 | Ac-LTF34F2$4rn6AYWAQhL$4a5SAA-NH2 |
| 299 | 1860 | Ac-LTF$4rn6AF4coohWAQhL$4a5SAA-NH2 |
| 300 | 1861 | Ac-LTF$4rn6AF4coohWAQhL$4a5SAA-NH2 |
| 301 | 1862 | Ac-LTF$4rn6AYWSQhL$4a5SAA-NH2 |
| 302 | 1863 | Ac-LTF$4rn6AYWSQhL$4a5SAA-NH2 |
| 303 | 1864 | Ac-LTF$4rn6EYWAQL$4a5AANleA-NH2 |
| 304 | 1865 | Ac-LTF34F2$4rn6AYWAQL$4a5AANleA-NH2 |
| 305 | 1866 | Ac-LTF$4rn6AF4coohWAQL$4a5AANleA-NH2 |
| 306 | 1867 | Ac-LTF$4rn6AYWSQL$4a5AANleA-NH2 |
| 307 | 1868 | Ac-LTF34F2$4rn6AYWAQhL$4a5AANleA-NH2 |
| 308 | 1869 | Ac-LTF34F2$4rn6AYWAQhL$4a5AANleA-NH2 |
| 309 | 1870 | Ac-LTF$4rn6AF4coohWAQhL$4a5AANleA-NH2 |
| 310 | 1871 | Ac-LTF$4rn6AF4coohWAQhL$4a5AANleA-NH2 |
| 311 | 1872 | Ac-LTF$4rn6AYWSQhL$4a5AANleA-NH2 |
| 312 | 1873 | Ac-LTF$4rn6AYWSQhL$4a5AANleA-NH2 |
| 313 | 1874 | Ac-LTF$4rn6AYWAQhL$4a5AAAAa-NH2 |
| 314 | 1875 | Ac-LTF$4rn6AYWAQhL$4a5AAAAa-NH2 |
| 315 | 1876 | Ac-LTF$4rn6AYWAQL$4a5AAAAAa-NH2 |
| 316 | 1877 | Ac-LTF$4rn6AYWAQL$4a5AAAAAAa-NH2 |
| 317 | 1878 | Ac-LTF$4rn6AYWAQL$4a5AAAAAAa-NH2 |
| 318 | 1879 | Ac-LTF$4rn6EYWAQhL$4a5AANleA-NH2 |
| 319 | 1880 | Ac-AATF$4rn6AYWAQL$4a5AANleA-NH2 |
| 320 | 1881 | Ac-LTF$4rn6AYWAQL$4a5AANleAA-NH2 |
| 321 | 1882 | Ac-ALTF$4rn6AYWAQL$4a5AANleAA-NH2 |
| 322 | 1883 | Ac-LTF$4rn6AYWAQCba$4a5AANleAA-NH2 |
| 323 | 1884 | Ac-LTF$4rn6AYWAQhL$4a5AANleAA-NH2 |
| 324 | 1885 | Ac-LTF$4rn6EYWAQCba$4a5SAAA-NH2 |
| 325 | 1886 | Ac-LTF$4rn6EYWAQCba$4a5SAAA-NH2 |
| 326 | 1887 | Ac-LTF$4rn6EYWAQCba$4a5SAAAA-NH2 |
| 327 | 1888 | Ac-LTF$4rn6EYWAQCba$4a5SAAAA-NH2 |
| 328 | 1889 | Ac-ALTF$4rn6EYWAQCba$4a5SAA-NH2 |
| 329 | 1890 | Ac-ALTF$4rn6EYWAQCba$4a5SAAA-NH2 |
| 330 | 1891 | Ac-ALTF$4rn6EYWAQCba$4a5SAA-NH2 |
| 331 | 1892 | Ac-LTF$4rn6EYWAQL$4a5AAAAAa-NH2 |
| 332 | 1893 | Ac-LTF$4rn6EY6clWAQCba$4a5SAA-NH2 |
| 333 | 1894 | Ac-LTF$4rn6EF4cooh6clWAQCba$4a5SANleA-NH2 |
| 334 | 1895 | Ac-LTF$4rn6EF4cooh6clWAQCba$4a5SANleA-NH2 |
| 335 | 1896 | Ac-LTF$4rn6EF4cooh6clWAQCba$4a5AAIa-NH2 |
| 336 | 1897 | Ac-LTF$4rn6EF4cooh6clWAQCba$4a5AAIa-NH2 |
| 337 | 1898 | Ac-LTF$4rn6AY6clWAQL$4a5AAAAAa-NH2 |
| 338 | 1899 | Ac-LTF$4rn6AY6clWAQL$4a5AAAAAa-NH2 |
| 339 | 1900 | Ac-F$4rn6AY6clWEAL$4a5AAAAAAa-NH2 |
| 340 | 1901 | Ac-ETF$4rn6EYWAQL$4a5AAAAAa-NH2 |
| 341 | 1902 | Ac-ETF$4rn6EYWAQL$4a5AAAAAa-NH2 |
| 342 | 1903 | Ac-LTF$4rn6EYWAQL$4a5AAAAAAa-NH2 |
| 343 | 1904 | Ac-LTF$4rn6EYWAQL$4a5AAAAAAa-NH2 |
| 344 | 1905 | Ac-LTF$4rn6AYWAQL$4a5AANleAAa-NH2 |
| 345 | 1906 | Ac-LTF$4rn6AYWAQL$4a5AANleAAa-NH2 |
| 346 | 1907 | Ac-LTF$4rn6EYWAQCba$4a5AAAAAa-NH2 |
| 347 | 1908 | Ac-LTF$4rn6EYWAQCba$4a5AAAAAa-NH2 |
| 348 | 1909 | Ac-LTF$4rn6EF4coohWAQCba$4a5AAAAAa-NH2 |
| 349 | 1910 | Ac-LTF$4rn6EF4coohWAQCba$4a5AAAAAa-NH2 |
| 350 | 1911 | Ac-LTF$4rn6EYWSQCba$4a5AAAAAa-NH2 |
| 351 | 1912 | Ac-LTF$4rn6EYWSQCba$4a5AAAAAa-NH2 |
| 352 | 1913 | Ac-LTF$4rn6EYWAQCba$4a5SAAa-NH2 |
| 353 | 1914 | Ac-LTF$4rn6EYWAQCba$4a5SAAa-NH2 |
| 354 | 1915 | Ac-ALTF$4rn6EYWAQCba$4a5SAAa-NH2 |
| 355 | 1916 | Ac-ALTF$4rn6EYWAQCba$4a5SAAa-NH2 |
| 356 | 1917 | Ac-ALTF$4rn6EYWAQCba$4a5SAAAa-NH2 |
| 357 | 1918 | Ac-ALTF$4rn6EYWAQCba$4a5SAAAa-NH2 |
| 358 | 1919 | Ac-AALTF$4rn6EYWAQCba$4a5SAAAa-NH2 |
| 359 | 1920 | Ac-AALTF$4rn6EYWAQCba$4a5SAAAa-NH2 |
| 360 | 1921 | Ac-RTF$4rn6EYWAQCba$4a5SAA-NH2 |
| 361 | 1922 | Ac-LRF$4rn6EYWAQCba$4a5SAA-NH2 |
| 362 | 1923 | Ac-LTF$4rn6EYWRQCba$4a5SAA-NH2 |
| 363 | 1924 | Ac-LTF$4rn6EYWARCba$4a5SAA-NH2 |
| 364 | 1925 | Ac-LTF$4rn6EYWAQCba$4a5RAA-NH2 |
| 365 | 1926 | Ac-LTF$4rn6EYWAQCba$4a5SRA-NH2 |
| 366 | 1927 | Ac-LTF$4rn6EYWAQCba$4a5SAR-NH2 |
| 367 | 1928 | 5-FAM-BaLTF$4rn6EYWAQCba$4a5SAA-NH2 |
| 368 | 1929 | 5-FAM-BaLTF$4rn6AYWAQL$4a5AANleA-NH2 |
| 369 | 1930 | Ac-LAF$4rn6EYWAQL$4a5AANleA-NH2 |
| 370 | 1931 | Ac-ATF$4rn6EYWAQL$4a5AANleA-NH2 |
| 371 | 1932 | Ac-AAF$4rn6EYWAQL$4a5AANleA-NH2 |
| 372 | 1933 | Ac-AAAF$4rn6EYWAQL$4a5AANleA-NH2 |
| 373 | 1934 | Ac-AAAAF$4rn6EYWAQL$4a5AANleA-NH2 |
| 374 | 1935 | Ac-AATF$4rn6EYWAQL$4a5AANleA-NH2 |
| 375 | 1936 | Ac-AALTF$4rn6EYWAQL$4a5AANleA-NH2 |
| 376 | 1937 | Ac-AAALTF$4rn6EYWAQL$4a5AANleA-NH2 |
| 377 | 1938 | Ac-LTF$4rn6EYWAQL$4a5AANleAA-NH2 |
| 378 | 1939 | Ac-ALTF$4rn6EYWAQL$4a5AANleAA-NH2 |
| 379 | 1940 | Ac-AALTF$4rn6EYWAQL$4a5AANleAA-NH2 |
| 380 | 1941 | Ac-LTF$4rn6EYWAQCba$4a5AANleAA-NH2 |
| 381 | 1942 | Ac-LTF$4rn6EYWAQhL$4a5AANleAA-NH2 |
| 382 | 1943 | Ac-ALTF$4rn6EYWAQhL$4a5AANleAA-NH2 |
| 383 | 1944 | Ac-LTF$4rn6ANmYWAQL$4a5AANleA-NH2 |
| 384 | 1945 | Ac-LTF$4rn6ANmYWAQL$4a5AANleA-NH2 |
| 385 | 1946 | Ac-LTF$4rn6AYNmWAQL$4a5AANleA-NH2 |
| 386 | 1947 | Ac-LTF$4rn6AYNmWAQL$4a5AANleA-NH2 |
| 387 | 1948 | Ac-LTF$4rn6AYAmwAQL$4a5AANleA-NH2 |
| 388 | 1949 | Ac-LTF$4rn6AYAmwAQL$4a5AANleA-NH2 |
| 389 | 1950 | Ac-LTF$4rn6AYWAibQL$4a5AANleA-NH2 |
| 390 | 1951 | Ac-LTF$4rn6AYWAibQL$4a5AANleA-NH2 |
| 391 | 1952 | Ac-LTF$4rn6AYWAQL$4a5AAibNleA-NH2 |
| 392 | 1953 | Ac-LTF$4rn6AYWAQL$4a5AAibNleA-NH2 |
| 393 | 1954 | Ac-LTF$4rn6AYWAQL$4a5AaNleA-NH2 |
| 394 | 1955 | Ac-LTF$4rn6AYWAQL$4a5AaNleA-NH2 |
| 395 | 1956 | Ac-LTF$4rn6AYWAQL$4a5ASarNleA-NH2 |
| 396 | 1957 | Ac-LTF$4rn6AYWAQL$4a5ASarNleA-NH2 |
| 397 | 1958 | Ac-LTF$4rn6AYWAQL$4a5AANleAib-NH2 |
| 398 | 1959 | Ac-LTF$4rn6AYWAQL$4a5AANleAib-NH2 |
| 399 | 1960 | Ac-LTF$4rn6AYWAQL$4a5AANleNmA-NH2 |
| 400 | 1961 | Ac-LTF$4rn6AYWAQL$4a5AANleNmA-NH2 |
| 401 | 1962 | Ac-LTF$4rn6AYWAQL$4a5AANleSar-NH2 |
| 402 | 1963 | Ac-LTF$4rn6AYWAQL$4a5AANleSar-NH2 |
| 403 | 1964 | Ac-LTF$4rn6AYWAQL$4a5AANleAAib-NH2 |
| 404 | 1965 | Ac-LTF$4rn6AYWAQL$4a5AANleAAib-NH2 |
| 405 | 1966 | Ac-LTF$4rn6AYWAQL$4a5AANleANmA-NH2 |
| 406 | 1967 | Ac-LTF$4rn6AYWAQL$4a5AANleANmA-NH2 |
| 407 | 1968 | Ac-LTF$4rn6AYWAQL$4a5AANleAa-NH2 |
| 408 | 1969 | Ac-LTF$4rn6AYWAQL$4a5AANleAa-NH2 |
| 409 | 1970 | Ac-LTF$4rn6AYWAQL$4a5AANleASar-NH2 |
| 410 | 1971 | Ac-LTF$4rn6AYWAQL$4a5AANleASar-NH2 |
| 413 | 1972 | Ac-LTF$4rn6Cou4YWAQL$4a5AANleA-NH2 |
| 414 | 1973 | Ac-LTF$4rn6Cou4YWAQL$4a5AANleA-NH2 |
| 415 | 1974 | Ac-LTF$4rn6AYWCou4QL$4a5AANleA-NH2 |
| 416 | 1975 | Ac-LTF$4rn6AYWAQL$4a5Cou4ANleA-NH2 |
| 417 | 1976 | Ac-LTF$4rn6AYWAQL$4a5Cou4ANleA-NH2 |
| 418 | 1977 | Ac-LTF$4rn6AYWAQL$4a5ACou4NleA-NH2 |
| 419 | 1978 | Ac-LTF$4rn6AYWAQL$4a5ACou4NleA-NH2 |
| 420 | 1979 | Ac-LTF$4rn6AYWAQL$4a5AANleA-OH |
| 421 | 1980 | Ac-LTF$4rn6AYWAQL$4a5AANleA-OH |
| 422 | 1981 | Ac-LTF$4rn6AYWAQL$4a5AANleA-NHnPr |
| 423 | 1982 | Ac-LTF$4rn6AYWAQL$4a5AANleA-NHnPr |
| 424 | 1983 | Ac-LTF$4rn6AYWAQL$4a5AANleA-NHnBu33Me |
| 425 | 1984 | Ac-LTF$4rn6AYWAQL$4a5AANleA-NHnBu33Me |
| 426 | 1985 | Ac-LTF$4rn6AYWAQL$4a5AANleA-NHHex |
| 427 | 1986 | Ac-LTF$4rn6AYWAQL$4a5AANleA-NHHex |
| 428 | 1987 | Ac-LTA$4rn6AYWAQL$4a5AANleA-NH2 |
| 429 | 1988 | Ac-LThL$4rn6AYWAQL$4a5AANleA-NH2 |
| 430 | 1989 | Ac-LTF$4rn6AYAAQL$4a5AANleA-NH2 |
| 431 | 1990 | Ac-LTF$4rn6AY2NalAQL$4a5AANleA-NH2 |
| 432 | 1991 | Ac-LTF$4rn6EYWCou4QCba$4a5SAA-NH2 |
| 433 | 1992 | Ac-LTF$4rn6EYWCou7QCba$4a5SAA-NH2 |
| 435 | 1993 | Dmaac-LTF$4rn6EYWAQCba$4a5SAA-NH2 |
| 436 | 1994 | Dmaac-LTF$4rn6AYWAQL$4a5AAAAAa-NH2 |
| 437 | 1995 | Dmaac-LTF$4rn6AYWAQL$4a5AAAAAa-NH2 |
| 438 | 1996 | Dmaac-LTF$4rn6EYWAQL$4a5AAAAAa-NH2 |
| 439 | 1997 | Dmaac-LTF$4rn6EYWAQL$4a5AAAAAa-NH2 |
| 440 | 1998 | Dmaac-LTF$4rn6EF4coohWAQCba$4a5AAIa-NH2 |
| 441 | 1999 | Dmaac-LTF$4rn6EF4coohWAQCba$4a5AAIa-NH2 |
| 442 | 2000 | Dmaac-LTF$4rn6AYWAQL$4a5AANleA-NH2 |
| 443 | 2001 | Dmaac-LTF$4rn6AYWAQL$4a5AANleA-NH2 |
| 444 | 2002 | Ac-LTF$4rn6AYWAQL$4a5AANleA-NH2 |
| 445 | 2003 | Ac-LTF$4rn6EYWAQL$4a5AAAAAa-NH2 |
| 446 | 2004 | Cou6BaLTF$4rn6EYWAQhL$4a5SAA-NH2 |
| 447 | 2005 | Cou8BaLTF$4rn6EYWAQhL$4a5SAA-NH2 |
| 448 | 2006 | Ac-LTF4I$4rn6EYWAQL$4a5AAAAAa-NH2 |

TABLE 17

| | SEQ | | Exact | Found | Calc | Calc | Calc |
|---|---|---|---|---|---|---|---|
| SP | ID NO: | Sequence | Mass | Mass | (M + 1)/1 | (M + 2)/2 | (M + 3)/3 |
| 449 | 2007 | Ac-LTF$4rn6AYWAQL$4a5AANleA-NH2 | 1812.01 | 907.89 | 1813.02 | 907.01 | 605.01 |
| 450 | 2008 | Ac-LTF$4rn6AYWAQL$4a5AAAAAa-NH2 | 1912.04 | 957.75 | 1913.05 | 957.03 | 638.35 |
| 451 | 2009 | Ac-LTF$4rn6EYWAQL$4a5AAAAAa-NH2 | 1970.04 | 986.43 | 1971.05 | 986.03 | 657.69 |
| 452 | 2010 | Ac-LTF$5rn6AYWAQL$5a5AAAAAa-NH2 | 1912.04 | 957.38 | 1913.05 | 957.03 | 638.35 |
| 153 | 2011 | Ac-LTF$4rn6EYWAQCba$4a5SAA-NH2 | 1784.93 | 894.38 | 1785.94 | 893.47 | 595.98 |
| 454 | 2012 | Ac-LTF$4rn4EYWAQCba$4a5SAA-NH2 | 1756.89 | 880.05 | 1757.9 | 879.45 | 586.64 |
| 455 | 2013 | Ac-LTF$4rn5EYWAQCba$4a5SAA-NH2 | 1770.91 | 887.08 | 1771.92 | 886.46 | 591.31 |
| 456 | 2014 | Ac-LTF$5rn6EYWAQCba$5a5SAA-NH2 | 1784.92 | 894.11 | 1785.93 | 893.47 | 595.98 |
| 457 | 2015 | Ac-LTF$4rn6EYWAQCba5I-$4a5SAA-NH2 | 1910.82 | 957.01 | 1911.83 | 956.42 | 637.95 |
| 459 | 2016 | Ac-LTA$5rn6EYWAQCba$5a5SAA-NH2 | 1708.89 | 856 | 1709.9 | 855.45 | 570.64 |
| 460 | 2017 | Ac-LTA$4rn6EYWAQCba$4a5SAA-NH2 | 1708.89 | 856 | 1709.9 | 855.45 | 570.64 |
| 461 | 2018 | 5-FAM-BaLTF$4rn6EYWAQCba$4a5SAA-NH2 | 2172 | 1087.81 | 2173.01 | 1087.01 | 725.01 |
| 462 | 2019 | 5-FAM-BaLTA$4rn6EYWAQCba$4a5SAA-NH2 | 2095.97 | 1049.79 | 2096.98 | 1048.99 | 699.66 |
| 463 | 2020 | 5-FAM-BaLTF$5rn6EYWAQCba$5a5SAA-NH2 | 2172 | 1087.53 | 2173.01 | 1087.01 | 725.01 |
| 464 | 2021 | 5-FAM-BaLTA$5rn6EYWAQCba$5a5SAA-NH2 | 2095.97 | 1049.98 | 2096.98 | 1048.99 | 699.66 |
| 465 | 2022 | Ac-LTF$4rn6EYWAQCba5Ph-$4a5SAA-NH2 | 1675.87 | 932.31 | 1676.88 | 931.48 | 559.63 |
| 466 | 2023 | Ac-LTF$4rn6EYWAQCba5Prp-$4a5SAA-NH2 | 1675.87 | 914.46 | 1676.88 | 913.48 | 559.63 |
| 467 | 2024 | Ac-LTF$4rn6AYWAAL$4a5AAAAAa-NH2 | 1855.01 | | 1856.02 | 928.51 | 619.34 |
| 468 | 2025 | Ac-LTF$4rn6EYWAQCba5penNH2-$4a5SAA-NH2 | 1675.87 | | 1676.88 | 838.94 | 559.63 |
| 469 | 2026 | Ac-LTF$4rn6EYWAQCba5BnzNH2-$4a5SAA-NH2 | 1675.87 | | 1676.88 | 838.94 | 559.63 |
| 470 | 2027 | Ac-LTF$4rn6EYWAQCba5prpOMe-$4a5SAA-NH2 | | 929.17 | | 928.48 | |
| 932 | 2028 | Ac-LTF$5rn6EYWAQL4Me$5a5AAAAAa-NH2 | 1926.05 | | 1927.06 | 964.03 | 643.02 |
| 933 | 2029 | Ac-LTF$5rn6EYWAQL4Ph$5a5AAAAAa-NH2 | 1988.07 | | 1989.07 | 995.04 | 663.70 |
| 934 | 2030 | Ac-LTF$5rn6EYWAQCba4Me$5a5SAANH2 | 1740.93 | | 1741.94 | 871.48 | 581.32 |
| 935 | 2031 | Ac-LTF$5rn6EYWAQCba4Ph$5a5SAANH2 | 1802.95 | | 1803.96 | 902.48 | 601.99 |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon crosslinker comprising one double bond. Amino acids represented as “$r5” are alpha-Me R5-pentenyl-alanine olefin amino acids connected by an all-carbon comprising one double bond. Amino acids represented as “$s8” are alpha-Me S8-octenyl-alanine olefin amino acids connected by an all-carbon crosslinker comprising one double bond. Amino acids represented as “$r8” are alpha-Me R8-octenyl-alanine olefin amino acids connected by an all-carbon crosslinker comprising one double bond. “Ahx” represents an aminocyclohexyl linker. The crosslinkers are linear all-carbon crosslinker comprising eight or eleven carbon atoms between the alpha carbons of each amino acid. Amino acids represented as “$/” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker. Amino acids represented as “Amw” are alpha-Me tryptophan amino acids. Amino acids represented as “Aml” are alpha-Me leucine amino acids. Amino acids represented as “Amf” are alpha-Me phenylalanine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “St//” are amino acids comprising two pentenyl-alanine olefin side chains that are not crosslinked. Amino acids represented as “% St” are amino acids comprising two pentenyl-alanine olefin side chains, each of which is crosslinked to another amino acid as indicated via fully saturated hydrocarbon crosslinks. Amino acids represented as “Ba” are beta-alanine. The lower-case character “e” or “z” within the designation of a crosslinked amino acid (e.g. “$er8” or “$zr8”) represents the configuration of the double bond (E or Z, re ectively). In other contexts, lower-case letters such as “a” or “f” represent D amino acids (e.g. D-alanine, or D-phenylalanine, respectively). Amino acids designated as “NmW” represent N-methyltryptophan. Amino acids designated as “NmY” represent N-methyltyrosine. Amino acids designated as “NmA” represent N-methylalanine. Amino acids designated as “Sar” represent sarcosine. Amino acids designated as “Cha” represent cyclohexyl alanine. Amino acids designated as “Cpg” represent cyclopentyl glycine. Amino acids designated as “Chg” represent cyclohexyl glycine. Amino acids designated as “Cba” represent cyclobutyl alanine. Amino acids designated as “F4I” represent 4-iodo phenylalanine. Amino acids designated as “F3Cl” represent 3-chloro phenylalanine. Amino acids designated as “F4cooh” represent 4-carboxy phenylalanine. Amino acids designated as “F34F2” represent 3,4-difluoro phenylalanine. Amino acids designated as “6clW” represent 6-chloro tryptophan. The designation “iso1” or “iso2” indicates that the peptidomimetic macrocycle is a single isomer. “Ac3c” represents an aminocyclopropane carboxylic acid residue.

Amino acids forming crosslinkers are represented according to the legend indicated below.

Stereochemistry at the alpha position of each amino acid is S unless otherwise indicated. Amino acids labeled “4Me” were prepared using an amino acid comprising an alkyne which was methyl-substituted (internal alkyne), resulting in triazole groups comprising a methyl group at the 4-position. Amino acids labeled “4Ph” were prepared using an amino acid comprising an alkyne which was phenyl-substituted (internal alkyne), resulting in triazole groups comprising a phenyl group at the 4-position. For azide amino acids, the number of carbon atoms indicated refers to the number of methylene units between the alpha carbon and the terminal azide. For alkyne amino acids, the number of carbon atoms indicated is the number of methylene units between the alpha position and the triazole moiety plus the two carbon atoms within the triazole group derived from the alkyne.

| | |
|---|---|
| $5n3 | Alpha-Me azide 1,5 triazole (3 carbon) |
| #5n3 | Alpha-H azide 1,5 triazole (3 carbon) |
| $4a5 | Alpha-Me alkyne 1,4 triazole (5 carbon) |
| $4a6 | Alpha-Me alkyne 1,4 triazole (6 carbon) |
| $5a5 | Alpha-Me alkyne 1,5 triazole (5 carbon) |
| $5a6 | Alpha-Me alkyne 1,5 triazole (6 carbon) |
| #4a5 | Alpha-H alkyne 1,4 triazole (5 carbon) |
| #5a5 | Alpha-H alkyne 1,5 triazole (5 carbon) |
| $5n5 | Alpha-Me azide 1,5 triazole (5 carbon) |
| $5n6 | Alpha-Me azide 1,5 triazole (6 carbon) |
| $4n5 | Alpha-Me azide 1,4 triazole (5 carbon) |
| $4n6 | Alpha-Me azide 1,4 triazole (6 carbon) |
| $4ra5 | Alpha-Me R-alkyne 1,4 triazole (5 carbon) |
| $4ra6 | Alpha-Me R-alkyne 1,4 triazole (6 carbon) |
| $4rn4 | Alpha-Me R-azide 1,4 triazole (4 carbon) |
| $4rn5 | Alpha-Me R-azide 1,4 triazole (5 carbon) |
| $4rn6 | Alpha-Me R-azide 1,4 triazole (6 carbon) |
| $5rn5 | Alpha-Me R-azide 1,5 triazole (5 carbon) |
| $5ra5 | Alpha-Me R-alkyne 1,5 triazole (5 carbon) |
| $5ra6 | Alpha-Me R-alkyne 1,5 triazole (6 carbon) |
| $5rn6 | Alpha-Me R-azide 1,5 triazole (6 carbon) |
| #5rn6 | Alpha-H R-azide 1,5 triazole (6 carbon) |
| $4rn5 | Alpha-Me R-azide 1,4 triazole (5 carbon) |
| #4rn5 | Alpha-H R-azide 1,4 triazole (5 carbon) |
| 4Me$5rn6 | Alpha-Me R-azide 1,5 triazole (6 carbon); |
| | 4-Me substituted triazole |
| 4Me$5a5 | Alpha-Me alkyne 1,5 triazole (5 carbon); |
| | 4-Me substituted triazole |
| 4Ph$5a5 | Alpha-Me alkyne 1,5 triazole (5 carbon); |
| | 4-phenyl substituted triazole |

Amino acids designated as “5I”, “5penNH2”, “5BnzNH2”, “5prpOMe”, “5Ph”, and “5prp”, refer to crosslinked amino acids of the type shown in the following exemplary peptidomimetic macrocycle (SEQ ID NO: 2032):

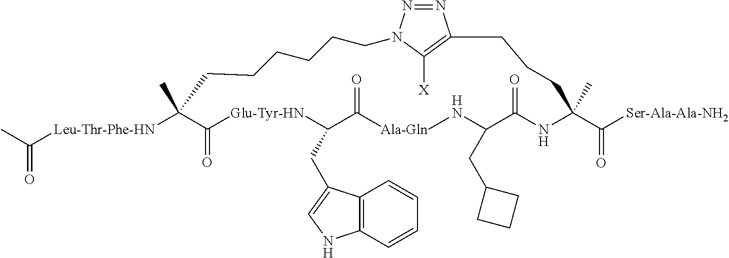

In the above structure, X is, for example, one of the following substituents:

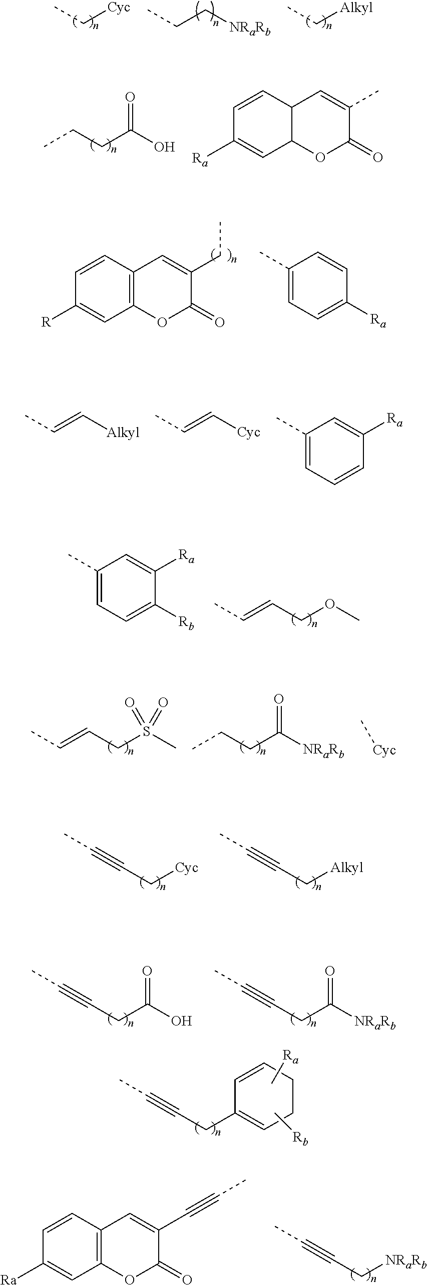

wherein “Cyc” is a suitable aryl, cycloalkyl, cycloalkenyl, heteroaryl, or heterocyclyl group, unsubstituted or optionally substituted with an Rₐ or R_{b} group as described above.

In some embodiments, the triazole substituent is chosen from the group consisting of:

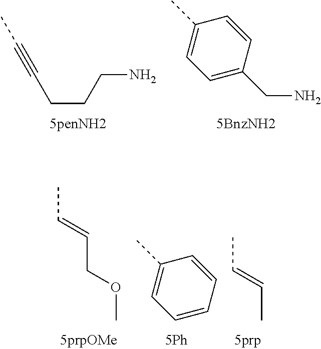

Table 18 shows exemplary peptidomimetic macrocycles:

TABLE 18
(SEQ ID NOS 2033-2038, respectively, in order of appearance)

| | Structure |
|---|---|
| SP-449 | 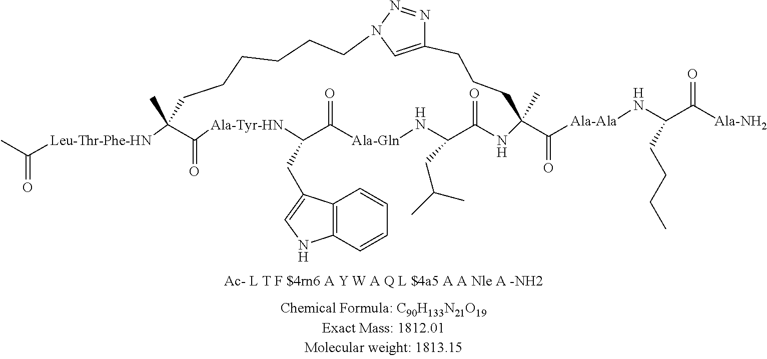 |
| SP-64 | 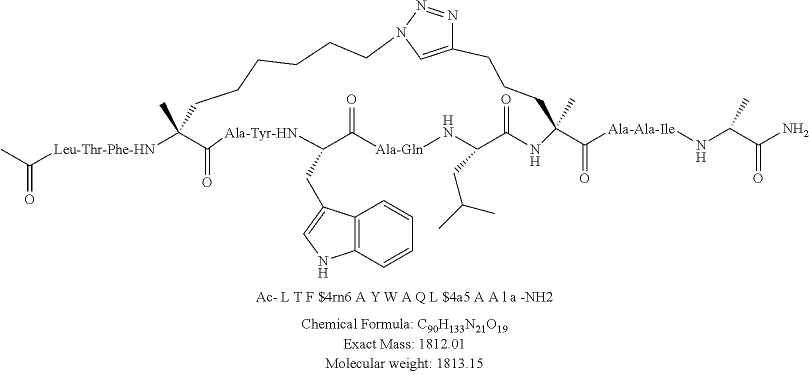 |
| SP-153 | 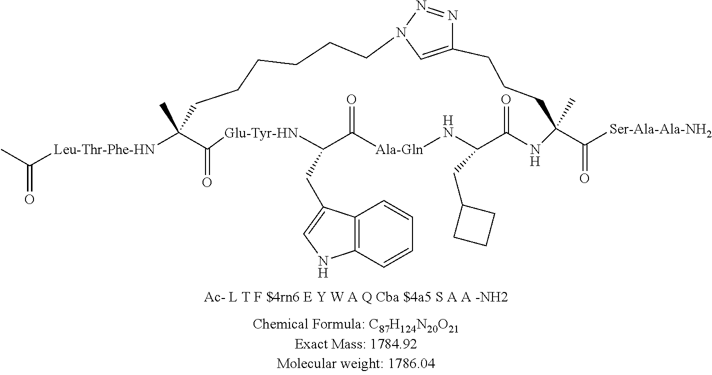 |
| SP-98 | 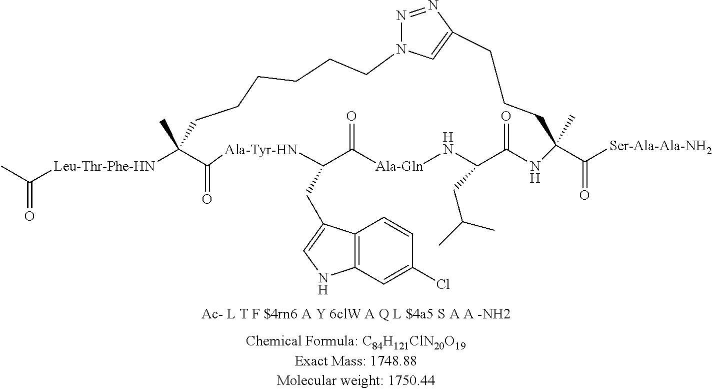 |
| SP-456 | 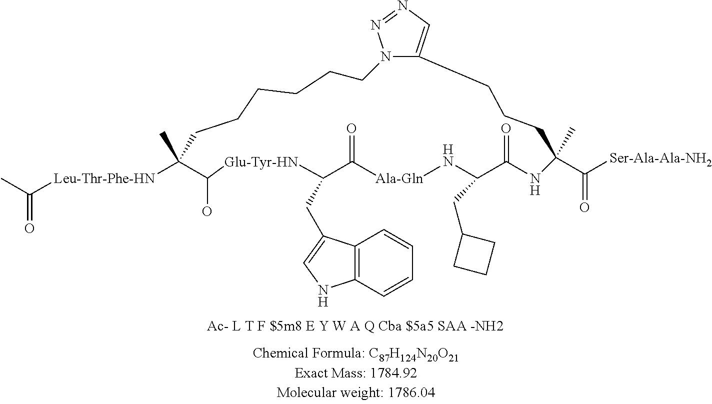 |
| SP-470 | 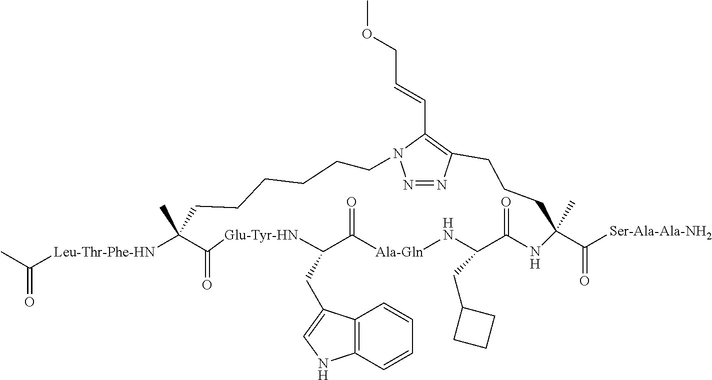 |

In some embodiments, peptidomimetic macrocycles exclude peptidomimetic macrocycles shown in Table 19. Peptides shown can comprise an N-terminal capping group such as acetyl or an additional linker such as beta-alanine between the capping group and the start of the peptide sequence. In some embodiments, peptidomimetic macrocycles do not comprise a peptidomimetic macrocycle structure as shown in Table 19.

TABLE 19

| | SEQ | |
|---|---|---|
| # | ID NO: | Sequence |
| 1 | 2039 | Ac-QSQQTF$5rn6NLWRLL$5a5QN-NH2 |
| 2 | 2040 | Ac-QSQQTF$4rn5NLWRLL$4a5QN-NH2 |
| 3 | 2041 | Ac-QSQQTF#5rn6NLWRLL#5a5QN-NH2 |
| 4 | 2042 | Ac-QSQQTF#4rn5NLWRLL#4a5QN-NH2 |
| 5 | 2043 | Ac-QSQQTF$5rn5NLWRLL$5a5QN-NH2 |
| 6 | 2044 | Ac-QSQQTF$5ra5NLWRLL$5n5QN-NH2 |
| 7 | 2045 | Ac-QSQQTF$5ra5NLWRLL$5n6QN-NH2 |
| 8 | 2046 | Ac-QSQQTF$4ra5NLWRLL$4n5QN-NH2 |
| 9 | 2047 | Ac-QSQQTF$4ra5NLWRLL$4n6QN-NH2 |
| 10 | 2048 | Ac-QSQQTF$4rn6NLWRLL$4a5QN-NH2 |
| 11 | 2049 | Ac-QSQQTF$5rn6NLWRLL$5a6QN-NH2 |
| 12 | 2050 | Ac-QSQQTF$5ra6NLWRLL$5n6QN-NH2 |
| 13 | 2051 | Ac-QSQQTF$4rn6NLWRLL$4a6QN-NH2 |
| 14 | 2052 | Ac-QSQQTF$4ra6NLWRLL$4n6QN-NH2 |
| 15 | 2053 | Ac-QSQQTF$4rn5NLWRLL$4a6QN-NH2 |
| 16 | 2054 | Ac-QSQQTF4Me$5rn6NLWRLL4Me$5a5QN-NH2 |
| 17 | 2055 | Ac-LTF$4ra5HYWAQL$4n6S-NH2 |
| 18 | 2056 | H-F$4rn6HYWAQL$4a5S-NH2 |
| 19 | 2057 | Ac-LTF$4rn6HYWAQL$4a5S-NH2 |
| 20 | 2058 | Ac-F$4rn6HYWAQL$4a5S-NH2 |
| 21 | 2059 | Ac-LTF$4rn6HYWAQL$4a6S-NH2 |
| 22 | 2060 | Ac-LTF$5ra5HYWAQL$5n6S-NH2 |
| 23 | 2061 | Ac-LTF$4rn6AYWAQL$4a5A-NH2 |
| 24 | 2062 | Ac-LTF$5ra5HYWAQL$5n6S-NH2 |
| 25 | 2063 | Ac-LTF$4rn6AYWAQL$4a5A-NH2 |
| 26 | 2064 | Ac-LTFEHYWAQLTS-NH2 |

The fully protected resin-bound peptides are synthesized on a Rink amide MBHA resin (loading 0.62 mmol/g) on a 0.1 mmol scale. Deprotection of the temporary Fmoc group is achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate preactivated Fmoc-amino acid derivative. All protected amino acids (1 mmol) were dissolved in NMP and activated with HCTU (1 mmol) and DIEA (1 mmol) prior to transfer of the coupling solution to the deprotected resin-bound peptide. After coupling was completed, the resin was extensively flow washed in preparation for the next deprotection/coupling cycle. Acetylation of the amino terminus was carried out in the presence of acetic anhydride/DIEA in NMP/NMM. The LC-MS analysis of a cleaved and deprotected sample obtained from an aliquot of the fully assembled resin-bound peptide was accomplished in order to verifying the completion of each coupling.

In a typical example, a peptide resin (0.1 mmol) was washed with DCM. Deprotection of the temporary Mmt group was achieved by 3×3 min treatments of the resin bound peptide with 2% TFA/DCM 5% TIPS, then 30 min treatments until no orange color is observed in the filtrate. In between treatments the resin was extensively flow washed with DCM. After complete removal of Mmt, the resin was washed with 5% DIEA/NMP solution 3× and considered ready for bisthioether coupling. Resin was loaded into a reaction vial. DCM/DMF 1/1 was added to the reaction vessel, followed by DIEA (2.4 eq). After mixing well for 5 minutes, 4,4′-Bis(bromomethyl)biphenyl (1.05 eq) (TCI America B1921) was added. The reaction was then mechanically agitated at room temperature overnight. Where needed, the reaction was allowed additional time to reach completion. A similar procedure may be used in the preparation of five-methylene, six-methylene or seven-methylene crosslinkers (“% c7”, “% c6”, or “% c5”).

The bisthioether resin-bound peptides were deprotected and cleaved from the solid support by treatment with TFA/H₂O/TIS (94/3/3 v/v) for 3 h at room temperature. After filtration of the resin the TFA solution was precipitated in cold diethyl ether and centrifuged to yield the desired product as a solid. The crude product was purified by preparative HPLC. Table 6 shows a list of peptidomimetic macrocycles.

TABLE 6

| | SEQ | |
|---|---|---|
| SP | ID NO: | Sequence |
| 471 | 2065 | Ac-F%cs7AYWEAc3cL%c7AAA-NH2 |
| 472 | 2066 | Ac-F%cs7AYWEAc3cL%c7AAibA-NH2 |
| 473 | 2067 | Ac-LTF%cs7AYWAQL%c7SANle-NH2 |
| 474 | 2068 | Ac-LTF%cs7AYWAQL%c7SAL-NH2 |
| 475 | 2069 | Ac-LTF%cs7AYWAQL%c7SAM-NH2 |
| 476 | 2070 | Ac-LTF%cs7AYWAQL%c7SAhL-NH2 |
| 477 | 2071 | Ac-LTF%cs7AYWAQL%c7SAF-NH2 |
| 478 | 2072 | Ac-LTF%cs7AYWAQL%c7SAI-NH2 |
| 479 | 2073 | Ac-LTF%cs7AYWAQL%c7SAChg-NH2 |
| 480 | 2074 | Ac-LTF%cs7AYWAQL%c7SAAib-NH2 |
| 481 | 2075 | Ac-LTF%cs7AYWAQL%c7SAA-NH2 |
| 482 | 2076 | Ac-LTF%cs7AYWA%c7L%c7S%c7Nle-NH2 |
| 483 | 2077 | Ac-LTF%cs7AYWA%c7L%c7S%c7A-NH2 |
| 484 | 2078 | Ac-F%cs7AYWEAc3cL%c7AANle-NH2 |
| 485 | 2079 | Ac-F%cs7AYWEAc3cL%c7AAL-NH2 |
| 486 | 2080 | Ac-F%cs7AYWEAc3cL%c7AAM-NH2 |
| 487 | 2081 | Ac-F%cs7AYWEAc3cL%c7AAhL-NH2 |
| 488 | 2082 | Ac-F%cs7AYWEAc3cL%c7AAF-NH2 |
| 489 | 2083 | Ac-F%cs7AYWEAc3cL%c7AAI-NH2 |
| 490 | 2084 | Ac-F%cs7AYWEAc3cL%c7AAChg-NH2 |
| 491 | 2085 | Ac-F%cs7AYWEAc3cL%c7AACha-NH2 |
| 492 | 2086 | Ac-F%cs7AYWEAc3cL%c7AAAib-NH2 |
| 493 | 2087 | Ac-LTF%cs7AYWAQL%c7AAAibV-NH2 |
| 494 | 2088 | Ac-LTF%cs7AYWAQL%c7AAAibV-NH2 |
| 495 | 2089 | Ac-LTF%cs7AYWAQL%c7SAibAA-NH2 |
| 496 | 2090 | Ac-LTF%cs7AYWAQL%c7SAibAA-NH2 |
| 497 | 2091 | Ac-HLTF%cs7HHWHQL%c7AANleNle-NH2 |
| 498 | 2092 | Ac-DLTF%cs7HHWHQL%c7RRLV-NH2 |
| 499 | 2093 | Ac-HHTF%cs7HHWHQL%c7AAML-NH2 |
| 500 | 2094 | Ac-F%cs7HHWHQL%c7RRDCha-NH2 |
| 501 | 2095 | Ac-F%cs7HHWHQL%c7HRFV-NH2 |
| 502 | 2096 | Ac-HLTF%cs7HHWHQL%c7AAhLA-NH2 |
| 503 | 2097 | Ac-DLTF%cs7HHWHQL%c7RRChgl-NH2 |
| 504 | 2098 | Ac-DLTF%cs7HHWHQL%c7RRChgl-NH2 |
| 505 | 2099 | Ac-HHTF%cs7HHWHQL%c7AAChav-NH2 |
| 506 | 2100 | Ac-F%cs7HHWHQL%c7RRDa-NH2 |
| 507 | 2101 | Ac-F%cs7HHWHQL%c7HRAibG-NH2 |
| 508 | 2102 | Ac-F%cs7AYWAQL%c7HHNleL-NH2 |
| 509 | 2103 | Ac-F%cs7AYWSAL%c7HQANle-NH2 |
| 510 | 2104 | Ac-F%cs7AYWVQL%c7QHChgl-NH2 |
| 511 | 2105 | Ac-F%cs7AYWTAL%c7QQNlev-NH2 |
| 512 | 2106 | Ac-F%cs7AYWYQL%c7HAibAa-NH2 |
| 513 | 2107 | Ac-LTF%cs7AYWAQL%c7HHLa-NH2 |
| 514 | 2108 | Ac-LTF%cs7AYWAQL%c7HHLa-NH2 |
| 515 | 2109 | Ac-LTF%cs7AYWAQL%c7HQNlev-NH2 |
| 516 | 2110 | Ac-LTF%cs7AYWAQL%c7HQNlev-NH2 |
| 517 | 2111 | Ac-LTF%cs7AYWAQL%c7QQMl-NH2 |
| 518 | 2112 | Ac-LTF%cs7AYWAQL%c7QQMl-NH2 |
| 519 | 2113 | Ac-LTF%cs7AYWAQL%c7HAibhLV-NH2 |
| 520 | 2114 | Ac-LTF%cs7AYWAQL%c7AHFA-NH2 |
| 521 | 2115 | Ac-HLTF%cs7HHWHQL%c7AANlel-NH2 |
| 522 | 2116 | Ac-DLTF%cs7HHWHQL%c7RRLa-NH2 |
| 523 | 2117 | Ac-HHTF%cs7HHWHQL%c7AAMv-NH2 |
| 524 | 2118 | Ac-F%cs7HHWHQL%c7RRDA-NH2 |
| 525 | 2119 | Ac-F%cs7HHWHQL%c7HRFCha-NH2 |
| 526 | 2120 | Ac-F%cs7AYWEAL%c7AA-NHAm |
| 527 | 2121 | Ac-F%cs7AYWEAL%c7AA-NHiAm |
| 528 | 2122 | Ac-F%cs7AYWEAL%c7AA-NHnPr3Ph |
| 529 | 2123 | Ac-F%cs7AYWEAL%c7AA-NHnBu33Me |
| 530 | 2124 | Ac-F%cs7AYWEAL%c7AA-NHnPr |
| 531 | 2125 | Ac-F%cs7AYWEAL%c7AA-NHnEt2Ch |
| 532 | 2126 | Ac-F%cs7AYWEAL%c7AA-NHnEt2Cp |
| 533 | 2127 | Ac-F%cs7AYWEAL%c7AA-NHHex |
| 534 | 2128 | Ac-LTF%cs7AYWAQL%c7AAIA-NH2 |
| 535 | 2129 | Ac-LTF%cs7AYWAQL%c7AAIA-NH2 |
| 536 | 2130 | Ac-LTF%cs7AYWAAL%c7AAMA-NH2 |
| 537 | 2131 | Ac-LTF%cs7AYWAAL%c7AAMA-NH2 |
| 538 | 2132 | Ac-LTF%cs7AYWAQL%c7AANleA-NH2 |
| 539 | 2133 | Ac-LTF%cs7AYWAQL%c7AANleA-NH2 |
| 540 | 2134 | Ac-LTF%cs7AYWAQL%c7AAIa-NH2 |
| 541 | 2135 | Ac-LTF%cs7AYWAQL%c7AAIa-NH2 |
| 542 | 2136 | Ac-LTF%cs7AYWAAL%c7AAMa-NH2 |
| 543 | 2137 | Ac-LTF%cs7AYWAAL%c7AAMa-NH2 |
| 544 | 2138 | Ac-LTF%cs7AYWAQL%c7AANlea-NH2 |
| 545 | 2139 | Ac-LTF%cs7AYWAQL%c7AANlea-NH2 |
| 546 | 2140 | Ac-LTF%cs7AYWAAL%c7AAIv-NH2 |
| 547 | 2141 | Ac-LTF%cs7AYWAAL%c7AAIv-NH2 |
| 548 | 2142 | Ac-LTF%cs7AYWAQL%c7AAMv-NH2 |
| 549 | 2143 | Ac-LTF%cs7AYWAAL%c7AANlev-NH2 |
| 550 | 2144 | Ac-LTF%cs7AYWAAL%c7AANlev-NH2 |
| 551 | 2145 | Ac-LTF%cs7AYWAQL%c7AAIl-NH2 |
| 552 | 2146 | Ac-LTF%cs7AYWAQL%c7AAIl-NH2 |
| 553 | 2147 | Ac-LTF%cs7AYWAAL%c7AAMl-NH2 |
| 554 | 2148 | Ac-LTF%cs7AYWAQL%c7AANlel-NH2 |
| 555 | 2149 | Ac-LTF%cs7AYWAQL%c7AANlel-NH2 |
| 556 | 2150 | Ac-F%cs7AYWEAL%c7AAMA-NH2 |
| 557 | 2151 | Ac-F%cs7AYWEAL%c7AANleA-NH2 |
| 558 | 2152 | Ac-F%cs7AYWEAL%c7AAIa-NH2 |
| 559 | 2153 | Ac-F%cs7AYWEAL%c7AAMa-NH2 |
| 560 | 2154 | Ac-F%cs7AYWEAL%c7AANlea-NH2 |
| 561 | 2155 | Ac-F%cs7AYWEAL%c7AAIv-NH2 |
| 562 | 2156 | Ac-F%cs7AYWEAL%c7AAMv-NH2 |
| 563 | 2157 | Ac-F%cs7AYWEAL%c7AANlev-NH2 |
| 564 | 2158 | Ac-F%cs7AYWEAL%c7AAIl-NH2 |
| 565 | 2159 | Ac-F%cs7AYWEAL%c7AAMl-NH2 |
| 566 | 2160 | Ac-F%cs7AYWEAL%c7AANlel-NH2 |
| 567 | 2161 | Ac-F%cs7AYWEAL%c7AANlel-NH2 |
| 568 | 2162 | Ac-LTF%cs7AY6clWAQL%c7SAA-NH2 |
| 569 | 2163 | Ac-LTF%cs7AY6clWAQL%c7SAA-NH2 |
| 570 | 2164 | Ac-WTF%cs7FYWSQL%c7AVAa-NH2 |
| 571 | 2165 | Ac-WTF%cs7FYWSQL%c7AVAa-NH2 |
| 572 | 2166 | Ac-WTF%cs7VYWSQL%c7AVA-NH2 |
| 573 | 2167 | Ac-WTF%cs7VYWSQL%c7AVA-NH2 |
| 574 | 2168 | Ac-WTF%cs7FYWSQL%c7SAAa-NH2 |
| 575 | 2169 | Ac-WTF%cs7FYWSQL%c7SAAa-NH2 |
| 576 | 2170 | Ac-WTF%cs7VYWSQL%c7AVAaa-NH2 |
| 577 | 2171 | Ac-WTF%cs7VYWSQL%c7AVAaa-NH2 |
| 578 | 2172 | Ac-LTF%cs7AYWAQL%c7AVG-NH2 |
| 579 | 2173 | Ac-LTF%cs7AYWAQL%c7AVG-NH2 |
| 580 | 2174 | Ac-LTF%cs7AYWAQL%c7AVQ-NH2 |
| 581 | 2175 | Ac-LTF%cs7AYWAQL%c7AVQ-NH2 |
| 582 | 2176 | Ac-LTF%cs7AYWAQL%c7SAa-NH2 |
| 583 | 2177 | Ac-LTF%cs7AYWAQL%c7SAa-NH2 |
| 584 | 2178 | Ac-LTF%cs7AYWAQhL%c7SAA-NH2 |
| 585 | 2179 | Ac-LTF%cs7AYWAQhL%c7SAA-NH2 |
| 586 | 2180 | Ac-LTF%cs7AYWEQLStSA%c7-NH2 |
| 587 | 2181 | Ac-LTF%cs7AYWAQL%c7SLA-NH2 |
| 588 | 2182 | Ac-LTF%cs7AYWAQL%c7SLA-NH2 |
| 589 | 2183 | Ac-LTF%cs7AYWAQL%c7SWA-NH2 |
| 590 | 2184 | Ac-LTF%cs7AYWAQL%c7SWA-NH2 |
| 591 | 2185 | Ac-LTF%cs7AYWAQL%c7SVS-NH2 |
| 592 | 2186 | Ac-LTF%cs7AYWAQL%c7SAS-NH2 |
| 593 | 2187 | Ac-LTF%cs7AYWAQL%c7SVG-NH2 |
| 594 | 2188 | Ac-ETF%cs7VYWAQL%c7SAa-NH2 |
| 595 | 2189 | Ac-ETF%cs7VYWAQL%c7SAA-NH2 |
| 596 | 2190 | Ac-ETF%cs7VYWAQL%c7SVA-NH2 |
| 597 | 2191 | Ac-ETF%cs7VYWAQL%c7SLA-NH2 |
| 598 | 2192 | Ac-ETF%cs7VYWAQL%c7SWA-NH2 |
| 599 | 2193 | Ac-ETF%cs7KYWAQL%c7SWA-NH2 |
| 600 | 2194 | Ac-ETF%cs7VYWAQL%c7SVS-NH2 |
| 601 | 2195 | Ac-ETF%cs7VYWAQL%c7SAS-NH2 |
| 602 | 2196 | Ac-ETF%cs7VYWAQL%c7SVG-NH2 |
| 603 | 2197 | Ac-LTF%cs7VYWAQL%c7SSa-NH2 |
| 604 | 2198 | Ac-ETF%cs7VYWAQL%c7SSa-NH2 |
| 605 | 2199 | Ac-LTF%cs7VYWAQL%c7SNa-NH2 |
| 606 | 2200 | Ac-ETF%cs7VYWAQL%c7SNa-NH2 |
| 607 | 2201 | Ac-LTF%cs7VYWAQL%c7SAa-NH2 |
| 608 | 2202 | Ac-LTF%cs7VYWAQL%c7SVA-NH2 |
| 609 | 2203 | Ac-LTF%cs7VYWAQL%c7SVA-NH2 |
| 610 | 2204 | Ac-LTF%cs7VYWAQL%c7SWA-NH2 |
| 611 | 2205 | Ac-LTF%cs7VYWAQL%c7SVS-NH2 |
| 612 | 2206 | Ac-LTF%cs7VYWAQL%c7SVS-NH2 |
| 613 | 2207 | Ac-LTF%cs7VYWAQL%c7SAS-NH2 |
| 614 | 2208 | Ac-LTF%cs7VYWAQL%c7SAS-NH2 |
| 615 | 2209 | Ac-LTF%cs7VYWAQL%c7SVG-NH2 |
| 616 | 2210 | Ac-LTF%cs7VYWAQL%c7SVG-NH2 |
| 617 | 2211 | Ac-LTF%cs7EYWAQCha%c7SAA-NH2 |
| 618 | 2212 | Ac-LTF%cs7EYWAQCha%c7SAA-NH2 |
| 619 | 2213 | Ac-LTF%cs7EYWAQCpg%c7SAA-NH2 |
| 620 | 2214 | Ac-LTF%cs7EYWAQCpg%c7SAA-NH2 |
| 621 | 2215 | Ac-LTF%cs7EYWAQF%c7SAA-NH2 |
| 622 | 2216 | Ac-LTF%cs7EYWAQF%c7SAA-NH2 |
| 623 | 2217 | Ac-LTF%cs7EYWAQCba%c7SAA-NH2 |
| 624 | 2218 | Ac-LTF%cs7EYWAQCba%c7SAA-NH2 |
| 625 | 2219 | Ac-LTF3Cl%cs7EYWAQL%c7SAA-NH2 |
| 626 | 2220 | Ac-LTF3Cl%cs7EYWAQL%c7SAA-NH2 |
| 627 | 2221 | Ac-LTF34F2%cs7EYWAQL%c7SAA-NH2 |
| 628 | 2222 | Ac-LTF34F2%cs7EYWAQL%c7SAA-NH2 |
| 629 | 2223 | Ac-LTF34F2%cs7EYWAQhL%c7SAA-NH2 |
| 630 | 2224 | Ac-LTF34F2%cs7EYWAQhL%c7SAA-NH2 |
| 631 | 2225 | Ac-ETF%cs7EYWAQL%c7SAA-NH2 |
| 632 | 2226 | Ac-LTF%cs7AYWVQL%c7SAA-NH2 |
| 633 | 2227 | Ac-LTF%cs7AHWAQL%c7SAA-NH2 |
| 634 | 2228 | Ac-LTF%cs7AEWAQL%c7SAA-NH2 |
| 635 | 2229 | Ac-LTF%cs7ASWAQL%c7SAA-NH2 |
| 636 | 2230 | Ac-LTF%cs7AEWAQL%c7SAA-NH2 |
| 637 | 2231 | Ac-LTF%cs7ASWAQL%c7SAA-NH2 |
| 638 | 2232 | Ac-LTF%cs7AF4coohWAQL%c7SAA-NH2 |
| 639 | 2233 | Ac-LTF%cs7AF4coohWAQL%c7SAA-NH2 |
| 640 | 2234 | Ac-LTF%cs7AHWAQL%c7AAIa-NH2 |
| 641 | 2235 | Ac-ITF%cs7FYWAQL%c7AAIa-NH2 |
| 642 | 2236 | Ac-ITF%cs7EHWAQL%c7AAIa-NH2 |
| 643 | 2237 | Ac-ITF%cs7EHWAQL%c7AAIa-NH2 |
| 644 | 2238 | Ac-ETF%cs7EHWAQL%c7AAIa-NH2 |
| 645 | 2239 | Ac-ETF%cs7EHWAQL%c7AAIa-NH2 |
| 646 | 2240 | Ac-LTF%cs7AHWVQL%c7AAIa-NH2 |
| 647 | 2241 | Ac-ITF%cs7FYWVQL%c7AAIa-NH2 |
| 648 | 2242 | Ac-ITF%cs7EYWVQL%c7AAIa-NH2 |
| 649 | 2243 | Ac-ITF%cs7EHWVQL%c7AAIa-NH2 |
| 650 | 2244 | Ac-LTF%cs7AEWAQL%c7AAIa-NH2 |
| 651 | 2245 | Ac-LTF%cs7AF4coohWAQL%c7AAIa-NH2 |
| 652 | 2246 | Ac-LTF%cs7AF4coohWAQL%c7AAIa-NH2 |
| 653 | 2247 | Ac-LTF%cs7AHWAQL%c7AHFA-NH2 |
| 654 | 2248 | Ac-ITF%cs7FYWAQL%c7AHFA-NH2 |
| 655 | 2249 | Ac-ITF%cs7FYWAQL%c7AHFA-NH2 |
| 656 | 2250 | Ac-ITF%cs7FHWAQL%c7AEFA-NH2 |
| 657 | 2251 | Ac-ITF%cs7FHWAQL%c7AEFA-NH2 |
| 658 | 2252 | Ac-ITF%cs7EHWAQL%c7AHFA-NH2 |
| 659 | 2253 | Ac-ITF%cs7EHWAQL%c7AHFA-NH2 |
| 660 | 2254 | Ac-LTF%cs7AHWVQL%c7AHFA-NH2 |
| 661 | 2255 | Ac-ITF%cs7FYWVQL%c7AHFA-NH2 |
| 662 | 2256 | Ac-ITF%cs7EYWVQL%c7AHFA-NH2 |
| 663 | 2257 | Ac-ITF%cs7EHWVQL%c7AHFA-NH2 |
| 664 | 2258 | Ac-ITF%cs7EHWVQL%c7AHFA-NH2 |
| 665 | 2259 | Ac-ETF%cs7EYWAAL%c7SAA-NH2 |
| 666 | 2260 | Ac-LTF%cs7AYWVAL%c7SAA-NH2 |
| 667 | 2261 | Ac-LTF%cs7AHWAAL%c7SAA-NH2 |
| 668 | 2262 | Ac-LTF%cs7AEWAAL%c7SAA-NH2 |
| 669 | 2263 | Ac-LTF%cs7AEWAAL%c7SAA-NH2 |
| 670 | 2264 | Ac-LTF%cs7ASWAAL%c7SAA-NH2 |
| 671 | 2265 | Ac-LTF%cs7ASWAAL%c7SAA-NH2 |
| 672 | 2266 | Ac-LTF%cs7AYWAAL%c7AAIa-NH2 |
| 673 | 2267 | Ac-LTF%cs7AYWAAL%c7AAIa-NH2 |
| 674 | 2268 | Ac-LTF%cs7AYWAAL%c7AHFA-NH2 |
| 675 | 2269 | Ac-LTF%cs7EHWAQL%c7AHIa-NH2 |
| 676 | 2270 | Ac-LTF%cs7EHWAQL%c7AHIa-NH2 |
| 677 | 2271 | Ac-LTF%cs7AHWAQL%c7AHIa-NH2 |
| 678 | 2272 | Ac-LTF%cs7EYWAQL%c7AHIa-NH2 |
| 679 | 2273 | Ac-LTF%cs7AYWAQL%c7AAFa-NH2 |
| 680 | 2274 | Ac-LTF%cs7AYWAQL%c7AAFa-NH2 |
| 681 | 2275 | Ac-LTF%cs7AYWAQL%c7AAWa-NH2 |
| 682 | 2276 | Ac-LTF%cs7AYWAQL%c7AAVa-NH2 |
| 683 | 2277 | Ac-LTF%cs7AYWAQL%c7AAVa-NH2 |
| 684 | 2278 | Ac-LTF%cs7AYWAQL%c7AALa-NH2 |
| 685 | 2279 | Ac-LTF%cs7AYWAQL%c7AALa-NH2 |
| 686 | 2280 | Ac-LTF%cs7EYWAQL%c7AAIa-NH2 |
| 687 | 2281 | Ac-LTF%cs7EYWAQL%c7AAIa-NH2 |
| 688 | 2282 | Ac-LTF%cs7EYWAQL%c7AAFa-NH2 |
| 689 | 2283 | Ac-LTF%cs7EYWAQL%c7AAFa-NH2 |
| 690 | 2284 | Ac-LTF%cs7EYWAQL%c7AAVa-NH2 |
| 691 | 2285 | Ac-LTF%cs7EYWAQL%c7AAVa-NH2 |
| 692 | 2286 | Ac-LTF%cs7EHWAQL%c7AAIa-NH2 |
| 693 | 2287 | Ac-LTF%cs7EHWAQL%c7AAIa-NH2 |
| 694 | 2288 | Ac-LTF%cs7EHWAQL%c7AAWa-NH2 |
| 695 | 2289 | Ac-LTF%cs7EHWAQL%c7AAWa-NH2 |
| 696 | 2290 | Ac-LTF%cs7EHWAQL%c7AALa-NH2 |
| 697 | 2291 | Ac-LTF%cs7EHWAQL%c7AALa-NH2 |
| 698 | 2292 | Ac-ETF%cs7EHWVQL%c7AALa-NH2 |
| 699 | 2293 | Ac-LTF%cs7AYWAQL%c7AAAa-NH2 |
| 700 | 2294 | Ac-LTF%cs7AYWAQL%c7AAAa-NH2 |
| 701 | 2295 | Ac-LTF%cs7AYWAQL%c7AAAibA-NH2 |
| 702 | 2296 | Ac-LTF%cs7AYWAQL%c7AAAibA-NH2 |
| 703 | 2297 | Ac-LTF%cs7AYWAQL%c7AAAAa-NH2 |
| 704 | 2298 | Ac-LTF%c7r5AYWAQL%c7s8AAIa-NH2 |
| 705 | 2299 | Ac-LTF%c7r5AYWAQL%c7s8SAA-NH2 |
| 706 | 2300 | Ac-LTF%cs7AYWAQCba%c7AANleA-NH2 |
| 707 | 2301 | Ac-ETF%cs7AYWAQCba%c7AANleA-NH2 |
| 708 | 2302 | Ac-LTF%cs7EYWAQCba%c7AANleA-NH2 |
| 709 | 2303 | Ac-LTF%cs7AYWAQCba%c7AWNleA-NH2 |
| 710 | 2304 | Ac-ETF%cs7AYWAQCba%c7AWNleA-NH2 |
| 711 | 2305 | Ac-LTF%cs7EYWAQCba%c7AWNleA-NH2 |
| 712 | 2306 | Ac-LTF%cs7EYWAQCba%c7SAFA-NH2 |
| 713 | 2307 | Ac-LTF34F2%cs7EYWAQCba%c7SANleA-NH2 |
| 714 | 2308 | Ac-LTF%cs7EF4coohWAQCba%c7SANleA-NH2 |
| 715 | 2309 | Ac-LTF%cs7EYWSQCba%c7SANleA-NH2 |
| 716 | 2310 | Ac-LTF%cs7EYWWQCba%c7SANleA-NH2 |
| 717 | 2311 | Ac-LTF%cs7EYWAQCba%c7AAIa-NH2 |
| 718 | 2312 | Ac-LTF34F2%cs7EYWAQCba%c7AAIa-NH2 |
| 719 | 2313 | Ac-LTF%cs7EF4coohWAQCba%c7AAIa-NH2 |
| 720 | 2314 | Pam-ETF%cs7EYWAQCba%c7SAA-NH2 |
| 721 | 2315 | Ac-LThF%cs7EFWAQCba%c7SAA-NH2 |
| 722 | 2316 | Ac-LTA%cs7EYWAQCba%c7SAA-NH2 |
| 723 | 2317 | Ac-LTF%cs7EYAAQCba%c7SAA-NH2 |
| 724 | 2318 | Ac-LTF%cs7EY2NalAQCba%c7SAA-NH2 |
| 725 | 2319 | Ac-LTF%cs7AYWAQCba%c7SAA-NH2 |
| 726 | 2320 | Ac-LTF%cs7EYWAQCba%c7SAF-NH2 |
| 727 | 2321 | Ac-LTF%cs7EYWAQCba%c7SAFa-NH2 |
| 728 | 2322 | Ac-LTF%cs7AYWAQCba%c7SAF-NH2 |
| 729 | 2323 | Ac-LTF34F2%cs7AYWAQCba%c7SAF-NH2 |
| 730 | 2324 | Ac-LTF%cs7AF4coohWAQCba%c7SAF-NH2 |
| 731 | 2325 | Ac-LTF%cs7EY6clWAQCba%c7SAF-NH2 |
| 732 | 2326 | Ac-LTF%cs7AYWSQCba%c7SAF-NH2 |
| 733 | 2327 | Ac-LTF%cs7AYWWQCba%c7SAF-NH2 |
| 734 | 2328 | Ac-LTF%cs7AYWAQCba%c7AAIa-NH2 |
| 735 | 2329 | Ac-LTF34F2%cs7AYWAQCba%c7AAIa-NH2 |
| 736 | 2330 | Ac-LTF%cs7AY6clWAQCba%c7AAIa-NH2 |
| 737 | 2331 | Ac-LTF%cs7AF4coohWAQCba%c7AAIa-NH2 |
| 738 | 2332 | Ac-LTF%cs7EYWAQCba%c7AAFa-NH2 |
| 739 | 2333 | Ac-LTF%cs7EYWAQCba%c7AAFa-NH2 |
| 740 | 2334 | Ac-ETF%cs7AYWAQCba%c7AWNlea-NH2 |
| 741 | 2335 | Ac-LTF%cs7EYWAQCba%c7AWNlea-NH2 |
| 742 | 2336 | Ac-ETF%cs7EYWAQCba%c7AWNlea-NH2 |
| 743 | 2337 | Ac-ETF%cs7EYWAQCba%c7AWNlea-NH2 |
| 744 | 2338 | Ac-LTF%cs7AYWAQCba%c7SAFa-NH2 |
| 745 | 2339 | Ac-LTF%cs7AYWAQCba%c7SAFa-NH2 |
| 746 | 2340 | Ac-ETF%cs7AYWAQL%c7AWNlea-NH2 |
| 747 | 2341 | Ac-LTF%cs7EYWAQL%c7AWNlea-NH2 |
| 748 | 2342 | Ac-ETF%cs7EYWAQL%c7AWNlea-NH2 |
| 749 | 2343 | Dmaac-LTF%cs7EYWAQhL%c7SAA-NH2 |
| 750 | 2344 | Hexac-LTF%cs7EYWAQhL%c7SAA-NH2 |
| 751 | 2345 | Napac-LTF%cs7EYWAQhL%c7SAA-NH2 |
| 752 | 2346 | Decac-LTF%cs7EYWAQhL%c7SAA-NH2 |
| 753 | 2347 | Admac-LTF%cs7EYWAQhL%c7SAA-NH2 |
| 754 | 2348 | Tmac-LTF%cs7EYWAQhL%c7SAA-NH2 |
| 755 | 2349 | Pam-LTF%cs7EYWAQhL%c7SAA-NH2 |
| 756 | 2350 | Ac-LTF%cs7AYWAQCba%c7AANleA-NH2 |
| 757 | 2351 | Ac-LTF34F2%cs7EYWAQCba%c7AAIa-NH2 |
| 758 | 2352 | Ac-LTF34F2%cs7EYWAQCba%c7SAA-NH2 |
| 759 | 2353 | Ac-LTF34F2%cs7EYWAQCba%c7SAA-NH2 |
| 760 | 2354 | Ac-LTF%cs7EF4coohWAQCba%c7SAA-NH2 |
| 761 | 2355 | Ac-LTF%cs7EF4coohWAQCba%c7SAA-NH2 |
| 762 | 2356 | Ac-LTF%cs7EYWSQCba%c7SAA-NH2 |
| 763 | 2357 | Ac-LTF%cs7EYWSQCba%c7SAA-NH2 |
| 764 | 2358 | Ac-LTF%cs7EYWAQhL%c7SAA-NH2 |
| 765 | 2359 | Ac-LTF%cs7AYWAQhL%c7SAF-NH2 |
| 766 | 2360 | Ac-LTF%cs7AYWAQhL%c7SAF-NH2 |
| 767 | 2361 | Ac-LTF34F2%cs7AYWAQhL%c7SAA-NH2 |
| 768 | 2362 | Ac-LTF34F2%cs7AYWAQhL%c7SAA-NH2 |
| 769 | 2363 | Ac-LTF%cs7AF4coohWAQhL%c7SAA-NH2 |
| 770 | 2364 | Ac-LTF%cs7AF4coohWAQhL%c7SAA-NH2 |
| 771 | 2365 | Ac-LTF%cs7AYWSQhL%c7SAA-NH2 |
| 772 | 2366 | Ac-LTF%cs7AYWSQhL%c7SAA-NH2 |
| 773 | 2367 | Ac-LTF%cs7EYWAQL%c7AANleA-NH2 |
| 774 | 2368 | Ac-LTF34F2%cs7AYWAQL%c7AANleA-NH2 |
| 775 | 2369 | Ac-LTF%cs7AF4coohWAQL%c7AANleA-NH2 |
| 776 | 2370 | Ac-LTF%cs7AYWSQL%c7AANleA-NH2 |
| 777 | 2371 | Ac-LTF34F2%cs7AYWAQhL%c7AANleA-NH2 |
| 778 | 2372 | Ac-LTF34F2%cs7AYWAQhL%c7AANleA-NH2 |
| 779 | 2373 | Ac-LTF%cs7AF4coohWAQhL%c7AANleA-NH2 |
| 780 | 2374 | Ac-LTF%cs7AF4coohWAQhL%c7AANleA-NH2 |
| 781 | 2375 | Ac-LTF%cs7AYWSQhL%c7AANleA-NH2 |
| 782 | 2376 | Ac-LTF%cs7AYWSQhL%c7AANleA-NH2 |
| 783 | 2377 | Ac-LTF%cs7AYWAQhL%c7AAAAa-NH2 |
| 784 | 2378 | Ac-LTF%cs7AYWAQhL%c7AAAAa-NH2 |
| 785 | 2379 | Ac-LTF%cs7AYWAQL%c7AAAAAa-NH2 |
| 786 | 2380 | Ac-LTF%cs7AYWAQL%c7AAAAAAa-NH2 |
| 787 | 2381 | Ac-LTF%cs7AYWAQL%c7AAAAAAa-NH2 |
| 788 | 2382 | Ac-LTF%cs7EYWAQhL%c7AANleA-NH2 |
| 789 | 2383 | Ac-AATF%cs7AYWAQL%c7AANleA-NH2 |
| 790 | 2384 | Ac-LTF%cs7AYWAQL%c7AANleAA-NH2 |
| 791 | 2385 | Ac-ALTF%cs7AYWAQL%c7AANleAA-NH2 |
| 792 | 2386 | Ac-LTF%cs7AYWAQCba%c7AANleAA-NH2 |
| 793 | 2387 | Ac-LTF%cs7AYWAQhL%c7AANleAA-NH2 |
| 794 | 2388 | Ac-LTF%cs7EYWAQCba%c7SAAA-NH2 |
| 795 | 2389 | Ac-LTF%cs7EYWAQCba%c7SAAA-NH2 |
| 796 | 2390 | Ac-LTF%cs7EYWAQCba%c7SAAAA-NH2 |
| 797 | 2391 | Ac-LTF%cs7EYWAQCba%c7SAAAA-NH2 |
| 798 | 2392 | Ac-ALTF%cs7EYWAQCba%c7SAA-NH2 |
| 799 | 2393 | Ac-ALTF%cs7EYWAQCba%c7SAAA-NH2 |
| 800 | 2394 | Ac-ALTF%cs7EYWAQCba%c7SAA-NH2 |
| 801 | 2395 | Ac-LTF%cs7EYWAQL%c7AAAAAa-NH2 |
| 802 | 2396 | Ac-LTF%cs7EY6clWAQCba%c7SAA-NH2 |
| 803 | 2397 | Ac-LTF%cs7EF4cooh6clWAQCba%c7SANleA-NH2 |
| 804 | 2398 | Ac-LTF%cs7EF4cooh6clWAQCba%c7SANleA-NH2 |
| 805 | 2399 | Ac-LTF%cs7EF4cooh6clWAQCba%c7AAIa-NH2 |
| 806 | 2400 | Ac-LTF%cs7EF4cooh6clWAQCba%c7AAIa-NH2 |
| 807 | 2401 | Ac-LTF%cs7AY6clWAQL%c7AAAAAa-NH2 |
| 808 | 2402 | Ac-LTF%cs7AY6clWAQL%c7AAAAAa-NH2 |
| 809 | 2403 | Ac-F%cs7AY6clWEAL%c7AAAAAAa-NH2 |
| 810 | 2404 | Ac-ETF%cs7EYWAQL%c7AAAAAa-NH2 |
| 811 | 2405 | Ac-ETF%cs7EYWAQL%c7AAAAAa-NH2 |
| 812 | 2406 | Ac-LTF%cs7EYWAQL%c7AAAAAAa-NH2 |
| 813 | 2407 | Ac-LTF%cs7EYWAQL%c7AAAAAAa-NH2 |
| 814 | 2408 | Ac-LTF%cs7AYWAQL%c7AANleAAa-NH2 |
| 815 | 2409 | Ac-LTF%cs7AYWAQL%c7AANleAAa-NH2 |
| 816 | 2410 | Ac-LTF%cs7EYWAQCba%c7AAAAAa-NH2 |
| 817 | 2411 | Ac-LTF%cs7EYWAQCba%c7AAAAAa-NH2 |
| 818 | 2412 | Ac-LTF%cs7EF4coohWAQCba%c7AAAAAa-NH2 |
| 819 | 2413 | Ac-LTF%cs7EF4coohWAQCba%c7AAAAAa-NH2 |
| 820 | 2414 | Ac-LTF%cs7EYWSQCba%c7AAAAAa-NH2 |
| 821 | 2415 | Ac-LTF%cs7EYWSQCba%c7AAAAAa-NH2 |
| 822 | 2416 | Ac-LTF%cs7EYWAQCba%c7SAAa-NH2 |
| 823 | 2417 | Ac-LTF%cs7EYWAQCba%c7SAAa-NH2 |
| 824 | 2418 | Ac-ALTF%cs7EYWAQCba%c7SAAa-NH2 |
| 825 | 2419 | Ac-ALTF%cs7EYWAQCba%c7SAAa-NH2 |
| 826 | 2420 | Ac-ALTF%cs7EYWAQCba%c7SAAAa-NH2 |
| 827 | 2421 | Ac-ALTF%cs7EYWAQCba%c7SAAAa-NH2 |
| 828 | 2422 | Ac-AALTF%cs7EYWAQCba%c7SAAAa-NH2 |
| 829 | 2423 | Ac-AALTF%cs7EYWAQCba%c7SAAAa-NH2 |
| 830 | 2424 | Ac-RTF%cs7EYWAQCba%c7SAA-NH2 |
| 831 | 2425 | Ac-LRF%cs7EYWAQCba%c7SAA-NH2 |
| 832 | 2426 | Ac-LTF%cs7EYWRQCba%c7SAA-NH2 |
| 833 | 2427 | Ac-LTF%cs7EYWARCba%c7SAA-NH2 |
| 834 | 2428 | Ac-LTF%cs7EYWAQCba%c7RAA-NH2 |
| 835 | 2429 | Ac-LTF%cs7EYWAQCba%c7SRA-NH2 |
| 836 | 2430 | Ac-LTF%cs7EYWAQCba%c7SAR-NH2 |
| 837 | 2431 | 5-FAM-BaLTF%cs7EYWAQCba%c7SAA-NH2 |
| 838 | 2432 | 5-FAM-BaLTF%cs7AYWAQL%c7AANleA-NH2 |
| 839 | 2433 | Ac-LAF%cs7EYWAQL%c7AANleA-NH2 |
| 840 | 2434 | Ac-ATF%cs7EYWAQL%c7AANleA-NH2 |
| 841 | 2435 | Ac-AAF%cs7EYWAQL%c7AANleA-NH2 |
| 842 | 2436 | Ac-AAAF%cs7EYWAQL%c7AANleA-NH2 |
| 843 | 2437 | Ac-AAAAF%cs7EYWAQL%c7AANleA-NH2 |
| 844 | 2438 | Ac-AATF%cs7EYWAQL%c7AANleA-NH2 |
| 845 | 2439 | Ac-AALTF%cs7EYWAQL%c7AANleA-NH2 |
| 846 | 2440 | Ac-AAALTF%cs7EYWAQL%c7AANleA-NH2 |
| 847 | 2441 | Ac-LTF%cs7EYWAQL%c7AANleAA-NH2 |
| 848 | 2442 | Ac-ALTF%cs7EYWAQL%c7AANleAA-NH2 |
| 849 | 2443 | Ac-AALTF%cs7EYWAQL%c7AANleAA-NH2 |
| 850 | 2444 | Ac-LTF%cs7EYWAQCba%c7AANleAA-NH2 |
| 851 | 2445 | Ac-LTF%cs7EYWAQhL%c7AANleAA-NH2 |
| 852 | 2446 | Ac-ALTF%cs7EYWAQhL%c7AANleAA-NH2 |
| 853 | 2447 | Ac-LTF%cs7ANmYWAQL%c7AANleA-NH2 |
| 854 | 2448 | Ac-LTF%cs7ANmYWAQL%c7AANleA-NH2 |
| 855 | 2449 | Ac-LTF%cs7AYNmWAQL%c7AANleA-NH2 |
| 856 | 2450 | Ac-LTF%cs7AYNmWAQL%c7AANleA-NH2 |
| 857 | 2451 | Ac-LTF%cs7AYAmwAQL%c7AANleA-NH2 |
| 858 | 2452 | Ac-LTF%cs7AYAmwAQL%c7AANleA-NH2 |
| 859 | 2453 | Ac-LTF%cs7AYWAibQL%c7AANleA-NH2 |
| 860 | 2454 | Ac-LTF%cs7AYWAibQL%c7AANleA-NH2 |
| 861 | 2455 | Ac-LTF%cs7AYWAQL%c7AAibNleA-NH2 |
| 862 | 2456 | Ac-LTF%cs7AYWAQL%c7AAibNleA-NH2 |
| 863 | 2457 | Ac-LTF%cs7AYWAQL%c7AaNleA-NH2 |
| 864 | 2458 | Ac-LTF%cs7AYWAQL%c7AaNleA-NH2 |
| 865 | 2459 | Ac-LTF%cs7AYWAQL%c7ASarNleA-NH2 |
| 866 | 2460 | Ac-LTF%cs7AYWAQL%c7ASarNleA-NH2 |
| 867 | 2461 | Ac-LTF%cs7AYWAQL%c7AANleAib-NH2 |
| 868 | 2462 | Ac-LTF%cs7AYWAQL%c7AANleAib-NH2 |
| 869 | 2463 | Ac-LTF%cs7AYWAQL%c7AANleNmA-NH2 |
| 870 | 2464 | Ac-LTF%cs7AYWAQL%c7AANleNmA-NH2 |
| 871 | 2465 | Ac-LTF%cs7AYWAQL%c7AANleSar-NH2 |
| 872 | 2466 | Ac-LTF%cs7AYWAQL%c7AANleSar-NH2 |
| 873 | 2467 | Ac-LTF%cs7AYWAQL%c7AANleAAib-NH2 |
| 874 | 2468 | Ac-LTF%cs7AYWAQL%c7AANleAAib-NH2 |
| 875 | 2469 | Ac-LTF%cs7AYWAQL%c7AANleANmA-NH2 |
| 876 | 2470 | Ac-LTF%cs7AYWAQL%c7AANleANmA-NH2 |
| 877 | 2471 | Ac-LTF%cs7AYWAQL%c7AANleAa-NH2 |
| 878 | 2472 | Ac-LTF%cs7AYWAQL%c7AANleAa-NH2 |
| 879 | 2473 | Ac-LTF%cs7AYWAQL%c7AANleASar-NH2 |
| 880 | 2474 | Ac-LTF%cs7AYWAQL%c7AANleASar-NH2 |
| 881 | 2475 | Ac-LTF%c7/r8AYWAQL%c7/AANleA-NH2 |
| 882 | 2476 | Ac-LTFAibAYWAQLAibAANleA-NH2 |
| 883 | 2477 | Ac-LTF%cs7Cou4YWAQL%c7AANleA-NH2 |
| 884 | 2478 | Ac-LTF%cs7Cou4YWAQL%c7AANleA-NH2 |
| 885 | 2479 | Ac-LTF%cs7AYWCou4QL%c7AANleA-NH2 |
| 886 | 2480 | Ac-LTF%cs7AYWAQL%c7Cou4ANleA-NH2 |
| 887 | 2481 | Ac-LTF%cs7AYWAQL%c7Cou4ANleA-NH2 |
| 888 | 2482 | Ac-LTF%cs7AYWAQL%c7ACou4NleA-NH2 |
| 889 | 2483 | Ac-LTF%cs7AYWAQL%c7ACou4NleA-NH2 |
| 890 | 2484 | Ac-LTF%cs7AYWAQL%c7AANleA-OH |
| 891 | 2485 | Ac-LTF%cs7AYWAQL%c7AANleA-OH |
| 892 | 2486 | Ac-LTF%cs7AYWAQL%c7AANleA-NHnPr |
| 893 | 2487 | Ac-LTF%cs7AYWAQL%c7AANleA-NHnPr |
| 894 | 2488 | Ac-LTF%cs7AYWAQL%c7AANleA-NHnBu33Me |
| 895 | 2489 | Ac-LTF%cs7AYWAQL%c7AANleA-NHnBu33Me |
| 896 | 2490 | Ac-LTF%cs7AYWAQL%c7AANleA-NHHex |
| 897 | 2491 | Ac-LTF%cs7AYWAQL%c7AANleA-NHHex |
| 898 | 2492 | Ac-LTA%cs7AYWAQL%c7AANleA-NH2 |
| 899 | 2493 | Ac-LThL%cs7AYWAQL%c7AANleA-NH2 |
| 900 | 2494 | Ac-LTF%cs7AYAAQL%c7AANleA-NH2 |
| 901 | 2495 | Ac-LTF%cs7AY2NalAQL%c7AANleA-NH2 |
| 902 | 2496 | Ac-LTF%cs7EYWCou4QCba%c7SAA-NH2 |
| 903 | 2497 | Ac-LTF%cs7EYWCou7QCba%c7SAA-NH2 |
| 904 | 2498 | Dmaac-LTF%cs7EYWAQCba%c7SAA-NH2 |
| 905 | 2499 | Dmaac-LTF%cs7AYWAQL%c7AAAAAa-NH2 |
| 906 | 2500 | Dmaac-LTF%cs7AYWAQL%c7AAAAAa-NH2 |
| 907 | 2501 | Dmaac-LTF%cs7EYWAQL%c7AAAAAa-NH2 |
| 908 | 2502 | Dmaac-LTF%cs7EYWAQL%c7AAAAAa-NH2 |
| 909 | 2503 | Dmaac-LTF%cs7EF4coohWAQCba%c7AAIa-NH2 |
| 910 | 2504 | Dmaac-LTF%cs7EF4coohWAQCba%c7AAIa-NH2 |
| 911 | 2505 | Dmaac-LTF%cs7AYWAQL%c7AANleA-NH2 |
| 912 | 2506 | Dmaac-LTF%cs7AYWAQL%c7AANleA-NH2 |
| 913 | 2507 | Cou6BaLTF%cs7EYWAQhL%c7SAA-NH2 |
| 914 | 2508 | Cou8BaLTF%cs7EYWAQhL%c7SAA-NH2 |
| 915 | 2509 | Ac-LTF4I%cs7EYWAQL%c7AAAAAa-NH2 |

Table 21 shows exemplary peptidomimetic macrocycles:

TABLE 21

| | SEQ | | Exact | Found | Calc | Calc | Calc |
|---|---|---|---|---|---|---|---|
| SP | ID NO: | Sequence | Mass | Mass | (M + 1)/1 | (M + 2)/2 | (M + 3)/3 |
| 916 | 2510 | Ac-LTF%cs7AYWAQL%c7AANleA-NH2 | 1808.94 | | 1809.95 | 905.48 | 603.99 |
| 917 | 2511 | Ac-LTF%cs7AYWAQL%c7AAAAAa-NH2 | 1908.96 | | 1909.97 | 955.49 | 637.33 |
| 918 | 2512 | Ac-LTF%csBphAYWAQL%cBphAANleA-NH2 | 1890.92 | | 1909.97 | 955.49 | 637.33 |
| 919 | 2513 | Ac-LTF%csBphAYWAQL%cBphAAAAAa-NH2 | 1990.92 | 996.88 | | | |
| 920 | 2514 | Ac-LTF%csBphEYWAQCba%cBphSAA-NH2 | 1865.16 | 933.45 | | 933.58 | |
| 921 | 2515 | Ac-LTF#cs7EYWAQCba#c7SAA-NH2 | 1753.82 | | 1754.83 | 877.92 | 585.61 |
| 922 | 2516 | Ac-LTF#csBphEYWAQCba#cBphSAA-NH2 | 1835.81 | | 1836.82 | 918.91 | 612.94 |
| 923 | 2517 | Ac-LTF%csBphEYWAQL%cBphAAAAAa-NH2 | | | | | |
| 924 | 2518 | Ac-LTF%cs5AYWAQL%c5AANleA-NH2 | | | | | |
| 925 | 2519 | Ac-LTF%cs5AYWAQL%c5AAAAAa-NH2 | | | | | |
| 926 | 2520 | Ac-LTF%cs6AYWAQL%c6AANleA-NH2 | | | | | |
| 927 | 2521 | Ac-LTF%cs6AYWAQL%c6AAAAAa-NH2 | | | | | |
| 928 | 2522 | Ac-LTF%cs6EYWAQL%c6AAAAAa-NH2 | 1894.94 | | 1895.96 | 948.48 | 632.66 |
| 929 | 2523 | Ac-LTF%cs5EYWAQL%c5AAAAAa-NH2 | 1880.93 | | 1881.94 | 941.47 | 627.98 |
| 930 | 2524 | Ac-LTF%cs6EYWAQCba%c6SAANH2 | 1709.83 | | 1710.84 | 855.92 | 570.95 |
| 931 | 2525 | Ac-LTF%cs5EYWAQCba%c5SAANH2 | 1695.81 | | 1696.82 | 848.92 | 566.28 |

Partial structures of selected exemplary peptidomimetic macrocycles are shown below:

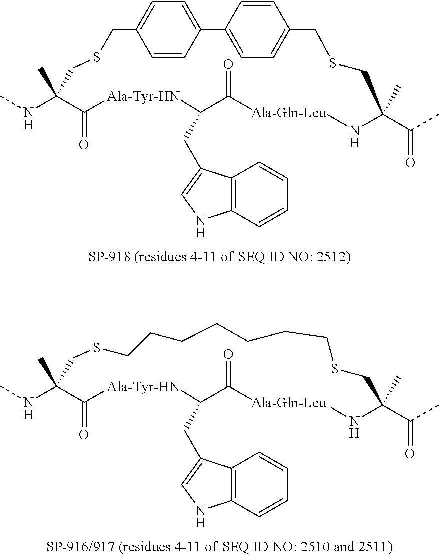

A structure of an exemplary peptidomimetic macrocycle is shown below:

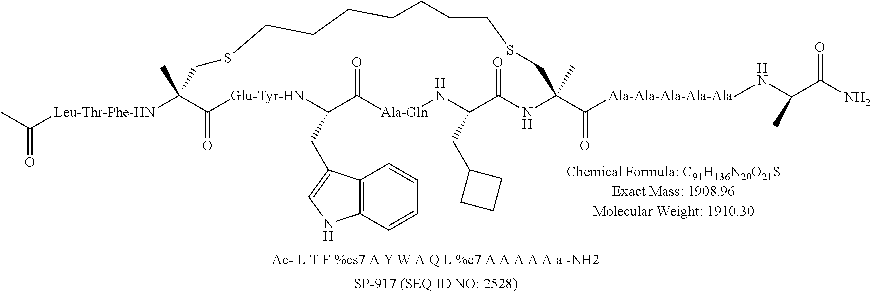

Another structure of an exemplary peptidomimetic macrocycle is shown below:

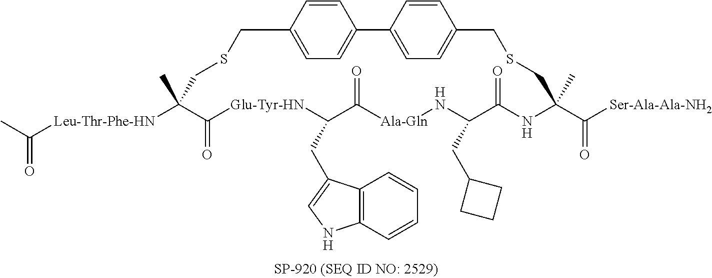

Amino acids represented as “#cs5” are D-cysteine connected by an i to i+7, five-methylene crosslinker to another thiol-containing amino acid. Amino acids represented as “#c5” are L-cysteine connected by an i to i+7, five-methylene crosslinker to another thiol-containing amino acid. Amino acids represented as “#cs6” are D-cysteine connected by an i to i+7, six-methylene crosslinker to another thiol-containing amino acid. Amino acids represented as “#c6” are L-cysteine connected by an i to i+7, six-methylene crosslinker to another thiol-containing amino acid. Amino acids represented as “#cs7” are D-cysteine connected by an i to i+7, seven-methylene crosslinker to another thiol-containing amino acid. Amino acids represented as “#c7” are L-cysteine connected by an i to i+7, seven-methylene crosslinker to another thiol-containing amino acid. Amino acids represented as “#cs8” are D-cysteine connected by an i to i+7, eight-methylene crosslinker to another thiol-containing amino acid. Amino acids represented as “#c8” are L-cysteine connected by an i to i+7, eight-methylene crosslinker to another thiol-containing amino acid. Amino acids represented as “% cs7” are alpha-methyl-D-cysteine connected by an i to i+7, seven-methylene crosslinker to another thiol-containing amino acid. Amino acids represented as “% c7” are alpha-methyl-L-cysteine connected by an i to i+7, seven-methylene crosslinker to another thiol-containing amino acid. Amino acids represented as “% cs8” are alpha-methyl-D-cysteine connected by an i to i+7, eight-methylene crosslinker to another thiol-containing amino acid. Amino acids represented as “% c8” are alpha-methyl-L-cysteine connected by an i to i+7, eight-methylene crosslinker to another thiol-containing amino acid. Amino acids represented as “% cs9” are alpha-methyl-D-cysteine connected by an i to i+7, nine-methylene crosslinker to another thiol-containing amino acid. Amino acids represented as “% c9” are alpha-methyl-L-cysteine connected by an i to i+7, nine-methylene crosslinker to another thiol-containing amino acid. Amino acids represented as “% cs10” are alpha-methyl-D-cysteine connected by an i to i+7, ten-methylene crosslinker to another thiol-containing amino acid. Amino acids represented as “% c10” are alpha-methyl-L-cysteine connected by an i to i+7, ten-methylene crosslinker to another thiol-containing amino acid. Amino acids represented as “pen8” are D-penicillamine connected by an i to i+7, eight-methylene crosslinker to another thiol-containing amino acid. Amino acids represented as “Pen8” are L-penicillamine connected by an i to i+7, eight-methylene crosslinker to another thiol-containing amino acid. Amino acids represented as “#csBph” are D-cysteine connected by an i to i+7, Bph (4,4′-bismethyl-biphenyl) crosslinker to another thiol-containing amino acid. Amino acids represented as “#cBph” are L-cysteine connected by an i to i+7, Bph (4,4′-bismethyl-biphenyl) crosslinker to another thiol-containing amino acid. Amino acids represented as “% csBph” are alpha-methyl-D-cysteine connected by an i to i+7, Bph (4,4′-bismethyl-biphenyl) crosslinker to another thiol-containing amino acid. Amino acids represented as “% cBph” are alpha-methyl-L-cysteine connected by an i to i+7, Bph (4,4′-bismethyl-biphenyl) crosslinker to another thiol-containing amino acid. Amino acids represented as “#csBpy” are D-cysteine connected by an i to i+7, Bpy (6,6′-bismethyl-[3,3′]bipyridine) crosslinker to another thiol-containing amino acid. Amino acids represented as “#cBpy” are L-cysteine connected by an i to i+7, Bpy (6,6′-bismethyl-[3,3′]bipyridine) crosslinker to another thiol-containing amino acid. Amino acids represented as “% csBpy” are alpha-methyl-D-cysteine connected by an i to i+7, Bpy (6,6′-bismethyl-[3,3′]bipyridine) crosslinker to another thiol-containing amino acid. Amino acids represented as “% cBpy” are alpha-methyl-L-cysteine connected by an i to i+7, Bpy (6,6′-bismethyl-[3,3′]bipyridine) crosslinker to another thiol-containing amino acid. The number of methylene units indicated above refers to the number of methylene units between the two thiol groups of the crosslinker.

In some embodiments, peptidomimetic macrocycles exclude peptidomimetic macrocycles shown in Table 22. Peptides shown can comprise an N-terminal capping group such as acetyl or an additional linker such as beta-alanine between the capping group and the start of the peptide sequence. In some embodiments, peptidomimetic macrocycles do not comprise a peptidomimetic macrocycle structure as shown in Table 22.

TABLE 22

| | SEQ | |
|---|---|---|
| # | ID NO: | Sequence |
| 1 | 2530 | QSQQTF%csNLWLL%cs6QN |
| 2 | 2531 | QSQQTF%csNLWLL%cs7QN |
| 3 | 2532 | QSQQTF%csNLWLL%cs8QN |
| 4 | 2533 | QSQQTF%csNLWLL%cs9QN |

In other embodiments, peptidomimetic macrocycles exclude peptidomimetic macrocycles shown in Table 23. In some embodiments, peptidomimetic macrocycles do not comprise a peptidomimetic macrocycle structure as shown in Table 23.

TABLE 23

| | SEQ | |
|---|---|---|
| Number | ID NO: | Sequence |
| 1 | 2534 | Ac-QSQQTF#cs5NLWRLL#c5QN-NH2 |
| 2 | 2535 | Ac-QSQQTF#cs6NLWRLL#c6QN-NH2 |
| 3 | 2536 | Ac-QSQQTF#cs7NLWRLL#c7QN-NH2 |
| 4 | 2537 | Ac-QSQQTF#cs8NLWRLL#c8QN-NH2 |
| 5 | 2538 | Ac-QSQQTF#cs9NLWRLL#c9QN-NH2 |
| 6 | 2539 | Ac-QSQQTF%cs8NLWRLL%c8QN-NH2 |
| 7 | 2540 | Ac-QSQQTF#cs8NLWRLLPen8QN-NH2 |
| 8 | 2541 | Ac-QSQQTF#c8NLWRLL#c8QN-NH2 |
| 9 | 2542 | Ac-QSQQTF#c8NLWRLL#cs8QN-NH2 |
| 10 | 2543 | Ac-QSQQTF#cs8NLWALL#c8AN-NH2 |
| 11 | 2544 | Ac-QAibQQTF#cs8NLWALL#c8AN-NH2 |
| 12 | 2545 | Ac-QAibQQTF#cs8ALWALL#c8AN-NH2 |
| 13 | 2546 | Ac-QSQQTFpen8NLWRLLPen8QN-NH2 |
| 14 | 2547 | Ac-QSQQTFpen8NLWRLL#c8QN-NH2 |
| 15 | 2548 | Ac-QSQQTF%cs9NLWRLL%c9QN-NH2 |
| 16 | 2549 | Ac-LTF#cs8HYWAQL#c8S-NH2 |
| 17 | 2550 | Ac-LTF#cs8HYWAQI#c8S-NH2 |
| 18 | 2551 | Ac-LTF#cs8HYWAQNle#c8S-NH2 |
| 19 | 2552 | Ac-LTF#cs8HYWAQL#c8A-NH2 |
| 20 | 2553 | Ac-LTF#cs8HYWAbuQL#c8S-NH2 |
| 21 | 2554 | Ac-LTF#cs8AYWAQL#c8S-NH2 |
| 22 | 2555 | Ac-LTF#cs8AYWAQL#c8A-NH2 |
| 23 | 2556 | Ac-LTF#cs8HYWAQLPen8S-NH2 |
| 24 | 2557 | Ac-LTFpen8HYWAQLPen8S-NH2 |
| 25 | 2558 | Ac-LTFpen8HYWAQL#c8S-NH2 |
| 26 | 2559 | Ac-LTF#cs7HYWAQL#hc7S-NH2 |
| 27 | 2560 | Ac-LTF%cs8HYWAQL%c8S-NH2 |
| 28 | 2561 | Ac-LTF%cs9HYWAQL%c9S-NH2 |
| 29 | 2562 | Ac-LTF%cs10HYWAQL%c10S-NH2 |
| 30 | 2563 | Ac-LTF%cs7HYWAQL%c7S-NH2 |
| 31 | 2564 | Ac-LTF%cs4BEBHYWAQL%c4BEBS-NH2 |
| 32 | 2565 | Ac-Fpen8AYWEAc3cL#c8A-NH2 |
| 33 | 2566 | Ac-F#cs8AYWEAc3cL#c8A-NH2 |
| 34 | 2567 | Ac-F%cs8AYWEAc3cL%c8A-NH2 |
| 35 | 2568 | Ac-LTFEHYWAQLTS-NH2 |

In other embodiments, peptidomimetic macrocycles exclude peptidomimetic macrocycles shown in Table 24 and disclosed in Muppidi et al., Chem. Commun. (2011) DOI: 10.1039/c1cc13320a. In some embodiments, peptidomimetic macrocycles do not comprise a peptidomimetic macrocycle structure as shown in Table 24.

TABLE 24

| | SEQ | |
|---|---|---|
| Number | ID NO: | Sequence |
| 1 | 2569 | LTFEHYWAQLTS |
| 2 | 2570 | LTFCHYWAQLCS |
| 3 | 2571 | LTF#cBphHYWAQL#cBphS |
| 4 | 2572 | LTF#cBpyHYWAQL#cBpyS |
| 5 | 2573 | LTFCRYWARLCS |
| 6 | 2574 | LTF#cBphRYWARL#cBphS |
| 7 | 2575 | LTF#cBpyRYWARL#cBpyS |
| 8 | 2576 | LTFcHYWAQLCS |
| 9 | 2577 | LTF#csBphHYWAQL#cBphS |
| 10 | 2578 | LTF#csBpyHYWAQL#csBpyS |
| 11 | 2579 | LTF#csBphRYWARL#cBphS |
| 12 | 2580 | LTF#csBpyRYWARL#cBpyS |

wherein C denotes L-cysteine and c denotes D-cysteine and #cBph, #cBpy, #csBph, and #csBpy are as defined herein.

### Example 3. Competition Binding ELISA (HDM2 & HDMX)

p53-His6 protein (30 nM/well) is coated overnight at room temperature in the wells of a 96-well Immulon plates. On the day of the experiment, plates are washed with 1×PBS-Tween 20 (0.05%) using an automated ELISA plate washer, blocked with ELISA Micro well Blocking for 30 minutes at room temperature; excess blocking agent is washed off by washing plates with 1×PBS-Tween 20 (0.05%). Peptides are diluted from 10 mM DMSO stocks to 500 μM working stocks in sterile water, further dilutions made in 0.5% DMSO to keep the concentration of DMSO constant across the samples. The peptides are added to wells at 2× desired concentrations in 50 μl volumes, followed by addition of diluted GST-HDM2 or GST-HMDX protein (final concentration: 10 nM). Samples are incubated at room temperature for 2h, plates are washed with PBS-Tween 20 (0.05%) prior to adding 100 μl of HRP-conjugated anti-GST antibody [Hypromatrix, INC] diluted to 0.5 μg/ml in HRP-stabilizing buffer. Post 30 min incubation with detection antibody, plates are washed and incubated with 100 μl per well of TMB-E Substrate solution up to 30 minutes; reactions are stopped using 1 M HCl and absorbance measured at 450 nm on micro plate reader. Data is analyzed using GraphPad PRISM software.

### Example 4. SJSA-1 Cell Viability Assay

SJSA1 cells are seeded at the density of 5000 cells/100 μl/well in 96-well plates a day prior to assay. On the day of study cells are washed once with Opti-MEM Media and 90 μL of the Opti-MEM Media is added to cells. Peptides are diluted from 10 mM DMSO stocks to 500 μM working stocks in sterile water, further dilutions made in 0.5% DMSO to keep the concentration of DMSO constant across the samples. The final concentration range μM will be 50, 25, 12.5, 6.25, 3.1, 1.56, 0.8 and 0 μM in 100 μL final volume per well for peptides. Final highest DMSO concentration is 0.5% and will be used as the negative control. Cayman Chemicals Cell-Based Assay (−)-Nutlin-3 (10 mM) is used as positive control. Nutlin was diluted using the same dilution scheme as peptides 10 μl of 10× desired concentrations is added to the appropriate well to achieve the final desired concentrations. Cells are then incubated with peptides for 20-24h at 37° C. in humidified 5% CO2 atmosphere. Post-incubation period, cell viability is measured using Promega Cell Titer-Glo reagents according to manufacturer′ instructions.

### Example 5. SJSA-1 p21 Up-Regulation Assay

SJSA1 cells are seeded at the density of 0.8 million cells/2 ml/well in 6-well plates a day prior to assay. On the day of study cells are washed once with Opti-MEM Media and 1350 μL of the Opti-MEM Media is added to cells. Peptides are diluted from 10 mM DMSO stocks to 500 μM working stocks in sterile water, further dilutions made in 0.5% DMSO to keep the concentration of DMSO constant across the samples. Final highest DMSO concentration is 0.5% and is used as the negative control. Cayman Chemicals Cell-Based Assay (−)-Nutlin-3 (10 mM) is used as positive control. Nutlin is diluted using the same dilution scheme as peptides 150 μl of 10× desired concentrations is added to the appropriate well to achieve the final desired concentrations. Cells are then incubated with peptides for 18-20 h at 37° C. in humidified 5% CO₂ atmosphere. Post-incubation period, cells are harvested, washed with 1×PBS (without Ca++/Mg++) and lysed in 1× Cell lysis buffer (Cell Signaling technologies 10× buffer diluted to 1× and supplemented with protease inhibitors and Phosphatase inhibitors) on ice for 30 min. Lysates are centrifuged at 13000 rpm speed in a microfuge at 40° C. for 8 min; clear supernatants are collected and stored at −80° C. until further use. Total protein content of the lysates is measured using BCA protein detection kit and BSA standards from Thermofisher. 25 μg of the total protein is used for p21 detection ELISA assay. Each condition is set in triplicate for ELISA plate. The ELISA assay protocol is followed as per the manufacturer's instructions. 25 μg total protein used for each well, and each well is set up in triplicate. Data is analyzed using GraphPad PRISM software.

### Example 6: p53 GRIP Assay

Thermo Scientific* BioImage p53-Hdm2 Redistribution Assay monitors the protein interaction with Hdm2 and cellular translocation of GFP-tagged p53 in response to drug compounds or other stimuli. Recombinant CHO-hIR cells stably express human p53(1-312) fused to the C-terminus of enhanced green fluorescent protein (EGFP) and PDE4A4-Hdm2(1-124), a fusion protein between PDE4A4 and Hdm2(1-124). They provide a ready-to-use assay system for measuring the effects of experimental conditions on the interaction of p53 and Hdm2. Imaging and analysis is performed with a HCS platform.

CHO-hIR cells are regularly maintained in Ham's F12 media supplemented with 1% Penicillin-Streptomycin, 0.5 mg/ml Geneticin, 1 mg/ml Zeocin and 10% FBS. Cells seeded into 96-well plates at the density of 7000 cells/100 μl per well 18-24 hours prior to running the assay using culture media. The next day, media is refreshed and PD177 is added to cells to the final concentration of 3 μM to activate foci formation. Control wells are kept without PD-177 solution. 24h post stimulation with PD177, cells are washed once with Opti-MEM Media and 50 μL of the Opti-MEM Media supplemented with PD-177(6 μM) is added to cells. Peptides are diluted from 10 mM DMSO stocks to 500 μM working stocks in sterile water, further dilutions made in 0.5% DMSO to keep the concentration of DMSO constant across the samples. Final highest DMSO concentration is 0.5% and is used as the negative control. Cayman Chemicals Cell-Based Assay (−)-Nutlin-3 (10 mM) is used as positive control. Nutlin was diluted using the same dilution scheme as peptides. 50 μl of 2× desired concentrations is added to the appropriate well to achieve the final desired concentrations. Cells are then incubated with peptides for 6 h at 37° C. in humidified 5% CO2 atmosphere. Post-incubation period, cells are fixed by gently aspirating out the media and adding 150 μl of fixing solution per well for 20 minutes at room temperature. Fixed cells are washed 4 times with 200 μl PBS per well each time. At the end of last wash, 100 μl of 1 μM Hoechst staining solution is added. Sealed plates incubated for at least 30 min in dark, washed with PBS to remove excess stain and PBS is added to each well. Plates can be stored at 4° C. in dark up to 3 days. The translocation of p53/HDM2 is imaged using Molecular translocation module on Cellomics Arrayscan instrument using 10× objective, XF-100 filter sets for Hoechst and GFP. The output parameters was Mean-CircRINGAveIntenRatio (the ratio of average fluorescence intensities of nucleus and cytoplasm, (well average)). The minimally acceptable number of cells per well used for image analysis was set to 500 cells.

### Example 7. Circular Dichroism (CD) Analysis of Alpha-Helicity

Peptide solutions were analyzed by CD spectroscopy using a Jasco J-815 spectropolarimeter (Jasco Inc., Easton, Md.) with the Jasco Spectra Manager Ver.2 system software. A Peltier temperature controller was used to maintain temperature control of the optical cell. Results are expressed as mean molar ellipticity [θ] (deg cm²dmol⁻¹) as calculated from the equation [θ]=θobs·MRW/10*1*c where θobs is the observed ellipticity in millidegrees, MRW is the mean residue weight of the peptide (peptide molecular weight/number of residues), 1 is the optical path length of the cell in centimeters, and c is the peptide concentration in mg/ml. Peptide concentrations were determined by amino acid analysis. Stock solutions of peptides were prepared in benign CD buffer (20 mM phosphoric acid, pH 2). The stocks were used to prepare peptide solutions of 0.05 mg/ml in either benign CD buffer or CD buffer with 50% trifluoroethanol (TFE) for analyses in a 10 mm pathlength cell. Variable wavelength measurements of peptide solutions were scanned at 4° C. from 195 to 250 nm, in 0.2 nm increments, and a scan rate 50 nm per minute. The average of six scans was reported.

Table 25 shows circular dichroism data for selected peptiomimetic macrocycles:

TABLE 25

| | Molar | Molar | Molar | | |
|---|---|---|---|---|---|
| | Ellip- | Ellip- | Ellip- | % Helix | % Helix |
| | ticity | ticity | ticity | 50% TFE | benign |
| | Benign | 50% TFE | TFE − Molar | compared | compared |
| | (222 in | (222 in | Ellipticity | to 50% TFE | to 50% TFE |
| SP# | 0% TFE) | 50% TFE) | Benign | parent (CD) | parent (CD) |
| 7 | 124 | −19921.4 | −20045.4 | 137.3 | −0.9 |
| 11 | −398.2 | −16623.4 | 16225.2 | 106.1 | 2.5 |
| 41 | −909 | −21319.4 | 20410.4 | 136 | 5.8 |
| 43 | −15334.5 | −18247.4 | 2912.9 | 116.4 | 97.8 |
| 69 | −102.6 | −21509.7 | −21407.1 | 148.2 | 0.7 |
| 71 | −121.2 | −17957 | −17835.9 | 123.7 | 0.8 |
| 154 | −916.2 | −30965.1 | −30048.9 | 213.4 | 6.3 |
| 230 | −213.2 | −17974 | −17760.8 | 123.9 | 1.5 |
| 233 | −477.9 | −19032.6 | −18554.7 | 131.2 | 3.3 |

### Example 8. Direct Binding Assay MDM2 with Fluorescence Polarization (FP)

The assay was performed according to the following general protocol:
1. Dilute MDM2 (In-house, 41 kD) into FP buffer (High salt buffer—200 mM NaCl, 5 mM CHAPS, pH 7.5) to make 10 μM working stock solution.
2. Add 30 μl of 10 μM of protein stock solution into A1 and B1 well of 96-well black HE microplate (Molecular Devices).
3. Fill in 30 μl of FP buffer into column A2 to A12, B2 to B12, C1 to C12, and D1 to D12.
4. 2 or 3 fold series dilution of protein stock from A1, B1 into A2, B2; A2, B2 to A3, B3; . . . to reach the single digit nM concentration at the last dilution point.
5. Dilute 1 mM (in 100% DMSO) of FAM labeled linear peptide with DMSO to 100 μM (dilution 1:10). Then, dilute from 100 μM to 10 μM with water (dilution 1:10) and then dilute with FP buffer from 10 μM to 40 nM (dilution 1:250). This is the working solution which will be a 10 nM concentration in well (dilution 1:4). Keep the diluted FAM labeled peptide in the dark until use.
6. Add 10 μl of 10 nM of FAM labeled peptide into each well and incubate, and read at different time points.

Kd with 5-FAM-BaLTFEHYWAQLTS-NH₂ (SEQ ID NO: 2581) is ˜13.38 nM.

### Example 9. Competitive Fluorescence Polarization Assay for MDM2

The assay was performed according to the following general protocol:
1. Dilute MDM2 (In-house, 41 kD) into FP buffer (High salt buffer-200 mM NaCl, 5 mM CHAPS, pH 7.5) to make 84 nM (2×) working stock solution.
2. Add 20 μl of 84 nM (2×) of protein stock solution into each well of 96-well black HE microplate (Molecular Devices).
3. Dilute 1 mM (in 100% DMSO) of FAM labeled linear peptide with DMSO to 100 μM (dilution 1:10). Then, dilute from 100 μM to 10 μM with water (dilution 1:10) and then dilute with FP buffer from 10 μM to 40 nM (dilution 1:250). This is the working solution which will be a 10 nM concentration in well (dilution 1:4). Keep the diluted FAM labeled peptide in the dark until use.
4. Make unlabeled peptide dose plate with FP buffer starting with 1 μM (final) of peptide and making 5 fold serial dilutions for 6 points using following dilution scheme.
5. Dilute 10 mM (in 100% DMSO) with DMSO to 5 mM (dilution 1:2). Then, dilute from 5 mM to 500 μM with H₂O (dilution 1:10) and then dilute with FP buffer from 500 μM to 20 μM (dilution 1:25). Making 5 fold serial dilutions from 4 μM (4×) for 6 points.
6. Transfer 10 μl of serial diluted unlabeled peptides to each well which is filled with 20 μl of 84 nM of protein.
7. Add 10 μl of 10 nM (4×) of FAM labeled peptide into each well and incubate for 3 hr to read.

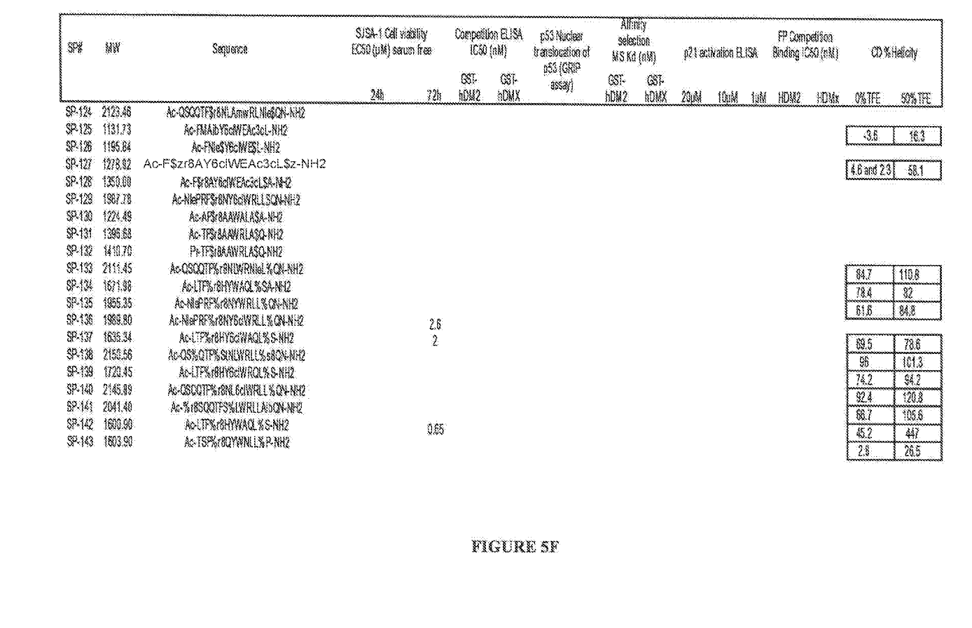

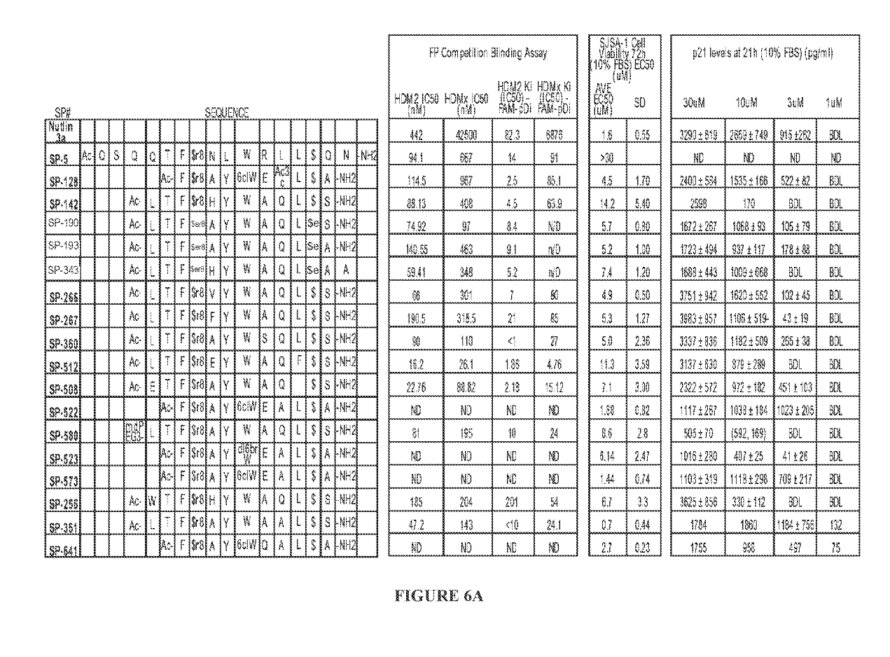

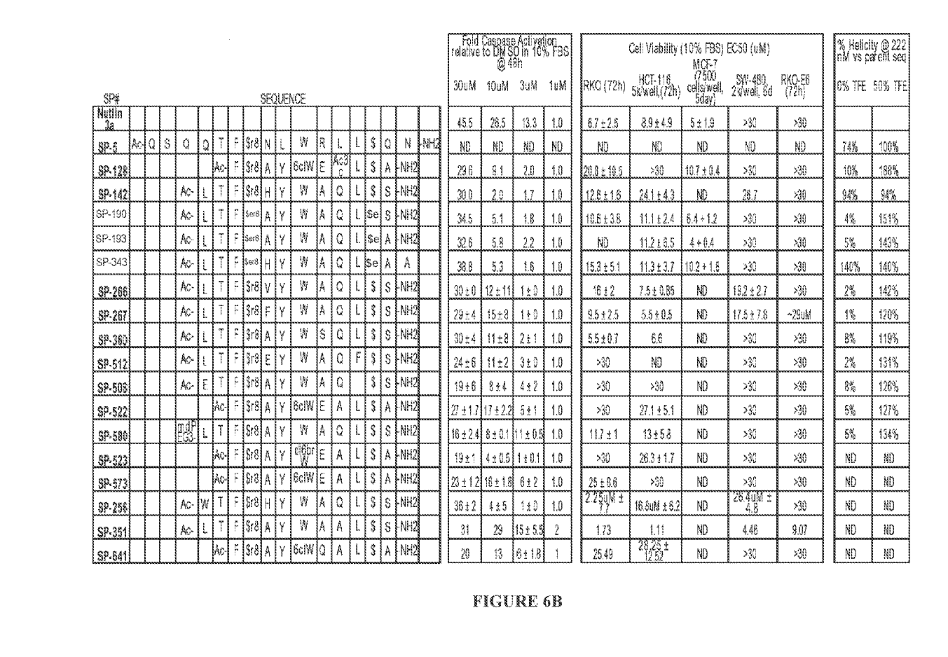

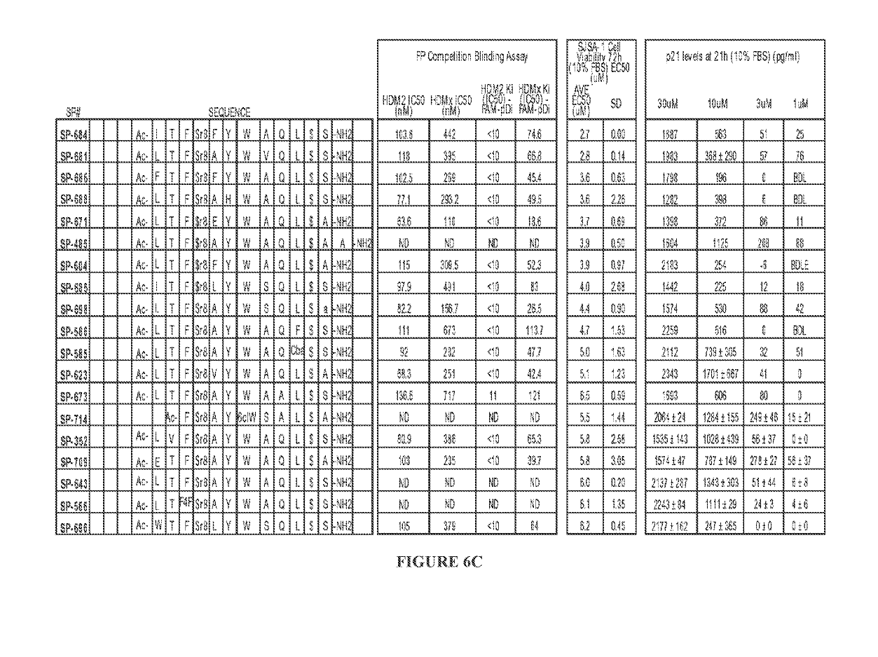

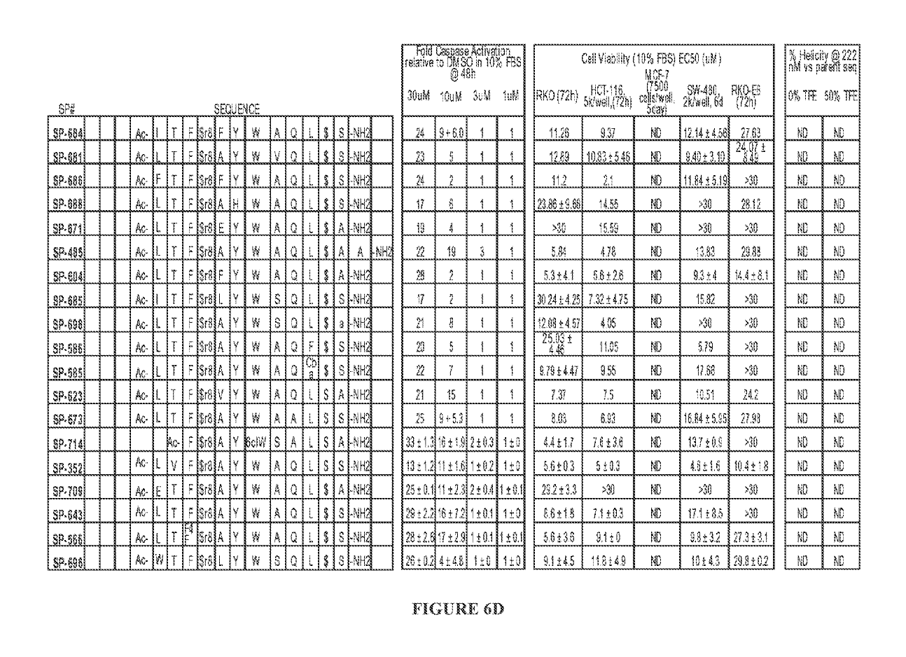

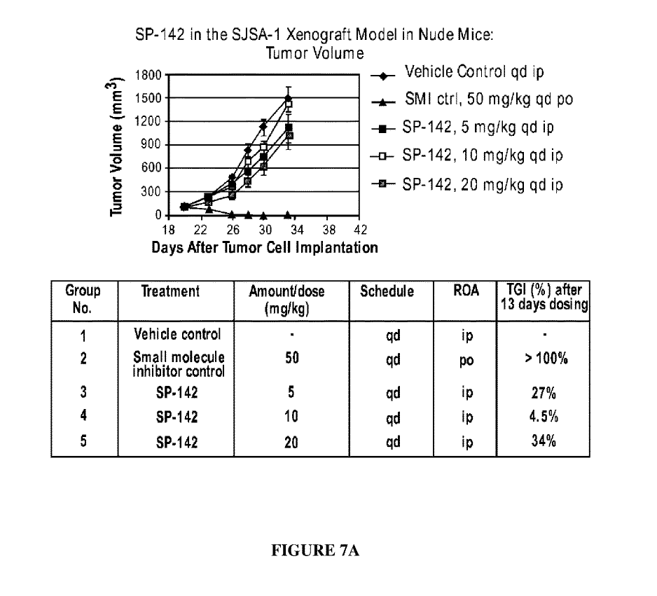

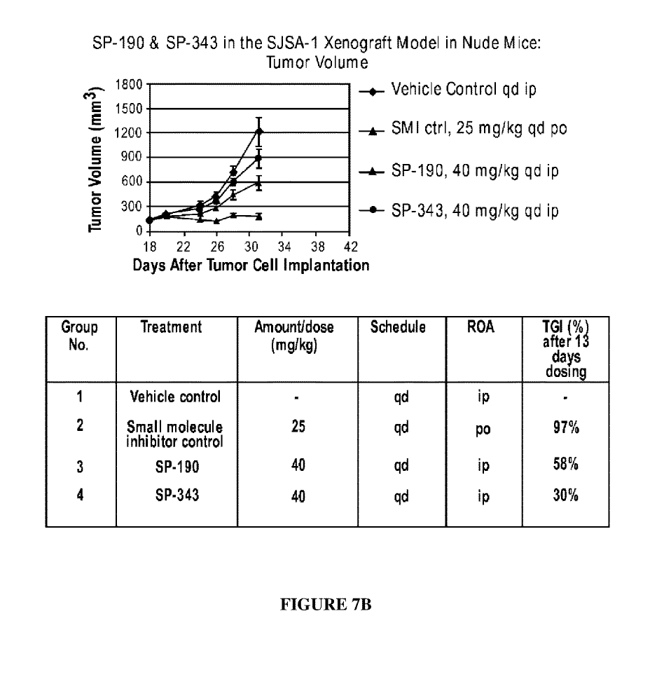

Results from Examples 8 and 9 are provided in HDM2 data in FIGS. 6A-D.

### Example 10. Direct Binding Assay MDMX with Fluorescence Polarization (FP)

The assay was performed according to the following general protocol:
1. Dilute MDMX (In-house, 40 kD) into FP buffer (High salt buffer—200 mM NaCl, 5 mM CHAPS, pH 7.5) to make 10 μM working stock solution.
2. Add 30 μl of 10 μM of protein stock solution into A1 and B1 well of 96-well black HE microplate (Molecular Devices).
3. Fill in 30 μl of FP buffer into column A2 to A12, B2 to B12, C1 to C12, and D1 to D12.
4. 2 or 3 fold series dilution of protein stock from A1, B1 into A2, B2; A2, B2 to A3, B3; . . . to reach the single digit nM concentration at the last dilution point.
5. Dilute 1 mM (in 100% DMSO) of FAM labeled linear peptide with DMSO to 100 μM (dilution 1:10). Then, dilute from 100 μM to 10 μM with water (dilution 1:10) and then dilute with FP buffer from 10 μM to 40 nM (dilution 1:250). This is the working solution which will be a 10 nM concentration in well (dilution 1:4). Keep the diluted FAM labeled peptide in the dark until use.
6. Add 10 μl of 10 nM of FAM labeled peptide into each well and incubate, and read at different time points.

Kd with 5-FAM-BaLTFEHYWAQLTS-NH₂ (SEQ ID NO: 2581) is −51 nM.

### Example 11. Competitive Fluorescence Polarization Assay MDMX

The assay was performed according to the following general protocol:
1. Dilute MDMX (In-house, 40 kD) into FP buffer (High salt buffer—200 mM NaCl, 5 mM CHAPS, pH 7.5.) to make 300 nM (2×) working stock solution.
2. Add 20 μl of 300 nM (2×) of protein stock solution into each well of 96-well black HE microplate (Molecular Devices)
3. Dilute 1 mM (in 100% DMSO) of FAM labeled linear peptide with DMSO to 100 μM (dilution 1:10). Then, dilute from 100 μM to 10 μM with water (dilution 1:10) and then dilute with FP buffer from 10 μM to 40 nM (dilution 1:250). This is the working solution which will be a 10 nM concentration in well (dilution 1:4). Keep the diluted FAM labeled peptide in the dark until use.
4. Make unlabeled peptide dose plate with FP buffer starting with 5 μM (final) of peptide and making 5 fold serial dilutions for 6 points using following dilution scheme.
5. Dilute 10 mM (in 100% DMSO) with DMSO to 5 mM (dilution 1:2). Then, dilute from 5 mM to 500 μM with H₂O (dilution 1:10) and then dilute with FP buffer from 500 μM to 20 μM (dilution 1:25). Making 5 fold serial dilutions from 20 μM (4×) for 6 points.
6. Transfer 10 μl of serial diluted unlabeled peptides to each well which is filled with 20 μl of 300 nM of protein.
7. Add 10 μl of 10 nM (4×) of FAM labeled peptide into each well and incubate for 3 hr to read.

Results from Examples 10 and 11 are provided in HDMX data in FIGS. 6A-D. Results from Example 11 is shown in Table 26A. The following scale is used: “+” represents a value greater than 1000 nM, “++” represents a value greater than 100 and less than or equal to 1000 nM, “+++” represents a value greater than 10 nM and less than or equal to 100 nM, and “++++” represents a value of less than or equal to 10 nM.

TABLE 26A

| SP# | IC50 (MDM2) | IC50 (MDMX) | Ki (MDM2) | Ki (MDMX) |
|---|---|---|---|---|
| 3 | ++ | ++ | +++ | +++ |
| 4 | +++ | ++ | ++++ | +++ |
| 5 | +++ | ++ | ++++ | +++ |
| 6 | ++ | ++ | +++ | +++ |
| 7 | +++ | +++ | ++++ | +++ |
| 8 | ++ | ++ | +++ | +++ |
| 9 | ++ | ++ | +++ | +++ |
| 10 | ++ | ++ | +++ | +++ |
| 11 | +++ | ++ | ++++ | +++ |
| 12 | + | + | +++ | ++ |
| 13 | ++ | ++ | +++ | ++ |
| 14 | +++ | +++ | ++++ | ++++ |
| 15 | +++ | ++ | +++ | +++ |
| 16 | +++ | +++ | ++++ | +++ |
| 17 | +++ | +++ | ++++ | +++ |
| 18 | +++ | +++ | ++++ | ++++ |
| 19 | ++ | +++ | +++ | +++ |
| 20 | ++ | ++ | +++ | +++ |
| 21 | ++ | +++ | +++ | +++ |
| 22 | +++ | +++ | ++++ | +++ |
| 23 | ++ | ++ | +++ | +++ |
| 24 | +++ | ++ | ++++ | +++ |
| 26 | +++ | ++ | ++++ | +++ |
| 28 | +++ | +++ | ++++ | +++ |
| 30 | ++ | ++ | +++ | +++ |
| 32 | +++ | ++ | ++++ | +++ |
| 38 | + | ++ | ++ | +++ |
| 39 | + | ++ | ++ | ++ |
| 40 | ++ | ++ | ++ | +++ |
| 41 | ++ | +++ | +++ | +++ |
| 42 | ++ | ++ | +++ | ++ |
| 43 | +++ | +++ | ++++ | +++ |
| 45 | +++ | +++ | ++++ | ++++ |
| 46 | +++ | +++ | ++++ | +++ |
| 47 | ++ | ++ | +++ | +++ |
| 48 | ++ | ++ | +++ | +++ |
| 49 | ++ | ++ | +++ | +++ |
| 50 | +++ | ++ | ++++ | +++ |
| 52 | +++ | +++ | ++++ | ++++ |
| 54 | ++ | ++ | +++ | +++ |
| 55 | + | + | ++ | ++ |
| 65 | +++ | ++ | ++++ | +++ |
| 68 | ++ | ++ | +++ | +++ |
| 69 | +++ | ++ | ++++ | +++ |
| 70 | ++ | ++ | ++++ | +++ |
| 71 | +++ | ++ | ++++ | +++ |
| 75 | +++ | ++ | ++++ | +++ |
| 77 | +++ | ++ | ++++ | +++ |
| 80 | +++ | ++ | ++++ | +++ |
| 81 | ++ | ++ | +++ | +++ |
| 82 | ++ | ++ | +++ | +++ |
| 85 | +++ | ++ | ++++ | +++ |
| 99 | ++++ | ++ | ++++ | +++ |
| 100 | ++ | ++ | ++++ | +++ |
| 101 | +++ | ++ | ++++ | +++ |
| 102 | ++ | ++ | ++++ | +++ |
| 103 | ++ | ++ | ++++ | +++ |
| 104 | +++ | ++ | ++++ | +++ |
| 105 | +++ | ++ | ++++ | +++ |
| 106 | ++ | ++ | +++ | +++ |
| 107 | ++ | ++ | +++ | +++ |
| 108 | +++ | ++ | ++++ | +++ |
| 109 | +++ | ++ | ++++ | +++ |
| 110 | ++ | ++ | ++++ | +++ |
| 111 | ++ | ++ | ++++ | +++ |
| 112 | ++ | ++ | +++ | +++ |
| 113 | ++ | ++ | +++ | +++ |
| 114 | +++ | ++ | ++++ | +++ |
| 115 | ++++ | ++ | ++++ | +++ |
| 116 | + | + | ++ | ++ |
| 118 | ++++ | ++ | ++++ | +++ |
| 120 | +++ | ++ | ++++ | +++ |
| 121 | ++++ | ++ | ++++ | +++ |
| 122 | ++++ | ++ | ++++ | +++ |
| 123 | ++++ | ++ | ++++ | +++ |
| 124 | ++++ | ++ | ++++ | +++ |
| 125 | ++++ | ++ | ++++ | +++ |
| 126 | ++++ | ++ | ++++ | +++ |
| 127 | ++++ | ++ | ++++ | +++ |
| 128 | ++++ | ++ | ++++ | +++ |
| 129 | ++++ | ++ | ++++ | +++ |
| 130 | ++++ | ++ | ++++ | +++ |
| 133 | ++++ | ++ | ++++ | +++ |
| 134 | ++++ | ++ | ++++ | +++ |
| 135 | ++++ | ++ | ++++ | +++ |
| 136 | ++++ | ++ | ++++ | +++ |
| 137 | ++++ | ++ | ++++ | +++ |
| 139 | ++++ | ++ | ++++ | +++ |
| 142 | ++++ | +++ | ++++ | +++ |
| 144 | ++++ | ++ | ++++ | +++ |
| 146 | ++++ | ++ | ++++ | +++ |
| 148 | ++++ | ++ | ++++ | +++ |
| 150 | ++++ | ++ | ++++ | +++ |
| 153 | ++++ | +++ | ++++ | +++ |
| 154 | ++++ | +++ | ++++ | ++++ |
| 156 | ++++ | ++ | ++++ | +++ |
| 158 | ++++ | ++ | ++++ | +++ |
| 160 | ++++ | ++ | ++++ | +++ |
| 161 | ++++ | ++ | ++++ | +++ |
| 166 | ++++ | ++ | ++++ | +++ |
| 167 | +++ | ++ | ++++ | ++ |
| 169 | ++++ | +++ | ++++ | +++ |
| 170 | ++++ | ++ | ++++ | +++ |
| 173 | ++++ | ++ | ++++ | +++ |
| 175 | ++++ | ++ | ++++ | +++ |
| 177 | +++ | ++ | ++++ | +++ |
| 180 | +++ | ++ | ++++ | +++ |
| 182 | ++++ | ++ | ++++ | +++ |
| 185 | +++ | + | ++++ | ++ |
| 186 | +++ | ++ | ++++ | +++ |
| 189 | +++ | ++ | ++++ | +++ |
| 192 | +++ | ++ | ++++ | +++ |
| 194 | +++ | ++ | ++++ | ++ |
| 196 | +++ | ++ | ++++ | +++ |
| 197 | ++++ | ++ | ++++ | +++ |
| 199 | +++ | ++ | ++++ | ++ |
| 201 | +++ | ++ | ++++ | ++ |
| 203 | +++ | ++ | ++++ | +++ |
| 204 | +++ | ++ | ++++ | +++ |
| 206 | +++ | ++ | ++++ | +++ |
| 207 | ++++ | ++ | ++++ | +++ |
| 210 | ++++ | ++ | ++++ | +++ |
| 211 | ++++ | ++ | ++++ | +++ |
| 213 | ++++ | ++ | ++++ | +++ |
| 215 | +++ | ++ | ++++ | +++ |
| 217 | ++++ | ++ | ++++ | +++ |
| 218 | ++++ | ++ | ++++ | +++ |
| 221 | ++++ | +++ | ++++ | +++ |
| 227 | ++++ | ++ | ++++ | +++ |
| 230 | ++++ | +++ | ++++ | ++++ |
| 232 | ++++ | ++ | ++++ | +++ |
| 233 | ++++ | +++ | ++++ | +++ |
| 236 | +++ | ++ | ++++ | +++ |
| 237 | +++ | ++ | ++++ | +++ |
| 238 | +++ | +++ | ++++ | +++ |
| 239 | +++ | ++ | +++ | +++ |
| 240 | +++ | ++ | ++++ | +++ |
| 241 | +++ | ++ | ++++ | +++ |
| 242 | +++ | ++ | ++++ | +++ |
| 243 | +++ | +++ | ++++ | +++ |
| 244 | +++ | +++ | ++++ | ++++ |
| 245 | +++ | +++ | ++++ | +++ |
| 246 | +++ | ++ | ++++ | +++ |
| 247 | +++ | +++ | ++++ | +++ |
| 248 | +++ | +++ | ++++ | +++ |
| 249 | +++ | +++ | ++++ | ++++ |
| 250 | ++ | + | ++ | + |
| 252 | ++ | + | ++ | + |
| 254 | +++ | ++ | ++++ | +++ |
| 255 | +++ | +++ | ++++ | +++ |
| 256 | +++ | +++ | ++++ | +++ |
| 257 | +++ | +++ | ++++ | +++ |
| 258 | +++ | ++ | ++++ | +++ |
| 259 | +++ | +++ | ++++ | +++ |
| 260 | +++ | +++ | ++++ | +++ |
| 261 | +++ | ++ | ++++ | +++ |
| 262 | +++ | ++ | ++++ | +++ |
| 263 | +++ | ++ | ++++ | +++ |
| 264 | +++ | +++ | ++++ | +++ |
| 266 | +++ | ++ | ++++ | +++ |
| 267 | +++ | +++ | ++++ | ++++ |
| 270 | ++++ | +++ | ++++ | +++ |
| 271 | ++++ | +++ | ++++ | ++++ |
| 272 | ++++ | +++ | ++++ | ++++ |
| 276 | +++ | +++ | ++++ | ++++ |
| 277 | +++ | +++ | ++++ | ++++ |
| 278 | +++ | +++ | ++++ | ++++ |
| 279 | ++++ | +++ | ++++ | +++ |
| 280 | +++ | ++ | ++++ | +++ |
| 281 | +++ | + | +++ | ++ |
| 282 | ++ | + | +++ | + |
| 283 | +++ | ++ | +++ | ++ |
| 284 | +++ | ++ | ++++ | +++ |
| 289 | +++ | +++ | ++++ | +++ |
| 291 | +++ | +++ | ++++ | ++++ |
| 293 | ++++ | +++ | ++++ | +++ |
| 306 | ++++ | ++ | ++++ | +++ |
| 308 | ++ | ++ | +++ | +++ |
| 310 | +++ | +++ | ++++ | +++ |
| 312 | +++ | ++ | +++ | +++ |
| 313 | ++++ | ++ | ++++ | +++ |
| 314 | ++++ | +++ | ++++ | ++++ |
| 315 | +++ | +++ | ++++ | +++ |
| 316 | ++++ | ++ | ++++ | +++ |
| 317 | +++ | ++ | +++ | +++ |
| 318 | +++ | ++ | +++ | +++ |
| 319 | +++ | ++ | +++ | ++ |
| 320 | +++ | ++ | +++ | ++ |
| 321 | +++ | ++ | ++++ | +++ |
| 322 | +++ | ++ | +++ | ++ |
| 323 | +++ | + | +++ | ++ |
| 328 | +++ | +++ | ++++ | +++ |
| 329 | +++ | +++ | ++++ | +++ |
| 331 | ++++ | +++ | ++++ | ++++ |
| 332 | ++++ | +++ | ++++ | ++++ |
| 334 | ++++ | +++ | ++++ | ++++ |
| 336 | ++++ | +++ | ++++ | ++++ |
| 339 | ++++ | ++ | ++++ | +++ |
| 341 | +++ | +++ | ++++ | ++++ |
| 343 | +++ | +++ | ++++ | ++++ |
| 347 | +++ | +++ | ++++ | +++ |
| 349 | ++++ | +++ | ++++ | ++++ |
| 351 | ++++ | +++ | ++++ | ++++ |
| 353 | ++++ | +++ | ++++ | ++++ |
| 355 | ++++ | +++ | ++++ | ++++ |
| 357 | ++++ | +++ | ++++ | ++++ |
| 359 | ++++ | +++ | ++++ | +++ |
| 360 | ++++ | ++++ | ++++ | ++++ |
| 363 | +++ | +++ | ++++ | ++++ |
| 364 | +++ | +++ | ++++ | ++++ |
| 365 | +++ | +++ | ++++ | ++++ |
| 366 | +++ | +++ | ++++ | +++ |
| 369 | ++ | ++ | +++ | +++ |
| 370 | +++ | +++ | ++++ | +++ |
| 371 | ++ | ++ | +++ | +++ |
| 372 | ++ | ++ | +++ | +++ |
| 373 | +++ | +++ | +++ | +++ |
| 374 | +++ | +++ | ++++ | ++++ |
| 375 | +++ | +++ | ++++ | ++++ |
| 376 | +++ | +++ | ++++ | ++++ |
| 377 | +++ | +++ | ++++ | +++ |
| 378 | +++ | +++ | ++++ | +++ |
| 379 | +++ | +++ | ++++ | +++ |
| 380 | +++ | +++ | ++++ | +++ |
| 381 | +++ | +++ | ++++ | +++ |
| 382 | +++ | +++ | ++++ | ++++ |
| 384 | ++ | + | ++ | + |
| 386 | ++ | + | ++ | + |
| 388 | ++ | +++ | +++ | ++++ |
| 390 | +++ | +++ | ++++ | +++ |
| 392 | +++ | +++ | ++++ | ++++ |
| 394 | ++++ | +++ | ++++ | ++++ |
| 396 | ++++ | ++++ | ++++ | ++++ |
| 398 | +++ | +++ | ++++ | +++ |
| 402 | ++++ | ++++ | ++++ | ++++ |
| 404 | +++ | +++ | ++++ | ++++ |
| 408 | +++ | +++ | ++++ | +++ |
| 410 | ++++ | ++++ | ++++ | ++++ |
| 411 | ++ | + | ++ | + |
| 412 | ++++ | +++ | ++++ | ++++ |
| 415 | ++++ | ++++ | ++++ | ++++ |
| 416 | +++ | +++ | ++++ | +++ |
| 417 | +++ | +++ | ++++ | +++ |
| 418 | ++++ | +++ | ++++ | ++++ |
| 419 | +++ | +++ | +++ | ++++ |
| 421 | ++++ | ++++ | ++++ | ++++ |
| 423 | +++ | +++ | ++++ | +++ |
| 425 | +++ | +++ | +++ | +++ |
| 427 | ++ | ++ | +++ | +++ |
| 432 | ++++ | +++ | ++++ | ++++ |
| 434 | +++ | +++ | ++++ | +++ |
| 435 | ++++ | +++ | ++++ | ++++ |
| 437 | +++ | +++ | ++++ | +++ |
| 439 | ++++ | +++ | ++++ | ++++ |
| 441 | ++++ | ++++ | ++++ | ++++ |
| 443 | +++ | +++ | ++++ | +++ |
| 445 | +++ | ++ | ++++ | +++ |
| 446 | +++ | + | ++++ | + |
| 447 | ++ | + | ++ | + |
| 551 | N/A | N/A | ++++ | +++ |
| 555 | N/A | N/A | ++++ | +++ |
| 556 | N/A | N/A | ++++ | +++ |
| 557 | N/A | N/A | +++ | +++ |
| 558 | N/A | N/A | +++ | +++ |
| 559 | N/A | N/A | +++ | +++ |
| 560 | N/A | N/A | + | + |
| 561 | N/A | N/A | ++++ | +++ |
| 562 | N/A | N/A | +++ | +++ |
| 563 | N/A | N/A | +++ | +++ |
| 564 | N/A | N/A | ++++ | +++ |
| 565 | N/A | N/A | +++ | +++ |
| 566 | N/A | N/A | ++++ | +++ |
| 567 | N/A | N/A | ++++ | +++ |
| 568 | N/A | N/A | ++++ | ++++ |
| 569 | N/A | N/A | ++++ | +++ |
| 570 | N/A | N/A | ++++ | +++ |
| 571 | N/A | N/A | ++++ | +++ |
| 572 | N/A | N/A | +++ | +++ |
| 573 | N/A | N/A | +++ | +++ |
| 574 | N/A | N/A | ++++ | +++ |
| 575 | N/A | N/A | ++++ | +++ |
| 576 | N/A | N/A | ++++ | +++ |
| 577 | N/A | N/A | ++++ | +++ |
| 578 | N/A | N/A | ++++ | +++ |
| 585 | N/A | N/A | +++ | +++ |
| 586 | N/A | N/A | ++++ | +++ |
| 587 | N/A | N/A | ++++ | ++++ |
| 589 | N/A | N/A | ++++ | |
| 594 | N/A | N/A | ++++ | ++++ |
| 596 | N/A | N/A | ++++ | +++ |
| 597 | N/A | N/A | ++++ | +++ |
| 598 | N/A | N/A | ++++ | +++ |
| 600 | N/A | N/A | ++++ | ++++ |
| 602 | N/A | N/A | ++++ | ++++ |
| 603 | N/A | N/A | ++++ | ++++ |
| 604 | N/A | N/A | +++ | +++ |
| 608 | N/A | N/A | ++++ | +++ |
| 609 | N/A | N/A | ++++ | +++ |
| 610 | N/A | N/A | ++++ | +++ |
| 611 | N/A | N/A | ++++ | +++ |
| 612 | N/A | N/A | ++++ | +++ |
| 613 | N/A | N/A | ++++ | +++ |
| 615 | N/A | N/A | ++++ | ++++ |
| 433 | N/A | N/A | ++++ | +++ |
| 686 | N/A | N/A | ++++ | +++ |
| 687 | N/A | N/A | ++ | ++ |
| 595 | N/A | N/A | + | N/A |
| 665 | N/A | N/A | +++ | N/A |
| 708 | N/A | N/A | +++ | +++ |
| 710 | N/A | N/A | +++ | +++ |
| 711 | N/A | N/A | +++ | ++ |
| 712 | N/A | N/A | ++++ | ++++ |
| 713 | N/A | N/A | ++++ | ++++ |
| 716 | N/A | N/A | ++++ | ++++ |
| 765 | + | + | | |
| 766 | +++ | + | | |
| 752 | ++ | + | | |
| 753 | +++ | + | | |
| 754 | ++ | + | | |
| 755 | ++++ | + | | |
| 756 | +++ | + | | |
| 757 | ++++ | + | | |
| 758 | +++ | + | | |

Results from Example 11 are also shown in Table 26B. The following scale is used for IC₅₀ and Ki values: “+” represents a value greater than 1000 nM, “++” represents a value greater than 100 and less than or equal to 1000 nM, “+++” represents a value greater than 10 nM and less than or equal to 100 nM, and “++++” represents a value of less than or equal to 10 nM.

TABLE 26B

| | | | | | SJSA-1 | IC₅₀ |
|---|---|---|---|---|---|---|
| SP | IC₅₀ (MDM2) | IC₅₀ (MDMX) | Ki (MDM2) | Ki (MDMX) | EC₅₀ (72 h) | Ratio |
| 449 | ++++ | ++++ | ++++ | ++++ | ++++ | |
| 450 | | | ++ | | +++ | |
| 451 | | | +++ | | +++ | |
| 452 | | | | | + | |
| 456 | | | ++++ | +++ | +++ | |
| 457 | | | ++++ | ++++ | ++++ | |
| 461 | | | | | +++ | |
| 459 | | | + | + | + | |
| 460 | | | + | + | + | |
| 463 | | | | | ++ | |
| 464 | | | | | + | |
| 153 | | | ++++ | +++ | ++++ | 1-29 |
| 465 | | | ++++ | ++++ | | |
| 466 | | | ++++ | ++++ | | |
| 470 | | | ++++ | ++++ | | |
| 916 | +++ | +++ | ++++ | ++++ | ++ | |
| 917 | +++ | +++ | ++++ | +++ | + | |
| 919 | | | | | +++ | |

### Example 12. Cell Viability Assay

The assay was performed according to the following general protocol:

Cell Plating: Trypsinize, count and seed cells at the pre-determined densities in 96-well plates a day prior to assay. Following cell densities are used for each cell line in use: SJSA-1: 7500 cells/well, RKO: 5000 cells/well, RKO-E6: 5000 cells/well, HCT-116: 5000 cells/well, SW-480: 2000 cells/well, and MCF-7: 5000 cells/well.

On the day of study, replace media with fresh media with 11% FBS (assay media) at room temperature. Add 180 μL of the assay media per well. Control wells with no cells, receive 200 μl media.

Peptide dilution: all dilutions are made at room temperature and added to cells at room temperature. Prepare 10 mM stocks of the peptides in DMSO. Serially dilute the stock using 1:3 dilution scheme to get 10, 3.3, 1.1, 0.33, 0.11, 0.03, 0.01 mM solutions using DMSO as diluents. Dilute the serially DMSO-diluted peptides 33.3 times using sterile water. This gives range of 10× working stocks. Also prepare DMSO/sterile water (3% DMSO) mix for control wells. Thus the working stocks concentration range μM will be 300, 100, 30, 10, 3, 1, 0.3 and 0 μM. Mix well at each dilution step using multichannel. Row H has controls. H1-H3 will receive 20 μl of assay media. H4-H9 will receive 20 μl of 3% DMSO-water vehicle. H10-H12 will have media alone control with no cells. Positive control: HDM2 small molecule inhibitor, Nutlin-3a (10 mM) is used as positive control. Nutlin was diluted using the same dilution scheme as peptides.

Addition of working stocks to cells: Add 20 μl of 10× desired concentration to appropriate well to achieve the final concentrations in total 200 μl volume in well. (20 μl of 300 μM peptide+180 μl of cells in media=30 μM final concentration in 200 μl volume in wells). Mix gently a few times using pipette. Thus final concentration range used will be 30, 10, 3, 1, 0.3, 0.1, 0.03 & 0 μM (for potent peptides further dilutions are included). Controls include wells that get no peptides but contain the same concentration of DMSO as the wells containing the peptides, and wells containing NO CELLS. Incubate for 72 hours at 37° C. in humidified 5% CO₂ atmosphere. The viability of cells is determined using MTT reagent from Promega. Viability of SJSA-1, RKO, RKO-E6, HCT-116 cells is determined on day 3, MCF-7 cells on day 5 and SW-480 cells on day 6. At the end of designated incubation time, allow the plates to come to room temperature. Remove 80 μl of assay media from each well. Add 15 μl of thawed MTT reagent to each well. Allow plate to incubate for 2 h at 37° C. in humidified 5% CO₂ atmosphere and add 100 μl solubilization reagent as per manufacturer's protocol. Incubate with agitation for 1h at room temperature and read on Synergy Biotek multiplate reader for absorbance at 570 nM. Analyze the cell viability against the DMSO controls using GraphPad PRISM analysis tools. Reagents: Invitrogen cell culture Media, Falcon 96-well clear cell culture treated plates (Nunc 353072), DMSO (Sigma D 2650), RPMI 1640 (Invitrogen 72400), and MTT (Promega G4000). Instruments: Multiplate Reader for Absorbance readout (Synergy 2).

Results from Examples 4 and 5 are provided in SJSA-1 EC₅₀ data in FIGS. 6A-D.

Results from cell viability assays are shown in Tables 27 and 28. The following scale is used: “+” represents a value greater than 30 μM, “++” represents a value greater than 15 μM and less than or equal to 30 μM, “+++” represents a value greater than 5 μM and less than or equal to 15 μM, and “++++” represents a value of less than or equal to 5 μM. “IC50 ratio” represents the ratio of average IC50 in p53+/+ cells relative to average IC50 in p53−/− cells.

TABLE 27

| SP# | SJSA-1 EC₅₀ (72 h) |
|---|---|
| 3 | +++ |
| 4 | +++ |
| 5 | ++++ |
| 6 | ++ |
| 7 | ++++ |
| 8 | +++ |
| 9 | +++ |
| 10 | +++ |
| 11 | ++++ |
| 12 | ++ |
| 13 | +++ |
| 14 | + |
| 15 | ++ |
| 16 | + |
| 17 | + |
| 18 | + |
| 19 | ++ |
| 20 | + |
| 21 | + |
| 22 | + |
| 24 | +++ |
| 26 | ++++ |
| 28 | + |
| 29 | + |
| 30 | + |
| 32 | ++ |
| 38 | + |
| 39 | + |
| 40 | + |
| 41 | + |
| 42 | + |
| 43 | ++ |
| 45 | + |
| 46 | + |
| 47 | + |
| 48 | + |
| 49 | +++ |
| 50 | ++++ |
| 52 | + |
| 54 | + |
| 55 | + |
| 65 | ++++ |
| 68 | ++++ |
| 69 | ++++ |
| 70 | ++++ |
| 71 | ++++ |
| 72 | ++++ |
| 74 | ++++ |
| 75 | ++++ |
| 77 | ++++ |
| 78 | ++ |
| 80 | ++++ |
| 81 | +++ |
| 82 | +++ |
| 83 | +++ |
| 84 | + |
| 85 | +++ |
| 99 | ++++ |
| 102 | +++ |
| 103 | +++ |
| 104 | +++ |
| 105 | +++ |
| 108 | +++ |
| 109 | +++ |
| 110 | +++ |
| 111 | ++ |
| 114 | ++++ |
| 115 | ++++ |
| 118 | ++++ |
| 120 | ++++ |
| 121 | ++++ |
| 122 | ++++ |
| 123 | ++++ |
| 124 | +++ |
| 125 | ++++ |
| 126 | ++++ |
| 127 | ++++ |
| 128 | +++ |
| 129 | ++ |
| 130 | ++++ |
| 131 | +++ |
| 132 | ++++ |
| 133 | +++ |
| 134 | +++ |
| 135 | +++ |
| 136 | ++ |
| 137 | +++ |
| 139 | ++++ |
| 142 | +++ |
| 144 | ++++ |
| 147 | ++++ |
| 148 | ++++ |
| 149 | ++++ |
| 150 | ++++ |
| 152 | +++ |
| 153 | ++++ |
| 154 | ++++ |
| 155 | ++ |
| 156 | +++ |
| 157 | +++ |
| 158 | +++ |
| 160 | ++++ |
| 161 | ++++ |
| 162 | +++ |
| 163 | +++ |
| 166 | ++ |
| 167 | +++ |
| 168 | ++ |
| 169 | ++++ |
| 170 | ++++ |
| 171 | ++ |
| 173 | +++ |
| 174 | ++++ |
| 175 | +++ |
| 176 | +++ |
| 177 | ++++ |
| 179 | +++ |
| 180 | +++ |
| 181 | +++ |
| 182 | ++++ |
| 183 | ++++ |
| 184 | +++ |
| 185 | +++ |
| 186 | ++ |
| 188 | ++ |
| 190 | ++++ |
| 192 | +++ |
| 193 | ++ |
| 194 | + |
| 195 | ++++ |
| 196 | ++++ |
| 197 | ++++ |
| 198 | ++ |
| 199 | +++ |
| 200 | +++ |
| 201 | ++++ |
| 202 | +++ |
| 203 | ++++ |
| 204 | ++++ |
| 205 | ++ |
| 206 | ++ |
| 207 | +++ |
| 208 | +++ |
| 209 | ++++ |
| 210 | +++ |
| 211 | ++++ |
| 213 | ++++ |
| 214 | ++++ |
| 215 | ++++ |
| 216 | ++++ |
| 217 | ++++ |
| 218 | ++++ |
| 219 | ++++ |
| 220 | +++ |
| 221 | ++++ |
| 222 | +++ |
| 223 | ++++ |
| 224 | ++ |
| 225 | +++ |
| 226 | ++ |
| 227 | +++ |
| 228 | ++++ |
| 229 | ++++ |
| 230 | ++++ |
| 231 | ++++ |
| 232 | ++++ |
| 233 | ++++ |
| 234 | ++++ |
| 235 | ++++ |
| 236 | ++++ |
| 237 | ++++ |
| 238 | ++++ |
| 239 | +++ |
| 240 | ++ |
| 241 | +++ |
| 242 | ++++ |
| 243 | ++++ |
| 244 | ++++ |
| 245 | ++++ |
| 246 | +++ |
| 247 | ++++ |
| 248 | ++++ |
| 249 | ++++ |
| 250 | ++ |
| 251 | + |
| 252 | + |
| 253 | + |
| 254 | +++ |
| 255 | +++ |
| 256 | ++ |
| 257 | +++ |
| 258 | +++ |
| 259 | ++ |
| 260 | ++ |
| 261 | ++ |
| 262 | +++ |
| 263 | ++ |
| 264 | ++++ |
| 266 | +++ |
| 267 | ++++ |
| 270 | ++ |
| 271 | ++ |
| 272 | ++ |
| 276 | ++ |
| 277 | ++ |
| 278 | ++ |
| 279 | ++++ |
| 280 | +++ |
| 281 | ++ |
| 282 | ++ |
| 283 | ++ |
| 284 | ++++ |
| 289 | ++++ |
| 290 | +++ |
| 291 | ++++ |
| 292 | ++++ |
| 293 | ++++ |
| 294 | ++++ |
| 295 | +++ |
| 296 | ++++ |
| 297 | +++ |
| 298 | ++++ |
| 300 | ++++ |
| 301 | ++++ |
| 302 | ++++ |
| 303 | ++++ |
| 304 | ++++ |
| 305 | ++++ |
| 306 | ++++ |
| 307 | +++ |
| 308 | ++++ |
| 309 | +++ |
| 310 | ++++ |
| 312 | ++++ |
| 313 | ++++ |
| 314 | ++++ |
| 315 | ++++ |
| 316 | ++++ |
| 317 | ++++ |
| 318 | ++++ |
| 319 | ++++ |
| 320 | ++++ |
| 321 | ++++ |
| 322 | ++++ |
| 323 | ++++ |
| 324 | ++++ |
| 326 | ++++ |
| 327 | ++++ |
| 328 | ++++ |
| 329 | ++++ |
| 330 | ++++ |
| 331 | ++++ |
| 332 | ++++ |
| 333 | ++ |
| 334 | +++ |
| 335 | ++++ |
| 336 | ++++ |
| 337 | ++++ |
| 338 | ++++ |
| 339 | ++++ |
| 340 | ++++ |
| 341 | ++++ |
| 342 | ++++ |
| 343 | ++++ |
| 344 | ++++ |
| 345 | ++++ |
| 346 | ++++ |
| 347 | ++++ |
| 348 | ++++ |
| 349 | ++++ |
| 350 | ++++ |
| 351 | ++++ |
| 352 | ++++ |
| 353 | ++++ |
| 355 | ++++ |
| 357 | ++++ |
| 358 | ++++ |
| 359 | ++++ |
| 360 | ++++ |
| 361 | +++ |
| 362 | ++++ |
| 363 | ++++ |
| 364 | ++++ |
| 365 | +++ |
| 366 | ++++ |
| 367 | ++++ |
| 368 | + |
| 369 | ++++ |
| 370 | ++++ |
| 371 | ++++ |
| 372 | +++ |
| 373 | +++ |
| 374 | ++++ |
| 375 | ++++ |
| 376 | ++++ |
| 377 | ++++ |
| 378 | ++++ |
| 379 | ++++ |
| 380 | ++++ |
| 381 | ++++ |
| 382 | ++++ |
| 386 | +++ |
| 388 | ++ |
| 390 | ++++ |
| 392 | +++ |
| 394 | +++ |
| 396 | +++ |
| 398 | +++ |
| 402 | +++ |
| 404 | +++ |
| 408 | ++++ |
| 410 | +++ |
| 411 | +++ |
| 412 | + |
| 421 | +++ |
| 423 | ++++ |
| 425 | ++++ |
| 427 | ++++ |
| 434 | +++ |
| 435 | ++++ |
| 436 | ++++ |
| 437 | ++++ |
| 438 | ++++ |
| 439 | ++++ |
| 440 | ++++ |
| 441 | ++++ |
| 442 | ++++ |
| 443 | ++++ |
| 444 | +++ |
| 445 | ++++ |
| 449 | ++++ |
| 551 | ++++ |
| 552 | ++++ |
| 554 | + |
| 555 | ++++ |
| 557 | ++++ |
| 558 | ++++ |
| 560 | + |
| 561 | ++++ |
| 562 | ++++ |
| 563 | ++++ |
| 564 | ++++ |
| 566 | ++++ |
| 567 | ++++ |
| 568 | +++ |
| 569 | ++++ |
| 571 | ++++ |
| 572 | ++++ |
| 573 | ++++ |
| 574 | ++++ |
| 575 | ++++ |
| 576 | ++++ |
| 577 | ++++ |
| 578 | ++++ |
| 585 | ++++ |
| 586 | ++++ |
| 587 | ++++ |
| 588 | ++++ |
| 589 | +++ |
| 432 | ++++ |
| 672 | + |
| 673 | ++ |
| 682 | + |
| 686 | + |
| 687 | + |
| 662 | ++++ |
| 663 | ++++ |
| 553 | +++ |
| 559 | ++++ |
| 579 | ++++ |
| 581 | ++++ |
| 582 | ++ |
| 582 | ++++ |
| 584 | +++ |
| 675 | ++++ |
| 676 | ++++ |
| 677 | + |
| 679 | ++++ |
| 700 | +++ |
| 704 | +++ |
| 591 | + |
| 706 | ++ |
| 695 | ++ |
| 595 | ++++ |
| 596 | ++++ |
| 597 | +++ |
| 598 | +++ |
| 599 | ++++ |
| 600 | ++++ |
| 601 | +++ |
| 602 | +++ |
| 603 | +++ |
| 604 | +++ |
| 606 | ++++ |
| 607 | ++++ |
| 608 | ++++ |
| 610 | ++++ |
| 611 | ++++ |
| 612 | ++++ |
| 613 | +++ |
| 614 | +++ |
| 615 | ++++ |
| 618 | ++++ |
| 619 | ++++ |
| 707 | ++++ |
| 620 | ++++ |
| 621 | ++++ |
| 622 | ++++ |
| 623 | ++++ |
| 624 | ++++ |
| 625 | ++++ |
| 626 | +++ |
| 631 | ++++ |
| 633 | ++++ |
| 634 | ++++ |
| 635 | +++ |
| 636 | +++ |
| 638 | + |
| 641 | +++ |
| 665 | ++++ |
| 708 | ++++ |
| 709 | +++ |
| 710 | + |
| 711 | ++++ |
| 712 | ++++ |
| 713 | ++++ |
| 714 | +++ |
| 715 | +++ |
| 716 | ++++ |
| 765 | + |
| 753 | + |
| 754 | + |
| 755 | + |
| 756 | + |
| 757 | ++++ |
| 758 | +++ |

TABLE 28

| | HCT-116 | RKO | RKO-E6 | SW480 | |
|---|---|---|---|---|---|
| | EC₅₀ | EC₅₀ | EC₅₀ | EC₅₀ | EC₅₀ |
| SP# | (72 h) | (72 h) | (72 h) | (6 days) | Ratio |
| 4 | ++++ | ++++ | +++ | ++++ | |
| 5 | ++++ | ++++ | +++ | ++++ | |
| 7 | ++++ | ++++ | +++ | ++++ | |
| 10 | ++++ | +++ | +++ | +++ | |
| 11 | ++++ | ++++ | ++ | +++ | |
| 50 | ++++ | ++++ | ++ | +++ | |
| 65 | +++ | +++ | +++ | +++ | |
| 69 | ++++ | ++++ | + | ++++ | |
| 70 | ++++ | ++++ | ++ | +++ | |
| 71 | ++++ | ++++ | +++ | +++ | |
| 81 | +++ | +++ | +++ | +++ | |
| 99 | ++++ | ++++ | +++ | ++++ | |
| 109 | ++++ | ++++ | ++ | +++ | |
| 114 | | +++ | + | +++ | |
| 115 | | +++ | + | +++ | 1-29 |
| 118 | +++ | ++++ | + | ++++ | |
| 120 | ++++ | ++++ | + | ++++ | |
| 121 | ++++ | ++++ | + | ++++ | |
| 122 | | +++ | + | +++ | 1-29 |
| 125 | +++ | +++ | + | + | |
| 126 | + | + | + | + | |
| 148 | | ++ | + | + | |
| 150 | | ++ | + | + | |
| 153 | +++ | | + | | |
| 154 | +++ | +++ | + | + | 30-49 |
| 158 | + | + | + | + | |
| 160 | +++ | + | + | + | 1-29 |
| 161 | +++ | + | + | + | |
| 175 | + | + | + | + | |
| 196 | ++++ | ++++ | +++ | ++++ | |
| 219 | ++++ | +++ | + | + | 1-29 |
| 233 | ++++ | | | | |
| 237 | ++++ | | + | + | |
| 238 | ++++ | | + | + | |
| 243 | ++++ | | + | + | |
| 244 | ++++ | | + | + | ≧50 |
| 245 | ++++ | | + | + | |
| 247 | ++++ | | + | + | |
| 249 | ++++ | ++++ | + | + | ≧50 |
| 255 | ++++ | | + | | |
| 291 | | | + | | |
| 293 | +++ | | + | | |
| 303 | +++ | | + | | 1-29 |
| 305 | | | + | | |
| 306 | ++++ | | + | | |
| 310 | ++++ | | + | | |
| 312 | ++++ | | | | |
| 313 | ++++ | | ++ | | |
| 314 | | | + | | |
| 315 | ++++ | ++++ | ++ | ++++ | ≧50 |
| 316 | ++++ | ++++ | + | +++ | ≧50 |
| 317 | +++ | | + | ++ | |
| 321 | ++++ | | + | | |
| 324 | +++ | | + | | |
| 325 | +++ | | | | |
| 326 | +++ | | + | | |
| 327 | +++ | | + | | |
| 328 | +++ | | ++ | | |
| 329 | ++++ | | + | | |
| 330 | | | + | | |
| 331 | ++++ | ++++ | + | + | ≧50 |
| 338 | ++++ | ++++ | ++ | +++ | |
| 341 | +++ | ++ | + | + | |
| 343 | +++ | | + | + | |
| 346 | ++++ | | + | + | |
| 347 | +++ | | + | + | |
| 349 | ++++ | +++ | + | + | 30-49 |
| 350 | ++++ | | + | + | |
| 351 | ++++ | +++ | + | + | 30-49 |
| 353 | ++ | ++ | + | + | |
| 355 | ++++ | ++ | + | + | 1-29 |
| 357 | ++++ | ++++ | + | + | |
| 358 | ++++ | ++ | + | + | |
| 359 | ++++ | ++ | + | + | |
| 367 | ++++ | | + | + | 30-49 |
| 386 | ++++ | ++++ | ++++ | ++++ | |
| 388 | ++ | ++ | + | +++ | 1-29 |
| 390 | ++++ | ++++ | +++ | ++++ | |
| 435 | +++ | ++ | + | | |
| 436 | ++++ | ++++ | ++ | | |
| 437 | ++++ | ++++ | ++ | ++++ | 30-49 |
| 440 | ++ | ++ | + | | |
| 442 | ++++ | ++++ | ++ | | |
| 444 | ++++ | ++++ | +++ | | |
| 445 | ++++ | +++ | + | + | ≧50 |
| 555 | | | | | ≧50 |
| 557 | | | | | ≧50 |
| 558 | | | | | 30-49 |
| 562 | | | | | 30-49 |
| 564 | | | | | 30-49 |
| 566 | | | | | 30-49 |
| 567 | | | | | ≧50 |
| 572 | | | | | ≧50 |
| 573 | | | | | 30-49 |
| 578 | | | | | 30-49 |
| 662 | | | | | ≧50 |
| 379 | | | | | 1-29 |
| 375 | | | | | 1-29 |
| 559 | | | | | ≧50 |
| 561 | | | | | 1-29 |
| 563 | | | | | 1-29 |
| 568 | | | | | 1-29 |
| 569 | | | | | 1-29 |
| 571 | | | | | 1-29 |
| 574 | | | | | 1-29 |
| 575 | | | | | 1-29 |
| 576 | | | | | 1-29 |
| 577 | | | | | 30-49 |
| 433 | | | | | 1-29 |
| 551 | | | | | 30-49 |
| 553 | | | | | 1-29 |
| 710 | | | | + | |
| 711 | | | | + | |
| 712 | | | | ++ | |
| 713 | | | | ++ | |
| 714 | | | | +++ | |
| 715 | | | | +++ | |
| 716 | | | | + | |

### Example 13. P21 ELISA Assay

The assay was performed according to the following general protocol:

Cell Plating: Trypsinize, count and seed SJSA1 cells at the density of 7500 cells/100 μl/well in 96-well plates a day prior to assay. On the day of study, replace media with fresh RPMI-11% FBS (assay media). Add 90 μL of the assay media per well. Control wells with no cells, receive 100 μl media.

Peptide dilution: Prepare 10 mM stocks of the peptides in DMSO. Serially dilute the stock using 1:3 dilution scheme to get 10, 3.3, 1.1, 0.33, 0.11, 0.03, 0.01 mM solutions using DMSO as diluents. Dilute the serially DMSO-diluted peptides 33.3 times using sterile water. This gives range of 10× working stocks. Also prepare DMSO/sterile water (3% DMSO) mix for control wells. Thus the working stocks concentration range μM will be 300, 100, 30, 10, 3, 1, 0.3 and 0 μM. Mix well at each dilution step using multichannel. Row H has controls. H1-H3 will receive 10 μl of assay media. H4-H9 will receive 10 μl of 3% DMSO-water vehicle. H10-H12 will have media alone control with no cells. Positive control: HDM2 small molecule inhibitor, Nutlin-3a (10 mM) is used as positive control. Nutlin was diluted using the same dilution scheme as peptides.

Addition of working stocks to cells: Add 10 μl of 10× desired concentration to appropriate well to achieve the final concentrations in total 100 μl volume in well. (10 μl of 300 μM peptide+90 μl of cells in media=30 μM final concentration in 100 μl volume in wells). Thus final concentration range used will be 30, 10, 3, 1, 0.3 & 0 μM. Controls will include wells that get no peptides but contain the same concentration of DMSO as the wells containing the peptides, and wells containing NO CELLS. 20 h-post incubation, aspirate the media; wash cells with 1×PBS (without Ca⁺⁺/Mg⁺⁺) and lyse in 60 μl of 1× Cell lysis buffer (Cell Signaling technologies 10× buffer diluted to 1× and supplemented with protease inhibitors and Phosphatase inhibitors) on ice for 30 min. Centrifuge plates in at 5000 rpm speed in at 4° C. for 8 min; collect clear supernatants and freeze at −80° C. until further use.

Protein Estimation: Total protein content of the lysates is measured using BCA protein detection kit and BSA standards from Thermofisher. Typically, about 6-7 μg protein is expected per well. Use 50 μl of the lysate per well to set up p21 ELISA. Human Total p21 ELISA: The ELISA assay protocol is followed as per the manufacturer's instructions. 50 μl lysate is used for each well, and each well is set up in triplicate. Reagents: Cell-Based Assay (−)-Nutlin-3 (10 mM): Cayman Chemicals—catalog #600034, OptiMEM, Invitrogen catalog #51985, Cell Signaling Lysis Buffer (10×), Cell signaling technology—catalog #9803, Protease inhibitor Cocktail tablets(mini), Roche Chemicals—catalog #04693124001, Phosphatase inhibitor Cocktail tablet, Roche Chemicals—catalog #04906837001, Human total p21 ELISA kit, R&D Systems, DYC1047-5, and STOP Solution (1 M HCl), Cell Signaling Technologies—catalog #7002. Instruments: Micro centrifuge—Eppendorf 5415D and Multiplate Reader for Absorbance readout (Synergy 2). Results from Example 13 are provided in p21 data in FIGS. 6A-D.

### Example 14. Caspase 3 Detection Assay

The assay was performed according to the following general protocol.

Cell Plating: Trypsinize, count and seed SJSA1 cells at the density of 7500 cells/100 μl/well in 96-well plates a day prior to assay. On the day of study, replace media with fresh RPMI-11% FBS (assay media). Add 180 μL of the assay media per well. Control wells with no cells, receive 200 μl media.

Peptide Dilution:
Prepare 10 mM stocks of the peptides in DMSO. Serially dilute the stock using 1:3 dilution scheme to get 10, 3.3, 1.1, 0.33, 0.11, 0.03, 0.01 mM solutions using DMSO as diluents. Dilute the serially DMSO-diluted peptides 33.3 times using sterile water. This gives range of 10× working stocks. Also prepare DMSO/sterile water (3% DMSO) mix for control wells.
Thus, the working stocks concentration range μM will be 300, 100, 30, 10, 3, 1, 0.3 and 0 μM. Mix well at each dilution step using multichannel. Add 20 μl of 10× working stocks to appropriate wells.
Row H has controls. H1-H3 will receive 20 μl of assay media. H4-H9 will receive 20 μl of 3% DMSO-water vehicle. H10-H12 will have media alone control with no cells.
Positive control: HDM2 small molecule inhibitor, Nutlin-3a (10 mM) is used as positive control. Nutlin was diluted using the same dilution scheme as peptides.

Addition of working stocks to cells:
Add 10 μl of 10× desired concentration to appropriate well to achieve the final concentrations in total 100 μl volume in well. (10 μl of 300 μM peptide+90 μl of cells in media=30 μM final concentration in 100 μl volume in wells). Thus final concentration range used will be 30, 10, 3, 1, 0.3 & 0 μM.
Controls will include wells that get no peptides but contain the same concentration of DMSO as the wells containing the peptides, and wells containing NO CELLS.
48 h-post incubation, aspirate 80 μl media from each well; add 100 μl Caspase3/7Glo assay reagent (Promega Caspase 3/7 glo assay system, G8092) per well, incubate with gentle shaking for 1 h at room temperature. Read on Synergy Biotek multiplate reader for luminescence. Data is analyzed as Caspase 3 activation over DMSO-treated cells.

Results from Examples 13 and 14 are provided in p21 data in FIGS. 6A-D and Table 29.

TABLE 29

| | caspase | caspase | caspase | caspase | caspase | p21 | p21 | p21 | p21 | p21 |
|---|---|---|---|---|---|---|---|---|---|---|
| SP# | 0.3 uM | 1 uM | 3 uM | 10 uM | 30 uM | 0.3 uM | 1 uM | 3 uM | 10 uM | 30 uM |
| 4 | | | 9 | 37 | 35 | | | 317 | 3049 | 3257 |
| 7 | 0.93 | 1.4 | 5.08 | 21.7 | 23.96 | | 18 | 368 | 1687 | 2306 |
| 8 | | | 1 | 19 | 25 | | | 34 | 972 | 2857 |
| 10 | 1 | | 1 | 17 | 32 | | 10 | 89 | 970 | 2250 |
| 11 | 1 | | 5 | 23 | 33.5 | | 140 | 350 | 2075.5 | 3154 |
| 26 | 1 | | 1 | 3 | 14 | | | | | |
| 50 | | | 8 | 29 | 29 | | 44 | 646 | 1923 | 1818 |
| 65 | 1 | | 6 | 28 | 34 | −69 | −24 | 122 | 843 | 1472 |
| 69 | 4.34 | 9.51 | 16.39 | 26.59 | 26.11 | 272 | 458.72 | 1281.39 | 2138.88 | 1447.22 |
| 70 | | | 1 | 9 | 26 | | −19 | 68 | 828 | 1871 |
| 71 | 0.95 | 1.02 | 3.68 | 14.72 | 23.52 | | 95 | 101 | 1204 | 2075 |
| 72 | 1 | | 1 | 4 | 10 | −19 | 57 | 282 | 772 | 1045 |
| 77 | 1 | | 2 | 19 | 23 | | | | | |
| 80 | 1 | | 2 | 13 | 20 | | | | | |
| 81 | 1 | | 1 | 6 | 21 | | 0 | 0 | 417 | 1649 |
| 99 | 1 | | 7 | 31 | 33 | −19 | 117 | 370 | 996 | 1398 |
| 109 | | | 4 | 16 | 25 | | 161 | 445 | 1221 | 1680 |
| 114 | 1 | | 6 | 28 | 34 | −21 | 11 | 116 | 742 | 910 |
| 115 | 1 | | 10 | 26 | 32 | −10 | 36 | 315 | 832 | 1020 |
| 118 | 1 | | 2 | 18 | 27 | −76 | −62 | −11 | 581 | 1270 |
| 120 | 2 | | 11 | 20 | 30 | −4 | 30 | 164 | 756 | 1349 |
| 121 | 1 | | 5 | 19 | 30 | 9 | 33 | 81 | 626 | 1251 |
| 122 | 1 | | 2 | 15 | 30 | −39 | −18 | 59 | 554 | 1289 |
| 123 | 1 | | 1 | 6 | 14 | | | | | |
| 125 | 1 | | 3 | 9 | 29 | 50 | 104 | 196 | 353 | 1222 |
| 126 | 1 | | 1 | 6 | 30 | −47 | −10 | 90 | 397 | 1443 |
| 127 | 1 | | 1 | 4 | 13 | | | | | |
| 130 | 1 | | 2 | 6 | 17 | | | | | |
| 139 | 1 | | 2 | 9 | 18 | | | | | |
| 142 | 1 | | 2 | 15 | 20 | | | | | |
| 144 | 1 | | 4 | 10 | 16 | | | | | |
| 148 | 1 | | 11 | 23 | 31 | −23 | 55 | 295 | 666 | 820 |
| 149 | 1 | | 2 | 4 | 10 | 35 | 331 | 601 | 1164 | 1540 |
| 150 | 2 | | 11 | 19 | 35 | −37 | 24 | 294 | 895 | 906 |
| 153 | 2 | | 10 | 15 | 20 | | | | | |
| 154 | 2.68 | 4 | 13.93 | 19.86 | 30.14 | 414.04 | 837.45 | 1622.42 | 2149.51 | 2156.98 |
| 158 | 1 | | 1.67 | 5 | 16.33 | −1.5 | 95 | 209.5 | 654 | 1665.5 |
| 160 | 2 | | 10 | 16 | 31 | −43 | 46 | 373 | 814 | 1334 |
| 161 | 2 | | 8 | 14 | 22 | 13 | 128 | 331 | 619 | 1078 |
| 170 | 1 | | 1 | 16 | 20 | | | | | |
| 175 | 1 | | 5 | 12 | 21 | −65 | 1 | 149 | 543 | 1107 |
| 177 | 1 | | 1 | 8 | 20 | | | | | |
| 183 | 1 | | 1 | 4 | 8 | −132 | −119 | −14 | 1002 | 818 |
| 196 | 1 | | 4 | 33 | 26 | −49 | −1 | 214 | 1715 | 687 |
| 197 | 1 | | 1 | 10 | 20 | | | | | |
| 203 | 1 | | 3 | 12 | 10 | 77 | 329 | 534 | 1805 | 380 |
| 204 | 1 | | 4 | 10 | 10 | 3 | 337 | 928 | 1435 | 269 |
| 218 | 1 | | 2 | 8 | 18 | | | | | |
| 219 | 1 | | 5 | 17 | 34 | 28 | 53 | 289 | 884 | 1435 |
| 221 | 1 | | 3 | 6 | 12 | 127 | 339 | 923 | 1694 | 1701 |
| 223 | 1 | | 1 | 5 | 18 | | | | | |
| 230 | 1 | | 2 | 3 | 11 | 245.5 | 392 | 882 | 1549 | 2086 |
| 233 | 6 | 8 | 17 | 22 | 23 | 2000 | 2489 | 3528 | 3689 | 2481 |
| 237 | 1 | | 5 | 9 | 15 | 0 | 0 | 2 | 284 | 421 |
| 238 | 1 | | 2 | 4 | 21 | 0 | 149 | 128 | 825 | 2066 |
| 242 | 1 | | 4 | 5 | 18 | 0 | 0 | 35 | 577 | 595 |
| 243 | 1 | | 2 | 5 | 23 | 0 | 0 | 0 | 456 | 615 |
| 244 | 1 | | 2 | 7 | 17 | 0 | 178 | 190 | 708 | 1112 |
| 245 | 1 | | 3 | 9 | 16 | 0 | 0 | 0 | 368 | 536 |
| 247 | 1 | | 3 | 11 | 24 | 0 | 0 | 49 | 492 | 699 |
| 248 | | | | | | 0 | 50 | 22 | 174 | 1919 |
| 249 | 2 | | 5 | 11 | 23 | 0 | 0 | 100 | 907 | 1076 |
| 251 | | | | | | 0 | 0 | 0 | 0 | 0 |
| 252 | | | | | | 0 | 0 | 0 | 0 | 0 |
| 253 | | | | | | 0 | 0 | 0 | 0 | 0 |
| 254 | 1 | 3 | 7 | 14 | 22 | 118 | 896 | 1774 | 3042 | 3035 |
| 286 | 1 | 4 | 11 | 20 | 22 | 481 | 1351 | 2882 | 3383 | 2479 |
| 287 | 1 | 1 | 3 | 11 | 23 | 97 | 398 | 986 | 2828 | 3410 |
| 315 | 11 | 14.5 | 25.5 | 32 | 34 | 2110 | 2209 | 2626 | 2965 | 2635 |
| 316 | 6.5 | 10.5 | 21 | 32 | 32.5 | 1319 | 1718 | 2848 | 2918 | 2540 |
| 317 | 3 | 4 | 9 | 26 | 35 | 551 | 624 | 776 | 1367 | 1076 |
| 331 | 4.5 | 8 | 11 | 14.5 | 30.5 | 1510 | 1649 | 2027 | 2319 | 2509 |
| 338 | 1 | 5 | 23 | 20 | 29 | 660.37 | 1625.38 | 3365.87 | 2897.62 | 2727 |
| 341 | 3 | 8 | 11 | 14 | 21 | 1325.62 | 1873 | 2039.75 | 2360.75 | 2574 |
| 343 | 1 | 1 | 2 | 5 | 29 | 262 | 281 | 450 | 570 | 1199 |
| 346 | | | | | | 235.86 | 339.82 | 620.36 | 829.32 | 1695.78 |
| 347 | 2 | 3 | 5 | 8 | 29 | 374 | 622 | 659 | 905 | 1567 |
| 349 | 1 | 8 | 11 | 16 | 24 | 1039.5 | 1598.88 | 1983.75 | 2191.25 | 2576.38 |
| 351 | 3 | 9 | 13 | 15 | 24 | 1350.67 | 1710.67 | 2030.92 | 2190.67 | 2668.54 |
| 353 | 1 | 2 | 5 | 7 | 30 | 390 | 490 | 709 | 931 | 1483 |
| 355 | 1 | 4 | 11 | 13 | 30 | 191 | 688 | 1122 | 1223 | 1519 |
| 357 | 2 | 7 | 11 | 15 | 23 | 539 | 777 | 1080 | 1362 | 1177 |
| 358 | 1 | 2 | 3 | 6 | 24 | 252 | 321 | 434 | 609 | 1192 |
| 359 | 3 | 9 | 11 | 13 | 23 | 1163.29 | 1508.79 | 1780.29 | 2067.67 | 2479.29 |
| 416 | | | | | | 33.74 | 39.82 | 56.57 | 86.78 | 1275.28 |
| 417 | | | | | | 0 | 0 | 101.13 | 639.04 | 2016.58 |
| 419 | | | | | | 58.28 | 97.36 | 221.65 | 1520.69 | 2187.94 |
| 432 | | | | | | 54.86 | 68.86 | 105.11 | 440.28 | 1594.4 |

### Example 15. X-Ray Co-Crystallography of Peptidomimetic Macrocycles in Complex with MDMX

For co-crystallization with peptide 46 (Table 9), a stoichiometric amount of compound from a 100 mM stock solution in DMSO was added to the zebrafish MDMX protein solution and allowed to sit overnight at 4° C. before setting up crystallization experiments. Procedures were similar to those described by Popowicz et al. with some variations, as noted below. Protein (residues 15-129, L46V/V95L) was obtained from an E. coli BL21(DE3) expression system using the pET15b vector. Cells were grown at 37° C. and induced with 1 mM IPTG at an OD₆₀₀ of 0.7. Cells were allowed to grow an additional 18 hr at 23° C. Protein was purified using Ni-NT Agarose followed by Superdex 75 buffered with 50 mM NaPO₄, pH 8.0, 150 mM NaCl, 2 mM TCEP and then concentrated to 24 mg/ml. The buffer was exchanged to 20 mM Tris, pH 8.0, 50 mM NaCl, 2 mM DTT for crystallization experiments. Initial crystals were obtained with the Nextal (Qiagen) AMS screen #94 and the final optimized reservoir was 2.6 M AMS, 75 mM Hepes, pH 7.5. Crystals grew routinely as thin plates at 4° C. and were cryo-protected by pulling them through a solution containing concentrated (3.4 M) malonate followed by flash cooling, storage, and shipment in liquid nitrogen.

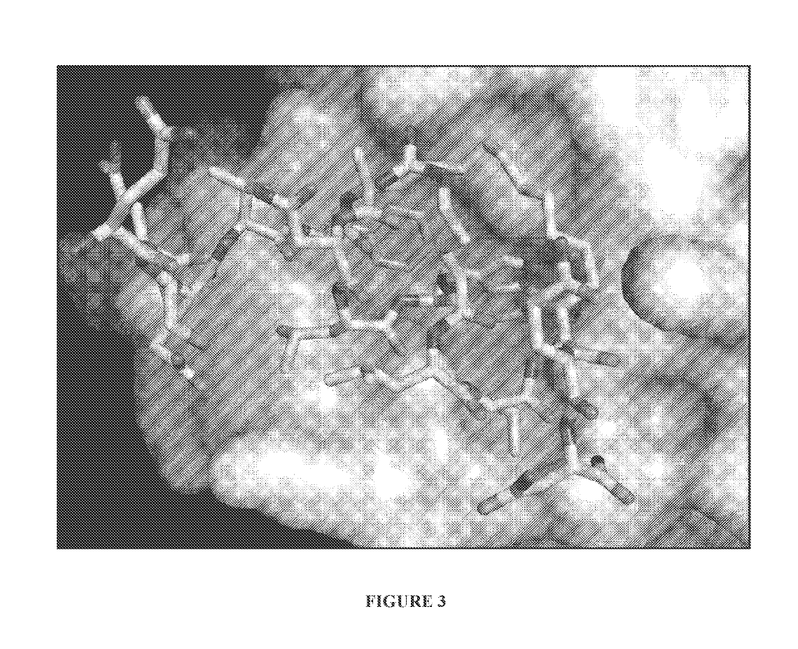

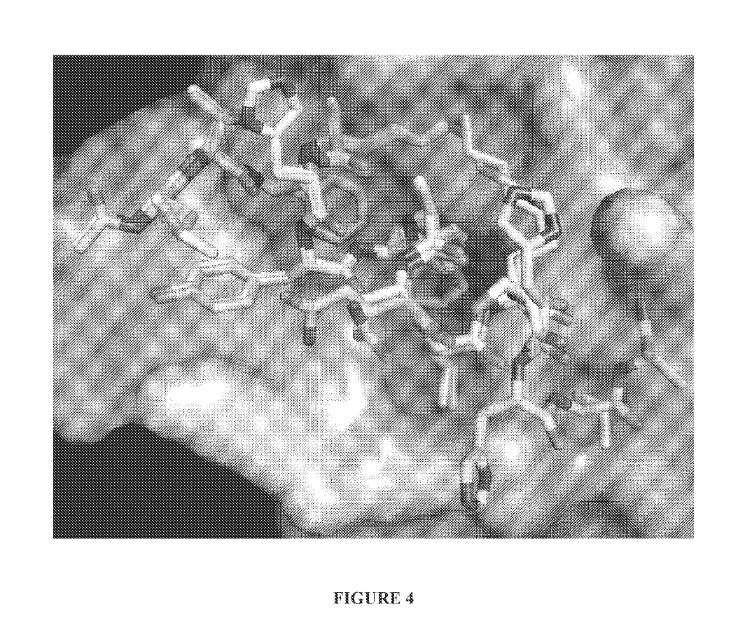

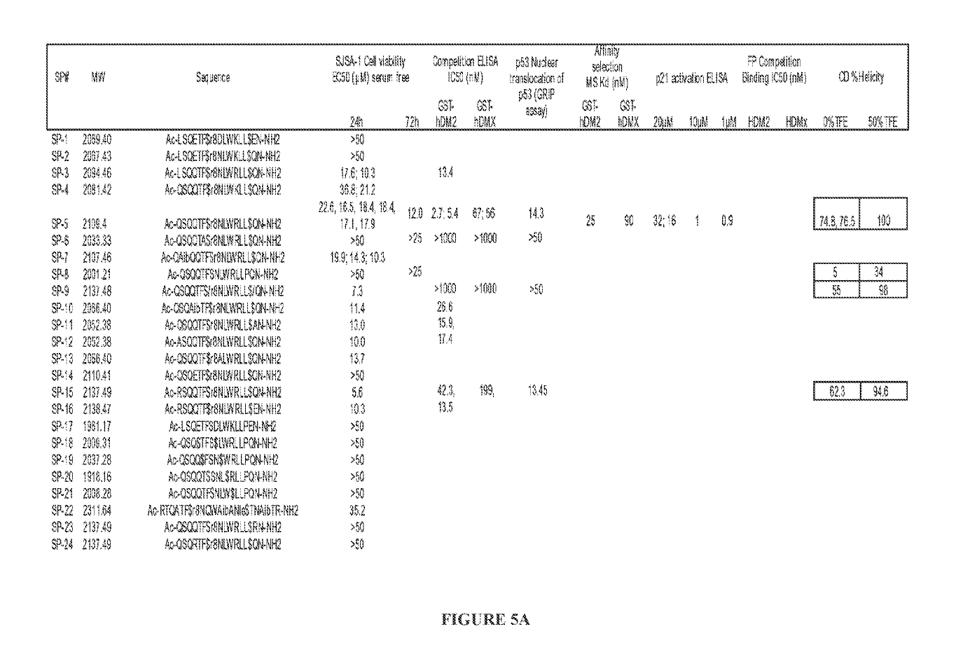

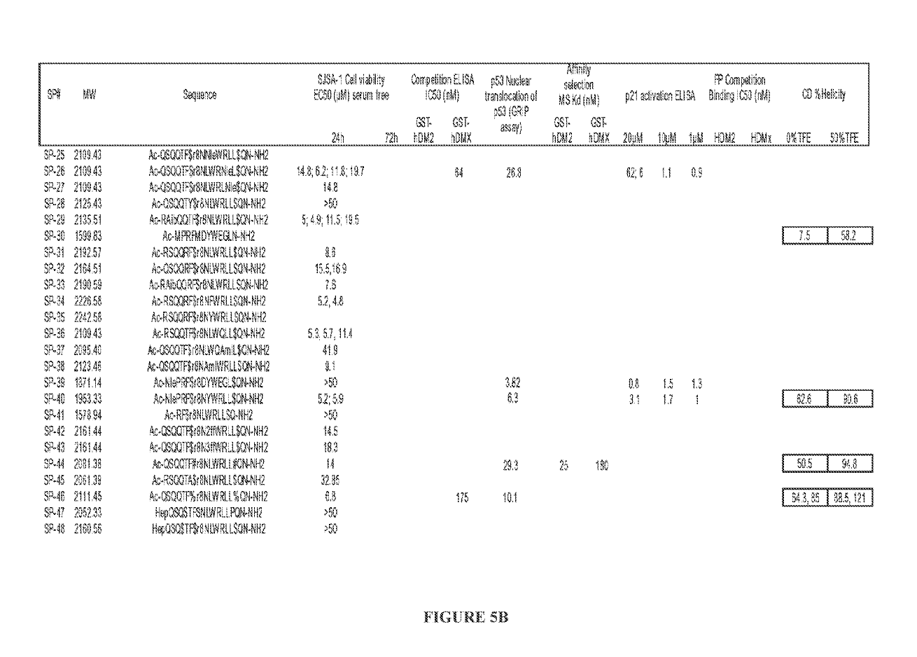

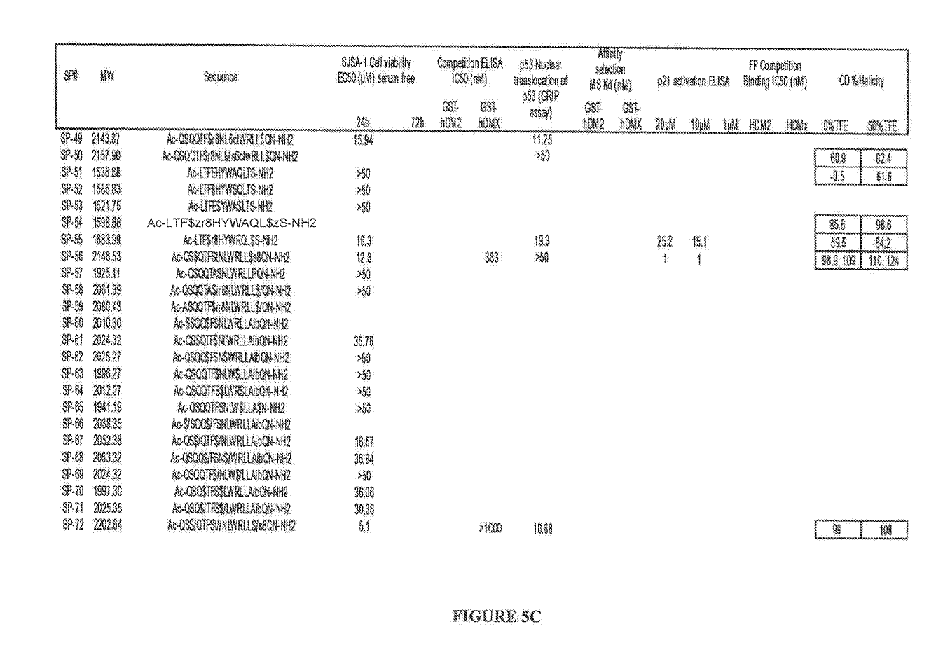

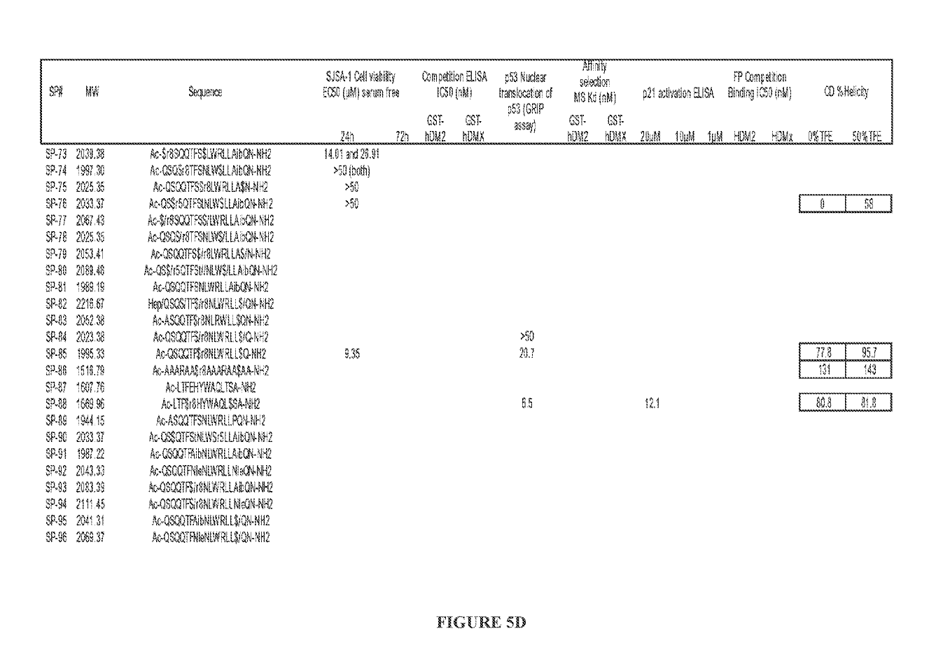

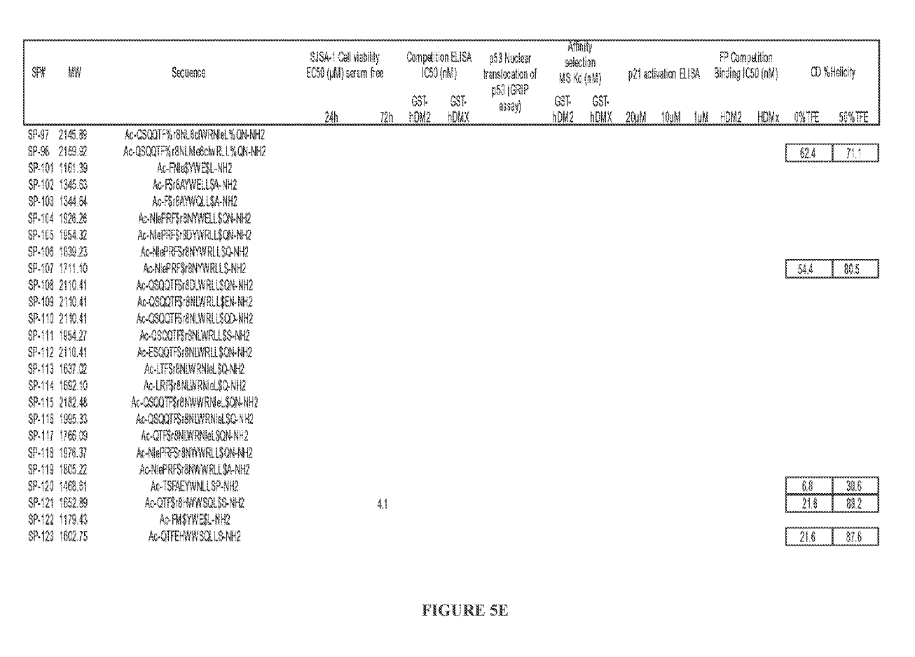

Data collection was performed at the APS at beamline 31-ID (SGX-CAT) at 100 K and wavelength 0.97929 Å. The beamline was equipped with a Rayonix 225-HE detector. For data collection, crystals were rotated through 180° in 1° increments using 0.8 second exposure times. Data were processed and reduced using Mosflm/scala (CCP4; see The CCP4 Suite: Programs for Protein Crystallography. Acta Crystallogr. D 50, 760-763 (1994); P. R. Evans. Joint CCP4 and ESF-EACBM Newsletter 33, 22-24 (1997)) in space group C2 (unit cell: a=109.2786, b=81.0836, c=30.9058 Å, α=90, β=89.8577, γ=90°). Molecular replacement with program Molrep (CCP4; see A. Vagin & A. Teplyakov. J. Appl. Cryst. 30, 1022-1025 (1997)) was perfomed with the MDMX component of the structure determined by Popowicz et al. (2Z5S; see G. M. Popowicz, A. Czarna, U. Rothweiler, A. Szwagierczak, M. Krajewski, L. Weber & T. A. Holak. Cell Cycle 6, 2386-2392 (2007)) and identified two molecules in the asymmetric unit. Initial refinement of just the two molecules of the zebrafish MDMX with program Refmac (CCP4; see G. N. Murshudov, A. A. Vagin & E. J. Dodson. Acta Crystallogr. D 53, 240-255 (1997)) resulted in an R-factor of 0.3424 (R_{free}=0.3712) and rmsd values for bonds (0.018 Å) and angles (1.698°). The electron density for the stapled peptide components, starting with Gln¹⁹ and including the entire aliphatic staple, was very clear. Further refinement with CNX (Accelrys) using data to 2.3 Å resolution resulted in a model (comprised of 1448 atoms from MDMX, 272 atoms from the stapled peptides and 46 water molecules) that is well refined (R_{f}=0.2601, R_{free}=0.3162, rmsd bonds=0.007 Å and rmsd angles=0.916°). Results from Example 15 are shown in FIGS. 3 and 4.

### Example 16. Cell Lysis by Peptidomimetic Macrocycles

SJSA-1 cells were plated out one day in advance in clear flat-bottom plates (Costar, catalog number 353072) at 7500 cells/well with 100 ul/well of growth media, leaving row H columns 10-12 empty for media alone. On the day of the assay, media was exchanged with RPMI 1% FBS media, 90 μl of media per well.

10 mM stock solutions of the peptidomimetic macrocycles were prepared in 100% DMSO. Peptidomimetic macrocycles were then diluted serially in 100% DMSO, and then further diluted 20-fold in sterile water to prepare working stock solutions in 5% DMSO/water of each peptidomimetic macrocycle at concentrations ranging from 500 μM to 62.5 uM.

10 μl of each compound was added to the 90 μl of SJSA-1 cells to yield final concentrations of 50 uM to 6.25 uM in 0.5% DMSO-containing media. The negative control (non-lytic) sample was 0.5% DMSO alone and positive control (lytic) samples include 10 uM Melittin and 1% Triton X-100.

Cell plates were incubated for 1 hour at 37° C. After the 1 hour incubation, the morphology of the cells is examined by microscope and then the plates were centrifuged at 1200 rpm for 5 minutes at room temperature. 40 μl of supernatant for each peptidomimetic macrocyle and control sample is transferred to clear assay plates. LDH release is measured using the LDH cytotoxicity assay kit from Caymen, catalog#1000882. Results are shown in Table 30.

TABLE 30

| | 6.25 uM % | 12.5 uM % | 25 uM % | 50 uM % |
|---|---|---|---|---|
| | Lysed cells | Lysed cells | Lysed cells | Lysed cells |
| SP# | (1 h LDH) | (1 h LDH) | (1 h LDH) | (1 h LDH) |
| 3 | 1 | 0 | 1 | 3 |
| 4 | −2 | 1 | 1 | 2 |
| 6 | 1 | 1 | 1 | 1 |
| 7 | 0 | 0 | 0 | 0 |
| 8 | −1 | 0 | 1 | 1 |
| 9 | −3 | 0 | 0 | 2 |
| 11 | −2 | 1 | 2 | 3 |
| 15 | 1 | 2 | 2 | 5 |
| 18 | 0 | 1 | 2 | 4 |
| 19 | 2 | 2 | 3 | 21 |
| 22 | 0 | −1 | 0 | 0 |
| 26 | 2 | 5 | −1 | 0 |
| 32 | 0 | 0 | 2 | 0 |
| 39 | 0 | −1 | 0 | 3 |
| 43 | 0 | 0 | −1 | −1 |
| 55 | 1 | 5 | 9 | 13 |
| 65 | 0 | 0 | 0 | 2 |
| 69 | 1 | 0.5 | −0.5 | 5 |
| 71 | 0 | 0 | 0 | 0 |
| 72 | 2 | 1 | 0 | 3 |
| 75 | −1 | 3 | 1 | 1 |
| 77 | −2 | −2 | 1 | −1 |
| 80 | 0 | 1 | 1 | 5 |
| 81 | 1 | 1 | 0 | 0 |
| 82 | 0 | 0 | 0 | 1 |
| 99 | 1.5 | 3 | 2 | 3.5 |
| 108 | 0 | 0 | 0 | 1 |
| 114 | 3 | −1 | 4 | 9 |
| 115 | 0 | 1 | −1 | 6 |
| 118 | 4 | 2 | 2 | 4 |
| 120 | 0 | −1 | 0 | 6 |
| 121 | 1 | 0 | 1 | 7 |
| 122 | 1 | 3 | 0 | 6 |
| 123 | −2 | 2 | 5 | 3 |
| 125 | 0 | 1 | 0 | 2 |
| 126 | 1 | 2 | 1 | 1 |
| 130 | 1 | 3 | 0 | −1 |
| 139 | −2 | −3 | −1 | −1 |
| 142 | 1 | 0 | 1 | 3 |
| 144 | 1 | 2 | −1 | 2 |
| 147 | 8 | 9 | 16 | 55 |
| 148 | 0 | 1 | −1 | 0 |
| 149 | 6 | 7 | 7 | 21 |
| 150 | −1 | −2 | 0 | 2 |
| 153 | 4 | 3 | 2 | 3 |
| 154 | −1 | −1.5 | −1 | −1 |
| 158 | 0 | −6 | −2 | |
| 160 | −1 | 0 | −1 | 1 |
| 161 | 1 | 1 | −1 | 0 |
| 169 | 2 | 3 | 3 | 7 |
| 170 | 2 | 2 | 1 | −1 |
| 174 | 5 | 3 | 2 | 5 |
| 175 | 3 | 2 | 1 | 0 |
| 177 | −1 | −1 | 0 | 1 |
| 182 | 0 | 2 | 3 | 6 |
| 183 | 2 | 1 | 0 | 3 |
| 190 | −1 | −1 | 0 | 1 |
| 196 | 0 | −2 | 0 | 3 |
| 197 | 1 | −4 | −1 | −2 |
| 203 | 0 | −1 | 2 | 2 |
| 204 | 4 | 3 | 2 | 0 |
| 211 | 5 | 4 | 3 | 1 |
| 217 | 2 | 1 | 1 | 2 |
| 218 | 0 | −3 | −4 | 1 |
| 219 | 0 | 0 | −1 | 2 |
| 221 | 3 | 3 | 3 | 11 |
| 223 | −2 | −2 | −4 | −1 |
| 230 | 0.5 | −0.5 | 0 | 3 |
| 232 | 6 | 6 | 5 | 5 |
| 233 | 2.5 | 4.5 | 3.5 | 6 |
| 237 | 0 | 3 | 7 | 55 |
| 243 | 4 | 23 | 39 | 64 |
| 244 | 0 | 1 | 0 | 4 |
| 245 | 1 | 14 | 11 | 56 |
| 247 | 0 | 0 | 0 | 4 |
| 249 | 0 | 0 | 0 | 0 |
| 254 | 11 | 34 | 60 | 75 |
| 279 | 6 | 4 | 5 | 6 |
| 280 | 5 | 4 | 6 | 18 |
| 284 | 5 | 4 | 5 | 6 |
| 286 | 0 | 0 | 0 | 0 |
| 287 | 0 | 6 | 11 | 56 |
| 316 | 0 | 1 | 0 | 1 |
| 317 | 0 | 1 | 0 | 0 |
| 331 | 0 | 0 | 0 | 0 |
| 335 | 0 | 0 | 0 | 1 |
| 336 | 0 | 0 | 0 | 0 |
| 338 | 0 | 0 | 0 | 1 |
| 340 | 0 | 2 | 0 | 0 |
| 341 | 0 | 0 | 0 | 0 |
| 343 | 0 | 1 | 0 | 0 |
| 347 | 0 | 0 | 0 | 0 |
| 349 | 0 | 0 | 0 | 0 |
| 351 | 0 | 0 | 0 | 0 |
| 353 | 0 | 0 | 0 | 0 |
| 355 | 0 | 0 | 0 | 0 |
| 357 | 0 | 0 | 0 | 0 |
| 359 | 0 | 0 | 0 | 0 |
| 413 | 5 | 3 | 3 | 3 |
| 414 | 3 | 3 | 2 | 2 |
| 415 | 4 | 4 | 2 | 2 |

### Example 17. MCF-7 Breast Cancer Study Using SP315, SP249 and SP154

A xenograft study was performed to test the efficacy of SP315, SP249 and SP154 in inhibiting tumor growth in athymic mice in the MCF-7 breast cancer xenograft model. A negative control stapled peptide. SP252, a point mutation of SP154 (F to A at position 19) was also tested in one group; this peptide had shown no activity in the SJSA-1 in vitro viability assay. Slow release 90 day 0.72 mg 17β-estradiol pellets (Innovative Research, Sarasota, Fla.) were implanted subcutaneously (sc) on the nape of the neck one day prior to tumor cell implantation (Day −1). On Day 0, MCF-7 tumor cells were implanted sc in the flank of female nude (Crl:NU-Foxn1nu) mice. On Day 18, the resultant sc tumors were measured using calipers to determine their length and width and the mice were weighed. The tumor sizes were calculated using the formula (length×width²)/2 and expressed as cubic millimeters (mm³). Mice with tumors smaller than 85.3 mm³ or larger than 417.4 mm³ were excluded from the subsequent group formation. Thirteen groups of mice, 10 mice per group, were formed by randomization such that the group mean tumor sizes were essentially equivalent (mean of groups±standard deviation of groups=180.7±17.5 mm³).

SP315, SP249, SP154 and SP252 dosing solutions were prepared from peptides formulated in a vehicle containing MPEG(2K)-DSPE at 50 mg/mL concentration in a 10 mM Histidine buffered saline at pH 7. This formulation was prepared once for the duration of the study. This vehicle was used as the vehicle control in the subsequent study.

Each group was assigned to a different treatment regimen. Group 1, as the vehicle negative control group, received the vehicle administered at 8 mL/kg body weight intravenously (iv) three times per week from Days 18-39. Groups 2 and 3 received SP154 as an iv injection at 30 mg/kg three times per week or 40 mg/kg twice a week, respectively. Group 4 received 6.7 mg/kg SP249 as an iv injection three times per week. Groups 5, 6, 7 and 8 received SP315 as an iv injection of 26.7 mg/kg three times per week, 20 mg/kg twice per week, 30 mg/kg twice per week, or 40 mg/kg twice per week, respectively. Group 9 received 30 mg/kg SP252 as an iv injection three times per week.

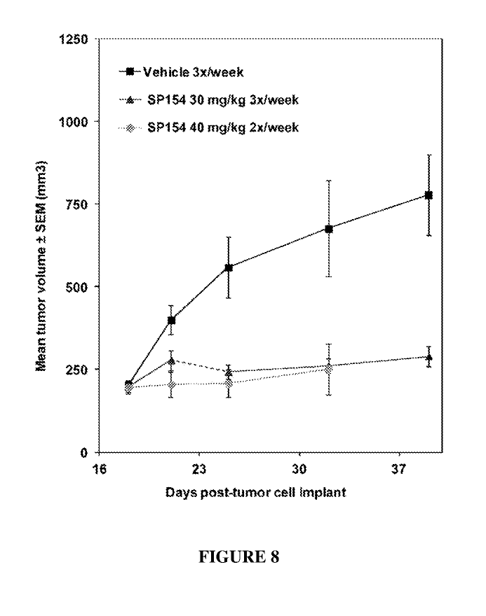

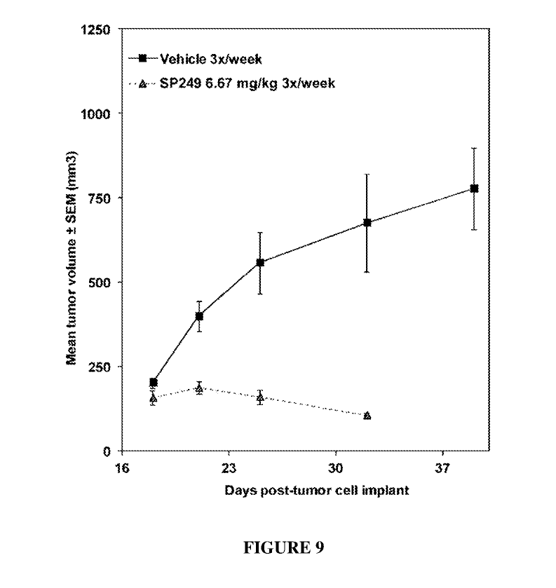

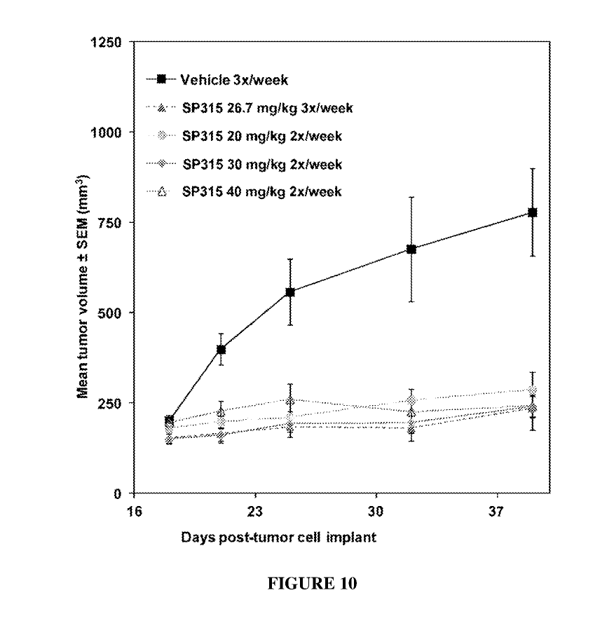

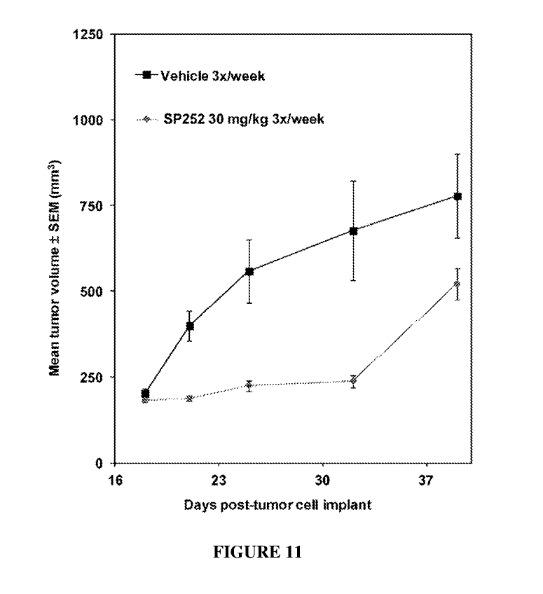

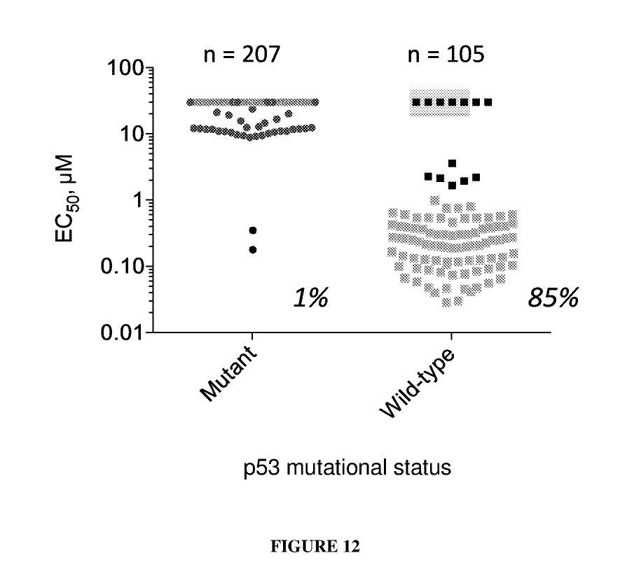

During the dosing period the mice were weighed and tumors measured 1-2 times per week. Results in terms of tumor volume are shown in FIGS. 8-11 and tumor growth inhibition compared with the vehicle group, body weight change and number of mice with ≧20% body weight loss or death are shown in Table 31. Tumor growth inhibition (TGI) was calculated as % TGI=100−[(TuVol^{Treated-day x}−TuVol^{Treated-day18})/(TuVol^{Vehicle negative control-day x}−TuVol^{Vehicle negative control-day}18)*100, where x=day that effect of treatment is being assessed. Group 1, the vehicle negative control group, showed good tumor growth rate for this tumor model.

For SP154, in the group dosed with 40 mg/kg twice a week 2 mice died during treatment, indicating that this dosing regimen was not tolerable. The dosing regimen of 30 mg/kg of SP154 three times per week was well-tolerated and yielded a TGI of 84%.

For SP249, the group dosed with 6.7 mg/kg three times per week 4 mice died during treatment, indicating that this dosing regimen was not tolerable.

All dosing regimens used for SP315 showed good tolerability, with no body weight loss or deaths noted. Dosing with 40 mg/kg of SP315 twice per week produced the highest TGI (92%). The dosing regimens of SP315 of 26.7 mg/kg three times per week, 20 mg/kg twice per week, 30 mg/kg twice per week produced TGI of 86, 82, and 85%, respectively.

For SP252, the point mutation of SP154 which shows no appreciable activity in in vitro assays, dosing with 30 mg/kg three times per week was well-tolerated with no body weight loss or deaths noted. While TGI of 88% was noted by Day 32, that TGI was reduced to 41% by Day 39.

Results from Example 17 are shown in FIGS. 8-11 and summarized in Table 31.

TABLE 31

| Group | | % BW | No. with ≧10% | No. with ≧20% | |
|---|---|---|---|---|---|
| Number | Treatment Group | Change | BW Loss | BW Loss or death | % TGI |
| 1 | Vehicle | +8.6 | 0/10 | 0/10 | — |
| 2 | SP154 30 mg/kg | +5.7 | 0/10 | 0/10 | *84 |
| | 3x/wk iv | | | | |
| 3 | SP154 40 mg/kg | N/A | 0/10 | 2/10 | Regimen |
| | 2x/wk iv | | | (2 deaths) | not |
| | | | | | tolerated |
| 4 | SP249 6.7 mg/kg | N/A | 6/10 | 4/10 | Regimen |
| | 3x/wk iv | | | | not |
| | | | | | tolerated |
| 5 | SP315 26.7 mg/kg | +3.7 | 0/10 | 0/10 | *86 |
| | 3x/wk iv | | | | |
| 6 | SP315 20 mg/kg | +3.9 | 0/10 | 0/10 | *82 |
| | 2x/wk iv | | | | |
| 7 | SP315 30 mg/kg | +8.0 | 0/10 | 0/10 | *85 |
| | 2x/wk iv | | | | |
| 8 | SP315 40 mg/kg | +2.1 | 0/10 | 0/10 | *92 |
| | 2x/wk iv | | | | |
| 9 | SP252 30 mg/kg | +3.3 | 0/10 | 0/10 | *41 |
| | 3x/wk iv | | | | |

*p ≦ 0.05 Vs Vehicle Control

### Example 18. Binding Affinity of Compound 1 to Human Mutant and Wild Type p53

A stapled peptidomimetic macrocycle of the invention, Compound 1, was used in human subjects to assess the binding affinity to the p53 protein variants from human cancer cell lines.

DNA obtained from cancer samples of candidate patients were sequenced to determine the entire p53 coding region, including TP53 exons, introns, and splice sites. Dysfunctional p53 was inferred from the identification of substitutions, indels, frameshift mutations, splice site mutation, insertions or deletions, copy number variants, large deletions, or polymorphisms. Minimum tumor content was 20%. Average read depth was about 750 reads/amplicon. The lower limit of detection was 5% mutant allele at an average read depth of ≧450 reads per amplicon. When the average read depth was <450 reads per amplicon, the limit of detection was 15% mutant allele.

Detection of TP53 gene copy number was based on the number of TP53 amplicon reads from a tumor compared with the average number reads across 14 normal DNA samples. Limits of the assay: the loss of one or more alleles can be determined if tumor content is >60% (99% sensitivity); the loss of two alleles can be determined if tumor sample content is >30% (99% sensitivity). More than 10% of tumors with wild type TP53 have a copy number less than 0.5 (<0.5).

The binding affinity of Compound 1 to p53, measured as the half maximal effective concentration (EC₅₀), was significantly greater for the wild type p53 compared to the mutant p53 as shown in FIG. 13. In general, Compound 1 preferentially bound wild type p53 over mutated p53. However, subpopulations of mutant p53 also exhibited strong affinity to Compound 1 and subpopulations of wild type p53 exhibited weak affinity to Compound 1.

What is claimed is:

1. A method for treating a condition in a subject in need thereof, the method comprising:
a) performing an assay to determine a mutational status of a gene that modulates the p53 pathway in the subject; and
b) administering to the subject a therapeutically-effective amount of a peptidomimetic macrocycle or a pharmaceutically-acceptable salt thereof.

2. The method of claim 1, wherein the peptidomimetic macrocycle comprises a sequence that is at least 60% identical to a subsequence of p53.

3. The method of claim 1, wherein the peptidomimetic macrocycle binds to MDM2, HDM2, MDMX, or HDMX.

4. The method of claim 1, wherein the gene is TP53.

5. The method of claim 1, wherein the mutational status relates to a mutation that is a frameshift.

6. The method of claim 1, wherein the mutational status relates to a mutation that is a splice site mutation.

7. The method of claim 1, wherein the mutational status relates to a mutation that is an insertion.

8. The method of claim 1, wherein the mutational status relates to a mutation that is a deletion.

9. The method of claim 1, wherein the mutational status relates to a mutation that is a substitution.

10. The method of claim 1, wherein the mutational status relates to a mutation that is a copy number loss.

11. The method of claim 1, wherein the mutational status relates to a mutation that is a single nucleotide polymorphism.

12. The method of claim 1, wherein the assay is next-generation sequencing.

13. The method of claim 1, wherein the assay is DNA sequencing.

14. The method of claim 1, wherein the assay is RNA sequencing.

15. The method of claim 1, wherein the condition is a cancer.

16. The method of claim 1, wherein the peptidomimetic macrocycle comprises an amino acid sequence that is at least about 60% identical to an amino acid sequence of the amino acid sequences in Tables 9-24.

17. The method of claim 1, wherein the peptidomimetic macrocycle comprises an amino acid sequence that is at least about 80% identical to an amino acid sequence of the amino acid sequences in Tables 9-24.

18. The method of claim 1, wherein the peptidomimetic macrocycle comprises an amino acid sequence that is at least about 90% identical to an amino acid sequence of the amino acid sequences in Tables 9-24.

19. The method of claim 1, wherein the peptidomimetic macrocycle comprises an amino acid sequence that is at least about 95% identical to an amino acid sequence of the amino acid sequences in Tables 9-24.

20. The method of claim 1, wherein the peptidomimetic macrocycle comprises an amino acid sequence that is an amino acid sequence of the amino acid sequences in Tables 9-24.

21. The method of claim 1, wherein the peptidomimetic macrocycle is an amino acid sequence of the amino acid sequences in Tables 9-24.
